(19) United States
(12) Patent Application Publication
(10) Pub. No.: US 2015/0218110 A1
(43) Pub. Date: Aug. 6, 2015

(54) USE OF SUBSTITUTED BENZODIAZEPINONES AND BENZAZEPINONES OR THE SALTS THEREOF AS ACTIVE SUBSTANCES AGAINST ABIOTIC PLANT STRESS

(71) Applicant: BAYER CROPSCIENCE AG, Monheim (DE)

(72) Inventors: Jens Frackenpohl, Frankfurt (DE); Ines Heinemann, Hofheim (DE); Thomas Mueller, Frankfurt (DE); Guido Bojack, Wiesbaden-Naurod (DE); Jan Dittgen, Frankfurt (DE); Pascal Von Koskull-Doering, Frankfurt (DE); Dirk Schmutzler, Hattersheim (DE); Martin Jeffrey Hills, Idstein (DE); Juan Pedro Ruiz-Santaella Moreno, Leverkusen (DE)

(21) Appl. No.: 14/424,284

(22) PCT Filed: Sep. 2, 2013
(86) PCT No.: PCT/EP2013/068112

§ 371 Date: Feb. 26, 2015

(30) Foreign Application Priority Data
Sep. 5, 2012 (EP) 12183164.8

(51) Int. Cl. C07D 243/14; A01N 43/62

(52) U.S. Cl. CPC C07D 243/14; A01N 43/62

(57) ABSTRACT

The use of substituted benzodiazepinones and benzazepinones of the general formula (I) or salts thereof

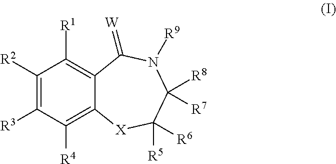

where the radicals in the general formula (I) correspond to the definitions given in the description,
for enhancing stress tolerance in plants to abiotic stress, for strengthening plant growth and/or for increasing plant yield, and to selected processes for preparing the compounds mentioned above.

The invention relates to the use of benzodiazepinones and benzazepinones or their respective salts as active compounds for increasing the stress tolerance in plants to abiotic stress, in particular for enhancing plant growth and/or for increasing plant yield.

It is known that certain substituted benzodiazepinones can be employed as inhibiting active compounds against bacterial mono-ADP ribosyltransferase toxins (cf. Antimicrobial Agents and Chemotherapy 2011, 55, 983). Moreover, it is known that substituted tricyclic benzodiazepinones and closely related structural analogs can be used as pharmaceutically active compounds for the treatment of neurodegenerative disorders, neurotoxic effects of strokes, diabetes or in cancer therapy (cf. WO200116136, WO2005012305, WO2007062413). WO2003057699 and DE19946289 likewise describe the pharmaceutical use of tricyclic benzodiazepinones, whereas WO2011008572 describes the use of quinuclidinyl-substituted dihydrobenzodiazepinoindazolones as 5-HT₃ receptor modulators.

The preparation of imidazobenzodiazepines and their inhibiting action on animal enzymes from the family of the poly(ADP-ribose)polymerase is described, for example, in J. Med. Chem. 2003, 46, 210 and in Bioorg. Med. Chem 2003, 11, 3695, whereas Synlett 2007, 1106 lists preparation methods for providing pyrrolobenzodiazepinones.

However, the use of the substituted benzodiazepinones and benzazepinones or their respective salts described in the patent applications and publications cited above for increasing stress tolerance in plants to abiotic stress, for enhancing plant growth and/or for increasing plant yield has hitherto not been described.

It is known that plants react to natural stress conditions, for example cold, heat, drought, injury, pathogenic attack (viruses, bacteria, fungi, insects), etc., but also to herbicides, with specific or unspecific defense mechanisms [Pflanzenbiochemie, pp. 393-462, Spektrum Akademischer Verlag, Heidelberg, Berlin, Oxford, Hans W. Heldt, 1996.; Biochemistry and Molecular Biology of Plants, pp. 1102-1203, American Society of Plant Physiologists, Rockville, Md., eds. Buchanan, Gruissem, Jones, 2000].

Numerous proteins in plants, and the genes that code for them, which are involved in defense reactions to abiotic stress (for example cold, heat, drought, salt, flooding) are known. Some of these form part of signal transduction chains (e.g. transcription factors, kinases, phosphatases) or cause a physiological response of the plant cell (e.g. ion transport, detoxification of reactive oxygen species). The signaling chain genes of the abiotic stress reaction include inter alia transcription factors of the DREB and CBF classes (Jaglo-Ottosen et al., 1998, Science 280: 104-106). Phosphatases of the ATPK and MP2C type are involved in the reaction to salt stress. In addition, in the event of salt stress, the biosynthesis of osmolytes such as proline or sucrose is frequently activated. This involves, for example, sucrose synthase and proline transporters (Hasegawa et al., 2000, Annu Rev Plant Physiol Plant Mol Biol 51: 463-499). The stress defense of the plants to cold and drought uses some of the same molecular mechanisms. There is a known accumulation of what are called late embryogenesis abundant proteins (LEA proteins), which include the dehydrins as an important class (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which detoxify the reactive oxygen species (ROS) which form in the event of oxidative stress (Kirch et al., 2005, Plant Mol Biol 57: 315-332).

Heat shock factors (HSF) and heat shock proteins (HSP) are activated in the event of heat stress and play a similar role here as chaperones to that of dehydrins in the event of cold and drought stress (Yu et al., 2005, Mol Cells 19: 328-333).

A number of plant-endogenous signaling substances involved in stress tolerance or pathogen defense are already known. Examples here include salicylic acid, benzoic acid, jasmonic acid or ethylene [Biochemistry and Molecular Biology of Plants, pp. 850-929, American Society of Plant Physiologists, Rockville, Md., eds. Buchanan, Gruissem, Jones, 2000]. Some of these substances or the stable synthetic derivatives and derived structures thereof are also effective on external application to plants or in seed dressing, and activate defense reactions which cause elevated stress tolerance or pathogen tolerance of the plant [Sembdner, and Parthier, 1993, Ann. Rev. Plant Physiol. Plant Mol. Biol. 44: 569-589].

It is additionally known that chemical substances can increase the tolerance of plants to abiotic stress. Such substances are applied either by seed dressing, by leaf spraying or by soil treatment. For instance, an increase in abiotic stress tolerance of crop plants by treatment with elicitors of systemic acquired resistance (SAR) or abscisic acid derivatives is described (Schading and Wei, WO-200028055, Abrams and Gusta, U.S. Pat. No. 5,201,931, Churchill et al., 1998, Plant Growth Regul 25: 35-45) or azibenzolar-S-methyl. In the case of use of fungicides, especially from the group of the strobilurins or of the succinate dehydrogenase inhibitors, similar effects are also observed, and are frequently also accompanied by an enhanced yield (Draber et al., DE-3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In addition, effects of growth regulators on the stress tolerance of crop plants have been described (Morrison and Andrews, 1992, J Plant Growth Regul 11: 113-117, RD-259027). In the event of osmotic stress, a protective effect resulting from application of osmolytes, for example glycine betaine or the biochemical precursors thereof, for example choline derivatives, has been observed (Chen et al., 2000, Plant Cell Environ 23: 609-618, Bergmann et al., DE-4103253). The effect of antioxidants, for example naphthols and xanthines, to increase abiotic stress tolerance in plants has also already been described (Bergmann et al., DD-277832, Bergmann et al., DD-277835). However, the molecular causes of the antistress action of these substances are largely unknown.

It is additionally known that the tolerance of plants to abiotic stress can be increased by a modification of the activity of endogenous poly-ADP-ribose polymerases (PARP) or poly-(ADP-ribose) glycohydrolases (PARG) (de Block et al., The Plant Journal, 2005, 41, 95; Levine et al., FEBS Lett. 1998, 440, 1; WO0004173; WO04090140).

It is thus known that plants possess several endogenous reaction mechanisms which can cause effective defense against a wide variety of harmful organisms and/or natural abiotic stress.

Since, however, the ecologic and economic demands on modern crop treatment compositions are increasing constantly, for example with respect to toxicity, selectivity, application rate, formation of residues and favorable manufacture, there is a constant need to develop novel crop treatment compositions which have advantages over those known, at least in some areas.

It was therefore an object of the present invention to provide further compounds which increase tolerance to abiotic stress in plants.

Accordingly, the present invention provides the use of substituted benzodiazepinones and benzazepinones of the general formula (I) or salts thereof

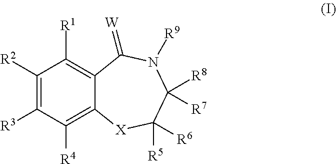

for increasing tolerance to abiotic stress in plants, where
R¹, R², R³ independently of one another represent hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, heteroarylalkoxy, arylalkoxy, heterocyclylalkoxy, cycloalkylalkyl, haloalkenyl, haloalkynyl, heterocyclylalkynyl, halocycloalkoxy, haloalkynyloxy, arylthio, heteroarylthio, alkylsulfinyl, haloalkylsulfinyl, arylsulfinyl, heteroarylsulfinyl, alkylsulfonyl, haloalkylsulfonyl, arylsulfonyl, heteroarylsulfonyl, thiocyanato, isothiocyanato, cycloalkylamino, cycloalkyl(alkyl)amino, alkenylamino, halocycloalkylalkynyl,
R⁴ represents hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, cycloalkylamino, arylamino, alkylcarbonylamino, cycloalkylcarbonylamino, arylcarbonylamino, heteroarylcarbonylamino, alkoxycarbonylamino, alkylcarbonyl(alkyl)amino, arylcarbonyl(alkyl)amino, cycloalkylcarbonyl(alkyl)amino, alkyl(alkyl)amino, haloalkyl(alkyl)amino, alkoxycarbonyl(alkyl)amino, alkoxycarbonylalkyl(alkyl)amino, alkylsulfonylamino, arylsulfonylamino, heteroarylsulfonylamino, cycloalkylsulfonylamino, alkylsulfonyl(alkyl)amino, cycloalkylsulfonyl(alkyl)amino, arylsulfonyl(alkyl)amino, heteroarylsulfonyl(alkyl)amino, alkylcarbonyl(alkyl)amino, arylcarbonyl(alkyl)amino, cycloalkylcarbonyl(alkyl)amino, heteroarylcarbonyl(alkyl)amino, alkenylamino, imino, arylimino, arylalkylimino, heteroarylimino, heterocyclylimino, haloalkylimino, alkylimino, cycloalkylimino, alkoxycarbonylimino, cycloalkoxycarbonylimino, cycloalkylalkoxycarbonylimino, arylalkoxycarbonylimino, alkylaminocarbonylimino, bisalkylaminocarbonylimino, alkylsulfinylimino, arylsulfinylimino, cycloalkylsulfinylimino, alkylthioimino, arylthioimino, cycloalkylthioimino, alkylsulfonylimino, arylsulfonylimino, cycloalkylsulfonylimino, optionally further substituted iminoalkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, alkyl, haloalkyl, cycloalkyl, aryl, alkenyl, alkynyl, heterocyclyl, heteroaryl, alkoxyalkyl or together form an exo methylene group which is optionally substituted further and part of a further ring,
R⁷, R⁸ independently of one another represent hydrogen, alkyl, haloalkyl, cycloalkyl, aryl, alkenyl, alkynyl, heterocyclyl, heteroaryl, alkoxyalkyl,
R⁹ represents hydrogen, hydroxy, alkyl, cycloalkyl, halogen, alkenylalkyl, alkynylalkyl, haloalkyl, alkoxyalkyl, alkynyl, alkenyl, cycloalkylalkyl, cyanoalkyl, nitroalkyl, arylalkyl, heteroarylalkyl, heterocyclylalkyl, aryl, alkylamino, alkylaminoalkyl, bisalkylaminoalkyl, aminocarbonylalkyl, alkylaminocarbonylalkyl, bisalkylaminocarbonylalkyl, alkoxycarbonylalkyl, hydroxycarbonylalkyl, alkylcarbonyl, cycloalkylcarbonyl, haloalkylcarbonyl, alkoxycarbonyl, alkenyloxycarbonyl, alkylaminocarbonyl, cyclopropylaminocarbonyl, alkylsulfonyl, arylsulfonyl, cycloalkylsulfonyl, arylalkylsulfonyl, alkenylsulfonyl, heteroarylsulfonyl, alkynylsulfonyl, alkylsulfinyl, arylsulfinyl, cycloalkylsulfinyl, alkenylsulfinyl, alkynylsulfinyl, arylsulfinyl, heteroarylsulfinyl, arylcarbonyl, heteroarylcarbonyl, alkoxycarbonylalkyl, alkenyloxycarbonylalkyl, hydroxycarbonylalkyl, cyanoalkylaminocarbonyl, alkynylaminocarbonyl, heterocyclylcarbonyl, heteroarylalkylaminocarbonyl, alkenyloxycarbonyl, cycloalkylalkoxycarbonyl, cycloalkylalkoxycarbonylcarbonyl, alkoxycarbonylcarbonyl, cycloalkylalkylaminocarbonyl, arylalkylaminocarbonyl or a negative charge,
R¹⁰ represents alkyl, optionally substituted alkenyl, optionally substituted iminoalkyl, optionally further substituted imino, optionally substituted alkylalkenyl, heteroarylalkenyl, arylalkyl, heteroarylalkyl, heterocyclylalkyl, alkoxycarbonylalkyl, cycloalkoxycarbonylalkyl, arylalkoxycarbonylalkyl, haloalkyl, aminoalkyl, bis-alkylaminoalkyl, alkoxyalkyl, alkylcarbonyl, arylcarbonyl, heteroarylcarbonyl, cycloalkylcarbonyl, alkoxycarbonyl, aryloxycarbonyl, cycloalkoxycarbonyl, alkylsulfonyl, cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford the general formula (Ia),

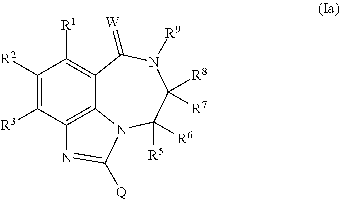

R⁴ and CR¹¹R¹² together with the atoms to which they are attached form a pyrrole ring which is substituted further by R¹³ and Q and thus together with the other substituents according to the definition afford the general formula (Ib),

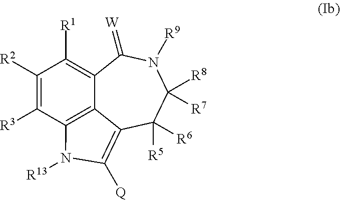

R⁵, R⁶ and N—R¹⁰ together with the atoms to which they are attached form a pyrrole ring which is optionally substituted further and thus together with the other substituents according to the definition afford the general formula (Ic),

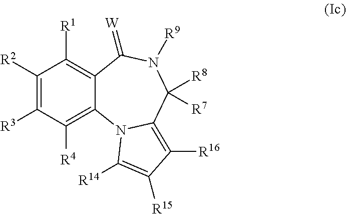

R⁴ and N—R¹⁰ together with the atoms to which they are attached form a pyrrole ring which is substituted further by R¹⁷ and Q and thus together with the other substituents according to the definition afford the general formula (Id),

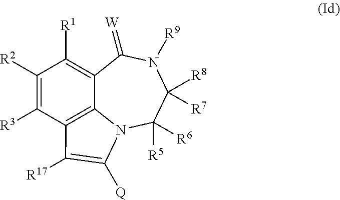

Q represents alkyl, haloalkyl, halocycloalkyl, alkoxyhaloalkyl, haloalkoxyhaloalkyl, alkylthiohaloalkyl, haloalkylthiohaloalkyl, alkoxyalkoxyhaloalkyl, bisalkylaminoalkoxyhaloalkyl, alkenyl, cycloalkyl, cycloalkylalkyl, cycloalkenyl, aryl, arylalkyl, heteroarylalkyl, heterocyclylalkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, arylalkenyl, heteroarylalkenyl, heterocyclylalkenyl, arylalkynyl, heteroarylalkynyl, heterocyclylalkynyl, cycloalkylalkynyl, alkylaminoalkyl, bis-alkylaminoalkyl, hydroxyalkyl, alkoxyalkyl, haloalkoxyalkyl, tris-[alkyl]silyloxyalkyl, bis-[alkyl]arylsilyloxyalkyl, bis-[alkyl]alkylsilyloxyalkyl, bis-alkylaminoalkoxyalkyl, alkoxyalkoxyalkyl, aryloxyalkyl, heteroaryloxyalkyl, alkylthioalkyl, haloalkylthioalkyl, arylthioalkyl, heteroarylthioalkyl, alkoxycarbonyl-N-heterocyclyl, arylalkoxycarbonyl-N-heterocyclyl, alkyl-N-heterocyclyl, alkylsulfonyl-N-heterocyclyl, arylsulfonyl-N-heterocyclyl, heteroarylsulfonyl-N-heterocyclyl, cycloalkylsulfonyl-N-heterocyclyl, haloalkylsulfonyl-N-heterocyclyl, alkylcarbonyl-N-heterocyclyl, arylcarbonyl-N-heterocyclyl, heteroarylcarbonyl-N-heterocyclyl, cycloalkylcarbonyl-N-heterocyclyl, cycloalkyl-N-heterocyclyl, aryl-N-heterocyclyl, arylalkyl-N-heterocyclyl, bis-alkylaminoalkyl-N-heterocyclyl, bis-alkylaminosulfonyl-N-heterocyclyl, heteroaryloxyaryl, heteroaryloxyheteroaryl, aryloxyheteroaryl, alkylsulfinyl, alkylthio, alkylsulfonyl, cycloalkylsulfinyl, cycloalkylthio, cycloalkylsulfonyl, arylsulfinyl, arylthio, arylsulfonyl, amino, alkylamino, bis-alkylamino, arylamino, arylalkylamino, cycloalkylamino, formyl, alkylcarbonyl, arylcarbonyl, iminoalkyl, alkyliminoalkyl, aryliminoalkyl, alkoxycarbonyl, cycloalkoxycarbonyl, cycloalkylalkoxycarbonyl, arylalkoxycarbonyl, arylalkylaminocarbonyl, aminocarbonyl, alkylaminocarbonyl, cycloalkylaminocarbonyl, bis-alkylaminocarbonyl, heterocyclyl-N-carbonyl, imino, alkylimino, arylimino, cycloalkylimino, cycloalkylalkylimino, hydroxyimino, alkoxyimino, cycloalkoxyimino, cycloalkylalkoxyimino, aryloxyimino, arylalkoxyimino, heteroarylalkoxyimino, heteroarylimino, heterocyclylimino, heterocyclylalkylimino, aminoimino, alkylaminoimino, arylaminoimino, heteroarylaminoimino, cycloalkylaminoimino, bis-alkylaminoimino, arylalkylaminoimino, aryl(alkyl)aminoimino, cycloalkyl(alkyl)aminoimino, cycloalkylalkylaminoimino, heterocyclylaminoimino, heteroarylalkoxyalkyl, arylalkoxyalkyl, heterocyclyl-N-alkyl, aryl(alkyl)aminoalkyl, arylalkyl(alkyl)aminoalkyl, alkoxycarbonylalkylaminoalkyl, alkoxycarbonylalkyl(alkyl)aminoalkyl, heteroaryl(alkyl)aminoalkyl, heteroarylalkyl(alkyl)aminoalkyl, cycloalkyl(alkyl)aminoalkyl, cycloalkylaminoalkyl, alkoxy(alkoxy)alkyl heteroarylalkoxyalkyl, arylalkoxyalkylaryl, heterocyclyl-N-alkylaryl, aryl(alkyl)aminoalkylaryl, arylalkyl(alkyl)aminoalkylaryl, alkoxycarbonylalkylaminoalkylaryl, alkoxycarbonylalkyl(alkyl)aminoalkylaryl, heteroaryl(alkyl)aminoalkylaryl, heteroarylalkyl(alkyl)aminoalkylaryl, cycloalkyl(alkyl)aminoalkylaryl, cycloalkylaminoalkylaryl, alkoxy(alkoxy)alkylaryl, alkynyl, alkylalkynyl, haloalkylalkynyl, alkylaminoalkylaryl, alkylaminoalkylarylalkyl, bis-alkylaminoalkylaryl, bis-alkylaminoalkylarylalkyl, heterocyclyl-N-alkylarylalkyl, alkoxycarbonyl-N-heterocyclyl-N-alkyl, arylalkoxycarbonyl-N-heterocyclyl-N-alkyl, alkyl-N-heterocyclyl-N-alkyl, alkylsulfonyl-N-heterocyclyl-N-alkyl, arylsulfonyl-N-heterocyclyl-N-alkyl, heteroarylsulfonyl-N-heterocyclyl-N-alkyl, cycloalkylsulfonyl-N-heterocyclyl-N-alkyl, haloalkylsulfonyl-N-heterocyclyl-N-alkyl, alkylcarbonyl-N-heterocyclyl-N-alkyl, arylcarbonyl-N-heterocyclyl-N-alkyl, heteroarylcarbonyl-N-heterocyclyl-N-alkyl, cycloalkylcarbonyl-N-heterocyclyl-N-alkyl, cycloalkyl-N-heterocyclyl-N-alkyl, alkoxycarbonyl-N-heterocyclylalkyl, arylalkoxycarbonyl-N-heterocyclylalkyl, alkyl-N-heterocyclylalkyl, alkylsulfonyl-N-heterocyclylalkyl, arylsulfonyl-N-heterocyclylalkyl, heteroarylsulfonyl-N-heterocyclylalkyl, cycloalkylsulfonyl-N-heterocyclylalkyl, haloalkylsulfonyl-N-heterocyclylalkyl, alkylcarbonyl-N-heterocyclylalkyl, arylcarbonyl-N-heterocyclylalkyl, heteroarylcarbonyl-N-heterocyclylalkyl, cycloalkylcarbonyl-N-heterocyclylalkyl, cycloalkyl-N-heterocyclylalkyl, arylalkylaminocarbonylaryl, aminocarbonylaryl, alkylaminocarbonylaryl, cycloalkylaminocarbonylaryl, bis-alkylaminocarbonylaryl, arylalkylaminocarbonylarylalkyl, aminocarbonylarylalkyl, alkylaminocarbonylarylalkyl, cycloalkylaminocarbonylarylalkyl, bis-alkylaminocarbonylarylalkyl, arylalkylaminoalkyl, heteroarylalkylaminoalkyl, alkylaminoalkoxyaryl, bis-alkylaminoalkoxyaryl,
R¹³ represents hydrogen, alkyl, cycloalkyl, cycloalkylalkyl, cycloalkenyl, cyanoalkyl, alkenylalkyl, haloalkyl, alkynylalkyl, arylalkyl, heteroarylalkyl, heterocyclylalkyl, aryl, alkylcarbonyl, cycloalkylcarbonyl, cycloalkylalkylcarbonyl, arylcarbonyl, arylalkylcarbonyl, heteroarylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, cycloalkylaminocarbonyl, bis-alkylaminocarbonyl, alkoxyalkyl alkoxycarbonylalkyl,
R¹⁴, R¹⁵, R¹⁶ independently of one another represent hydrogen, alkyl, cycloalkyl, cycloalkylalkyl, haloalkyl, aryl, alkoxycarbonyl, hydroxycarbonyl, aminocarbonyl, alkylaminocarbonyl, cycloalkylaminocarbonyl,
R¹⁷ represents hydrogen, alkyl, cycloalkyl, haloalkyl, arylalkyl, heteroarylalkyl, heterocyclylalkyl, aryl, heteroaryl, heterocyclyl, formyl, imino, hydroxyimino, alkoxyimino, cycloalkoxyimino, halogen, alkoxycarbonyl, hydroxyalkyl, alkoxyalkyl, alkylcarbonyloxyalkyl.

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts will comprise the conjugated base of the acid as the anion.

Suitable substituents present in deprotonated form, such as, for example, sulfonic acids or carboxylic acids, may form inner salts with groups which for their part can be protonated, such as amino groups.

The compounds of the formula (I) used in accordance with the invention and salts thereof are referred to hereinafter as “compounds of the general formula (I)”.

Preference is given to the use of compounds of the general formula (I) in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
R⁴ represents hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis-[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₃-C₈)-cycloalkylamino, arylamino, (C₁-C₈)-alkylcarbonylamino, (C₃-C₈)-cycloalkylcarbonylamino, arylcarbonylamino, heteroarylcarbonylamino, (C₁-C₈)-alkoxycarbonylamino, (C₁-C₈)-alkylcarbonyl[(C₁-C₈)-alkyl]amino, arylcarbonyl[(C₁-C₈)-alkyl]amino, (C₃-C₈)-cycloalkylcarbonyl[(C₁-C₈)-alkyl]amino, (C₁-C₈)-alkyl[(C₁-C₈)-alkyl]amino, (C₁-C₈)-haloalkyl-[(C₁-C₈)-alkyl]amino, (C₁-C₈)-alkoxycarbonyl-[(C₁-C₈)-alkyl]amino, (C₁-C₈)-alkoxycarbonyl-(C₁-C₈)-alkyl-[(C₁-C₈)-alkyl]amino, (C₁-C₈)-alkylsulfonylamino, arylsulfonylamino, heteroarylsulfonylamino, (C₃-C₈)-cycloalkylsulfonylamino, (C₁-C₈)-alkylsulfonyl-[(C₁-C₈)-alkyl]amino, (C₃-C₈)-cycloalkylsulfonyl-[(C₁-C₈)-alkyl]amino, arylsulfonyl-[(C₁-C₈)-alkyl]amino, heteroarylsulfonyl[(C₁-C₈)-alkyl]amino, (C₁-C₈)-alkylcarbonyl[(C₁-C₈)-alkyl]amino, arylcarbonyl-[(C₁-C₈)-alkyl]amino, (C₃-C₈)-cycloalkylcarbonyl-[(C₁-C₈)-alkyl]amino, heteroarylcarbonyl-[(C₁-C₈)-alkyl]amino, (C₂-C₈)-alkenylamino, imino, arylimino, aryl-(C₁-C₈)-alkylimino, heteroarylimino, heterocyclylimino, (C₁-C₈)-haloalkylimino, (C₁-C₈)-alkylimino, (C₃-C₈)-cycloalkylimino, (C₁-C₈)-alkoxycarbonylimino, (C₃-C₈)-cycloalkoxycarbonylimino, (C₃-C₈)-cycloalkyl-(C₁-C₈)-alkoxycarbonylimino, aryl-(C₁-C₈)-alkoxycarbonylimino, (C₁-C₈)-alkylaminocarbonylimino, bis-[(C₁-C₈)-alkyl]aminocarbonylimino, (C₁-C₈)-alkylsulfinylimino, arylsulfinylimino, (C₃-C₈)-cycloalkylsulfinylimino, (C₁-C₈)-alkylthioimino, arylthioimino, (C₃-C₈)-cycloalkylthioimino, (C₁-C₈)-alkylsulfonylimino, arylsulfonylimino, (C₃-C₈)-cycloalkylsulfonylimino, optionally further substituted imino-(C₁-C₈)-alkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
R¹⁰ represents (C₁-C₈)-alkyl, optionally substituted (C₂-C₈)-alkenyl, optionally substituted imino-(C₁-C₈)-alkyl, optionally further substituted imino, optionally substituted (C₁-C₈)-alkyl-(C₂-C₈)-alkenyl, heteroaryl-(C₂-C₈)-alkenyl, aryl-(C₁-C₈)-alkyl, heteroaryl-(C₁-C₈)-alkyl, heterocyclyl-(C₁-C₈)-alkyl, (C₁-C₈)-alkoxycarbonyl-(C₁-C₈)-alkyl, (C₃-C₈)-cycloalkoxycarbonyl-(C₁-C₈)-alkyl, aryl-(C₁-C₈)-alkoxycarbonyl-(C₁-C₈)-alkyl, (C₁-C₈)-haloalkyl, amino-(C₁-C₈)-alkyl, bis-[(C₁-C₈)-alkyl]amino-(C₁-C₈)-alkyl, (C₁-C₈)-alkoxy-(C₁-C₈)-alkyl, (C₁-C₈)-alkylcarbonyl, arylcarbonyl, heteroarylcarbonyl, (C₃-C₈)-cycloalkylcarbonyl, (C₁-C₈)-alkoxycarbonyl, aryloxycarbonyl, (C₃-C₈)-cycloalkoxycarbonyl, (C₁-C₈)-alkylsulfonyl, (C₃-C₈)-cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford the general formula (Ia),

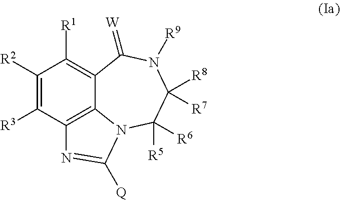

R⁴ and CR¹¹R¹² together with the atoms to which they are attached form a pyrrole ring which is substituted further by R¹³ and Q and thus together with the other substituents according to the definition afford the general formula (Ib),

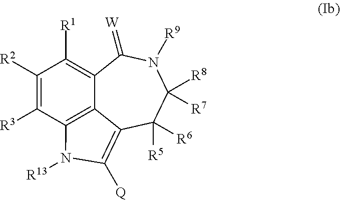

R⁵, R⁶ and N—R¹⁰ together with the atoms to which they are attached form a pyrrole ring which is optionally substituted further and thus together with the other substituents according to the definition afford the general formula (Ic),

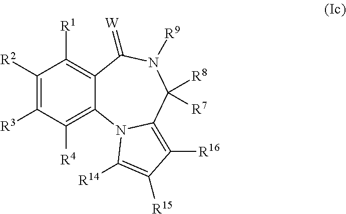

R⁴ and N—R¹⁰ together with the atoms to which they are attached form a pyrrole ring which is substituted further by R¹⁷ and Q and thus together with the other substituents according to the definition afford the general formula (Id),

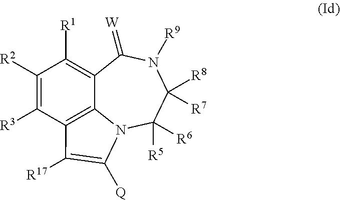

Q represents (C₁-C₈)-alkyl, (C₁-C₈)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl, (C₂-C₈)-alkenyl, (C₃-C₈)-cycloalkyl, (C₃-C₈)-cycloalkyl-(C₁-C₈)-alkyl, (C₄-C₈)-cycloalkenyl, aryl, aryl-(C₁-C₈)-alkyl, heteroaryl-(C₁-C₈)-alkyl, heterocyclyl-(C₁-C₈)-alkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₂-C₈)-alkynyl, (C₃-C₈)-cycloalkyl-(C₂-C₈)-alkynyl, (C₁-C₈)-alkylamino-(C₁-C₈)-alkyl, bis-[(C₁-C₈)-alkyl]amino-(C₁-C₈)-alkyl, hydroxy-(C₁-C₈)-alkyl, (C₁-C₈)-alkoxy-(C₁-C₈)-alkyl, (C₁-C₈)-haloalkoxy-(C₁-C₈)-alkyl, tris-[(C₁-C₈)-alkyl]silyloxy-(C₁-C₈)-alkyl, bis-[(C₁-C₈)-alkyl]arylsilyloxy-(C₁-C₈)-alkyl, bis-[(C₁-C₈)-alkyl]-(C₁-C₈)-alkylsilyloxy-(C₁-C₈)-alkyl, bis-[(C₁-C₈)-alkyl]amino-(C₁-C₈)-alkoxy-(C₁-C₈)-alkyl, (C₁-C₈)-alkoxy-(C₁-C₈)-alkoxy-(C₁-C₈)-alkyl, aryloxy-(C₁-C₈)-alkyl, heteroaryloxy-(C₁-C₈)-alkyl, (C₁-C₈)-alkylthio-(C₁-C₈)-alkyl, (C₁-C₈)-haloalkylthio-(C₁-C₈)-alkyl, arylthio-(C₁-C₈)-alkyl, heteroarylthio-(C₁-C₈)-alkyl, (C₁-C₈)-alkoxycarbonyl-N-heterocyclyl, aryl-(C₁-C₈)-alkoxycarbonyl-N-heterocyclyl, (C₁-C₈)-alkyl-N-heterocyclyl, (C₁-C₈)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₈)-haloalkylsulfonyl-N-heterocyclyl, (C₁-C₈)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₃-C₈)-cycloalkyl-N-heterocyclyl, aryl-N-heterocyclyl, aryl-(C₁-C₈)-alkyl-N-heterocyclyl, bis-[(C₁-C₈)-alkyl]amino-(C₁-C₈)-alkyl-N-heterocyclyl, bis-[(C₁-C₈)-alkyl]aminosulfonyl-N-heterocyclyl, heteroaryloxyaryl, heteroaryloxyheteroaryl, aryloxyheteroaryl, (C₁-C₈)-alkylsulfinyl, (C₁-C₈)-alkylthio, (C₁-C₈)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₈)-alkylamino, bis-[(C₁-C₈)-alkyl]amino, arylamino, aryl-(C₁-C₈)-alkylamino, (C₃-C₈)-cycloalkylamino, formyl, (C₁-C₈)-alkylcarbonyl, arylcarbonyl, imino-(C₁-C₈)-alkyl, (C₁-C₈)-alkylimino-(C₁-C₈)-alkyl, arylimino-(C₁-C₈)-alkyl, (C₁-C₈)-alkoxycarbonyl, (C₃-C₈)-cycloalkoxycarbonyl, (C₃-C₈)-cycloalkyl-(C₁-C₈)-alkoxycarbonyl, aryl-(C₁-C₈)-alkoxycarbonyl, aryl-(C₁-C₈)-alkylaminocarbonyl, aminocarbonyl, (C₁-C₈)-alkylaminocarbonyl, (C₃-C₈)-cycloalkylaminocarbonyl, bis-[(C₁-C₈)-alkyl]aminocarbonyl heterocyclyl-N-carbonyl, imino, (C₁-C₈)-alkylimino, arylimino, (C₃-C₈)-cycloalkylimino, (C₃-C₈)-cycloalkyl-(C₁-C₈)-alkylimino, hydroxyimino, (C₁-C₈)-alkoxyimino, (C₃-C₈)-cycloalkoxyimino, (C₃-C₈)-cycloalkyl-(C₁-C₈)-alkoxyimino, aryloxyimino, aryl-(C₁-C₈)-alkoxyimino, heteroaryl-(C₁-C₈)-alkoxyimino, heteroarylimino, heterocyclylimino, heterocyclyl-(C₁-C₈)-alkylimino, aminoimino, (C₁-C₈)-alkylaminoimino, arylaminoimino, heteroarylaminoimino, (C₃-C₈)-cycloalkylaminoimino, bis-[(C₁-C₈)-alkyl]aminoimino, aryl-(C₁-C₈)-alkylaminoimino, aryl[(C₁-C₈)-alkyl]aminoimino, (C₃-C₈)-cycloalkyl[(C₁-C₈)-alkyl]aminoimino, (C₃-C₈)-cycloalkyl-(C₁-C₈)-alkylaminoimino, heterocyclylaminoimino, heteroaryl-(C₁-C₈)-alkoxy-(C₁-C₈)-alkyl, aryl-(C₁-C₈)-alkoxy-(C₁-C₈)-alkyl, heterocyclyl-N—(C₁-C₈)-alkyl, aryl[(C₁-C₈)-alkyl]amino-(C₁-C₈)-alkyl, aryl-(C₁-C₈)-alkyl[(C₁-C₈)-alkyl]amino-(C₁-C₈)-alkyl, (C₁-C₈)-alkoxycarbonyl-(C₁-C₈)-alkylamino-(C₁-C₈)-alkyl, (C₁-C₈)-alkoxycarbonyl-(C₁-C₈)-alkyl[(C₁-C₈)-alkyl]amino-(C₁-C₈)-alkyl, heteroaryl[(C₁-C₈)-alkyl]amino-(C₁-C₈)-alkyl, heteroaryl-(C₁-C₈)-alkyl[(C₁-C₈)-alkyl]amino-(C₁-C₈)-alkyl, (C₃-C₈)-cycloalkyl[(C₁-C₈)-alkyl]amino-(C₁-C₈)-alkyl, (C₃-C₈)-cycloalkylamino-(C₁-C₈)-alkyl, (C₁-C₈)-alkoxy[(C₁-C₈)-alkoxy]-(C₁-C₈)-alkyl, heteroaryl-(C₁-C₈)-alkoxy-(C₁-C₈)-alkyl, aryl-(C₁-C₈)-alkoxy-(C₁-C₈)-alkylaryl, heterocyclyl-N—(C₁-C₈)-alkylaryl, aryl[(C₁-C₈)-alkyl]amino-(C₁-C₈)-alkylaryl, aryl-(C₁-C₈)-alkyl[(C₁-C₈)-alkyl]amino-(C₁-C₈)-alkylaryl, (C₁-C₈)-alkoxycarbonyl-(C₁-C₈)-alkylamino-(C₁-C₈)-alkylaryl, (C₁-C₈)-alkoxycarbonyl-(C₁-C₈)-alkyl[(C₁-C₈)-alkyl]amino-(C₁-C₈)-alkylaryl, heteroaryl[(C₁-C₈)-alkyl]amino-(C₁-C₈)-alkylaryl, heteroaryl-(C₁-C₈)-alkyl[(C₁-C₈)-alkyl]amino-(C₁-C₈)-alkylaryl, (C₃-C₈)-cycloalkyl[(C₁-C₈)-alkyl]amino-(C₁-C₈)-alkylaryl, (C₃-C₈)-cycloalkylamino-(C₁-C₈)-alkylaryl, (C₁-C₈)-alkoxy[(C₁-C₈)-alkoxy]-(C₁-C₈)-alkylaryl, (C₂-C₈)-alkynyl, (C₁-C₈)-alkyl-(C₂-C₈)-alkynyl, (C₁-C₈)-haloalkyl-(C₂-C₈)-alkynyl, (C₁-C₈)-alkylamino-(C₁-C₈)-alkylaryl, (C₁-C₈)-alkylamino-(C₁-C₈)-alkylaryl-(C₁-C₈)-alkyl, bis-[(C₁-C₈)-alkyl]amino-(C₁-C₈)-alkylaryl, bis-[(C₁-C₈)-alkyl]amino-(C₁-C₈)-alkylaryl-(C₁-C₈)-alkyl, heterocyclyl-N—(C₁-C₈)-alkylaryl-(C₁-C₈)-alkyl, (C₁-C₈)-alkoxycarbonyl-N-heterocyclyl-N—(C₁-C₈)-alkyl, aryl-(C₁-C₈)-alkoxycarbonyl-N-heterocyclyl-N—(C₁-C₈)-alkyl, (C₁-C₈)-alkyl-N-heterocyclyl-N—(C₁-C₈)-alkyl, (C₁-C₈)-alkylsulfonyl-N-heterocyclyl-N—(C₁-C₈)-alkyl, arylsulfonyl-N-heterocyclyl-N—(C₁-C₈)-alkyl, heteroarylsulfonyl-N-heterocyclyl-N—(C₁-C₈)-alkyl, (C₃-C₈)-cycloalkylsulfonyl-N-heterocyclyl-N—(C₁-C₈)-alkyl, (C₁-C₈)-haloalkylsulfonyl-N-heterocyclyl-N—(C₁-C₈)-alkyl, alkylcarbonyl-N-heterocyclyl-N-alkyl, arylcarbonyl-N-heterocyclyl-N—(C₁-C₈)-alkyl, heteroarylcarbonyl-N-heterocyclyl-N—(C₁-C₈)-alkyl, (C₃-C₈)-cycloalkylcarbonyl-N-heterocyclyl-N—(C₃-C₈)-alkyl, (C₃-C₈)-cycloalkyl-N-heterocyclyl-N—(C₁-C₈)-alkyl, (C₁-C₈)-alkoxycarbonyl-N-heterocyclyl-(C₁-C₈)-alkyl, aryl-(C₁-C₈)-alkoxycarbonyl-N-heterocyclyl-(C₁-C₈)-alkyl, (C₁-C₈)-alkyl-N-heterocyclyl-(C₁-C₈)-alkyl, (C₁-C₈)-alkylsulfonyl-N-heterocyclyl-(C₁-C₈)-alkyl, arylsulfonyl-N-heterocyclyl-(C₃-C₈)-alkyl, heteroarylsulfonyl-N-heterocyclyl-(C₁-C₈)-alkyl, (C₃-C₈)-cycloalkylsulfonyl-N-heterocyclyl-(C₁-C₈)-alkyl, (C₁-C₈)-haloalkylsulfonyl-N-heterocyclyl-(C₁-C₈)-alkyl, (C₁-C₈)-alkylcarbonyl-N-heterocyclyl-(C₁-C₈)-alkyl, arylcarbonyl-N-heterocyclyl-(C₁-C₈)-alkyl, heteroarylcarbonyl-N-heterocyclyl-(C₁-C₈)-alkyl, (C₃-C₈)-cycloalkylcarbonyl-N-heterocyclyl-(C₁-C₈)-alkyl, (C₃-C₈)-cycloalkyl-N-heterocyclyl-(C₁-C₈)-alkyl, aryl-(C₁-C₈)-alkylaminocarbonylaryl, aminocarbonylaryl, (C₁-C₈)-alkylaminocarbonylaryl, (C₃-C₈)-cycloalkylaminocarbonylaryl, bis-[(C₁-C₈)-alkyl]aminocarbonylaryl, aryl-(C₁-C₈)-alkylaminocarbonylaryl-(C₁-C₈)-alkyl, aminocarbonylaryl-(C₁-C₈)-alkyl, (C₁-C₈)-alkylaminocarbonylaryl-(C₁-C₈)-alkyl, (C₃-C₈)-cycloalkylaminocarbonylaryl-(C₁-C₈)-alkyl, bis-[(C₁-C₈)-alkyl]aminocarbonylaryl-(C₁-C₈)-alkyl, aryl-(C₁-C₈)-alkylamino-(C₁-C₈)-alkyl, heteroaryl-(C₁-C₈)-alkylamino-(C₁-C₈)-alkyl, (C₁-C₈)-alkylamino-(C₁-C₈)-alkoxyaryl, bis-[(C₁-C₈)-alkyl]amino-(C₁-C₈)-alkoxyaryl,
R¹³ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylcarbonyl, (C₃-C₈)-cycloalkylcarbonyl, (C₃-C₈)-cycloalkyl-(C₁-C₈)-alkylcarbonyl, arylcarbonyl, aryl-(C₁-C₈)-alkylcarbonyl, heteroarylcarbonyl, (C₁-C₈)-alkoxycarbonyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, bis-[(C₁-C₈)-alkyl]aminocarbonyl, (C₁-C₈)-alkoxy-(C₁-C₈)-alkyl, (C₁-C₈)-alkoxycarbonyl-(C₁-C₈)-alkyl,
R¹⁴, R¹⁵, R¹⁶ independently of one another represent hydrogen, (C₁-C₈)-alkyl, (C₃-C₈)-cycloalkyl, (C₃-C₈)-cycloalkyl-(C₁-C₈)-alkyl, (C₁-C₈)-haloalkyl, aryl, (C₁-C₈)-alkoxycarbonyl, (C₁-C₈)-hydroxycarbonyl, aminocarbonyl, (C₁-C₈)-alkylaminocarbonyl, (C₃-C₈)-cycloalkylaminocarbonyl,
R¹⁷ represents hydrogen, (C₁-C₈)-alkyl, (C₃-C₈)-cycloalkyl, (C₁-C₈)-haloalkyl, aryl-(C₁-C₈)-alkyl, heteroaryl-(C₁-C₈)-alkyl, heterocyclyl-(C₁-C₈)-alkyl, aryl, heteroaryl, heterocyclyl, formyl, imino, hydroxyimino, (C₁-C₈)-alkoxyimino, (C₃-C₈)-cycloalkoxyimino, halogen, (C₁-C₈)-alkoxycarbonyl, hydroxy-(C₁-C₈)-alkyl, (C₁-C₈)-alkoxy-(C₁-C₈)-alkyl, (C₁-C₈)-alkylcarbonyloxy-(C₁-C₈)-alkyl.

Particular preference is given to the use of compounds of the general formula (I) in which
R¹, R², R³ independently of one another represent hydrogen, halogen, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, aryl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroraryloxy, (C₁-C₇)-alkoxy-(C₁-C₇)-alkoxy, (C₂-C₇)-alkynyl-(C₁-C₇)-alkoxy, (C₂-C₇)-alkenyloxy, bis[(C₁-C₇)-alkyl]amino-(C₁-C₇)-alkoxy, tris-[(C₁-C₇)-alkyl]silyl, bis-[(C₁-C₇)-alkyl]arylsilyl, bis-[(C₁-C₇)-alkyl]-(C₁-C₇)-alkylsilyl, tris-[(C₁-C₇)-alkyl]silylalkynyl, aryl-(C₂-C₇)-alkynyl, heteroaryl-(C₂-C₇)-alkynyl, (C₁-C₇)-alkyl-(C₁-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis-[(C₁-C₇)-alkyl]amino, (C₁-C₇)-alkylcarbonylamino, (C₃-C₇)-cycloalkylcarbonylamino, arylcarbonylamino, (C₁-C₇)-alkoxycarbonylamino, heteroaryl-(C₁-C₇)-alkoxy, aryl-(C₁-C₇)-alkoxy, heterocyclyl-(C₁-C₇)-alkoxy, (C₃-C₇)-cycloalkyl-(C₁-C₇)-alkyl, (C₂-C₇)-haloalkenyl, (C₂-C₇)-haloalkynyl, heterocyclyl-(C₂-C₇)-alkynyl, (C₃-C₇)-halocycloalkoxy, (C₂-C₇)-haloalkynyloxy, arylthio, heteroarylthio, (C₁-C₇)-alkylsulfinyl, (C₁-C₇)-haloalkylsulfinyl, arylsulfinyl, heteroarylsulfinyl, (C₁-C₇)-alkylsulfonyl, (C₁-C₇)-haloalkylsulfonyl, arylsulfonyl, heteroarylsulfonyl, thiocyanato, isothiocyanato, (C₃-C₇)-cycloalkylamino, (C₃-C₇)-cycloalkyl[(C₁-C₇)-alkyl]amino, (C₂-C₇)-alkenylamino, (C₁-C₇)-halocycloalkyl-(C₂-C₇)-alkynyl,
R⁴ represents hydrogen, halogen, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, aryl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroraryloxy, (C₁-C₇)-alkoxy-(C₁-C₇)-alkoxy, (C₂-C₇)-alkynyl-(C₁-C₇)-alkoxy, (C₂-C₇)-alkenyloxy, bis-[(C₁-C₇)-alkyl]amino-(C₁-C₇)-alkoxy, tris-[(C₁-C₇)-alkyl]silyl, bis-[(C₁-C₇)-alkyl]arylsilyl, bis-[(C₁-C₇)-alkyl]-(C₁-C₇)-alkylsilyl, tris-[(C₁-C₇)-alkyl]silyl-(C₂-C₇)-alkynyl, aryl-(C₂-C₇)-alkynyl, heteroaryl-(C₂-C₇)-alkynyl, (C₁-C₇)-alkyl-(C₂-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis-[(C₁-C₇)-alkyl]amino, (C₃-C₇)-cycloalkylamino, arylamino, (C₁-C₇)-alkylcarbonylamino, (C₃-C₇)-cycloalkylcarbonylamino, arylcarbonylamino, heteroarylcarbonylamino, (C₁-C₇)-alkoxycarbonylamino, (C₁-C₇)-alkylcarbonyl[(C₁-C₇)-alkyl]amino, arylcarbonyl[(C₁-C₇)-alkyl]amino, (C₃-C₇)-cycloalkylcarbonyl[(C₁-C₇)-alkyl]amino, (C₁-C₇)-alkyl[(C₁-C₇)-alkyl]amino, (C₁-C₇)-haloalkyl-[(C₁-C₇)-alkyl]amino, (C₁-C₇)-alkoxycarbonyl-[(C₁-C₇)-alkyl]amino, (C₁-C₇)-alkoxycarbonyl-(C₁-C₇)-alkyl-[(C₁-C₇)-alkyl]amino, (C₁-C₇)-alkylsulfonylamino, arylsulfonylamino, heteroarylsulfonylamino, (C₃-C₇)-cycloalkylsulfonylamino, (C₁-C₇)-alkylsulfonyl-[(C₁-C₇)-alkyl]amino, (C₃-C₇)-cycloalkylsulfonyl-[(C₁-C₇)-alkyl]amino, arylsulfonyl-[(C₁-C₇)-alkyl]amino, heteroarylsulfonyl[(C₁-C₇)-alkyl]amino, (C₁-C₇)-alkylcarbonyl[(C₁-C₇)-alkyl]amino, arylcarbonyl-[(C₁-C₇)-alkyl]amino, (C₃-C₇)-cycloalkylcarbonyl-[(C₁-C₇)-alkyl]amino, heteroarylcarbonyl-[(C₁-C₇)-alkyl]amino, (C₂-C₇)-alkenylamino, imino, arylimino, aryl-(C₁-C₇)-alkylimino, heteroarylimino, heterocyclylimino, (C₁-C₇)-haloalkylimino, (C₁-C₇)-alkylimino, (C₃-C₇)-cycloalkylimino, (C₁-C₇)-alkoxycarbonylimino, (C₃-C₇)-cycloalkoxycarbonylimino, (C₃-C₇)-cycloalkyl-(C₁-C₇)-alkoxycarbonylimino, aryl-(C₁-C₇)-alkoxycarbonylimino, (C₁-C₇)-alkylaminocarbonylimino, bis-[(C₁-C₇)-alkyl]aminocarbonylimino, (C₁-C₇)-alkylsulfinylimino, arylsulfinylimino, (C₃-C₇)-cycloalkylsulfinylimino, (C₁-C₇)-alkylthioimino, arylthioimino, (C₃-C₇)-cycloalkylthioimino, (C₁-C₇)-alkylsulfonylimino, arylsulfonylimino, (C₃-C₇)-cycloalkylsulfonylimino, optionally further substituted imino-(C₁-C₇)-alkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, (C₁-C₇)-alkyl, (C₁-C₇)-haloalkyl, (C₃-C₇)-cycloalkyl, aryl, (C₂-C₇)-alkenyl, (C₂-C₇)-alkynyl, heterocyclyl, heteroaryl, (C₁-C₇)-alkoxy-(C₁-C₇)-alkyl or together form an exo methylene group which is optionally substituted further and part of a further ring,
R⁷, R⁸ independently of one another represent hydrogen, (C₁-C₇)-alkyl, (C₁-C₇)-haloalkyl, (C₃-C₇)-cycloalkyl, aryl, (C₂-C₇)-alkenyl, (C₁-C₇)-alkynyl, heterocyclyl, heteroaryl, (C₁-C₇)-alkoxy-(C₁-C₇)-alkyl,
R⁹ represents hydrogen, hydroxy, (C₁-C₇)-alkyl, (C₃-C₇)-cycloalkyl, halogen, (C₂-C₇)-alkenyl-(C₁-C₇)-alkyl, (C₂-C₇)-alkynyl-(C₁-C₇)-alkyl, (C₁-C₇)-haloalkyl, (C₁-C₇)-alkoxy-(C₁-C₇)-alkyl, (C₂-C₇)-alkynyl, (C₂-C₇)-alkenyl, (C₃-C₇)-cycloalkyl-(C₁-C₇)-alkyl, cyano-(C₁-C₇)-alkyl, nitro-(C₁-C₇)-alkyl, aryl-(C₁-C₇)-alkyl, heteroaryl-(C₁-C₇)-alkyl, heterocyclyl-(C₁-C₇)-alkyl, aryl, (C₁-C₇)-alkylamino, (C₁-C₇)-alkylamino-(C₁-C₇)-alkyl, bis-[(C₁-C₇)-alkyl]amino-(C₁-C₇)-alkyl, aminocarbonyl-(C₁-C₇)-alkyl, (C₁-C₇)-alkylaminocarbonyl-(C₁-C₇)-alkyl, bis-[(C₁-C₇)-alkyl]aminocarbonyl-(C₁-C₇)-alkyl, (C₁-C₇)-alkoxycarbonyl-(C₁-C₇)-alkyl, hydroxycarbonyl-(C₁-C₇)-alkyl, (C₁-C₇)-alkylcarbonyl, (C₃-C₇)-cycloalkylcarbonyl, (C₁-C₇)-haloalkylcarbonyl, (C₁-C₇)-alkoxycarbonyl, (C₂-C₇)-alkenyloxycarbonyl, (C₁-C₇)-alkylaminocarbonyl, (C₃-C₇)-cyclopropylaminocarbonyl, (C₁-C₇)-alkylsulfonyl, arylsulfonyl, (C₁-C₇)-cycloalkylsulfonyl, aryl-(C₁-C₇)-alkylsulfonyl, (C₂-C₇)-alkenylsulfonyl, heteroarylsulfonyl, (C₂-C₇)-alkynylsulfonyl, (C₁-C₇)-alkylsulfinyl, arylsulfinyl, (C₃-C₇)-cycloalkylsulfinyl, (C₂-C₇)-alkenylsulfinyl, (C₂-C₇)-alkynylsulfinyl, arylsulfinyl, heteroarylsulfinyl, arylcarbonyl, heteroarylcarbonyl, (C₁-C₇)-alkoxycarbonyl-(C₁-C₇)-alkyl, (C₂-C₇)-alkenyloxycarbonyl-(C₁-C₇)-alkyl, hydroxycarbonyl-(C₁-C₇)-alkyl, cyano-(C₁-C₇)-alkylaminocarbonyl, (C₂-C₇)-alkynylaminocarbonyl, heterocyclylcarbonyl, heteroaryl-(C₁-C₇)-alkylaminocarbonyl, (C₂-C₇)-alkenyloxycarbonyl, (C₃-C₇)-cycloalkyl-(C₁-C₇)-alkoxycarbonyl, (C₃-C₇)-cycloalkyl-(C₁-C₇)-alkoxycarbonylcarbonyl, (C₁-C₇)-alkoxycarbonylcarbonyl, (C₃-C₇)-cycloalkyl-(C₁-C₇)-alkylaminocarbonyl, aryl-(C₁-C₇)-alkylaminocarbonyl or a negative charge,
R¹⁰ represents (C₁-C₇)-alkyl, optionally substituted (C₂-C₇)-alkenyl, optionally substituted imino-(C₁-C₇)-alkyl, optionally further substituted imino, optionally substituted (C₁-C₇)-alkyl-(C₂-C₇)-alkenyl, heteroaryl-(C₂-C₇)-alkenyl, aryl-(C₁-C₇)-alkyl, heteroaryl-(C₁-C₇)-alkyl, heterocyclyl-(C₁-C₇)-alkyl, (C₁-C₇)-alkoxycarbonyl-(C₁-C₇)-alkyl, (C₃-C₇)-cycloalkoxycarbonyl-(C₁-C₇)-alkyl, aryl-(C₁-C₇)-alkoxycarbonyl-(C₁-C₇)-alkyl, (C₁-C₇)-haloalkyl, amino-(C₁-C₇)-alkyl, bis-[(C₁-C₇)-alkyl]amino-(C₁-C₇)-alkyl, (C₁-C₇)-alkoxy-(C₁-C₇)-alkyl, (C₁-C₇)-alkylcarbonyl, arylcarbonyl, heteroarylcarbonyl, (C₃-C₇)-cycloalkylcarbonyl, (C₁-C₇)-alkoxycarbonyl, aryloxycarbonyl, (C₃-C₇)-cycloalkoxycarbonyl, (C₁-C₇)-alkylsulfonyl, (C₃-C₇)-cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford the general formula (Ia),

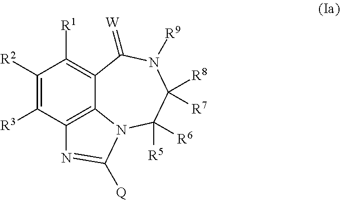

R⁴ and CR¹¹R¹² together with the atoms to which they are attached form a pyrrole ring which is substituted further by R¹³ and Q and thus together with the other substituents according to the definition afford the general formula (Ib),

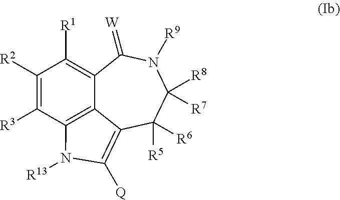

R⁵, R⁶ and N—R¹⁰ together with the atoms to which they are attached form a pyrrole ring which is optionally substituted further and thus together with the other substituents according to the definition afford the general formula (Ic),

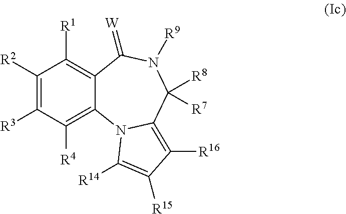

R⁴ and N—R¹⁰ together with the atoms to which they are attached form a pyrrole ring which is substituted further by R¹⁷ and Q and thus together with the other substituents according to the definition afford the general formula (Id),

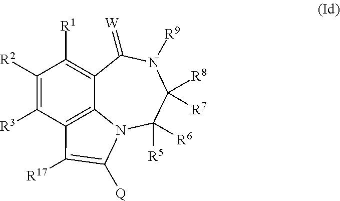

Q represents (C₁-C₇)-alkyl, (C₁-C₇)-haloalkyl, (C₃-C₇)-halocycloalkyl, (C₁-C₇)-alkoxy-(C₁-C₇)-haloalkyl, (C₁-C₇)-haloalkoxy-(C₁-C₇)-haloalkyl, (C₁-C₇)-alkylthio-(C₁-C₇)-haloalkyl, (C₁-C₇)-haloalkylthio-(C₁-C₇)-haloalkyl, (C₁-C₇)-alkoxy-(C₁-C₇)-alkoxy-(C₁-C₇)-haloalkyl, bis-[(C₁-C₇)-alkyl]amino-(C₁-C₇)-alkoxy-(C₁-C₇)-haloalkyl, (C₂-C₇)-alkenyl, (C₃-C₇)-cycloalkyl, (C₃-C₇)-cycloalkyl-(C₁-C₇)-alkyl, (C₄-C₇)-cycloalkenyl, optionally further substituted phenyl, aryl-(C₁-C₇)-alkyl, heteroaryl-(C₁-C₇)-alkyl, heterocyclyl-(C₁-C₇)-alkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₂-C₇)-alkynyl, (C₃-C₇)-cycloalkyl-(C₂-C₇)-alkynyl, (C₁-C₇)-alkylamino-(C₁-C₇)-alkyl, bis-[(C₁-C₇)-alkyl]amino-(C₁-C₇)-alkyl, hydroxy-(C₁-C₇)-alkyl, (C₁-C₇)-alkoxy-(C₁-C₇)-alkyl, (C₁-C₇)-haloalkoxy-(C₁-C₇)-alkyl, tris-[(C₁-C₇)-alkyl]silyloxy-(C₁-C₇)-alkyl, bis-[(C₁-C₇)-alkyl]arylsilyloxy-(C₁-C₇)-alkyl, bis-[(C₁-C₇)-alkyl]-(C₁-C₇)-alkylsilyloxy-(C₁-C₇)-alkyl, bis-[(C₁-C₇)-alkyl]amino-(C₁-C₇)-alkoxy-(C₁-C₇)-alkyl, (C₁-C₇)-alkoxy-(C₁-C₇)-alkoxy-(C₁-C₇)-alkyl, aryloxy-(C₁-C₇)-alkyl, heteroaryloxy-(C₁-C₇)-alkyl, (C₁-C₇)-alkylthio-(C₁-C₇)-alkyl, (C₁-C₇)-haloalkylthio-(C₁-C₇)-alkyl, arylthio-(C₁-C₇)-alkyl, heteroarylthio-(C₁-C₇)-alkyl, (C₁-C₇)-alkoxycarbonyl-N-heterocyclyl, aryl-(C₁-C₇)-alkoxycarbonyl-N-heterocyclyl, (C₁-C₇)-alkyl-N-heterocyclyl, (C₁-C₇)-alkylsulfonyl-N-heterocyclyl, arylsulfonyl-N-heterocyclyl, heteroarylsulfonyl-N-heterocyclyl (C₃-C₇)-cycloalkylsulfonyl-N-heterocyclyl, (C₁-C₇)-haloalkylsulfonyl-N-heterocyclyl, (C₁-C₇)-alkylcarbonyl-N-heterocyclyl, arylcarbonyl-N-heterocyclyl, heteroarylcarbonyl-N-heterocyclyl, (C₃-C₇)-cycloalkylcarbonyl-N-heterocyclyl, (C₃-C₇)-cycloalkyl-N-heterocyclyl, aryl-N-heterocyclyl, aryl-(C₁-C₇)-alkyl-N-heterocyclyl, bis-[(C₁-C₇)-alkyl]amino-(C₁-C₇)-alkyl-N-heterocyclyl, bis-[(C₁-C₇)-alkyl]aminosulfonyl-N-heterocyclyl, heteroaryloxyaryl, heteroaryloxyheteroaryl, aryloxyheteroaryl, (C₁-C₇)-alkylsulfinyl, (C₁-C₇)-alkylthio, (C₁-C₇)-alkylsulfonyl, (C₃-C₇)-cycloalkylsulfinyl, (C₃-C₇)-cycloalkylthio, (C₃-C₇)-cycloalkylsulfonyl, arylsulfinyl, arylthio, arylsulfonyl, amino, (C₁-C₇)-alkylamino, bis-[(C₁-C₇)-alkyl]amino, arylamino, aryl-(C₁-C₇)-alkylamino, (C₃-C₇)-cycloalkylamino, formyl, (C₁-C₇)-alkylcarbonyl, arylcarbonyl, imino-(C₁-C₇)-alkyl, (C₁-C₇)-alkylimino-(C₁-C₇)-alkyl, arylimino-(C₁-C₇)-alkyl, (C₁-C₇)-alkoxycarbonyl, (C₃-C₇)-cycloalkoxycarbonyl, (C₃-C₇)-cycloalkyl-(C₁-C₇)-alkoxycarbonyl, aryl-(C₁-C₇)-alkoxycarbonyl, aryl-(C₁-C₇)-alkylaminocarbonyl, aminocarbonyl, (C₁-C₇)-alkylaminocarbonyl, (C₃-C₇)-cycloalkylaminocarbonyl, bis-[(C₁-C₇)-alkyl]aminocarbonyl heterocyclyl-N-carbonyl, imino, (C₁-C₇)-alkylimino, arylimino, (C₃-C₇)-cycloalkylimino, (C₃-C₇)-cycloalkyl-(C₁-C₇)-alkylimino, hydroxyimino, (C₁-C₇)-alkoxyimino, (C₃-C₇)-cycloalkoxyimino, (C₃-C₇)-cycloalkyl-(C₁-C₇)-alkoxyimino, aryloxyimino, aryl-(C₁-C₇)-alkoxyimino, heteroaryl-(C₁-C₇)-alkoxyimino, heteroarylimino, heterocyclylimino, heterocyclyl-(C₁-C₇)-alkylimino, aminoimino, (C₁-C₇)-alkylaminoimino, arylaminoimino, heteroarylaminoimino, (C₃-C₇)-cycloalkylaminoimino, bis-[(C₁-C₇)-alkyl]aminoimino, aryl-(C₁-C₇)-alkylaminoimino, aryl[(C₁-C₇)-alkyl]aminoimino, (C₃-C₇)-cycloalkyl[(C₁-C₇)-alkyl]aminoimino, (C₃-C₇)-cycloalkyl-(C₁-C₇)-alkylaminoimino, heterocyclylaminoimino, heteroaryl-(C₁-C₇)-alkoxy-(C₁-C₇)-alkyl, aryl-(C₁-C₇)-alkoxy-(C₁-C₇)-alkyl, heterocyclyl-N—(C₁-C₇)-alkyl, aryl[(C₁-C₇)-alkyl]amino-(C₁-C₇)-alkyl, aryl-(C₁-C₇)-alkyl[(C₁-C₇)-alkyl]amino-(C₁-C₇)-alkyl, (C₁-C₇)-alkoxycarbonyl-(C₁-C₇)-alkylamino-(C₁-C₇)-alkyl, (C₁-C₇)-alkoxycarbonyl-(C₁-C₇)-alkyl[(C₁-C₇)-alkyl]amino-(C₁-C₇)-alkyl, heteroaryl[(C₁-C₇)-alkyl]amino-(C₁-C₇)-alkyl, heteroaryl-(C₁-C₇)-alkyl[(C₁-C₇)-alkyl]amino-(C₁-C₇)-alkyl, (C₃-C₇)-cycloalkyl[(C₁-C₇)-alkyl]amino-(C₁-C₇)-alkyl, (C₃-C₇)-cycloalkylamino-(C₁-C₇)-alkyl, (C₁-C₇)-alkoxy[(C₁-C₇)-alkoxy]-(C₁-C₇)-alkyl, heteroaryl-(C₁-C₇)-alkoxy-(C₁-C₇)-alkyl, aryl-(C₁-C₇)-alkoxy-(C₁-C₇)-alkylaryl, heterocyclyl-N—(C₁-C₇)-alkylaryl, aryl[(C₁-C₇)-alkyl]amino-(C₁-C₇)-alkylaryl, aryl-(C₁-C₇)-alkyl[(C₁-C₇)-alkyl]amino-(C₁-C₇)-alkylaryl, (C₁-C₇)-alkoxycarbonyl-(C₁-C₇)-alkylamino-(C₁-C₇)-alkylaryl, (C₁-C₇)-alkoxycarbonyl-(C₁-C₇)-alkyl[(C₁-C₇)-alkyl]amino-(C₁-C₇)-alkylaryl, heteroaryl[(C₁-C₇)-alkyl]amino-(C₁-C₇)-alkylaryl, heteroaryl-(C₁-C₇)-alkyl[(C₁-C₇)-alkyl]amino-(C₁-C₇)-alkylaryl, (C₃-C₇)-cycloalkyl[(C₁-C₇)-alkyl]amino-(C₁-C₇)-alkylaryl, (C₃-C₇)-cycloalkylamino-(C₁-C₇)-alkylaryl, (C₁-C₇)-alkoxy[(C₁-C₇)-alkoxy]-(C₁-C₇)-alkylaryl, (C₂-C₇)-alkynyl, (C₁-C₇)-alkyl-(C₂-C₇)-alkynyl, (C₁-C₇)-haloalkyl-(C₂-C₇)-alkynyl, (C₁-C₇)-alkylamino-(C₁-C₇)-alkylaryl, (C₁-C₇)-alkylamino-(C₁-C₇)-alkylaryl-(C₁-C₇)-alkyl, bis-[(C₁-C₇)-alkyl]amino-(C₁-C₇)-alkylaryl, bis-[(C₁-C₇)-alkyl]amino-(C₁-C₇)-alkylaryl-(C₁-C₇)-alkyl, heterocyclyl-N—(C₁-C₇)-alkylaryl-(C₁-C₇)-alkyl, (C₁-C₇)-alkoxycarbonyl-N-heterocyclyl-N—(C₁-C₇)-alkyl, aryl-(C₁-C₇)-alkoxycarbonyl-N-heterocyclyl-N—(C₁-C₇)-alkyl, (C₁-C₇)-alkyl-N-heterocyclyl-N—(C₁-C₇)-alkyl, (C₁-C₇)-alkylsulfonyl-N-heterocyclyl-N—(C₁-C₇)-alkyl, arylsulfonyl-N-heterocyclyl-N—(C₁-C₇)-alkyl, heteroarylsulfonyl-N-heterocyclyl-N—(C₁-C₇)-alkyl, (C₃-C₇)-cycloalkylsulfonyl-N-heterocyclyl-N—(C₁-C₇)-alkyl, (C₁-C₇)-haloalkylsulfonyl-N-heterocyclyl-N—(C₁-C₇)-alkyl, alkylcarbonyl-N-heterocyclyl-N-alkyl, arylcarbonyl-N-heterocyclyl-N—(C₁-C₇)-alkyl, heteroarylcarbonyl-N-heterocyclyl-N—(C₁-C₇)-alkyl, (C₃-C₇)-cycloalkylcarbonyl-N-heterocyclyl-N—(C₁-C₇)-alkyl, (C₃-C₇)-cycloalkyl-N-heterocyclyl-N—(C₁-C₇)-alkyl, (C₁-C₇)-alkoxycarbonyl-N-heterocyclyl-(C₁-C₇)-alkyl, aryl-(C₁-C₇)-alkoxycarbonyl-N-heterocyclyl-(C₁-C₇)-alkyl, (C₁-C₇)-alkyl-N-heterocyclyl-(C₁-C₇)-alkyl, (C₁-C₇)-alkylsulfonyl-N-heterocyclyl-(C₁-C₇)-alkyl, arylsulfonyl-N-heterocyclyl-(C₁-C₇)-alkyl, heteroarylsulfonyl-N-heterocyclyl-(C₁-C₇)-alkyl, (C₃-C₇)-cycloalkylsulfonyl-N-heterocyclyl-(C₁-C₇)-alkyl, (C₁-C₇)-haloalkylsulfonyl-N-heterocyclyl-(C₁-C₇)-alkyl, (C₁-C₇)-alkylcarbonyl-N-heterocyclyl-(C₁-C₇)-alkyl, arylcarbonyl-N-heterocyclyl-(C₁-C₇)-alkyl, heteroarylcarbonyl-N-heterocyclyl-(C₁-C₇)-alkyl, (C₃-C₇)-cycloalkylcarbonyl-N-heterocyclyl-(C₁-C₇)-alkyl, (C₃-C₇)-cycloalkyl-N-heterocyclyl-(C₁-C₇)-alkyl, aryl-(C₁-C₇)-alkylaminocarbonylaryl, aminocarbonylaryl, (C₁-C₇)-alkylaminocarbonylaryl, (C₃-C₇) cycloalkylaminocarbonylaryl, bis-[(C₁-C₇)-alkyl]aminocarbonylaryl, aryl-(C₁-C₇)-alkylaminocarbonylaryl-(C₁-C₇)-alkyl, aminocarbonylaryl-(C₁-C₇)-alkyl, (C₁-C₇)-alkylaminocarbonylaryl-(C₁-C₇)-alkyl, (C₃-C₇)-cycloalkylaminocarbonylaryl-(C₁-C₇)-alkyl, bis-[(C₁-C₇)-alkyl]aminocarbonylaryl-(C₁-C₇)-alkyl, aryl-(C₁-C₇)-alkylamino-(C₁-C₇)-alkyl, heteroaryl-(C₁-C₇)-alkylamino-(C₁-C₇)-alkyl, (C₁-C₇)-alkylamino-(C₁-C₇)-alkoxyaryl, bis-[(C₁-C₇)-alkyl]amino-(C₁-C₇)-alkoxyaryl,
R¹³ represents hydrogen, (C₁-C₇)-alkyl, (C₃-C₇)-cycloalkyl, (C₃-C₇)-cycloalkyl-(C₁-C₇)-alkyl, (C₄-C₇)-cycloalkenyl, cyano-(C₁-C₇)-alkyl, (C₂-C₇)-alkenyl-(C₁-C₇)-alkyl, (C₁-C₇)-haloalkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, aryl, (C₁-C₇)-alkylcarbonyl, (C₃-C₇)-cycloalkylcarbonyl, (C₃-C₇)-cycloalkyl-(C₁-C₇)-alkylcarbonyl, arylcarbonyl, aryl-(C₁-C₇)-alkylcarbonyl, heteroarylcarbonyl, (C₁-C₇)-alkoxycarbonyl, (C₁-C₇)-alkylsulfonyl, (C₃-C₇)-cycloalkylsulfonyl, arylsulfonyl, heteroarylsulfonyl, (C₁-C₇)-alkoxycarbonylcarbonyl, aryl-(C₁-C₇)-alkoxycarbonylcarbonyl, (C₁-C₇)-alkylaminothiocarbonyl, (C₁-C₇)-alkylaminocarbonyl, (C₃-C₇)-cycloalkylaminocarbonyl, bis-[(C₁-C₇)-alkyl]aminocarbonyl, (C₁-C₇)-alkoxy-(C₁-C₇)-alkyl, (C₁-C₇)-alkoxycarbonyl-(C₁-C₇)-alkyl,
R¹⁴, R¹⁵, R¹⁶ independently of one another represent hydrogen, (C₁-C₇)-alkyl, (C₃-C₇)-cycloalkyl, (C₃-C₇)-cycloalkyl-(C₁-C₇)-alkyl, (C₁-C₇)-haloalkyl, aryl, (C₁-C₇)-alkoxycarbonyl, (C₁-C₇)-hydroxycarbonyl, aminocarbonyl, (C₁-C₇)-alkylaminocarbonyl, (C₃-C₇)-cycloalkylaminocarbonyl,
R¹⁷ represents hydrogen, (C₁-C₇)-alkyl, (C₃-C₇)-cycloalkyl, (C₁-C₇)-haloalkyl, aryl-(C₁-C₇)-alkyl, heteroaryl-(C₁-C₇)-alkyl, heterocyclyl-(C₁-C₇)-alkyl, aryl, heteroaryl, heterocyclyl, formyl, imino, hydroxyimino, (C₁-C₇)-alkoxyimino, (C₃-C₇)-cycloalkoxyimino, halogen, (C₁-C₇)-alkoxycarbonyl, hydroxy-(C₁-C₇)-alkyl, (C₁-C₇)-alkoxy-(C₁-C₇)-alkyl, (C₁-C₇)-alkylcarbonyloxy-(C₁-C₇)-alkyl.

Very particular preference is given to the use of compounds of the general formula (I) in which
R¹, R², R³ independently of one another represent hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, optionally substituted phenyl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silylalkynyl, aryl-(C₂-C₆)-alkynyl, heteroaryl-(C₂-C₆)-alkynyl, (C₁-C₆)-alkyl-(C₁-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₁-C₆)-alkylcarbonylamino, (C₃-C₆)-cycloalkylcarbonylamino, arylcarbonylamino, (C₁-C₆)-alkoxycarbonylamino, heteroaryl-(C₁-C₆)-alkoxy, aryl-(C₁-C₆)-alkoxy, heterocyclyl-(C₁-C₆)-alkoxy, (C₃-C₆)-cycloalkyl-(C₁-C₆)-alkyl, (C₂-C₆)-haloalkenyl, (C₂-C₆)-haloalkynyl, heterocyclyl-(C₂-C₆)-alkynyl, (C₃-C₆)-halocycloalkoxy, (C₂-C₆)-haloalkynyloxy, arylthio, heteroarylthio, (C₁-C₆)-alkylsulfinyl, (C₁-C₆)-haloalkylsulfinyl, arylsulfinyl, heteroarylsulfinyl, (C₁-C₆)-alkylsulfonyl, (C₁-C₆)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₆)-alkyl]amino, (C₂-C₆)-alkenylamino, (C₁-C₆)-halocycloalkyl-(C₂-C₆)-alkynyl,
R⁴ represents hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, optionally substituted phenyl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis-[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silyl-(C₂-C₆)-alkynyl, aryl-(C₂-C₆)-alkynyl, heteroaryl-(C₂-C₆)-alkynyl, (C₁-C₆)-alkyl-(C₂-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₃-C₆)-cycloalkylamino, arylamino, (C₁-C₆)-alkylcarbonylamino, (C₃-C₆)-cycloalkylcarbonylamino, arylcarbonylamino, heteroarylcarbonylamino, (C₁-C₆)-alkoxycarbonylamino, (C₁-C₆)-alkylcarbonyl[(C₁-C₆)-alkyl]amino, arylcarbonyl[(C₁-C₆)-alkyl]amino, (C₃-C₆)-cycloalkylcarbonyl[(C₁-C₆)-alkyl]amino, (C₁-C₆)-alkyl[(C₁-C₆)-alkyl]amino, (C₁-C₆)-haloalkyl-[(C₁-C₆)-alkyl]amino, (C₁-C₆)-alkoxycarbonyl-[(C₁-C₆)-alkyl]amino, (C₁-C₆)-alkoxycarbonyl-(C₁-C₆)-alkyl-[(C₁-C₆)-alkyl]amino, (C₁-C₆)-alkylsulfonylamino, arylsulfonylamino, heteroarylsulfonylamino, (C₃-C₆)-cycloalkylsulfonylamino, (C₁-C₆)-alkylsulfonyl-[(C₁-C₆)-alkyl]amino, (C₃-C₆)-cycloalkylsulfonyl-[(C₁-C₆)-alkyl]amino, arylsulfonyl-[(C₁-C₆)-alkyl]amino, heteroarylsulfonyl[(C₁-C₆)-alkyl]amino, (C₁-C₆)-alkylcarbonyl[(C₁-C₆)-alkyl]amino, arylcarbonyl-[(C₁-C₆)-alkyl]amino, (C₃-C₆)-cycloalkylcarbonyl-[(C₁-C₆)-alkyl]amino, heteroarylcarbonyl-[(C₁-C₆)-alkyl]amino, (C₂-C₆)-alkenylamino, imino, arylimino, aryl-(C₁-C₆)-alkylimino, heteroarylimino, heterocyclylimino, (C₁-C₆)-haloalkylimino, (C₁-C₆)-alkylimino, (C₃-C₆)-cycloalkylimino, (C₁-C₆)-alkoxycarbonylimino, (C₃-C₆)-cycloalkoxycarbonylimino, (C₃-C₆)-cycloalkyl-(C₁-C₆)-alkoxycarbonylimino, aryl-(C₁-C₆)-alkoxycarbonylimino, (C₁-C₆)-alkylaminocarbonylimino, bis-[(C₁-C₆)-alkyl]aminocarbonylimino, (C₁-C₆)-alkylsulfinylimino, arylsulfinylimino, (C₃-C₆)-cycloalkylsulfinylimino, (C₁-C₆)-alkylthioimino, arylthioimino, (C₃-C₆)-cycloalkylthioimino, (C₁-C₆)-alkylsulfonylimino, arylsulfonylimino, (C₃-C₆)-cycloalkylsulfonylimino, optionally further substituted imino-(C₁-C₆)-alkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, (C₁-C₆)-alkyl, (C₁-C₆)-haloalkyl, (C₃-C₆)-cycloalkyl, optionally substituted phenyl, (C₂-C₆)-alkenyl, (C₂-C₆)-alkynyl, heterocyclyl, heteroaryl, (C₁-C₆)-alkoxy-(C₁-C₆)-alkyl or together form an exo methylene group which is optionally substituted further and part of a further ring,
R⁷, R⁸ independently of one another represent hydrogen, (C₁-C₆)-alkyl, (C₁-C₆)-haloalkyl, (C₃-C₆)-cycloalkyl, optionally substituted phenyl, (C₂-C₆)-alkenyl, (C₁-C₆)-alkynyl, heterocyclyl, heteroaryl, (C₁-C₆)-alkoxy-(C₁-C₆)-alkyl,
R⁹ represents hydrogen, hydroxy, (C₁-C₆)-alkyl, (C₃-C₆)-cycloalkyl, halogen, (C₂-C₆)-alkenyl-(C₁-C₆)-alkyl, (C₂-C₆)-alkynyl-(C₁-C₆)-alkyl, (C₁-C₆)-haloalkyl, (C₁-C₆)-alkoxy-(C₁-C₆)-alkyl, (C₂-C₆)-alkynyl, (C₂-C₆)-alkenyl, (C₃-C₆)-cycloalkyl-(C₁-C₆)-alkyl, cyano-(C₁-C₆)-alkyl, nitro-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylamino, (C₁-C₆)-alkylamino-(C₁-C₆)-alkyl, bis-[(C₁-C₆)-alkyl]amino-(C₁-C₆)-alkyl, aminocarbonyl-(C₁-C₆)-alkyl, (C₁-C₆)-alkylaminocarbonyl-(C₁-C₆)-alkyl, bis-[(C₁-C₆)-alkyl]aminocarbonyl-(C₁-C₆)-alkyl, (C₁-C₆)-alkoxycarbonyl-(C₁-C₆)-alkyl, hydroxycarbonyl-(C₁-C₆)-alkyl, (C₁-C₆)-alkylcarbonyl, (C₃-C₆)-cycloalkylcarbonyl, (C₁-C₆)-haloalkylcarbonyl, (C₁-C₆)-alkoxycarbonyl, (C₂-C₆)-alkenyloxycarbonyl, (C₁-C₆)-alkylaminocarbonyl, (C₃-C₆)-cyclopropylaminocarbonyl, (C₁-C₆)-alkylsulfonyl, arylsulfonyl, (C₁-C₆)-cycloalkylsulfonyl, aryl-(C₁-C₆)-alkylsulfonyl, (C₂-C₆)-alkenylsulfonyl, heteroarylsulfonyl, (C₂-C₆)-alkynylsulfonyl, (C₁-C₆)-alkylsulfinyl, arylsulfinyl, (C₃-C₆)-cycloalkylsulfinyl, (C₂-C₆)-alkenylsulfinyl, (C₂-C₆)-alkynylsulfinyl, arylsulfinyl, heteroarylsulfinyl, arylcarbonyl, heteroarylcarbonyl, (C₁-C₆)-alkoxycarbonyl-(C₁-C₆)-alkyl, (C₂-C₆)-alkenyloxycarbonyl-(C₁-C₆)-alkyl, hydroxycarbonyl-(C₁-C₆)-alkyl, cyano-(C₁-C₆)-alkylaminocarbonyl, (C₂-C₆)-alkynylaminocarbonyl, heterocyclylcarbonyl, heteroaryl-(C₁-C₆)-alkylaminocarbonyl, (C₂-C₆)-alkenyloxycarbonyl, (C₃-C₆)-cycloalkyl-(C₁-C₆)-alkoxycarbonyl, (C₃-C₆)-cycloalkyl-(C₁-C₆)-alkoxycarbonylcarbonyl, (C₁-C₆)-alkoxycarbonylcarbonyl, (C₃-C₆)-cycloalkyl-(C₁-C₆)-alkylaminocarbonyl, aryl-(C₁-C₆)-alkylaminocarbonyl or a negative charge,
R¹⁰ represents (C₁-C₆)-alkyl, optionally substituted (C₂-C₆)-alkenyl, optionally substituted imino-(C₁-C₆)-alkyl, optionally further substituted imino, optionally substituted (C₁-C₆)-alkyl-(C₂-C₆)-alkenyl, heteroaryl-(C₂-C₆)-alkenyl, aryl-(C₁-C₆)-alkyl, heteroaryl-(C₁-C₆)-alkyl, heterocyclyl-(C₁-C₆)-alkyl, (C₁-C₆)-alkoxycarbonyl-(C₁-C₆)-alkyl, (C₃-C₆)-cycloalkoxycarbonyl-(C₁-C₆)-alkyl, aryl-(C₁-C₆)-alkoxycarbonyl-(C₁-C₆)-alkyl, (C₁-C₆)-haloalkyl, amino-(C₁-C₆)-alkyl, bis-[(C₁-C₆)-alkyl]amino-(C₁-C₆)-alkyl, (C₁-C₆)-alkoxy-(C₁-C₆)-alkyl, (C₁-C₆)-alkylcarbonyl, arylcarbonyl, heteroarylcarbonyl, (C₃-C₆)-cycloalkylcarbonyl, (C₁-C₆)-alkoxycarbonyl, aryloxycarbonyl, (C₃-C₆)-cycloalkoxycarbonyl, (C₁-C₆)-alkylsulfonyl, (C₃-C₆)-cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford the general formula (Ia),

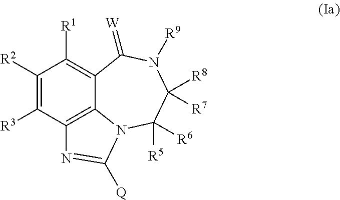

R⁴ and CR¹¹R¹² together with the atoms to which they are attached form a pyrrole ring which is substituted further by R¹³ and Q and thus together with the other substituents according to the definition afford the general formula (Ib),

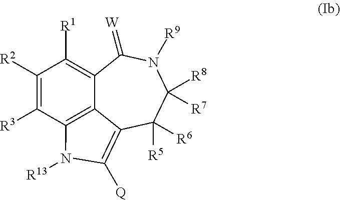

R⁵, R⁶ and N—R¹⁰ together with the atoms to which they are attached form a pyrrole ring which is optionally substituted further and thus together with the other substituents according to the definition afford the general formula (Ic),

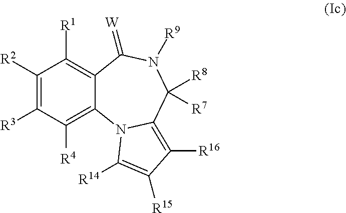

R⁴ and N—R¹⁰ together with the atoms to which they are attached form a pyrrole ring which is substituted further by R¹⁷ and Q and thus together with the other substituents according to the definition afford the general formula (Id),

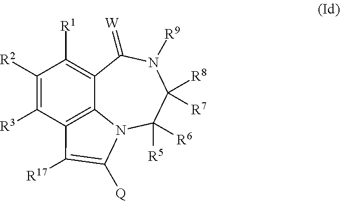

Q represents (C₁-C₆)-alkyl, (C₁-C₆)-haloalkyl, (C₃-C₆)-halocycloalkyl, (C₁-C₆)-alkoxy-(C₁-C₆)-haloalkyl, (C₁-C₆)-haloalkoxy-(C₁-C₆)-haloalkyl, (C₁-C₆)-alkylthio-(C₁-C₆)-haloalkyl, (C₁-C₆)-haloalkylthio-(C₁-C₆)-haloalkyl, (C₁-C₆)-alkoxy-(C₁-C₆)-alkoxy-(C₁-C₆)-haloalkyl, bis-[(C₁-C₆)-alkyl]amino-(C₁-C₆)-alkoxy-(C₁-C₆)-haloalkyl, (C₂-C₆)-alkenyl, (C₃-C₆)-cycloalkyl, (C₃-C₆)-cycloalkyl-(C₁-C₆)-alkyl, (C₄-C₆)-cycloalkenyl, optionally further substituted phenyl, aryl-(C₁-C₆)-alkyl, heteroaryl-(C₁-C₆)-alkyl, heterocyclyl-(C₁-C₆)-alkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, aryl-(C₂-C₆)-alkenyl, heteroaryl-(C₂-C₆)-alkenyl, heterocyclyl-(C₂-C₆)-alkenyl, aryl-(C₂-C₆)-alkynyl, heteroaryl-(C₂-C₆)-alkynyl, heterocyclyl-(C₂-C₆)-alkynyl, (C₃-C₆)-cycloalkyl-(C₂-C₆)-alkynyl, (C₁-C₆)-alkylamino-(C₁-C₆)-alkyl, bis-[(C₁-C₆)-alkyl]amino-(C₁-C₆)-alkyl, hydroxy-(C₁-C₆)-alkyl, (C₁-C₆)-alkoxy-(C₁-C₆)-alkyl, (C₁-C₆)-haloalkoxy-(C₁-C₆)-alkyl, tris-[(C₁-C₆)-alkyl]silyloxy-(C₁-C₆)-alkyl, bis-[(C₁-C₆)-alkyl]arylsilyloxy-(C₁-C₆)-alkyl, bis-[(C₁-C₆)-alkyl]-(C₁-C₆)-alkylsilyloxy-(C₁-C₆)-alkyl, bis-[(C₁-C₆)-alkyl]amino-(C₁-C₆)-alkoxy-(C₁-C₆)-alkyl, (C₁-C₆)-alkoxy-(C₁-C₆)-alkoxy-(C₁-C₆)-alkyl, aryloxy-(C₁-C₆)-alkyl, heteroaryloxy-(C₁-C₆)-alkyl, (C₁-C₆)-alkylthio-(C₁-C₆)-alkyl, (C₁-C₆)-haloalkylthio-(C₁-C₆)-alkyl, arylthio-(C₁-C₆)-alkyl, heteroarylthio-(C₁-C₆)-alkyl, (C₁-C₆)-alkoxycarbonyl-N-heterocyclyl, aryl-(C₁-C₆)-alkoxycarbonyl-N-heterocyclyl, (C₁-C₆)-alkyl-N-heterocyclyl, (C₁-C₆)-alkylsulfonyl-N-heterocyclyl, arylsulfonyl-N-heterocyclyl, heteroarylsulfonyl-N-heterocyclyl, (C₃-C₆)-cycloalkylsulfonyl-N-heterocyclyl, (C₁-C₆)-haloalkylsulfonyl-N-heterocyclyl, (C₁-C₆)-alkylcarbonyl-N-heterocyclyl, arylcarbonyl-N-heterocyclyl, heteroarylcarbonyl-N-heterocyclyl, (C₃-C₆)-cycloalkylcarbonyl-N-heterocyclyl, (C₃-C₆)-cycloalkyl-N-heterocyclyl, aryl-N-heterocyclyl, aryl-(C₁-C₆)-alkyl-N-heterocyclyl, bis-[(C₁-C₆)-alkyl]amino-(C₁-C₆)-alkyl-N-heterocyclyl, bis-[(C₁-C₆)-alkyl]aminosulfonyl-N-heterocyclyl, heteroaryloxyaryl, heteroaryloxyheteroaryl, aryloxyheteroaryl, (C₁-C₆)-alkylsulfinyl, (C₁-C₆)-alkylthio, (C₁-C₆)-alkylsulfonyl, (C₃-C₆)-cycloalkylsulfinyl, (C₃-C₆)-cycloalkylthio, (C₃-C₆)-cycloalkylsulfonyl, arylsulfinyl, arylthio, arylsulfonyl, amino, (C₁-C₆)-alkylamino, bis-[(C₁-C₆)-alkyl]amino, arylamino, aryl-(C₁-C₆)-alkylamino, (C₃-C₆)-cycloalkylamino, formyl, (C₁-C₆)-alkylcarbonyl, arylcarbonyl, imino-(C₁-C₆)-alkyl, (C₁-C₆)-alkylimino-(C₁-C₆)-alkyl, arylimino-(C₁-C₆)-alkyl, (C₁-C₆)-alkoxycarbonyl, (C₃-C₆)-cycloalkoxycarbonyl, (C₃-C₆)-cycloalkyl-(C₁-C₆)-alkoxycarbonyl, aryl-(C₁-C₆)-alkoxycarbonyl, aryl-(C₁-C₆)-alkylaminocarbonyl, aminocarbonyl, (C₁-C₆)-alkylaminocarbonyl, (C₃-C₆)-cycloalkylaminocarbonyl, bis-[(C₁-C₆)-alkyl]aminocarbonyl heterocyclyl-N-carbonyl, imino, (C₁-C₆)-alkylimino, arylimino, (C₃-C₆)-cycloalkylimino, (C₃-C₆)-cycloalkyl-(C₁-C₆)-alkylimino, hydroxyimino, (C₁-C₆)-alkoxyimino, (C₃-C₆)-cycloalkoxyimino, (C₃-C₆)-cycloalkyl-(C₁-C₆)-alkoxyimino, aryloxyimino, aryl-(C₁-C₆)-alkoxyimino, heteroaryl-(C₁-C₆)-alkoxyimino, heteroarylimino, heterocyclylimino, heterocyclyl-(C₁-C₆)-alkylimino, aminoimino, (C₁-C₆)-alkylaminoimino, arylaminoimino, heteroarylaminoimino, (C₃-C₆)-cycloalkylaminoimino, bis-[(C₁-C₆)-alkyl]aminoimino, aryl-(C₁-C₆)-alkylaminoimino, aryl[(C₁-C₆)-alkyl]aminoimino, (C₃-C₆)-cycloalkyl[(C₁-C₆)-alkyl]aminoimino, (C₃-C₆)-cycloalkyl-(C₁-C₆)-alkylaminoimino, heterocyclylaminoimino, heteroaryl-(C₁-C₆)-alkoxy-(C₁-C₆)-alkyl, aryl-(C₁-C₆)-alkoxy-(C₁-C₆)-alkyl, heterocyclyl-N—(C₁-C₆)-alkyl, aryl[(C₁-C₆)-alkyl]amino-(C₁-C₆)-alkyl, aryl-(C₁-C₆)-alkyl[(C₁-C₆)-alkyl]amino-(C₁-C₆)-alkyl, (C₁-C₆)-alkoxycarbonyl-(C₁-C₆)-alkylamino-(C₁-C₆)-alkyl, (C₁-C₆)-alkoxycarbonyl-(C₁-C₆)-alkyl[(C₁-C₆)-alkyl]amino-(C₁-C₆)-alkyl, heteroaryl[(C₁-C₆)-alkyl]amino-(C₁-C₆)-alkyl, heteroaryl-(C₁-C₆)-alkyl[(C₁-C₆)-alkyl]amino-(C₁-C₆)-alkyl, (C₃-C₆)-cycloalkyl[(C₁-C₆)-alkyl]amino-(C₁-C₆)-alkyl, (C₃-C₆)-cycloalkylamino-(C₁-C₆)-alkyl, (C₁-C₆)-alkoxy[(C₁-C₆)-alkoxy]-(C₁-C₆)-alkyl, heteroaryl-(C₁-C₆)-alkoxy-(C₁-C₆)-alkyl, aryl-(C₁-C₆)-alkoxy-(C₁-C₆)-alkylaryl, heterocyclyl-N—(C₁-C₆)-alkylaryl, aryl[(C₁-C₆)-alkyl]amino-(C₁-C₆)-alkylaryl, aryl-(C₁-C₆)-alkyl[(C₁-C₆)-alkyl]amino-(C₁-C₆)-alkylaryl, (C₁-C₆)-alkoxycarbonyl-(C₁-C₆)-alkylamino-(C₁-C₆)-alkylaryl, (C₁-C₆)-alkoxycarbonyl-(C₁-C₆)-alkyl[(C₁-C₆)-alkyl]amino-(C₁-C₆)-alkylaryl, heteroaryl[(C₁-C₆)-alkyl]amino-(C₁-C₆)-alkylaryl, heteroaryl-(C₁-C₆)-alkyl[(C₁-C₆)-alkyl]amino-(C₁-C₆)-alkylaryl, (C₃-C₆)-cycloalkyl[(C₁-C₆)-alkyl]amino-(C₁-C₆)-alkylaryl, (C₃-C₆)-cycloalkylamino-(C₁-C₆)-alkylaryl, (C₁-C₆)-alkoxy[(C₁-C₆)-alkoxy]-(C₁-C₆)-alkylaryl, (C₂-C₆)-alkynyl, (C₁-C₆)-alkyl-(C₂-C₆)-alkynyl, (C₁-C₆)-haloalkyl-(C₂-C₆)-alkynyl, (C₁-C₆)-alkylamino-(C₁-C₆)-alkylaryl, (C₁-C₆)-alkylamino-(C₁-C₆)-alkylaryl-(C₁-C₆)-alkyl, bis-[(C₁-C₆)-alkyl]amino-(C₁-C₆)-alkylaryl, bis-[(C₁-C₆)-alkyl]amino-(C₁-C₆)-alkylaryl-(C₁-C₆)-alkyl, heterocyclyl-N—(C₁-C₆)-alkylaryl-(C₁-C₆)-alkyl, (C₁-C₆)-alkoxycarbonyl-N-heterocyclyl-N—(C₁-C₆)-alkyl, aryl-(C₁-C₆)-alkoxycarbonyl-N-heterocyclyl-N—(C₁-C₆)-alkyl, (C₁-C₆)-alkyl-N-heterocyclyl-N—(C₁-C₆)-alkyl, (C₁-C₆)-alkylsulfonyl-N-heterocyclyl-N—(C₁-C₆)-alkyl, arylsulfonyl-N-heterocyclyl-N—(C₁-C₆)-alkyl, heteroarylsulfonyl-N-heterocyclyl-N—(C₁-C₆)-alkyl, (C₃-C₆)-cycloalkylsulfonyl-N-heterocyclyl-N—(C₁-C₆)-alkyl, (C₁-C₆)-haloalkylsulfonyl-N-heterocyclyl-N—(C₁-C₆)-alkyl, alkylcarbonyl-N-heterocyclyl-N-alkyl, arylcarbonyl-N-heterocyclyl-N—(C₁-C₆)-alkyl, heteroarylcarbonyl-N-heterocyclyl-N—(C₁-C₆)-alkyl, (C₃-C₆)-cycloalkylcarbonyl-N-heterocyclyl-N—(C₁-C₆)-alkyl, (C₃-C₆)-cycloalkyl-N-heterocyclyl-N—(C₁-C₆)-alkyl, (C₁-C₆)-alkoxycarbonyl-N-heterocyclyl-(C₁-C₆)-alkyl, aryl-(C₁-C₆)-alkoxycarbonyl-N-heterocyclyl-(C₁-C₆)-alkyl, (C₁-C₆)-alkyl-N-heterocyclyl-(C₁-C₆)-alkyl, (C₁-C₆)-alkylsulfonyl-N-heterocyclyl-(C₁-C₆)-alkyl, arylsulfonyl-N-heterocyclyl-(C₁-C₆)-alkyl, heteroarylsulfonyl-N-heterocyclyl-(C₁-C₆)-alkyl, (C₃-C₆)-cycloalkylsulfonyl-N-heterocyclyl-(C₁-C₆)-alkyl, (C₁-C₆)-haloalkylsulfonyl-N-heterocyclyl-(C₁-C₆)-alkyl, (C₁-C₆)-alkylcarbonyl-N-heterocyclyl-(C₁-C₆)-alkyl, arylcarbonyl-N-heterocyclyl-(C₁-C₆)-alkyl, heteroarylcarbonyl-N-heterocyclyl-(C₁-C₆)-alkyl, (C₃-C₆)-cycloalkylcarbonyl-N-heterocyclyl-(C₁-C₆)-alkyl, (C₃-C₆)-cycloalkyl-N-heterocyclyl-(C₁-C₆)-alkyl, aryl-(C₁-C₆)-alkylaminocarbonylaryl, aminocarbonylaryl, (C₁-C₆)-alkylaminocarbonylaryl, (C₃-C₆)-cycloalkylaminocarbonylaryl, bis-[(C₁-C₆)-alkyl]aminocarbonylaryl, aryl-(C₁-C₆)-alkylaminocarbonylaryl-(C₁-C₆)-alkyl, aminocarbonylaryl-(C₁-C₆)-alkyl, (C₁-C₆)-alkylaminocarbonylaryl-(C₁-C₆)-alkyl, (C₃-C₆)-cycloalkylaminocarbonylaryl-(C₁-C₆)-alkyl, bis-[(C₁-C₆)-alkyl]aminocarbonylaryl-(C₁-C₆)-alkyl, aryl-(C₁-C₆)-alkylamino-(C₁-C₆)-alkyl, heteroaryl-(C₁-C₆)-alkylamino-(C₁-C₆)-alkyl, (C₁-C₆)-alkylamino-(C₁-C₆)-alkoxyaryl, bis-[(C₁-C₆)-alkyl]amino-(C₁-C₆)-alkoxyaryl,
R¹³ represents hydrogen, (C₁-C₆)-alkyl, (C₃-C₆)-cycloalkyl, (C₃-C₆)-cycloalkyl-(C₁-C₆)-alkyl, (C₄-C₆)-cycloalkenyl, cyano-(C₁-C₆)-alkyl, (C₂-C₆)-alkenyl-(C₁-C₆)-alkyl, (C₁-C₆)-haloalkyl, (C₂-C₆)-alkynyl-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylcarbonyl, (C₃-C₆)-cycloalkylcarbonyl, (C₃-C₆)-cycloalkyl-(C₁-C₆)-alkylcarbonyl, arylcarbonyl, aryl-(C₁-C₆)-alkylcarbonyl, heteroarylcarbonyl, (C₁-C₆)-alkoxycarbonyl, (C₁-C₆)-alkylsulfonyl, (C₃-C₆)-cycloalkylsulfonyl, arylsulfonyl, heteroarylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, (C₃-C₆)-cycloalkylaminocarbonyl, bis-[(C₁-C₆)-alkyl]aminocarbonyl, (C₁-C₆)-alkoxy-(C₁-C₆)-alkyl, (C₁-C₆)-alkoxycarbonyl-(C₁-C₆)-alkyl,
R¹⁴, R¹⁵, R¹⁶ independently of one another represent hydrogen, (C₁-C₆)-alkyl, (C₃-C₆)-cycloalkyl, (C₃-C₆)-cycloalkyl-(C₁-C₆)-alkyl, (C₁-C₆)-haloalkyl, aryl, (C₁-C₆)-alkoxycarbonyl, (C₁-C₆)-hydroxycarbonyl, aminocarbonyl, (C₁-C₆)-alkylaminocarbonyl, (C₃-C₆)-cycloalkylaminocarbonyl,
R¹⁷ represents hydrogen, (C₁-C₆)-alkyl, (C₃-C₆)-cycloalkyl, (C₁-C₆)-haloalkyl, aryl-(C₁-C₆)-alkyl, heteroaryl-(C₁-C₆)-alkyl, heterocyclyl-(C₁-C₆)-alkyl, aryl, heteroaryl, heterocyclyl, formyl, imino, hydroxyimino, (C₁-C₆)-alkoxyimino, (C₃-C₆)-cycloalkoxyimino, halogen, (C₁-C₆)-alkoxycarbonyl, hydroxy-(C₁-C₆)-alkyl, (C₁-C₆)-alkoxy-(C₁-C₆)-alkyl, (C₁-C₆)-alkylcarbonyloxy-(C₁-C₆)-alkyl.

The definitions of radicals stated above in general terms or in areas of preference apply both to the end products of the formula (I) and correspondingly to the starting materials or intermediates required in each case for preparation. These radical definitions can be combined with one another as desired, i.e. including combinations between the given preferred ranges.

Essentially, the haloalkyl-substituted benzodiazepinones and benzazepinones of the general formula (Ia) mentioned above are likewise as yet unknown in the prior art. The invention therefore also provides haloalkyl-substituted benzodiazepinones of the general formula (Ia) or salts thereof

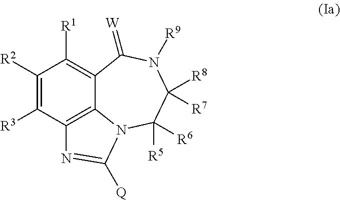

in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
W represents oxygen, sulfur,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
Q represents (C₁-C₁₂)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl,
   except for 2-(trifluoromethyl)-5,6-dihydroimidazo[4,5,1-jk][1,4]benzodiazepin-7(4H)-one and 2-(chloromethyl)-5,6-dihydroimidazo[4,5,1-jk][1,4]benzodiazepin-7(4H)-one.

Preference is given to compounds of the general formula (Ia) in which
R¹, R², R³ independently of one another represent hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, aryl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silylalkynyl, aryl-(C₂-C₆)-alkynyl, heteroaryl-(C₂-C₆)-alkynyl, (C₁-C₆)-alkyl-(C₁-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₁-C₆)-alkylcarbonylamino, (C₃-C₆)-cycloalkylcarbonylamino, arylcarbonylamino, (C₁-C₆)-alkoxycarbonylamino, heteroaryl-(C₁-C₆)-alkoxy, aryl-(C₁-C₆)-alkoxy, heterocyclyl-(C₁-C₆)-alkoxy, (C₃-C₆)-cycloalkyl-(C₁-C₆)-alkyl, (C₂-C₆)-haloalkenyl, (C₂-C₆)-haloalkynyl, heterocyclyl-(C₂-C₆)-alkynyl, (C₃-C₆)-halocycloalkoxy, (C₂-C₆)-haloalkynyloxy, arylthio, heteroarylthio, (C₁-C₆)-alkylsulfinyl, (C₁-C₆)-haloalkylsulfinyl, arylsulfinyl, heteroarylsulfinyl, (C₁-C₆)-alkylsulfonyl, (C₁-C₆)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₆)-alkyl]amino, (C₂-C₆)-alkenylamino, (C₁-C₆)-halocycloalkyl-(C₂-C₆)-alkynyl,
W represents oxygen, sulfur,
R⁵, R⁶ independently of one another represent hydrogen, (C₁-C₆)-alkyl, (C₁-C₆)-haloalkyl, (C₃-C₆)-cycloalkyl, aryl, (C₂-C₆)-alkenyl, (C₂-C₆)-alkynyl, heterocyclyl, heteroaryl, (C₁-C₆)-alkoxy-(C₁-C₆)-alkyl or together form an exo methylene group which is optionally substituted further and part of a further ring,
R⁷, R⁸ independently of one another represent hydrogen, (C₁-C₆)-alkyl, (C₁-C₆)-haloalkyl, (C₃-C₆)-cycloalkyl, aryl, (C₂-C₆)-alkenyl, (C₁-C₆)-alkynyl, heterocyclyl, heteroaryl, (C₁-C₆)-alkoxy-(C₁-C₆)-alkyl,
R⁹ represents hydrogen, hydroxy, (C₁-C₆)-alkyl, (C₃-C₆)-cycloalkyl, halogen, (C₂-C₆)-alkenyl-(C₁-C₆)-alkyl, (C₂-C₆)-alkynyl-(C₁-C₆)-alkyl, (C₁-C₆)-haloalkyl, (C₁-C₆)-alkoxy-(C₁-C₆)-alkyl, (C₂-C₆)-alkynyl, (C₂-C₆)-alkenyl, (C₃-C₆)-cycloalkyl-(C₁-C₆)-alkyl, cyano-(C₁-C₆)-alkyl, nitro-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylamino, (C₁-C₆)-alkylamino-(C₁-C₆)-alkyl, bis-[(C₁-C₆)-alkyl]amino-(C₁-C₆)-alkyl, aminocarbonyl-(C₁-C₆)-alkyl, (C₁-C₆)-alkylaminocarbonyl-(C₁-C₆)-alkyl, bis-[(C₁-C₆)-alkyl]aminocarbonyl-(C₁-C₆)-alkyl, (C₁-C₆)-alkoxycarbonyl-(C₁-C₆)-alkyl, hydroxycarbonyl-(C₁-C₆)-alkyl, (C₁-C₆)-alkylcarbonyl, (C₃-C₆)-cycloalkylcarbonyl, (C₁-C₆)-haloalkylcarbonyl, (C₁-C₆)-alkoxycarbonyl, (C₂-C₆)-alkenyloxycarbonyl, (C₁-C₆)-alkylaminocarbonyl, (C₃-C₆)-cyclopropylaminocarbonyl, (C₁-C₆)-alkylsulfonyl, arylsulfonyl, (C₁-C₆)-cycloalkylsulfonyl, aryl-(C₁-C₆)-alkylsulfonyl, (C₂-C₆)-alkenylsulfonyl, heteroarylsulfonyl, (C₂-C₆)-alkynylsulfonyl, (C₁-C₆)-alkylsulfinyl, arylsulfinyl, (C₃-C₆)-cycloalkylsulfinyl, (C₂-C₆)-alkenylsulfinyl, (C₂-C₆)-alkynylsulfinyl, arylsulfinyl, heteroarylsulfinyl, arylcarbonyl, heteroarylcarbonyl, (C₁-C₆)-alkoxycarbonyl-(C₁-C₆)-alkyl, (C₂-C₆)-alkenyloxycarbonyl-(C₁-C₆)-alkyl, hydroxycarbonyl-(C₁-C₆)-alkyl, cyano-(C₁-C₆)-alkylaminocarbonyl, (C₂-C₆)-alkynylaminocarbonyl, heterocyclylcarbonyl, heteroaryl-(C₁-C₆)-alkylaminocarbonyl, (C₂-C₆)-alkenyloxycarbonyl, (C₃-C₆)-cycloalkyl-(C₁-C₆)-alkoxycarbonyl, (C₃-C₆)-cycloalkyl-(C₁-C₆)-alkoxycarbonylcarbonyl, (C₁-C₆)-alkoxycarbonylcarbonyl, (C₃-C₆)-cycloalkyl-(C₁-C₆)-alkylaminocarbonyl, aryl-(C₁-C₆)-alkylaminocarbonyl or a negative charge,
Q represents (C₁-C₁₂)-haloalkyl, (C₃-C₆)-halocycloalkyl, (C₁-C₆)-alkoxy-(C₁-C₆)-haloalkyl, (C₁-C₆)-haloalkoxy-(C₁-C₆)-haloalkyl, (C₁-C₆)-alkylthio-(C₁-C₆)-haloalkyl, (C₁-C₆)-haloalkylthio-(C₁-C₆)-haloalkyl, (C₁-C₆)-alkoxy-(C₁-C₆)-alkoxy-(C₁-C₆)-haloalkyl, bis-[(C₁-C₆)-alkyl]amino-(C₁-C₆)-alkoxy-(C₁-C₆)-haloalkyl,
   except for 2-(trifluoromethyl)-5,6-dihydroimidazo[4,5,1-jk][1,4]benzodiazepin-7(4H)-one and 2-(chloromethyl)-5,6-dihydroimidazo[4,5,1-jk][1,4]benzodiazepin-7(4H)-one.

The invention therefore also provides haloalkyl-substituted azepinoindolones of the general formula (Ib) or salts thereof

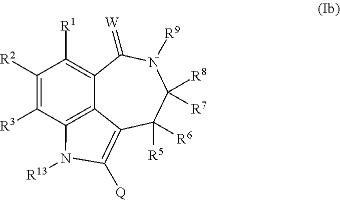

in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
W represents oxygen, sulfur,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
R¹³ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylcarbonyl, (C₃-C₈)-cycloalkylcarbonyl, (C₃-C₈)-cycloalkyl-(C₁-C₈)-alkylcarbonyl, arylcarbonyl, aryl-(C₁-C₈)-alkylcarbonyl, heteroarylcarbonyl, (C₁-C₈)-alkoxycarbonyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, bis-[(C₁-C₈)-alkyl]aminocarbonyl, (C₁-C₈)-alkoxy-(C₁-C₈)-alkyl, (C₁-C₈)-alkoxycarbonyl-(C₁-C₈)-alkyl;
Q represents (C₁-C₁₂)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl.

Preference is given to compounds of the general formula (Ib) in which
R¹, R², R³ independently of one another represent hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, optionally substituted phenyl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silylalkynyl, aryl-(C₂-C₆)-alkynyl, heteroaryl-(C₂-C₆)-alkynyl, (C₁-C₆)-alkyl-(C₁-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₁-C₆)-alkylcarbonylamino, (C₃-C₆)-cycloalkylcarbonylamino, arylcarbonylamino, (C₁-C₆)-alkoxycarbonylamino, heteroaryl-(C₁-C₆)-alkoxy, aryl-(C₁-C₆)-alkoxy, heterocyclyl-(C₁-C₆)-alkoxy, (C₃-C₆)-cycloalkyl-(C₁-C₆)-alkyl, (C₂-C₆)-haloalkenyl, (C₂-C₆)-haloalkynyl, heterocyclyl-(C₂-C₆)-alkynyl, (C₃-C₆)-halocycloalkoxy, (C₂-C₆)-haloalkynyloxy, arylthio, heteroarylthio, (C₁-C₆)-alkylsulfinyl, (C₁-C₆)-haloalkylsulfinyl, arylsulfinyl, heteroarylsulfinyl, (C₁-C₆)-alkylsulfonyl, (C₁-C₆)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₆)-alkyl]amino, (C₂-C₆)-alkenylamino, (C₁-C₆)-halocycloalkyl-(C₂-C₆)-alkynyl,
W represents oxygen, sulfur,
R⁵, R⁶ independently of one another represent hydrogen, (C₁-C₆)-alkyl, (C₁-C₆)-haloalkyl, (C₃-C₆)-cycloalkyl, aryl, (C₂-C₆)-alkenyl, (C₂-C₆)-alkynyl, heterocyclyl, heteroaryl, (C₁-C₆)-alkoxy-(C₁-C₆)-alkyl or together form an exo methylene group which is optionally substituted further and part of a further ring,
R⁷, R⁸ independently of one another represent hydrogen, (C₁-C₆)-alkyl, (C₁-C₆)-haloalkyl, (C₃-C₆)-cycloalkyl, aryl, (C₂-C₆)-alkenyl, (C₁-C₆)-alkynyl, heterocyclyl, heteroaryl, (C₁-C₆)-alkoxy-(C₁-C₆)-alkyl,
R⁹ represents hydrogen, hydroxy, (C₁-C₆)-alkyl, (C₃-C₆)-cycloalkyl, halogen, (C₂-C₆)-alkenyl-(C₁-C₆)-alkyl, (C₂-C₆)-alkynyl-(C₁-C₆)-alkyl, (C₁-C₆)-haloalkyl, (C₁-C₆)-alkoxy-(C₁-C₆)-alkyl, (C₂-C₆)-alkynyl, (C₂-C₆)-alkenyl, (C₃-C₆)-cycloalkyl-(C₁-C₆)-alkyl, cyano-(C₁-C₆)-alkyl, nitro-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylamino, (C₁-C₆)-alkylamino-(C₁-C₆)-alkyl, bis-[(C₁-C₆)-alkyl]amino-(C₁-C₆)-alkyl, aminocarbonyl-(C₁-C₆)-alkyl, (C₁-C₆)-alkylaminocarbonyl-(C₁-C₆)-alkyl, bis-[(C₁-C₆)-alkyl]aminocarbonyl-(C₁-C₆)-alkyl, (C₁-C₆)-alkoxycarbonyl-(C₁-C₆)-alkyl, hydroxycarbonyl-(C₁-C₆)-alkyl, (C₁-C₆)-alkylcarbonyl, (C₃-C₆)-cycloalkylcarbonyl, (C₁-C₆)-haloalkylcarbonyl, (C₁-C₆)-alkoxycarbonyl, (C₂-C₆)-alkenyloxycarbonyl, (C₁-C₆)-alkylaminocarbonyl, (C₃-C₆)-cyclopropylaminocarbonyl, (C₁-C₆)-alkylsulfonyl, arylsulfonyl, (C₁-C₆)-cycloalkylsulfonyl, aryl-(C₁-C₆)-alkylsulfonyl, (C₂-C₆)-alkenylsulfonyl, heteroarylsulfonyl, (C₂-C₆)-alkynylsulfonyl, (C₁-C₆)-alkylsulfinyl, arylsulfinyl, (C₃-C₆)-cycloalkylsulfinyl, (C₂-C₆)-alkenylsulfinyl, (C₂-C₆)-alkynylsulfinyl, arylsulfinyl, heteroarylsulfinyl, arylcarbonyl, heteroarylcarbonyl, (C₁-C₆)-alkoxycarbonyl-(C₁-C₆)-alkyl, (C₂-C₆)-alkenyloxycarbonyl-(C₁-C₆)-alkyl, hydroxycarbonyl-(C₁-C₆)-alkyl, cyano-(C₁-C₆)-alkylaminocarbonyl, (C₂-C₆)-alkynylaminocarbonyl, heterocyclylcarbonyl, heteroaryl-(C₁-C₆)-alkylaminocarbonyl, (C₂-C₆)-alkenyloxycarbonyl, (C₃-C₆)-cycloalkyl-(C₁-C₆)-alkoxycarbonyl, (C₃-C₆)-cycloalkyl-(C₁-C₆)-alkoxycarbonylcarbonyl, (C₁-C₆)-alkoxycarbonylcarbonyl, (C₃-C₆)-cycloalkyl-(C₁-C₆)-alkylaminocarbonyl, aryl-(C₁-C₆)-alkylaminocarbonyl or a negative charge,
R¹³ represents hydrogen, (C₁-C₆)-alkyl, (C₃-C₆)-cycloalkyl, (C₃-C₆)-cycloalkyl-(C₁-C₆)-alkyl, (C₄-C₆)-cycloalkenyl, cyano-(C₁-C₆)-alkyl, (C₂-C₆)-alkenyl-(C₁-C₆)-alkyl, (C₁-C₆)-haloalkyl, (C₂-C₆)-alkynyl-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylcarbonyl, (C₃-C₆)-cycloalkylcarbonyl, (C₃-C₆)-cycloalkyl-(C₁-C₆)-alkylcarbonyl, arylcarbonyl, aryl-(C₁-C₆)-alkylcarbonyl, heteroarylcarbonyl, (C₁-C₆)-alkoxycarbonyl, (C₁-C₆)-alkylsulfonyl, (C₃-C₆)-cycloalkylsulfonyl, arylsulfonyl, heteroarylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, (C₃-C₆)-cycloalkylaminocarbonyl, bis-[(C₁-C₆)-alkyl]aminocarbonyl, (C₁-C₆)-alkoxy-(C₁-C₆)-alkyl, (C₁-C₆)-alkoxycarbonyl-(C₁-C₆)-alkyl,
Q represents (C₁-C₈)-haloalkyl, (C₃-C₆)-halocycloalkyl, (C₁-C₆)-alkoxy-(C₁-C₆)-haloalkyl, (C₁-C₆)-haloalkoxy-(C₁-C₆)-haloalkyl, (C₁-C₆)-alkylthio-(C₁-C₆)-haloalkyl, (C₁-C₆)-haloalkylthio-(C₁-C₆)-haloalkyl, (C₁-C₆)-alkoxy-(C₁-C₆)-alkoxy-(C₁-C₆)-haloalkyl, bis-[(C₁-C₆)-alkyl]amino-(C₁-C₆)-alkoxy-(C₁-C₆)-haloalkyl.

The invention therefore also provides haloalkyl-substituted diazepinoindolones of the general formula (Id) or salts thereof

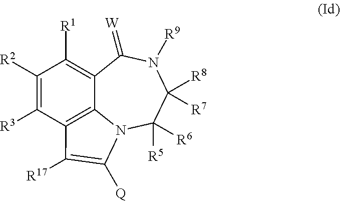

in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
W represents oxygen, sulfur,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
R¹⁷ represents hydrogen, (C₁-C₈)-alkyl, (C₃-C₈)-cycloalkyl, (C₁-C₈)-haloalkyl, aryl-(C₁-C₈)-alkyl, heteroaryl-(C₁-C₈)-alkyl, heterocyclyl-(C₁-C₈)-alkyl, aryl, heteroaryl, heterocyclyl, formyl, imino, hydroxyimino, (C₁-C₈)-alkoxyimino, (C₃-C₈)-cycloalkoxyimino, halogen, (C₁-C₈)-alkoxycarbonyl, hydroxy-(C₁-C₈)-alkyl, (C₁-C₈)-alkoxy-(C₁-C₈)-alkyl, (C₁-C₈)-alkylcarbonyloxy-(C₁-C₈)-alkyl,
Q represents (C₁-C₁₂)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl.

Preference is given to compounds of the general formula (Id) in which
R¹, R², R³ independently of one another represent hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, optionally substituted phenyl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silylalkynyl, aryl-(C₂-C₆)-alkynyl, heteroaryl-(C₂-C₆)-alkynyl, (C₁-C₆)-alkyl-(C₁-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₁-C₆)-alkylcarbonylamino, (C₃-C₆)-cycloalkylcarbonylamino, arylcarbonylamino, (C₁-C₆)-alkoxycarbonylamino, heteroaryl-(C₁-C₆)-alkoxy, aryl-(C₁-C₆)-alkoxy, heterocyclyl-(C₁-C₆)-alkoxy, (C₃-C₆)-cycloalkyl-(C₁-C₆)-alkyl, (C₂-C₆)-haloalkenyl, (C₂-C₆)-haloalkynyl, heterocyclyl-(C₂-C₆)-alkynyl, (C₃-C₆)-halocycloalkoxy, (C₂-C₆)-haloalkynyloxy, arylthio, heteroarylthio, (C₁-C₆)-alkylsulfinyl, (C₁-C₆)-haloalkylsulfinyl, arylsulfinyl, heteroarylsulfinyl, (C₁-C₆)-alkylsulfonyl, (C₁-C₆)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₆)-alkyl]amino, (C₂-C₆)-alkenylamino, (C₁-C₆)-halocycloalkyl-(C₂-C₆)-alkynyl,
W represents oxygen, sulfur,
R⁵, R⁶ independently of one another represent hydrogen, (C₁-C₆)-alkyl, (C₁-C₆)-haloalkyl, (C₃-C₆)-cycloalkyl, aryl, (C₂-C₆)-alkenyl, (C₂-C₆)-alkynyl, heterocyclyl, heteroaryl, (C₁-C₆)-alkoxy-(C₁-C₆)-alkyl or together form an exo methylene group which is optionally substituted further and part of a further ring,
R⁷, R⁶ independently of one another represent hydrogen, (C₁-C₆)-alkyl, (C₁-C₆)-haloalkyl, (C₃-C₆)-cycloalkyl, aryl, (C₂-C₆)-alkenyl, (C₁-C₆)-alkynyl, heterocyclyl, heteroaryl, (C₁-C₆)-alkoxy-(C₁-C₆)-alkyl,
R⁹ represents hydrogen, hydroxy, (C₁-C₆)-alkyl, (C₃-C₆)-cycloalkyl, halogen, (C₂-C₆)-alkenyl-(C₁-C₆)-alkyl, (C₂-C₆)-alkynyl-(C₁-C₆)-alkyl, (C₁-C₆)-haloalkyl, (C₁-C₆)-alkoxy-(C₁-C₆)-alkyl, (C₂-C₆)-alkynyl, (C₂-C₆)-alkenyl, (C₃-C₆)-cycloalkyl-(C₁-C₆)-alkyl, cyano-(C₁-C₆)-alkyl, nitro-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylamino, (C₁-C₆)-alkylamino-(C₁-C₆)-alkyl, bis-[(C₁-C₆)-alkyl]amino-(C₁-C₆)-alkyl, aminocarbonyl-(C₁-C₆)-alkyl, (C₁-C₆)-alkylaminocarbonyl-(C₁-C₆)-alkyl, bis-[(C₁-C₆)-alkyl]aminocarbonyl-(C₁-C₆)-alkyl, (C₁-C₆)-alkoxycarbonyl-(C₁-C₆)-alkyl, hydroxycarbonyl-(C₁-C₆)-alkyl, (C₁-C₆)-alkylcarbonyl, (C₃-C₆)-cycloalkylcarbonyl, (C₁-C₆)-haloalkylcarbonyl, (C₁-C₆)-alkoxycarbonyl, (C₂-C₆)-alkenyloxycarbonyl, (C₁-C₆)-alkylaminocarbonyl, (C₃-C₆)-cyclopropylaminocarbonyl, (C₁-C₆)-alkylsulfonyl, arylsulfonyl, (C₁-C₆)-cycloalkylsulfonyl, aryl-(C₁-C₆)-alkylsulfonyl, (C₂-C₆)-alkenylsulfonyl, heteroarylsulfonyl, (C₂-C₆)-alkynylsulfonyl, (C₁-C₆)-alkylsulfinyl, arylsulfinyl, (C₃-C₆)-cycloalkylsulfinyl, (C₂-C₆)-alkenylsulfinyl, (C₂-C₆)-alkynylsulfinyl, arylsulfinyl, heteroarylsulfinyl, arylcarbonyl, heteroarylcarbonyl, (C₁-C₆)-alkoxycarbonyl-(C₁-C₆)-alkyl, (C₂-C₆)-alkenyloxycarbonyl-(C₁-C₆)-alkyl, hydroxycarbonyl-(C₁-C₆)-alkyl, cyano-(C₁-C₆)-alkylaminocarbonyl, (C₂-C₆)-alkynylaminocarbonyl, heterocyclylcarbonyl, heteroaryl-(C₁-C₆)-alkylaminocarbonyl, (C₂-C₆)-alkenyloxycarbonyl, (C₃-C₆)-cycloalkyl-(C₁-C₆)-alkoxycarbonyl, (C₃-C₆)-cycloalkyl-(C₁-C₆)-alkoxycarbonylcarbonyl, (C₁-C₆)-alkoxycarbonylcarbonyl, (C₃-C₆)-cycloalkyl-(C₁-C₆)-alkylaminocarbonyl, aryl-(C₁-C₆)-alkylaminocarbonyl or a negative charge,
R¹⁷ represents hydrogen, (C₁-C₆)-alkyl, (C₃-C₆)-cycloalkyl, (C₁-C₆)-haloalkyl, aryl-(C₁-C₆)-alkyl, heteroaryl-(C₁-C₆)-alkyl, heterocyclyl-(C₁-C₆)-alkyl, aryl, heteroaryl, heterocyclyl, formyl, imino, hydroxyimino, (C₁-C₆)-alkoxyimino, (C₃-C₆)-cycloalkoxyimino, halogen, (C₁-C₆)-alkoxycarbonyl, hydroxy-(C₁-C₆)-alkyl, (C₁-C₆)-alkoxy-(C₁-C₆)-alkyl, (C₁-C₆)-alkylcarbonyloxy-(C₁-C₆)-alkyl,
Q represents (C₁-C₈)-haloalkyl, (C₃-C₆)-halocycloalkyl, (C₁-C₆)-alkoxy-(C₁-C₆)-haloalkyl, (C₁-C₆)-haloalkoxy-(C₁-C₆)-haloalkyl, (C₁-C₆)-alkylthio-(C₁-C₆)-haloalkyl, (C₁-C₆)-haloalkylthio-(C₁-C₆)-haloalkyl, (C₁-C₆)-alkoxy-(C₁-C₆)-alkoxy-(C₁-C₆)-haloalkyl, bis-[(C₁-C₆)-alkyl]amino-(C₁-C₆)-alkoxy-(C₁-C₆)-haloalkyl.

With regard to the compounds according to the invention, the terms used above and further below will be elucidated. These are familiar to the person skilled in the art and especially have the definitions elucidated hereinafter:

According to the invention, “arylsulfonyl” represents optionally substituted phenylsulfonyl or optionally substituted polycyclic arylsulfonyl, here especially optionally substituted naphthylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “cycloalkylsulfonyl”—alone or as part of a chemical group—is optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “alkylsulfonyl”—alone or as part of a chemical group—is straight-chain or branched alkylsulfonyl, preferably having 1 to 8 or having 1 to 6 carbon atoms, for example methylsulfonyl, ethylsulfonyl, n-propylsulfonyl, isopropylsulfonyl, n-butylsulfonyl, isobutylsulfonyl, sec-butylsulfonyl and tert-butylsulfonyl.

According to the invention, “heteroarylsulfonyl” represents optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—is straight-chain or branched S-alkyl, preferably having 1 to 8 or having 1 to 6 carbon atoms, for example methylthio, ethylthio, n-propylthio, Isopropylthio, n-butylthio, isobutylthio, sec-butylthio and tert-butylthio. Alkenylthio is an alkenyl radical bonded via a sulfur atom, alkynylthio is an alkynyl radical bonded via a sulfur atom, cycloalkylthio is a cycloalkyl radical bonded via a sulfur atom, and cycloalkenylthio is a cycloalkenyl radical bonded via a sulfur atom.

“Alkoxy” is an alkyl radical attached via an oxygen atom, alkenyloxy is an alkenyl radical attached via an oxygen atom, alkynyloxy is an alkynyl radical attached via an oxygen atom, cycloalkyloxy is a cycloalkyl radical attached via an oxygen atom, and cycloalkenyloxy is a cycloalkenyl radical attached via an oxygen atom.

The term “aryl” means an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Here, preferred aryl substituents are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bis-alkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. In the case of optionally substituted heterocyclyl, spirocyclic systems are also included, for example 1-oxa-5-azaspiro[2.3]hexyl. Unless defined differently, the heterocyclic ring contains preferably 3 to 9 ring atoms and in particular 3 to 6 ring atoms and one or more, preferably 1 to 4 and in particular 1, 2 or 3 heteroatoms in the heterocyclic ring, preferably from the group consisting of N, O and S, although no two oxygen atoms should be directly adjacent, for example, with one heteroatom from the group consisting of N, O and S, 1- or 2- or 3-pyrrolidinyl, 3,4-dihydro-2H-pyrrol-2- or 3-yl, 2,3-dihydro-1H-pyrrol-1- or 2- or 3- or 4- or 5-yl; 2,5-dihydro-1H-pyrrol-1- or 2- or 3-yl, 1- or 2- or 3- or 4-piperidinyl; 2,3,4,5-tetrahydropyridin-2- or 3- or 4- or 5-yl or 6-yl; 1,2,3,6-tetrahydropyridin-1- or 2- or 3- or 4- or 5- or 6-yl; 1,2,3,4-tetrahydropyridin-1- or 2- or 3- or 4- or 5- or 6-yl; 1,4-dihydropyridin-1- or 2- or 3- or 4-yl; 2,3-dihydropyridin-2- or 3- or 4- or 5- or 6-yl; 2,5-dihydropyridin-2- or 3- or 4- or 5- or 6-yl, 1- or 2- or 3- or 4-azepanyl; 2,3,4,5-tetrahydro-1H-azepin-1- or 2- or 3- or 4- or 5- or 6- or 7-yl; 2,3,4,7-tetrahydro-1H-azepin-1- or 2- or 3- or 4- or 5- or 6- or 7-yl; 2,3,6,7-tetrahydro-1H-azepin-1- or 2- or 3- or 4-yl; 3,4,5,6-tetrahydro-2H-azepin-2- or 3- or 4- or 5- or 6- or 7-yl; 4,5-dihydro-1H-azepin-1- or 2- or 3- or 4-yl; 2,5-dihydro-1H-azepin-1- or -2- or 3- or 4- or 5- or 6- or 7-yl; 2,7-dihydro-1H-azepin-1- or -2- or 3- or 4-yl; 2,3-dihydro-1H-azepin-1- or -2- or 3- or 4- or 5- or 6- or 7-yl; 3,4-dihydro-2H-azepin-2- or 3- or 4- or 5- or 6- or 7-yl; 3,6-dihydro-2H-azepin-2- or 3- or 4- or 5- or 6- or 7-yl; 5,6-dihydro-2H-azepin-2- or 3- or 4- or 5- or 6- or 7-yl; 4,5-dihydro-3H-azepin-2- or 3- or 4- or 5- or 6- or 7-yl; 1H-azepin-1- or -2- or 3- or 4- or 5- or 6- or 7-yl; 2H-azepin-2- or 3- or 4- or 5- or 6- or 7-yl; 3H-azepin-2- or 3- or 4- or 5- or 6- or 7-yl; 4H-azepin-2- or 3- or 4- or 5- or 6- or 7-yl, 2- or 3-oxolanyl (=2- or 3-tetrahydrofuranyl); 2,3-dihydrofuran-2- or 3- or 4- or 5-yl; 2,5-dihydrofuran-2- or 3-yl, 2- or 3- or 4-oxanyl (=2- or 3- or 4-tetrahydropyranyl); 3,4-dihydro-2H-pyran-2- or 3- or 4- or 5- or 6-yl; 3,6-dihydro-2H-pyran-2- or 3- or 4- or 5- or 6-yl; 2H-pyran-2- or 3- or 4- or 5- or 6-yl; 4H-pyran-2- or 3- or 4-yl, 2- or 3- or 4-oxepanyl; 2,3,4,5-tetrahydrooxepin-2- or 3- or 4- or 5- or 6- or 7-yl; 2,3,4,7-tetrahydrooxepin-2- or 3- or 4- or 5- or 6- or 7-yl; 2,3,6,7-tetrahydrooxepin-2- or 3- or 4-yl; 2,3-dihydrooxepin-2- or 3- or 4- or 5- or 6- or 7-yl; 4,5-dihydrooxepin-2- or 3- or 4-yl; 2,5-dihydrooxepin-2- or 3- or 4- or 5- or 6- or 7-yl; oxepin-2- or 3- or 4- or 5- or 6- or 7-yl; 2- or 3-tetrahydrothiophenyl; 2,3-dihydrothiophen-2- or 3- or 4- or 5-yl; 2,5-dihydrothiophen-2- or 3-yl; tetrahydro-2H-thiopyran-2- or 3- or 4-yl; 3,4-dihydro-2H-thiopyran-2- or 3- or 4- or 5- or 6-yl; 3,6-dihydro-2H-thiopyran-2- or 3- or 4- or 5- or 6-yl; 2H-thiopyran-2- or 3- or 4- or 5- or 6-yl; 4H-thiopyran-2- or 3- or 4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group consisting of N, O and S, such as, for example, 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyriazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl. Structural example of heterocycles which are optionally substituted further are also listed below:

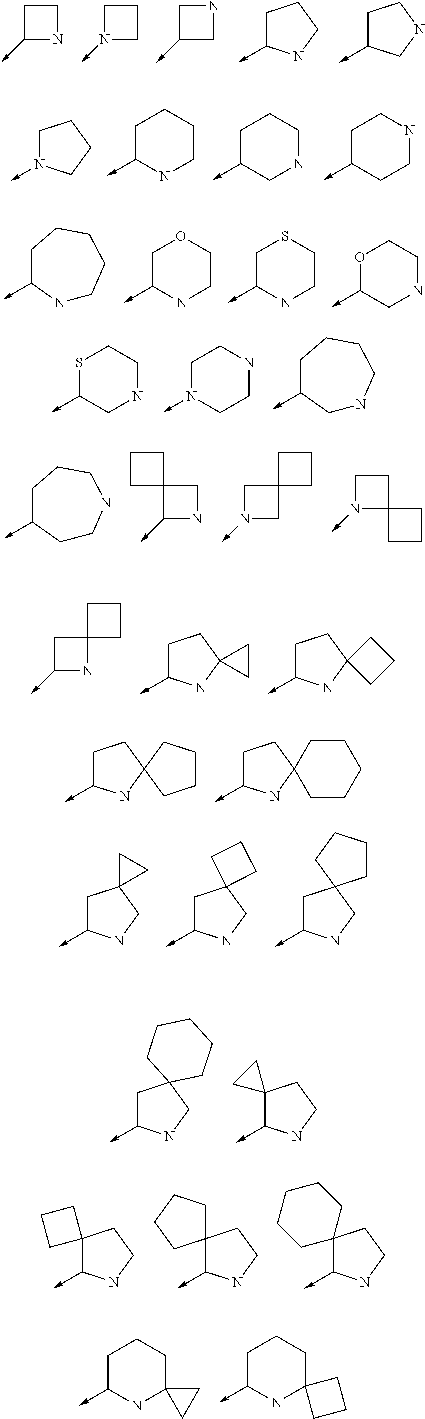

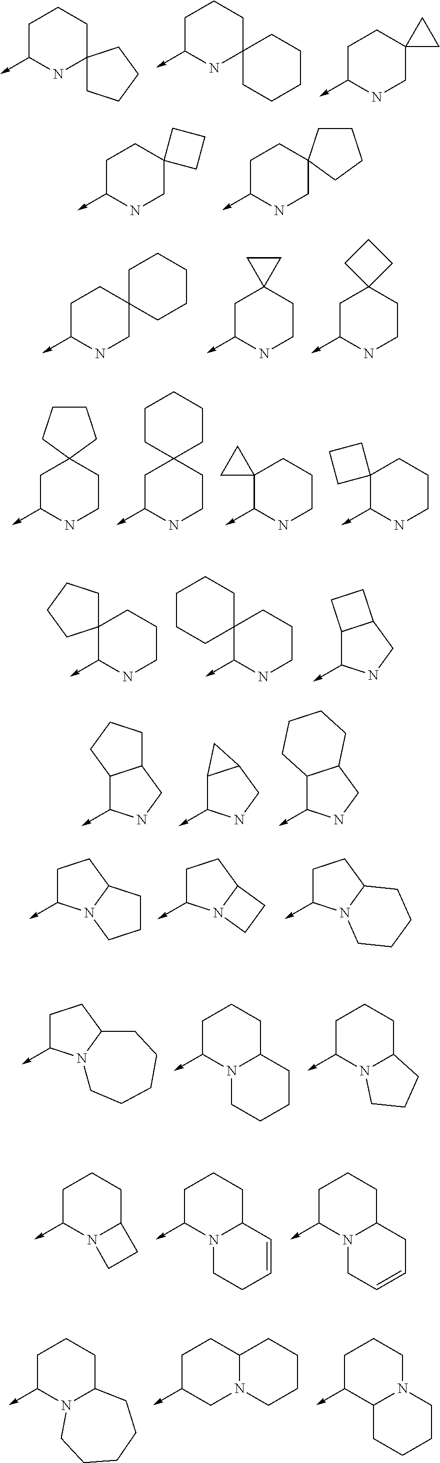

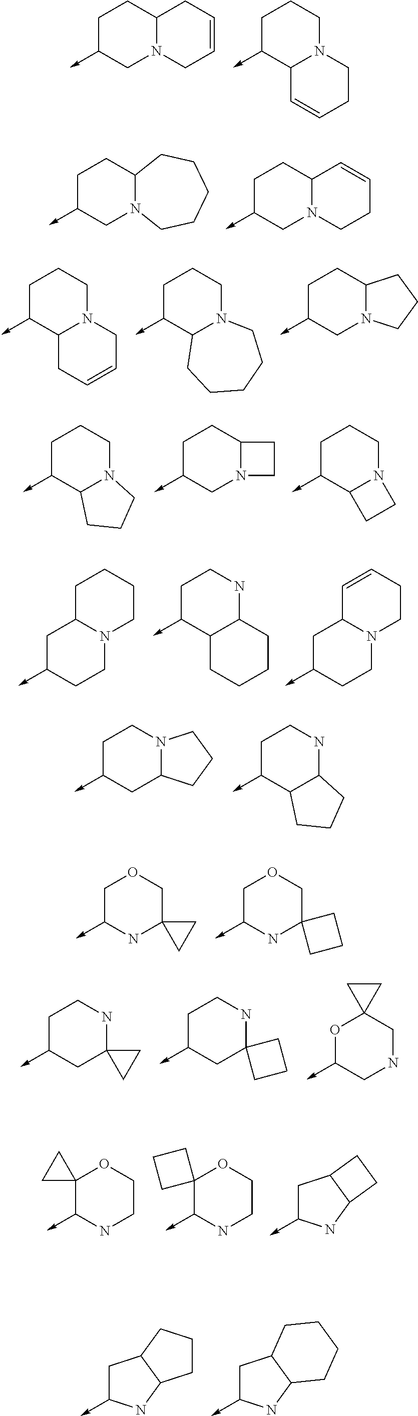

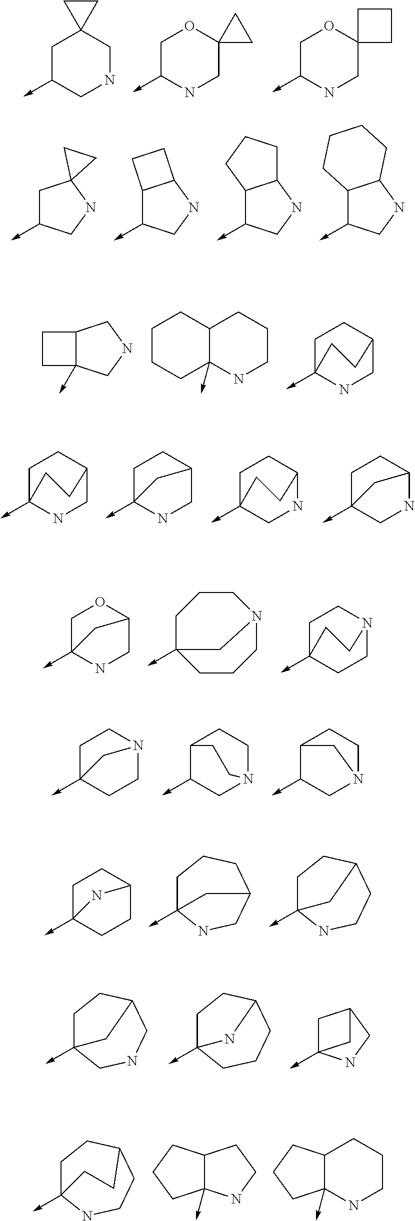

The heterocycles listed above are preferably substituted, for example, by hydrogen, halogen, alkyl, haloalkyl, hydroxy, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, tris-alkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bis-alkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bis-alkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the substituents specified further below, and additionally also oxo and thioxo. The oxo group as a substituent on a ring carbon atom is then, for example, a carbonyl group in the heterocyclic ring. As a result, lactones and lactams are preferably also included. The oxo group may also be present on the ring heteroatoms, which can exist in various oxidation states, for example on N and S, in which case they form, for example, the divalent groups N(O), S(O) (also SO for short) and S(O)2 (also SO2 for short) in the heterocyclic ring. In the case of —N(O)— and —S(O)— groups, both enantiomers in each case are included.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups according to the invention may also be substituted by one or more identical or different radicals. If two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” means, for example, fluorine, chlorine, bromine or iodine. If the term is used for a radical, “halogen” means, for example, a fluorine, chlorine, bromine or iodine atom.

According to the invention, “alkyl” means a straight-chain or branched open-chain, saturated hydrocarbon radical which is optionally mono- or polysubstituted, preferably unsubstituted. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine.

“Haloalkyl”, “-alkenyl” and “-alkynyl” are alkyl, alkenyl and alkynyl, respectively, which are partially or fully substituted by identical or different halogen atoms, for example monohaloalkyl such as CH₂CH₂Cl, CH₂CH₂Br, CHClCH₃, CH₂Cl, CH₂F; perhaloalkyl such as CCl₃, CClF₂, CFCl₂, CF₂CClF₂, CF₂CClFCF₃; polyhaloalkyl such as CH₂CHFCl, CF₂CClFH, CF₂CBrFH, CH₂CF₃; here, the term perhaloalkyl also comprises the term perfluoroalkyl.

Partly fluorinated alkyl means a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

Partly fluorinated haloalkyl means a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partly fluorinated haloalkyl also includes full substitution of the straight or branched chain by halogen including at least one fluorine atom.

Haloalkoxy is, for example, OCF₃, OCHF₂, OCH₂F, OCF₂CF₃, OCH₂CF₃ and OCH₂CH₂Cl; the situation is equivalent for haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl is, for example, vinyl which may optionally be substituted by further alkyl radicals, for example prop-1-en-1-yl, but-1-en-1-yl, allyl, 1-methylprop-2-en-1-yl, 2-methylprop-2-en-1-yl, but-2-en-1-yl, 1-methylbut-3-en-1-yl and 1-methylbut-2-en-1-yl, 2-methylprop-1-en-1-yl, 1-methylprop-1-en-1-yl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl, pentenyl, 2-methylpentenyl or hexenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-alkynyl is, for example, ethynyl, propargyl, 1-methylprop-2-yn-1-yl, 2-butynyl, 2-pentynyl or 2-hexynyl, preferably propargyl, but-2-yn-1-yl, but-3-yn-1-yl or 1-methylbut-3-yn-1-yl.

The term “cycloalkyl” means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene. Optionally substituted cycloalkyl also includes polycyclic aliphatic systems, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl (norbornyl), bicyclo[2.2.2]octan-2-yl, adamantan-1-yl and adamantan-2-yl. The term “(C₃C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

“Cycloalkenyl” means a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, means the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene is a carbocyclic radical attached via a double bond.

The term “stannyl” represents a further-substituted radical containing a tin atom; “germanyl” analogously represents a further-substituted radical containing a germanium atom. “Zirconyl” represents a further-substituted radical containing a zirconium atom. “Hafnyl” represents a further-substituted radical containing a hafnium atom. “Boryl”, “borolanyl” and “borinanyl” represent further-substituted and optionally cyclic groups each containing a boron atom. “Plumbanyl” represents a further-substituted radical containing a lead atom. “Hydrargyl” represents a further-substituted radical containing a mercury atom. “Alanyl” represents a further-substituted radical containing an aluminum atom. “Magnesyl” represents a further-substituted radical containing a magnesium atom. “Zincyl” represents a further-substituted radical containing a zinc atom.

Depending on the nature of the substituents and the way in which they are attached, the compounds of the general formula (I) may be present as stereoisomers. The formula (I) embraces all possible stereoisomers defined by the specific three-dimensional form thereof, such as enantiomers, diastereomers, Z and E isomers. If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomer excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

### Synthesis:

Substituted benzodiazepinones and benzazepinones can be prepared by known processes (cf. J. Med. Chem. 2003, 46, 210; Bioorg. Med. Chem. 2003, 11, 3695; J. Med. Chem. 2004, 47, 5467; Synlett 2007, 1106; WO200116136; WO2003057699; DE19946289; WO2005012305). Various literature preparation routes were used to form the core structure, and some were optimized (see Scheme 1). Selected detailed synthesis examples are cited in the next section. Here, the synthesis routes employed and investigated for the preparation of substituted benzodiazepinones proceed from commercially available or easily preparable 2-halo-3-nitrobenzoic acids. The optionally further substituted 2-halo-3-nitrobenzoic acid in question can be converted with the aid of a suitable acid chloride (e.g. thionyl chloride or oxalyl chloride) and a suitable alcohol (e.g. methanol or ethanol) into the corresponding benzoic ester. The optionally further substituted 2-halo-3-nitrobenzoic ester thus obtained is then, by reaction with an optionally further substituted diaminoethane using a suitable base (e.g. sodium carbonate or potassium carbonate) in a polar-aprotic solvent (e.g. isopropanol, n-propanol, 1-butanol, 2-butanol), converted into an optionally further substituted 9-nitro-5H-1,4-benzodiazepin-5-one, which is reduced either with hydrogen in the presence of palladium on carbon in a suitable solvent or with tin(II) chloride to an optionally further substituted 9-amino-5H-1,4-benzodiazepin-5-one (Scheme 1). The radicals R¹, R², R³, R⁴, R⁵, R⁶, R⁷, R⁸ and Q mentioned in Scheme 1 below have the meanings defined above.

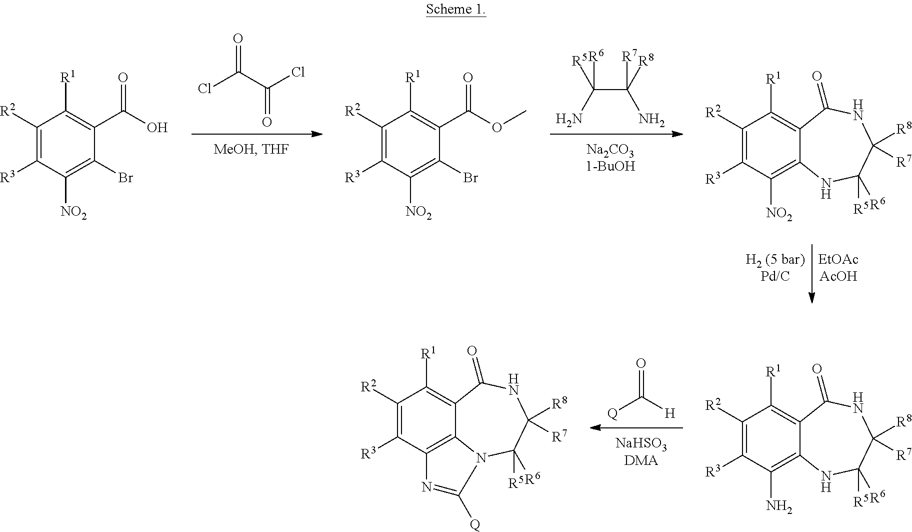

In the next step, the optionally further substituted 9-amino-5H-1,4-benzodiazepin-5-one obtained in this manner can be converted via various reaction variants, e.g. condensation with a carboxylic acid, with an aldehyde or an amidoxime, into the desired substituted imidazobenzodiazepinone. Any functional groups present at the substituents of the imidazolyl moiety can then be reacted further using suitable reagents. Thus, for example, terephthalaldehyde monodiethyl acetal can be reacted by condensation with an optionally substituted 9-amino-5H-1,4-benzodiazepin-5-one to give the desired target compound, and the acetal group can then be cleaved with a suitable acid (e.g. sulfuric acid in a suitable protic solvent) to give an aldehyde group. The aldehyde group in question can be converted via sodium cyanoborohydride-mediated reductive amination into the corresponding amines or via hydride-mediated reduction into the corresponding alcohol.

The radicals R¹, R², R³, R⁴, R⁵, R⁶, R⁷, R⁸ and Q mentioned in Scheme 2 below have the meanings defined above. Moreover, in Scheme 2 methyl and ethyl as substituents are shown in an exemplary manner as representatives for the groups according to the invention.

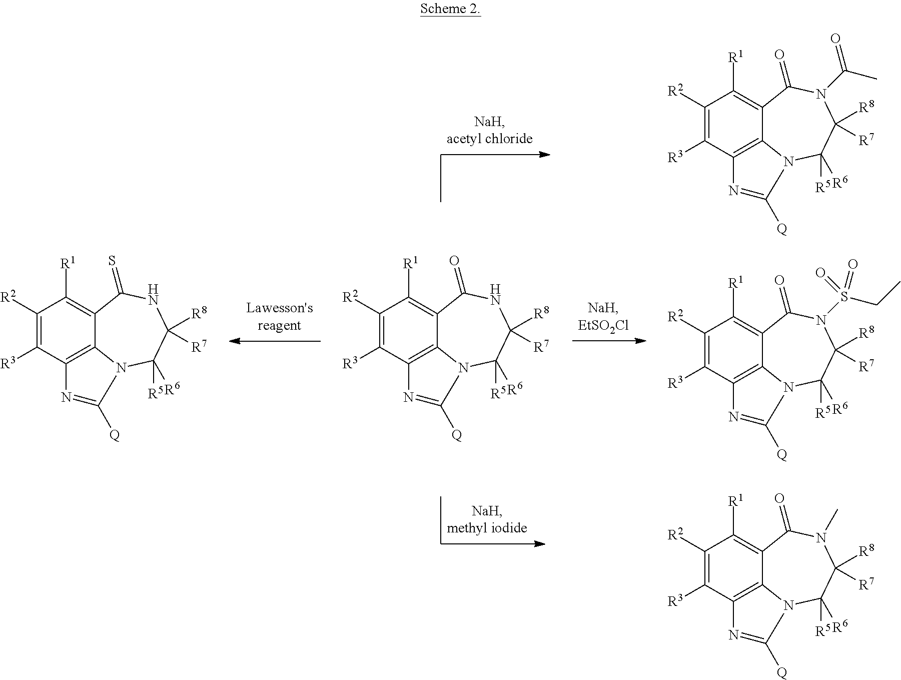

Functionalization of the benzodiazepinone-NH group is achieved by deprotonation using a suitable base, e.g. sodium hydride in an aprotic solvent, and subsequent reaction with a suitable electrophile, e.g. an acyl chloride, a sulfonyl chloride, an alkyl halide or a chloroformate. Moreover, the amide group of the imidazobenzodiazepinones prepared according to the invention can be converted into the corresponding thioamide using 2,4-bis-(4-methoxyphenyl)-1,3,2,4-dithiadiphosphetane 2,4-disulfide (Scheme 2). Substituted 3,4-dihydro[1,4]diazepino[6,7,1-hi]indol-1 (2H)-ones according to the invention can be prepared in a multistage synthesis starting with 2-iodoaniline. Here, optionally further substituted 2-iodoaniline is N-alkylated with the aid of propiolactone and then, via a Friedel-Crafts acylation, converted into an intermediate substituted 2,3-dihydroquinolin-4(1H)-one. The substituted 2,3-dihydroquinolin-4(1H)-one in question is then converted with the aid of sodium azide into the corresponding optionally further substituted 9-iodo-1,2,3,4-tetrahydro-5H-1,4-benzodiazepin-5-one. Transition metal-mediated coupling with a suitable substituted alkyne using a suitable palladium catalyst (e.g. bistriphenylphosphinepalladium dichloride) and a suitable copper salt (e.g. CuI) gives an optionally further substituted 9-alkynyl-1,2,3,4-tetrahydro-5H-1,4-benzodiazepin-5-one which, in a further transition metal-catalyzed reaction with the aid of a suitable palladium catalyst (e.g. palladium(II) chlorid) in a suitable polar-aprotic solvent (e.g. acetonitrile) is converted into the desired optionally further substituted 3,4-dihydro[1,4]diazepino[6,7,1-hi]indol-1(2H)-one (Scheme 3). The radicals R¹, R², R³, R⁴, R⁵, R⁶, R⁷, R⁸ and Q mentioned in Scheme 3 below have the meanings defined above.

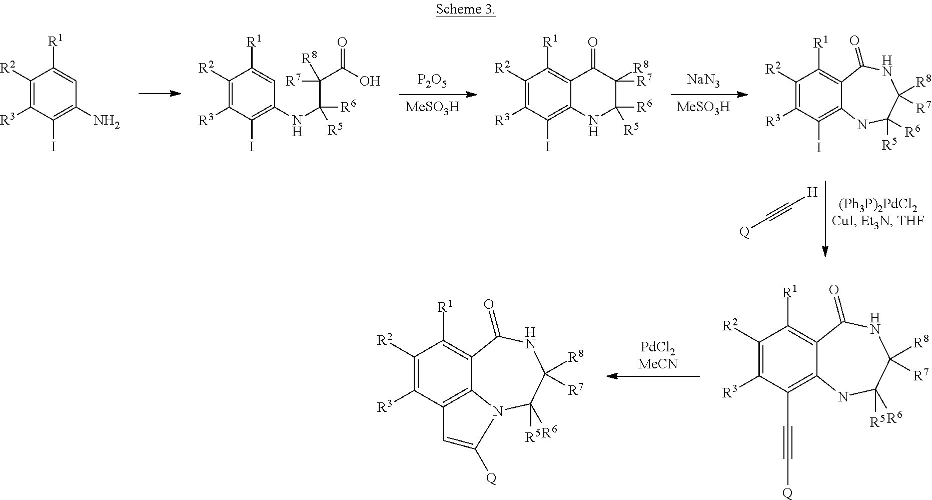

Optionally further substituted 4,5-dihydro-6H-pyrrolo[1,2-a][1,4]benzodiazepin-6-ones according to the invention can be prepared via a multistage synthesis route starting with optionally further substituted aminomethylfuranes. By reacting optionally further substituted 2-nitrobenzoic acids with thionyl chloride or oxalyl chloride and subsequent reaction with the optionally further substituted aminomethylfurans in question using a suitable amine base (e.g. triethylamine or diisopropylethylamine) in a suitable polar-aprotic solvent (e.g. tetrahydrofuran), it is possible to obtain substituted 2-nitrobenzamides as intermediates which, in the next reaction step, are converted by Raney nickel-mediated reduction using hydrazine in a suitable polar-protic solvent (e.g. ethanol) into the corresponding substituted 2-aminobenzamides (Scheme 4). The radicals R¹, R², R³, R⁴, R⁷, R⁸, R⁹, R¹⁴, R¹⁵, R¹⁶ mentioned in Scheme 4 below have the meanings defined above.

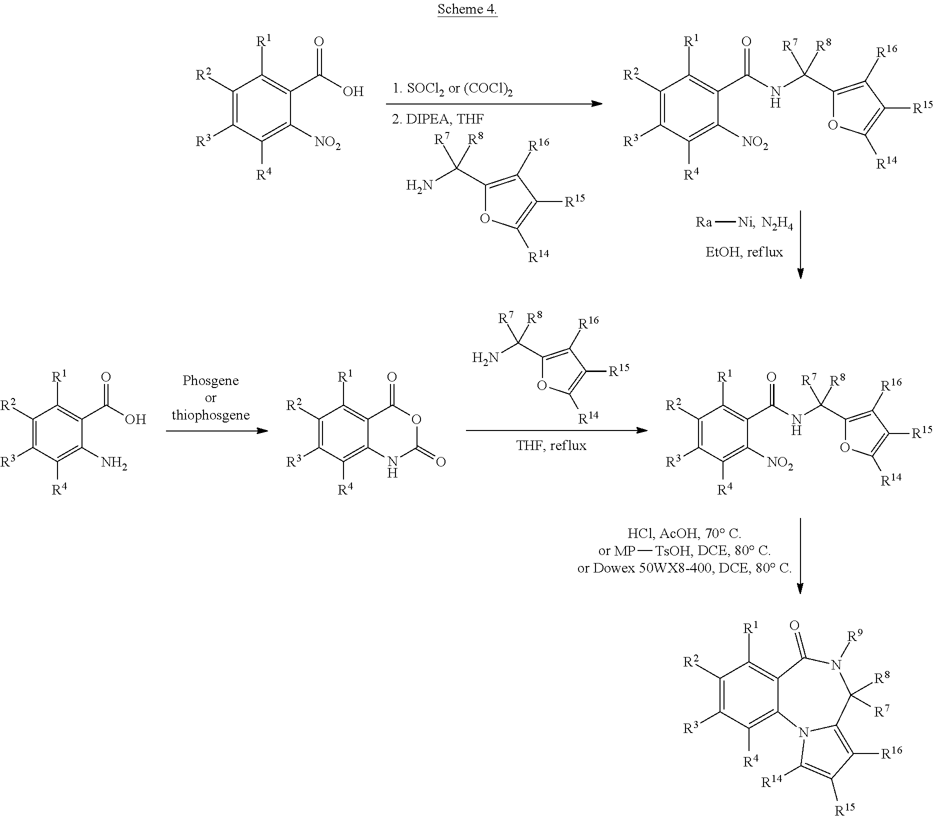

The reaction of optionally further substituted 2-aminobenzoic acids with phosgene or triphosgene and the subsequent reaction with optionally further substituted aminomethylfurans in a suitable aprotic solvent (e.g. tetrahydrofuran) under reflux conditions offers an alternative synthetic access to the desired substituted 2-aminobenzamide intermediates. By acid-mediated cyclization of the resulting substituted 2-aminobenzamides with the aid of a suitable reagent (e.g. hydrochloric acid, polymer-supported p-toluenesulfonic acid or DOWEX 50WX8-400) at elevated temperature in a suitable solvent (e.g. acetic acid or dichloroethane), it is possible to obtain the desired optionally further substituted 4,5-dihydro-6H-pyrrolo[1,2-a][1,4]benzodiazepin-6-ones according to the invention (Scheme 4).

The ¹H NMR, ¹³C NMR and ¹⁹F-NMR spectroscopic data which are reported for the chemical examples described in the paragraphs which follow (400 MHz for ¹H NMR and 150 MHz for ¹³C NMR and 375 MHz for ¹⁹F-NMR, solvent: CDCl₃, CD₃OD or d₆-DMSO, internal standard: tetramethylsilane δ=0.00 ppm), were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets, tt=triplet of triplets.

### SYNTHESIS EXAMPLES

### No. A1-1: 9-Nitro-1,2,3,4-tetrahydro-5H-1,4-benzodiazepin-5-one

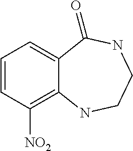 [SMILES: O=C1NCCNc2c1cccc2[N+](=O)[O-]]

At room temperature, 2-bromo-3-nitrobenzoic acid (1.0 g, 3.65 mmol) was dissolved in abs. tetrahydrofuran (10 ml) under argon. After 5 min, oxalyl chloride (1.59 ml, 18.20 mmol) was slowly added dropwise with vigorous stirring. The resulting reaction solution was heated to 70° C. and stirred at this temperature for 2 h. After cooling to room temperature, the solvent was concentrated under reduced pressure. The residue that remained was taken up in methanol (10 ml), stirred at room temperature for 2 h and the solvent was then removed under reduced pressure. This gave methyl 2-bromo-3-nitrobenzoate as a colorless solid (1.0 g, 95% of theory). At room temperature and under argon, methyl 2-bromo-3-nitrobenzoate (500 mg, 1.92 mmol) was dissolved in n-butanol (3.0 ml), and sodium carbonate (203 mg, 1.92 mmol) and ethylenediamine (0.13 ml, 1.92 mmol) were added. The resulting reaction solution was then stirred at a temperature of 80° C. for 6 h, when an orange precipitate was observed. After cooling to room temperature, the precipitate obtained was filtered off with suction and washed repeatedly with mother liquor. Subsequent drying gave 9-nitro-1,2,3,4-tetrahydro-5H-1,4-benzodiazepin-5-one in the form of a light-orange solid (350 mg, 87% of theory). ¹H-NMR (400 MHz, d₆-DMSO δ, ppm) 8.70 (br. t, 1H, NH), 8.39 (br. t, 1H, NH), 8.23 (d, 1H), 8.14 (d, 1H), 6.75 (dd, 1H), 3.64 (m, 2H), 3.34 (m, 2H).

### No. A1-41: 9-Amino-1,2,3,4-tetrahydro-5H-1,4-benzodiazepin-5-one

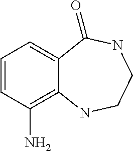 [SMILES: Nc1cccc2c1NCCNC2=O]

9-Nitro-1,2,3,4-tetrahydro-5H-1,4-benzodiazepin-5-one (5.0 g, 24.1 mmol) was taken up in a mixture of ethyl acetate (75 ml) and acetic acid (15 ml), a catalytic amount of palladium on carbon (10% wet, 1.0 g, 0.96 mmol) was added and hydrogen at a pressure of 3.5 bar was applied in a laboratory high pressure reactor. After the hydrogen uptake had gone to completion, the catalyst was filtered off, the solvent was removed under reduced pressure and the residue was triturated with ethyl acetate. Filtration with suction of the precipitated crude product gave 9-amino-1,2,3,4-tetrahydro-5H-1,4-benzodiazepin-5-one in the form of a slightly yellowish solid (3.5 g, 81% of theory). ¹H-NMR (400 MHz, d₆-DMSO δ, ppm) 7.88 (br. t, 1H, NH), 7.03 (d, 1H), 6.68 (d, 1H), 6.46 (dd, 1H), 4.96 (br. s, 2H, NH), 3.59 (br. t, 1H, NH), 3.41 (m, 2H), 3.20 (m, 2H).

### No. B1-448: 2-(Difluoromethyl)-5,6-dihydroimidazo[4,5,1-jk][1,4]benzodiazepin-7(4H)-one

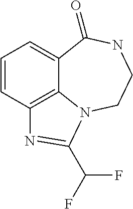 [SMILES: O=C1NCCn2c(C(F)F)nc3cccc1c32]

9-Amino-1,2,3,4-tetrahydro-5H-1,4-benzodiazepin-5-one (100 mg, 0.56 mmol) was taken up in difluoroacetic acid (3 ml) and, with stirring, heated to reflux temperature (120° C.) for 6 h. The reaction solution was then cooled to room temperature, carefully made basic (pH 8-9) using saturated sodium bicarbonate solution and repeatedly extracted vigorously with ethyl acetate. The combined organic phases were dried over magnesium sulfate, filtered and concentrated. Purification of the resulting crude product by column chromatography gave 2-(difluoromethyl)-5,6-dihydroimidazo[4,5,1-jk][1,4]benzodiazepin-7(4H)-one in the form of a colorless solid (70 mg, 49% of theory). ¹H-NMR (400 MHz, d₆-DMSO δ, ppm) 8.50 (t, 1H, NH), 8.01 (m, 2H), 7.52-7.30 (t, 1H), 7.42 (t, 1H), 4.5 (m, 2H), 3.65 (m, 2H).

No. B1-38: 2-(4-Chlorophenyl)-5,6-dihydroimidazo[4,5,1-jk][1,4]benzodiazepin-7(4H)-one

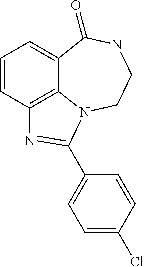 [SMILES: O=C1NCCn2c(-c3ccc(Cl)cc3)nc3cccc1c32]

9-Amino-1,2,3,4-tetrahydro-5H-1,4-benzodiazepin-5-one (50 mg, 0.28 mmol) and 4-chlorobenzaldehyde (44 mg, 0.31 mmol) were dissolved in N,N-dimethylacetamide (2 ml) and heated at reflux temperature with stirring for 2 h. The reaction solution was then cooled to room temperature, carefully made basic (pH 8-9) using saturated sodium bicarbonate solution and repeatedly extracted vigorously with dichloromethane. The combined organic phases were dried over magnesium sulfate, filtered and concentrated. Purification of the resulting crude product by column chromatography gave 2-(4-chlorophenyl)-5,6-dihydroimidazo[4,5,1-jk][1,4]benzodiazepin-7(4H)-one in the form of a colorless solid (60 mg, 71% of theory). ¹H-NMR (400 MHz, CDCl_{3 δ, ppm)} 8.15 (dd, 1H), 8.04 (dd, 1H), 7.75 (dd, 2H), 7.55 (dd, 2H), 7.45 (m, 2H), 4.50 (m, 2H), 3.75 (m, 2H).

### No. E42-4: 8,9-Dimethoxy-1-methyl-4,5-dihydro-6H-pyrrolo[1,2-a][1,4]benzodiazepin-6-one

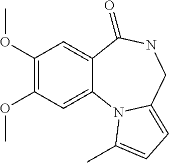 [SMILES: COc1cc2c(cc1OC)-n1c(C)ccc1CNC2=O]

A solution of veratroyl chloride (10 mmol) in abs. Toluene (30 ml) was added dropwise to a solution of methylfurfurylamine (12 mmol) in abs. toluene (30 ml) over a period of 20 minutes. Under argon, the resulting reaction mixture was then stirred at room temperature for about 2 h, saturated sodium bicarbonate solution was added after the reaction had gone to completion and the mixture was extracted thoroughly. The organic phase was separated off, washed with water, dried over sodium sulfate, filtered and concentrated under reduced pressure. Purification of the resulting crude product by column chromatography gave 4,5-dimethoxy-N-[(5-methyl-2-furyl)methyl]-2-nitrobenzamide in the form of a slightly yellowish solid. 4,5-Dimethoxy-N-[(5-methyl-2-furyl)methyl]-2-nitrobenzamide (4 mmol), hydrazine hydrate (1.4 ml) and Raney nickel (0.95 g) were combined in ethanol (60 ml) and then stirred under reflux conditions for 30 min. After cooling to room temperature, the reaction mixture was filtered off through Celite, the filter cake was washed with ethanol and the filtrate was concentrated under reduced pressure. Purification of the resulting crude product by column chromatography gave 4,5-dimethoxy-N-[(5-methyl-2-furyl)methyl]-2-aminobenzamide in the form of a colorless solid. 4,5-Dimethoxy-N-[(5-methyl-2-furyl)methyl]-2-aminobenzamide (2 mmol), glacial acetic acid (10 ml) and conc. HCl (1.5 ml) were then combined and stirred at a temperature of 70° C. for 3 h. After the reaction had gone to completion, the reaction mixture was added to ice-water and adjusted to pH 7 using sat. sodium bicarbonate solution. The precipitated solid was filtered off with suction, giving 8,9-dimethoxy-1-methyl-4,5-dihydro-6H-pyrrolo[1,2-a][1,4]benzodiazepin-6-one (288 mg, 53% of theory). ¹H-NMR (300 MHz, CDCl₃ δ, ppm) 7.45 (s, 1H), 6.82 (s, 1H), 6.75 (br. s, 1H, NH), 6.10 (m, 2H), 4.15 (m, 2H), 3.93 (d, 6H), 2.33 (s, 3H).

The compounds listed below are obtained analogously to the preparation examples given above and referred to at the appropriate place and taking into account the general information regarding the preparation of substituted benzodiazepinones and benzazepinones of the general formula (I).

A1. Compounds A1-1 to A1-300 of the general formula (I) in which R¹, R², R³ and R⁹ represent hydrogen and X, W, R⁴, R⁵, R⁶, R⁷, R⁸ correspond to the definitions (Nos 1 bis 300; corresponding to compounds A1-1 to A1-300) in Table 1 below. An arrow in a definition given in Table 1 for R⁵ and R⁷ represents a bond of the radical in question to the core structure (I); in this case, the two groups R⁵ and R⁷ together form a saturated or partially saturated ring.

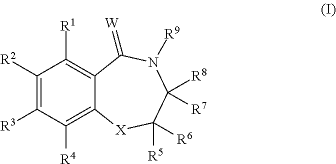

TABLE 1

| No. | R⁴ | W | X | R⁵ | R⁶ | R⁷ | R⁸ |
|---|---|---|---|---|---|---|---|
| 1 | NO₂ | O | N—H | H | H | H | H |
| 2 | NO₂ | O | N—H | CH₃ | H | H | H |
| 3 | NO₂ | O | N—H | H | H | CH₃ | H |
| 4 | NO₂ | O | N—H | CH₃ | CH₃ | H | H |
| 5 | NO₂ | O | N—H | CH₃ | CH₃ | CH₃ | CH₃ |
| 6 | NO₂ | O | N—H | CH₃ | H | CH₃ | H |
| 7 | NO₂ | O | N—H | Ph | H | Ph | H |
| 8 | NO₂ | O | N—H | p-Cl—Ph | H | p-Cl—Ph | H |
| 9 | NO₂ | O | N—H | o-Cl—Ph | H | o-Cl—Ph | H |
| 10 | NO₂ | O | N—H | p-F—Ph | H | p-F—Ph | H |
| 11 | NO₂ | O | N—H | p-Br—Ph | H | p-Br—Ph | H |
| 12 | NO₂ | O | N—H | p-Me—Ph | H | p-OMe—Ph | H |
| 13 | NO₂ | O | N—H | p-OMe—Ph | H | p-Me—Ph | H |
| 14 | NO₂ | O | N—H | p-CN—Ph | H | p-CN—Ph | H |
| 15 | NO₂ | O | N—H | p-NO₂—Ph | H | p-NO₂—Ph | H |
| 16 | NO₂ | O | N—H | p-OH—Ph | H | p-OH—Ph | H |
| 17 | NO₂ | O | N—H | p-OMe—Ph | p-OMe—Ph | i-Pr | H |
| 18 | NO₂ | O | N—H | c-Pent | H | c-Pent | H |
| 19 | NO₂ | O | N—H | c-Hex | H | c-Hex | H |
| 20 | NO₂ | O | N—H | 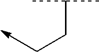 | H | 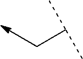 | H |
| 21 | NO₂ | O | N—H | 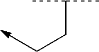 | H | 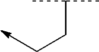 | H |
| 22 | NO₂ | O | N—H | 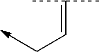 | H | 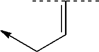 | H |
| 23 | NO₂ | O | N—H | Et | H | H | H |
| 24 | NO₂ | O | N—H | H | H | Et | H |
| 25 | NO₂ | O | N—H | i-Pr | H | H | H |
| 26 | NO₂ | O | N—H | H | H | i-Pr | H |
| 27 | NO₂ | O | N—H | c-Pr | H | H | H |
| 28 | NO₂ | O | N—H | H | H | c-Pr | H |
| 29 | NO₂ | S | N—H | H | H | H | H |
| 30 | NO₂ | S | N—H | CH₃ | H | H | H |
| 31 | NO₂ | O | N—H | 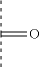 | | H | H |
| 32 | NO₂ | O | N—H | 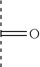 | | CH₃ | H |
| 33 | NO₂ | O | N—H | 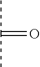 | | Et | H |
| 34 | NO₂ | O | N—H | 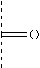 | | i-Pr | H |
| 35 | NO₂ | O | N—H | 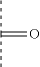 | | sec-Bu | H |
| 36 | NO₂ | O | N—H | 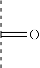 | | Ph | H |
| 37 | NO₂ | O | N—H | 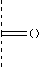 | | Bn | H |
| 38 | NO₂ | O | N—H | H | H | 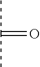 | |
| 39 | NO₂ | S | N—H | 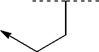 | H | 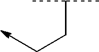 | H |
| 40 | NO₂ | S | N—H | Ph | H | Ph | H |
| 41 | NH₂ | O | N—H | H | H | H | H |
| 42 | NH₂ | O | N—H | CH₃ | H | H | H |
| 43 | NH₂ | O | N—H | H | H | CH₃ | H |
| 44 | NH₂ | O | N—H | CH₃ | CH₃ | H | H |
| 45 | NH₂ | O | N—H | CH₃ | CH₃ | CH₃ | CH₃ |
| 46 | NH₂ | O | N—H | CH₃ | H | CH₃ | H |
| 47 | NH₂ | O | N—H | Ph | H | Ph | H |
| 48 | NH₂ | O | N—H | p-Cl—Ph | H | p-Cl—Ph | H |
| 49 | NH₂ | O | N—H | o-Cl—Ph | H | o-Cl—Ph | H |
| 50 | NH₂ | O | N—H | p-F—Ph | H | p-F—Ph | H |
| 51 | NH₂ | O | N—H | p-Br—Ph | H | p-Br—Ph | H |
| 52 | NH₂ | O | N—H | p-Me—Ph | H | p-OMe—Ph | H |
| 53 | NH₂ | O | N—H | p-OMe—Ph | H | p-Me—Ph | H |
| 54 | NH₂ | O | N—H | p-CN—Ph | H | p-CN—Ph | H |
| 55 | NH₂ | O | N—H | p-NO₂—Ph | H | p-NO₂—Ph | H |
| 56 | NH₂ | O | N—H | p-OH—Ph | H | p-OH—Ph | H |
| 57 | NH₂ | O | N—H | p-OMe—Ph | p-OMe—Ph | i-Pr | H |
| 58 | NH₂ | O | N—H | c-Pent | H | c-Pent | H |
| 59 | NH₂ | O | N—H | c-Hex | H | c-Hex | H |
| 60 | NH₂ | O | N—H | 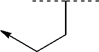 | H | 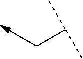 | H |
| 61 | NH₂ | O | N—H | 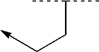 | H | 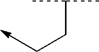 | H |
| 62 | NH₂ | O | N—H | 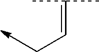 | H | 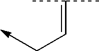 | H |
| 63 | NH₂ | O | N—H | Et | H | H | H |
| 64 | NH₂ | O | N—H | H | H | Et | H |
| 65 | NH₂ | O | N—H | i-Pr | H | H | H |
| 66 | NH₂ | O | N—H | H | H | i-Pr | H |
| 67 | NH₂ | O | N—H | c-Pr | H | H | H |
| 68 | NH₂ | O | N—H | H | H | c-Pr | H |
| 69 | NH₂ | S | N—H | H | H | H | H |
| 70 | NH₂ | S | N—H | CH₃ | H | H | H |
| 71 | NH₂ | O | N—H | 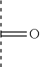 | | H | H |
| 72 | NH₂ | O | N—H | 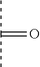 | | CH₃ | H |
| 73 | NH₂ | O | N—H | 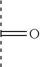 | | Et | H |
| 74 | NH₂ | O | N—H | 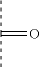 | | i-Pr | H |
| 75 | NH₂ | O | N—H | 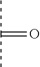 | | sec-Bu | H |
| 76 | NH₂ | O | N—H | 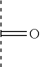 | | Ph | H |
| 77 | NH₂ | O | N—H | 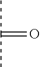 | | Bn | H |
| 78 | NH₂ | O | N—H | H | H | 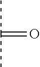 | |
| 79 | NH₂ | S | N—H | 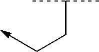 | H | 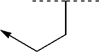 | H |
| 80 | NH₂ | S | N—H | Ph | H | Ph | H |
| 81 | Cl | O | N—H | H | H | H | H |
| 82 | Cl | O | N—H | CH₃ | H | H | H |
| 83 | Cl | O | N—H | H | H | CH₃ | H |
| 84 | Cl | O | N—H | CH₃ | CH₃ | H | H |
| 85 | Cl | O | N—H | CH₃ | CH₃ | CH₃ | CH₃ |
| 86 | Cl | O | N—H | CH₃ | H | CH₃ | H |
| 87 | Cl | O | N—H | Ph | H | Ph | H |
| 88 | Cl | O | N—H | p-Cl—Ph | H | p-Cl—Ph | H |
| 89 | Cl | O | N—H | o-Cl—Ph | H | o-Cl—Ph | H |
| 90 | Cl | O | N—H | p-F—Ph | H | p-F—Ph | H |
| 91 | Cl | O | N—H | p-Br—Ph | H | p-Br—Ph | H |
| 92 | Cl | O | N—H | p-Me—Ph | H | p-OMe—Ph | H |
| 93 | Cl | O | N—H | p-OMe—Ph | H | p-Me—Ph | H |
| 94 | Cl | O | N—H | p-CN—Ph | H | p-CN—Ph | H |
| 95 | Cl | O | N—H | p-NO₂—Ph | H | p-NO₂—Ph | H |
| 96 | Cl | O | N—H | p-OH—Ph | H | p-OH—Ph | H |
| 97 | Cl | O | N—H | p-OMe—Ph | p-OMe—Ph | i-Pr | H |
| 98 | Cl | O | N—H | c-Pent | H | c-Pent | H |
| 99 | Cl | O | N—H | c-Hex | H | c-Hex | H |
| 100 | Cl | O | N—H | 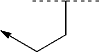 | H | 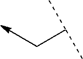 | H |
| 101 | Cl | O | N—H | 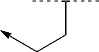 | H | 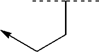 | H |
| 102 | Cl | O | N—H | 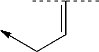 | H | 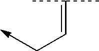 | H |
| 103 | Cl | O | N—H | Et | H | H | H |
| 104 | Cl | O | N—H | H | H | Et | H |
| 105 | Cl | O | N—H | i-Pr | H | H | H |
| 106 | Cl | O | N—H | H | H | i-Pr | H |
| 107 | Cl | O | N—H | c-Pr | H | H | H |
| 108 | Cl | O | N—H | H | H | c-Pr | H |
| 109 | Cl | S | N—H | H | H | H | H |
| 110 | Cl | S | N—H | CH₃ | H | H | H |
| 111 | Cl | O | N—H | 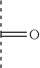 | | H | H |
| 112 | Cl | O | N—H | 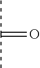 | | CH₃ | H |
| 113 | Cl | O | N—H | 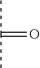 | | Et | H |
| 114 | Cl | O | N—H | 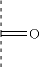 | | i-Pr | H |
| 115 | Cl | O | N—H | 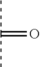 | | sec-Bu | H |
| 116 | Cl | O | N—H | 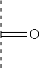 | | Ph | H |
| 117 | Cl | O | N—H | 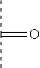 | | Bn | H |
| 118 | Cl | O | N—H | H | H | 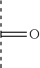 | |
| 119 | Cl | S | N—H | 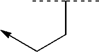 | H | 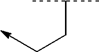 | H |
| 120 | Cl | S | N—H | Ph | H | Ph | H |
| 121 | I | O | N—H | H | H | H | H |
| 122 | I | O | N—H | CH₃ | H | H | H |
| 123 | I | O | N—H | H | H | CH₃ | H |
| 124 | I | O | N—H | CH₃ | CH₃ | H | H |
| 125 | I | O | N—H | CH₃ | CH₃ | CH₃ | CH₃ |
| 126 | I | O | N—H | CH₃ | H | CH₃ | H |
| 127 | I | O | N—H | Ph | H | Ph | H |
| 128 | I | O | N—H | p-Cl—Ph | H | p-Cl—Ph | H |
| 129 | I | O | N—H | o-Cl—Ph | H | o-Cl—Ph | H |
| 130 | I | O | N—H | p-F—Ph | H | p-F—Ph | H |
| 131 | I | O | N—H | p-Br—Ph | H | p-Br—Ph | H |
| 132 | I | O | N—H | p-Me—Ph | H | p-OMe—Ph | H |
| 133 | I | O | N—H | p-OMe—Ph | H | p-Me—Ph | H |
| 134 | I | O | N—H | p-CN—Ph | H | p-CN—Ph | H |
| 135 | I | O | N—H | p-NO₂—Ph | H | p-NO₂—Ph | H |
| 136 | I | O | N—H | p-OH—Ph | H | p-OH—Ph | H |
| 137 | I | O | N—H | p-OMe—Ph | p-OMe—Ph | i-Pr | H |
| 138 | I | O | N—H | c-Pent | H | c-Pent | H |
| 139 | I | O | N—H | c-Hex | H | c-Hex | H |
| 140 | I | O | N—H | 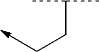 | H | 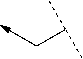 | H |
| 141 | I | O | N—H | 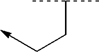 | H | 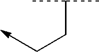 | H |
| 142 | I | O | N—H | 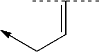 | H | 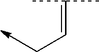 | H |
| 143 | I | O | N—H | Et | H | H | H |
| 144 | I | O | N—H | H | H | Et | H |
| 145 | I | O | N—H | i-Pr | H | H | H |
| 146 | I | O | N—H | H | H | i-Pr | H |
| 147 | I | O | N—H | c-Pr | H | H | H |
| 148 | I | O | N—H | H | H | c-Pr | H |
| 149 | I | S | N—H | H | H | H | H |
| 150 | I | S | N—H | CH₃ | H | H | H |
| 151 | I | O | N—H | 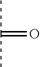 | | H | H |
| 152 | I | O | N—H | 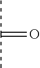 | | CH₃ | H |
| 153 | I | O | N—H | 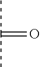 | | Et | H |
| 154 | I | O | N—H | 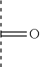 | | i-Pr | H |
| 155 | I | O | N—H | 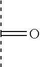 | | sec-Bu | H |
| 156 | I | O | N—H | 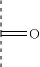 | | Ph | H |
| 157 | I | O | N—H | 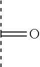 | | Bn | H |
| 158 | I | O | N—H | H | H | 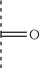 | |
| 159 | I | S | N—H | 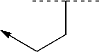 | H | 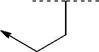 | H |
| 160 | I | S | N—H | Ph | H | Ph | H |
| 161 | 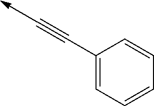 | O | N—H | H | H | H | H |
| 162 | 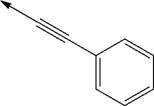 | O | N—H | CH₃ | H | H | H |
| 163 | 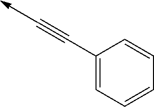 | O | N—H | H | H | CH₃ | H |
| 164 | 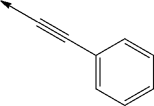 | O | N—H | CH₃ | CH₃ | H | H |
| 165 | 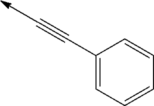 | O | N—H | CH₃ | CH₃ | CH₃ | CH₃ |
| 166 | 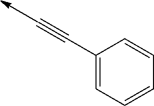 | O | N—H | CH₃ | H | CH₃ | H |
| 167 | 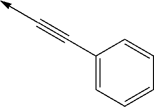 | O | N—H | Ph | H | Ph | H |
| 168 | 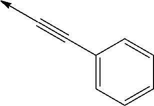 | O | N—H | p-Cl—Ph | H | p-Cl—Ph | H |
| 169 | 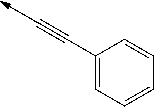 | O | N—H | o-Cl—Ph | H | o-Cl—Ph | H |
| 170 | 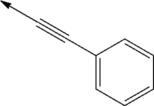 | O | N—H | p-F—Ph | H | p-F—Ph | H |
| 171 | 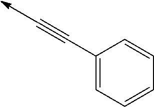 | O | N—H | p-Br—Ph | H | p-Br—Ph | H |
| 172 | 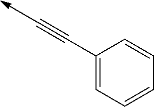 | O | N—H | p-Me—Ph | H | p-OMe—Ph | H |
| 173 | 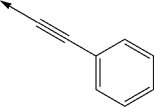 | O | N—H | p-OMe—Ph | H | p-Me—Ph | H |
| 174 | 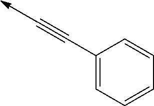 | O | N—H | p-CN—Ph | H | p-CN—Ph | H |
| 175 | 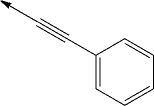 | O | N—H | p-NO₂—Ph | H | p-NO₂—Ph | H |
| 176 | 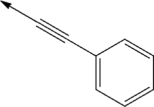 | O | N—H | p-OH—Ph | H | p-OH—Ph | H |
| 177 | 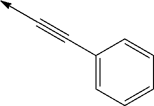 | O | N—H | p-OMe—Ph | p-OMe—Ph | i-Pr | H |
| 178 | 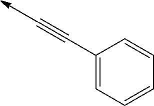 | O | N—H | c-Pent | H | c-Pent | H |
| 179 | 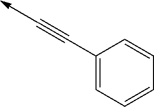 | O | N—H | c-Hex | H | c-Hex | H |
| 180 | 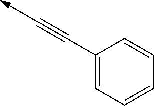 | O | N—H | 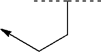 | H | 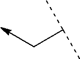 | H |
| 181 | 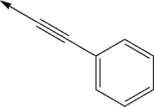 | O | N—H | 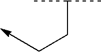 | H | 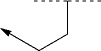 | H |
| 182 | 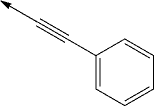 | O | N—H | 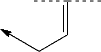 | H | 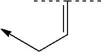 | H |
| 183 | 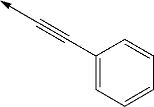 | O | N—H | Et | H | H | H |
| 184 | 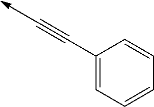 | O | N—H | H | H | Et | H |
| 185 | 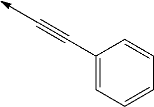 | O | N—H | i-Pr | H | H | H |
| 186 | 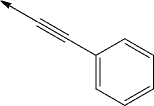 | O | N—H | H | H | i-Pr | H |
| 187 | 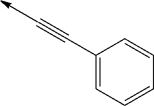 | O | N—H | c-Pr | H | H | H |
| 188 | 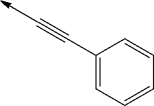 | O | N—H | H | H | c-Pr | H |
| 189 | 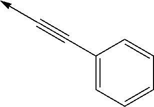 | S | N—H | H | H | H | H |
| 190 | 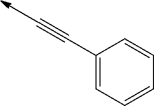 | S | N—H | CH₃ | H | H | H |
| 191 | 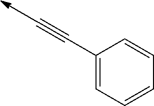 | O | N—H | 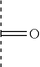 | | H | H |
| 192 | 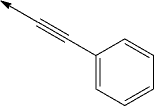 | O | N—H | 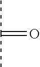 | | CH₃ | H |
| 193 | 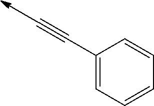 | O | N—H | 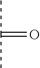 | | Et | H |
| 194 | 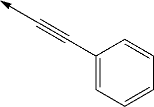 | O | N—H | 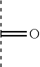 | | i-Pr | H |
| 195 | 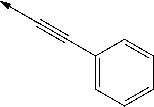 | O | N—H | 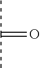 | | sec-Bu | H |
| 196 | 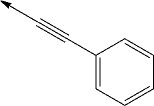 | O | N—H | 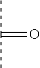 | | Ph | H |
| 197 | 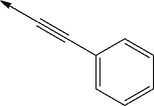 | O | N—H | 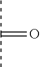 | | Bn | H |
| 198 | 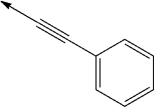 | O | N—H | H | H | 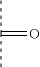 | |
| 199 | 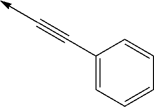 | S | N—H | 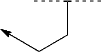 | H | 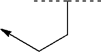 | H |
| 200 | 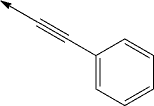 | S | N—H | Ph | H | Ph | H |
| 201 | 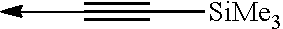 | O | N—H | H | H | H | H |
| 202 | 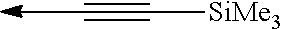 | O | N—H | CH₃ | H | H | H |
| 203 | 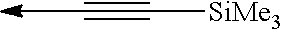 | O | N—H | H | H | CH₃ | H |
| 204 | 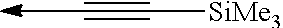 | O | N—H | CH₃ | CH₃ | H | H |
| 205 | 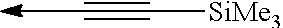 | O | N—H | CH₃ | CH₃ | CH₃ | CH₃ |
| 206 | 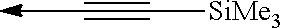 | O | N—H | CH₃ | H | CH₃ | H |
| 207 | 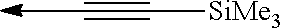 | O | N—H | Ph | H | Ph | H |
| 208 | 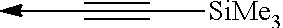 | O | N—H | p-Cl—Ph | H | p-Cl—Ph | H |
| 209 | 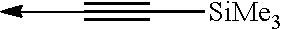 | O | N—H | o-Cl—Ph | H | o-Cl—Ph | H |
| 210 | 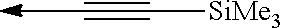 | O | N—H | p-F—Ph | H | p-F—Ph | H |
| 211 | 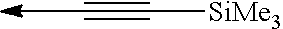 | O | N—H | p-Br—Ph | H | p-Br—Ph | H |
| 212 | 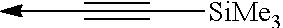 | O | N—H | p-Me—Ph | H | p-OMe—Ph | H |
| 213 | 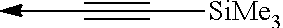 | O | N—H | p-OMe—Ph | H | p-Me—Ph | H |
| 214 | 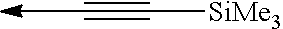 | O | N—H | p-CN—Ph | H | p-CN—Ph | H |
| 215 | 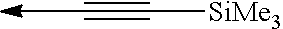 | O | N—H | p-NO₂—Ph | H | p-NO₂—Ph | H |
| 216 | 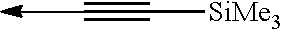 | O | N—H | p-OH—Ph | H | p-OH—Ph | H |
| 217 | 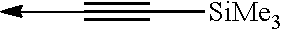 | O | N—H | p-OMe—Ph | p-OMe—Ph | i-Pr | H |
| 218 | 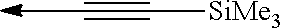 | O | N—H | c-Pent | H | c-Pent | H |
| 219 | 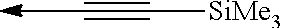 | O | N—H | c-Hex | H | c-Hex | H |
| 220 | 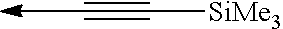 | O | N—H | 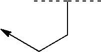 | H | 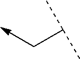 | H |
| 221 | 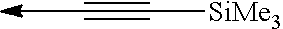 | O | N—H | 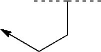 | H | 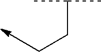 | H |
| 222 | 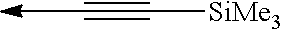 | O | N—H | 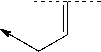 | H | 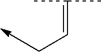 | H |
| 223 | 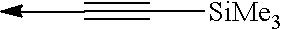 | O | N—H | Et | H | H | H |
| 224 | 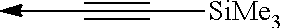 | O | N—H | H | H | Et | H |
| 225 | 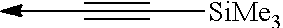 | O | N—H | i-Pr | H | H | H |
| 226 | 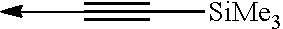 | O | N—H | H | H | i-Pr | H |
| 227 | 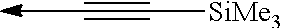 | O | N—H | c-Pr | H | H | H |
| 228 | 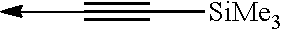 | O | N—H | H | H | c-Pr | H |
| 229 | 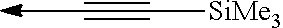 | S | N—H | H | H | H | H |
| 230 | 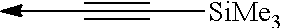 | S | N—H | CH₃ | H | H | H |
| 231 | 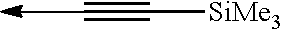 | O | N—H | 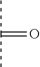 | | H | H |
| 232 | 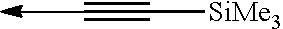 | O | N—H | 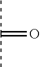 | | CH₃ | H |
| 233 | 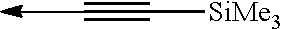 | O | N—H | 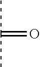 | | Et | H |
| 234 | 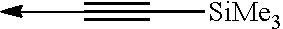 | O | N—H | 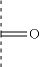 | | i-Pr | H |
| 235 | 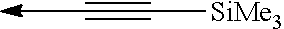 | O | N—H | 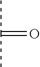 | | sec-Bu | H |
| 236 | 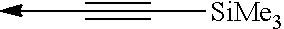 | O | N—H | 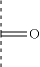 | | Ph | H |
| 237 | 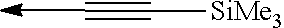 | O | N—H | 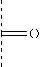 | | Bn | H |
| 238 | 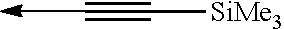 | O | N—H | H | H | 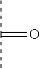 | |
| 239 | 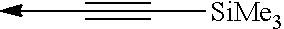 | S | N—H | 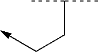 | H | 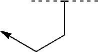 | H |
| 240 | 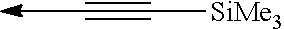 | S | N—H | Ph | H | Ph | H |
| 241 | 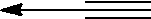 | O | N—H | H | H | H | H |
| 242 | 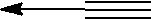 | O | N—H | CH₃ | H | H | H |
| 243 | 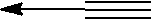 | O | N—H | H | H | CH₃ | H |
| 244 | 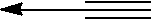 | O | N—H | CH₃ | CH₃ | H | H |
| 245 | 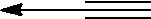 | O | N—H | CH₃ | CH₃ | CH₃ | CH₃ |
| 246 | 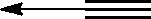 | O | N—H | CH₃ | H | CH₃ | H |
| 247 | 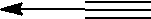 | O | N—H | Ph | H | Ph | H |
| 248 | 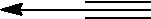 | O | N—H | p-Cl—Ph | H | p-Cl—Ph | H |
| 249 | 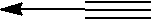 | O | N—H | o-Cl—Ph | H | o-Cl—Ph | H |
| 250 | 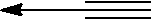 | O | N—H | p-F—Ph | H | p-F—Ph | H |
| 251 | 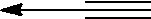 | O | N—H | p-Br—Ph | H | p-Br—Ph | H |
| 252 | 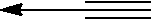 | O | N—H | p-Me—Ph | H | p-OMe—Ph | H |
| 253 | 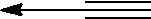 | O | N—H | p-OMe—Ph | H | p-Me—Ph | H |
| 254 | 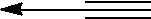 | O | N—H | p-CN—Ph | H | p-CN—Ph | H |
| 255 | 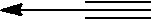 | O | N—H | p-NO₂—Ph | H | p-NO₂—Ph | H |
| 256 | 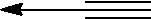 | O | N—H | p-OH—Ph | H | p-OH—Ph | H |
| 257 | 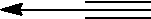 | O | N—H | p-OMe—Ph | p-OMe—Ph | i-Pr | H |
| 258 | 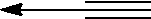 | O | N—H | c-Pent | H | c-Pent | H |
| 259 | 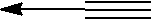 | O | N—H | c-Hex | H | c-Hex | H |
| 260 | 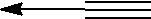 | O | N—H | 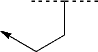 | H | 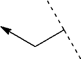 | H |
| 261 | 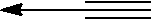 | O | N—H | 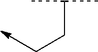 | H | 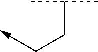 | H |
| 262 | 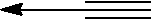 | O | N—H | 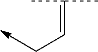 | H | 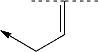 | H |
| 263 | 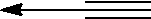 | O | N—H | Et | H | H | H |
| 264 | 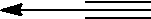 | O | N—H | H | H | Et | H |
| 265 | 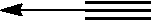 | O | N—H | i-Pr | H | H | H |
| 266 | 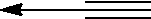 | O | N—H | H | H | i-Pr | H |
| 267 | 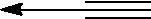 | O | N—H | c-Pr | H | H | H |
| 268 | 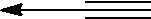 | O | N—H | H | H | c-Pr | H |
| 269 | 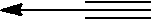 | S | N—H | H | H | H | H |
| 270 | 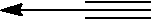 | S | N—H | CH₃ | H | H | H |
| 271 | 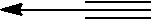 | O | N—H | 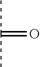 | | H | H |
| 272 | 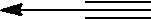 | O | N—H | 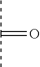 | | CH₃ | H |
| 273 | 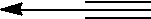 | O | N—H | 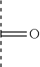 | | Et | H |
| 274 | 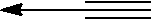 | O | N—H | 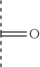 | | i-Pr | H |
| 275 | 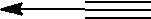 | O | N—H | 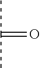 | | sec-Bu | H |
| 276 | 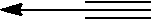 | O | N—H | 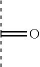 | | Ph | H |
| 277 | 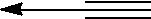 | O | N—H | 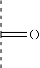 | | Bn | H |
| 278 | 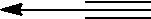 | O | N—H | H | H | 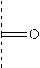 | |
| 279 | 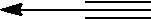 | S | N—H | 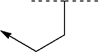 | H | 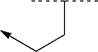 | H |
| 280 | 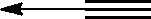 | S | N—H | Ph | H | Ph | H |
| 281 | 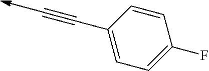 | O | N—H | H | H | H | H |
| 282 | 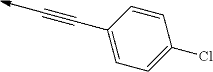 | O | N—H | H | H | H | H |
| 283 | 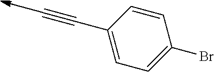 | O | N—H | H | H | H | H |
| 284 | 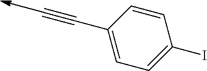 | O | N—H | H | H | H | H |
| 285 | 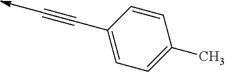 | O | N—H | H | H | H | H |
| 286 | 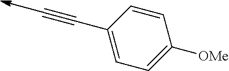 | O | N—H | H | H | H | H |
| 287 | 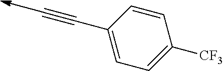 | O | N—H | H | H | H | H |
| 288 | 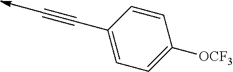 | O | N—H | H | H | H | H |
| 289 | 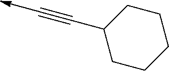 | O | N—H | H | H | H | H |
| 290 | 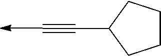 | O | N—H | H | H | H | H |
| 291 | 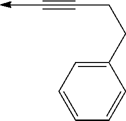 | O | N—H | H | H | H | H |
| 292 | 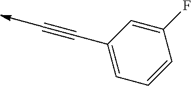 | O | N—H | H | H | H | H |
| 293 | 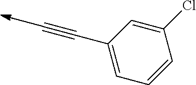 | O | N—H | H | H | H | H |
| 294 | 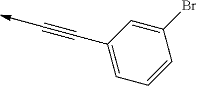 | O | N—H | H | H | H | H |
| 295 | 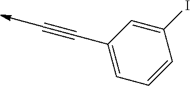 | O | N—H | H | H | H | H |
| 296 | 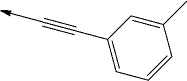 | O | N—H | H | H | H | H |
| 297 | 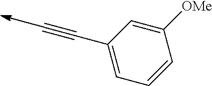 | O | N—H | H | H | H | H |
| 298 | 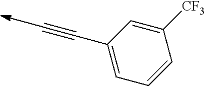 | O | N—H | H | H | H | H |
| 299 | 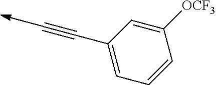 | O | N—H | H | H | H | H |
| 300 | 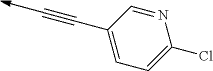 | O | N—H | H | H | H | H |

A2. Compounds A2-1 to A2-300 of the general formula (I) in which in which R¹ represents fluorine, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A2-1 to A2-300).
A3. Compounds A3-1 to A3-300 of the general formula (I) in which in which R¹ represents chlorine, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A3-1 to A3-300).
A4. Compounds A4-1 to A4-300 of the general formula (I) in which in which R¹ represents bromine, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A4-1 to A4-300).
A5. Compounds A5-1 to A5-300 of the general formula (I) in which in which R¹ represents methyl, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A5-1 to A5-300).
A6. Compounds A6-1 to A6-300 of the general formula (I) in which in which R¹ represents trifluoromethyl, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A6-1 to A6-300).
A7. Compounds A7-1 to A7-300 of the general formula (I) in which in which R¹ represents methoxy, R², R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A7-1 to A7-300).
A8. Compounds A8-1 to A8-300 of the general formula (I) in which in which R² represents fluorine, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A8-1 to A8-300).
A9. Compounds A9-1 to A9-300 of the general formula (I) in which in which R² represents chlorine, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A9-1 to A9-300).
A10. Compounds A10-1 to A10-300 of the general formula (I) in which in which R² represents bromine, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A10-1 to A10-300).
A11. Compounds A11-1 to A11-300 of the general formula (I) in which in which R² represents methyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A11-1 to A11-300).
A12. Compounds A12-1 to A12-300 of the general formula (I) in which in which R² represents trifluoromethyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A12-1 to A12-300).
A13. Compounds A13-1 to A13-300 of the general formula (I) in which in which R² represents methoxy, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A13-1 to A13-300).
A14. Compounds A14-1 to A14-300 of the general formula (I) in which in which R² represents trifluoromethoxy, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A14-1 to A14-300).
A15. Compounds A15-1 to A15-300 of the general formula (I) in which in which R² represents ethyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A15-1 to A15-300).
A16. Compounds A16-1 to A16-300 of the general formula (I) in which in which R² represents isopropyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A16-1 to A16-300).
A17. Compounds A17-1 to A17-300 of the general formula (I) in which in which R² represents cyclopropyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A17-1 to A17-300).
A18. Compounds A18-1 to A18-300 of the general formula (I) in which in which R² represents phenyl, R¹, R³ and R⁹ represent hydrogen and X, W, R⁴, R⁵, R⁶, R⁷, R⁸ for the individual compound in question correspond to the radical definitions given in Table 1 (Nos 1 to 300; corresponding to Compounds A18-1 to A18-300).
A19. Compounds A19-1 to A19-300 of the general formula (I) in which in which R³ represents fluorine, R¹, R² and R⁹ represent hydrogen and X, W, R⁴, R⁵, R⁶, R⁷, R⁸ for the individual compound in question correspond to the radical definitions given in Table 1 (Nos 1 to 300; corresponding to Compounds A19-1 to A19-300).
A20. Compounds A20-1 to A20-300 of the general formula (I) in which in which R³ represents methyl, R¹, R² and R⁹ represent hydrogen and X, W, R⁴, R⁵, R⁶, R⁷, R⁸ for the individual compound in question correspond to the radical definitions given in Table 1 (Nos 1 to 300; corresponding to Compounds A20-1 to A20-300).
B1. Compounds B1-1 to B1-949 of the general formula (Ia) in which R¹, R², R³ and R⁹ represent hydrogen and Q, W, R⁵, R⁶, R⁷, R⁸ correspond to the definitions (Nos 1 to 949; corresponding to Compounds B1-1 to B1-949) in Table 2 below. An arrow in a definition given in Table 2 for R⁵ and R⁷ represents a bond of the radical in question to the core structure (I); in this case, the two groups R⁵ and R⁷ together form a saturated or partially saturated ring.

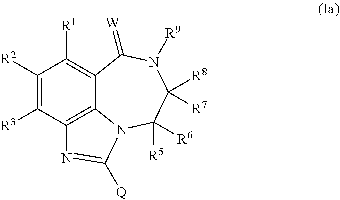

TABLE 2

| No. | Q | W | R⁵ | R⁶ | R⁷ | R⁸ |
|---|---|---|---|---|---|---|
| 1 | CH₃ | O | H | H | H | H |
| 2 | CH₃ | S | H | H | H | H |
| 3 | CH₃ | O | CH₃ | H | H | H |
| 4 | CH₃ | O | CH₃ | H | CH₃ | H |
| 5 | CH₃ | O | Ph | H | Ph | H |
| 6 | CH₃ | O | 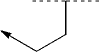 | H | 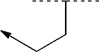 | H |
| 7 | CH₃ | O | 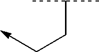 | H | 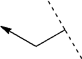 | H |
| 8 | CH₃ | O | 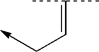 | H | 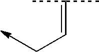 | H |
| 9 | CH₃ | O | H | H | CH₃ | H |
| 10 | CH₃ | O | c-Hex | H | c-Hex | H |
| 11 | 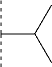 | O | H | H | H | H |
| 12 | 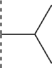 | S | H | H | H | H |
| 13 | 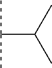 | O | CH₃ | H | H | H |
| 14 | 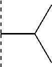 | O | CH₃ | H | CH₃ | H |
| 15 | 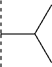 | O | Ph | H | Ph | H |
| 16 | 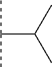 | O | 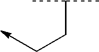 | H | 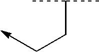 | H |
| 17 | 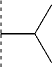 | O | 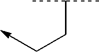 | H | 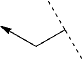 | H |
| 18 | 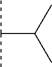 | O | 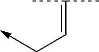 | H | 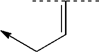 | H |
| 19 | 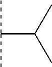 | O | H | H | CH₃ | H |
| 20 | 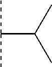 | O | c-Hex | H | c-Hex | H |
| 21 | 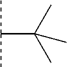 | O | H | H | H | H |
| 22 | 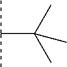 | S | H | H | H | H |
| 23 | 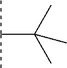 | O | CH₃ | H | H | H |
| 24 | 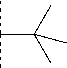 | O | CH₃ | H | CH₃ | H |
| 25 | 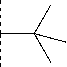 | O | Ph | H | Ph | H |
| 26 | 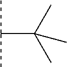 | O | 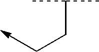 | H | 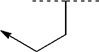 | H |
| 27 | 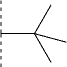 | O | 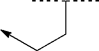 | H | 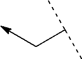 | H |
| 28 | 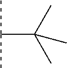 | O | 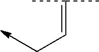 | H | 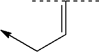 | H |
| 29 | 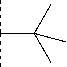 | O | H | H | CH₃ | H |
| 30 | 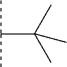 | O | c-Hex | H | c-Hex | H |
| 31 | 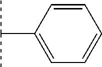 | O | H | H | H | H |
| 32 | 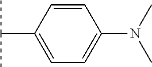 | O | H | H | H | H |
| 33 | 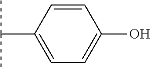 | O | H | H | H | H |
| 34 | 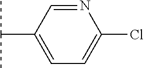 | O | H | H | H | H |
| 35 | 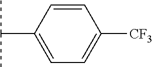 | O | H | H | H | H |
| 36 | 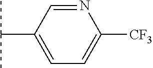 | O | H | H | H | H |
| 37 | 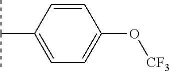 | O | H | H | H | H |
| 38 | 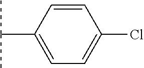 | O | H | H | H | H |
| 39 | 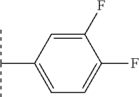 | O | H | H | H | H |
| 40 | 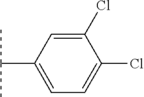 | O | H | H | H | H |
| 41 | 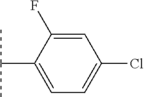 | O | H | H | H | H |
| 42 | 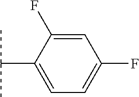 | O | H | H | H | H |
| 43 | 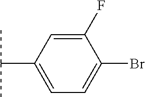 | O | H | H | H | H |
| 44 | 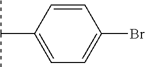 | O | H | H | H | H |
| 45 | 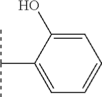 | O | H | H | H | H |
| 46 | 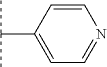 | O | H | H | H | H |
| 47 | 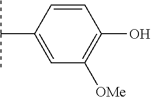 | O | H | H | H | H |
| 48 | 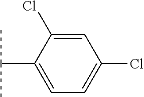 | O | H | H | H | H |
| 49 | 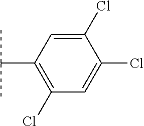 | O | H | H | H | H |
| 50 | 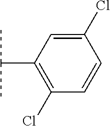 | O | H | H | H | H |
| 51 | 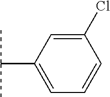 | O | H | H | H | H |
| 52 | 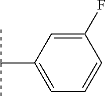 | O | H | H | H | H |
| 53 | 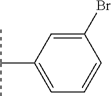 | O | H | H | H | H |
| 54 | 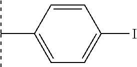 | O | H | H | H | H |
| 55 | 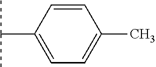 | O | H | H | H | H |
| 56 | 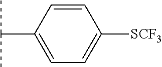 | O | H | H | H | H |
| 57 | 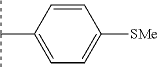 | O | H | H | H | H |
| 58 | 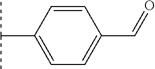 | O | H | H | H | H |
| 59 | 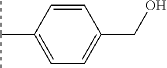 | O | H | H | H | H |
| 60 | 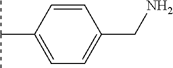 | O | H | H | H | H |
| 61 | 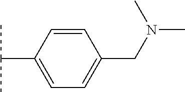 | O | H | H | H | H |
| 62 | 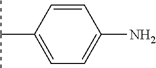 | O | H | H | H | H |
| 63 | 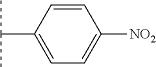 | O | H | H | H | H |
| 64 | 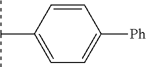 | O | H | H | H | H |
| 65 | 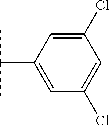 | O | H | H | H | H |
| 66 | 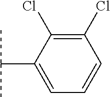 | O | H | H | H | H |
| 67 | 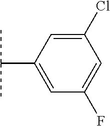 | O | H | H | H | H |
| 68 | 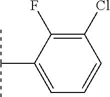 | O | H | H | H | H |
| 69 | 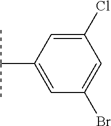 | O | H | H | H | H |
| 70 | 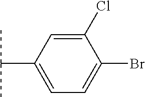 | O | H | H | H | H |
| 71 | 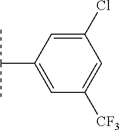 | O | H | H | H | H |
| 72 | 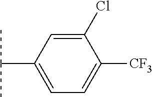 | O | H | H | H | H |
| 73 | 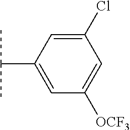 | O | H | H | H | H |
| 74 | 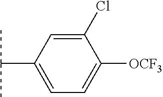 | O | H | H | H | H |
| 75 | 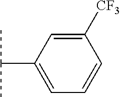 | O | H | H | H | H |
| 76 | 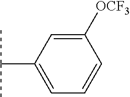 | O | H | H | H | H |
| 77 | 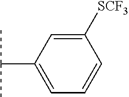 | O | H | H | H | H |
| 78 | 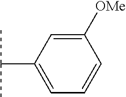 | O | H | H | H | H |
| 79 | 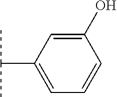 | O | H | H | H | H |
| 80 | 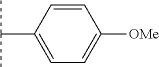 | O | H | H | H | H |
| 81 | 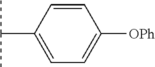 | O | H | H | H | H |
| 82 | 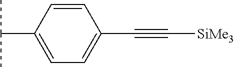 | O | H | H | H | H |
| 83 | 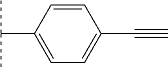 | O | H | H | H | H |
| 84 | 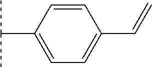 | O | H | H | H | H |
| 85 | 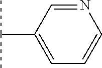 | O | H | H | H | H |
| 86 | 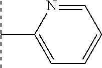 | O | H | H | H | H |
| 87 | 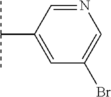 | O | H | H | H | H |
| 88 | 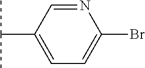 | O | H | H | H | H |
| 89 | 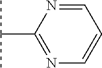 | O | H | H | H | H |
| 90 | 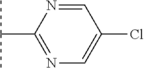 | O | H | H | H | H |
| 91 | 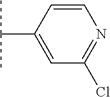 | O | H | H | H | H |
| 92 | 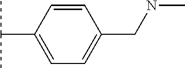 | O | H | H | H | H |
| 93 | 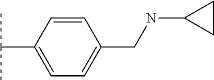 | O | H | H | H | H |
| 94 | 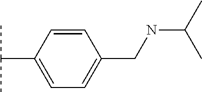 | O | H | H | H | H |
| 95 | 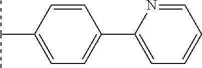 | O | H | H | H | H |
| 96 | 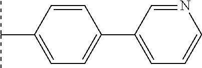 | O | H | H | H | H |
| 97 | 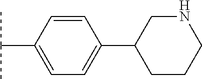 | O | H | H | H | H |
| 98 | 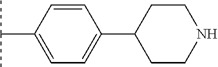 | O | H | H | H | H |
| 99 | 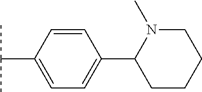 | O | H | H | H | H |
| 100 | 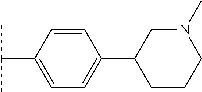 | O | H | H | H | H |
| 101 | 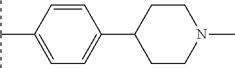 | O | H | H | H | H |
| 102 | 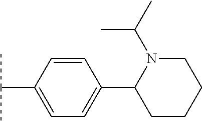 | O | H | H | H | H |
| 103 | 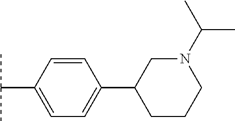 | O | H | H | H | H |
| 104 | 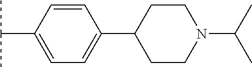 | O | H | H | H | H |
| 105 | 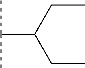 | O | H | H | H | H |
| 106 | 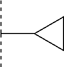 | O | H | H | H | H |
| 107 | 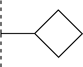 | O | H | H | H | H |
| 108 | 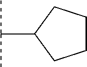 | O | H | H | H | H |
| 109 | 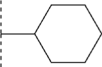 | O | H | H | H | H |
| 110 | 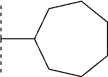 | O | H | H | H | H |
| 111 | 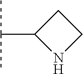 | O | H | H | H | H |
| 112 | 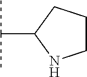 | O | H | H | H | H |
| 113 | 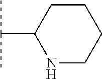 | O | H | H | H | H |
| 114 | 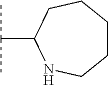 | O | H | H | H | H |
| 115 | 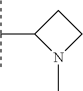 | O | H | H | H | H |
| 116 | 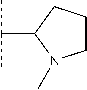 | O | H | H | H | H |
| 117 | 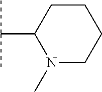 | O | H | H | H | H |
| 118 | 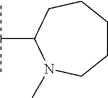 | O | H | H | H | H |
| 119 | 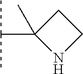 | O | H | H | H | H |
| 120 | 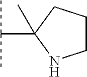 | O | H | H | H | H |
| 121 | 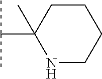 | O | H | H | H | H |
| 122 | 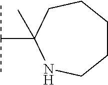 | O | H | H | H | H |
| 123 | 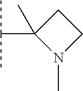 | O | H | H | H | H |
| 124 | 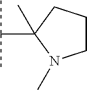 | O | H | H | H | H |
| 125 | 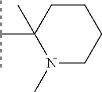 | O | H | H | H | H |
| 126 | 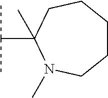 | O | H | H | H | H |
| 127 | 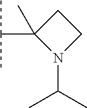 | O | H | H | H | H |
| 128 | 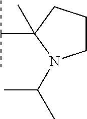 | O | H | H | H | H |
| 129 | 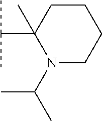 | O | H | H | H | H |
| 130 | 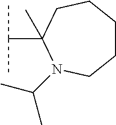 | O | H | H | H | H |
| 131 | 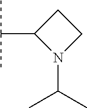 | O | H | H | H | H |
| 134 | 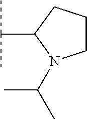 | O | H | H | H | H |
| 135 | 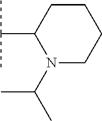 | O | H | H | H | H |
| 136 | 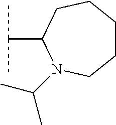 | O | H | H | H | H |
| 137 | 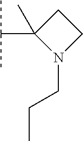 | O | H | H | H | H |
| 138 | 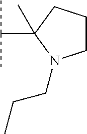 | O | H | H | H | H |
| 139 | 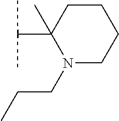 | O | H | H | H | H |
| 140 | 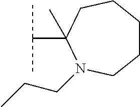 | O | H | H | H | H |
| 141 | 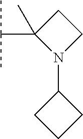 | O | H | H | H | H |
| 142 | 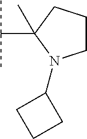 | O | H | H | H | H |
| 143 | 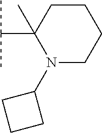 | O | H | H | H | H |
| 144 | 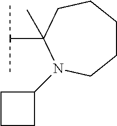 | O | H | H | H | H |
| 145 | 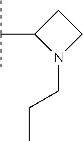 | O | H | H | H | H |
| 146 | 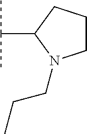 | O | H | H | H | H |
| 147 | 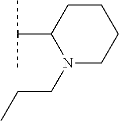 | O | H | H | H | H |
| 148 | 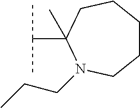 | O | H | H | H | H |
| 149 | 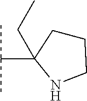 | O | H | H | H | H |
| 150 | 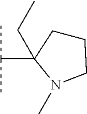 | O | H | H | H | H |
| 151 | 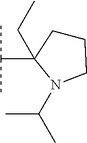 | O | H | H | H | H |
| 152 | 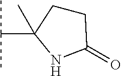 | O | H | H | H | H |
| 153 | 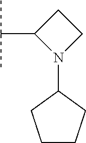 | O | H | H | H | H |
| 154 | 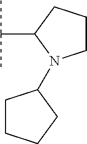 | O | H | H | H | H |
| 155 | 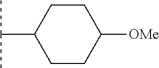 | O | H | H | H | H |
| 156 | 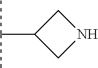 | O | H | H | H | H |
| 157 | 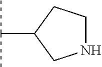 | O | H | H | H | H |
| 158 | 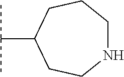 | O | H | H | H | H |
| 159 | 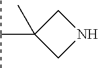 | O | H | H | H | H |
| 160 | 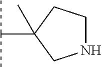 | O | H | H | H | H |
| 161 | 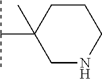 | O | H | H | H | H |
| 162 | 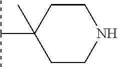 | O | H | H | H | H |
| 163 | 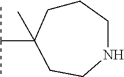 | O | H | H | H | H |
| 164 | 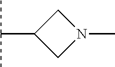 | O | H | H | H | H |
| 165 | 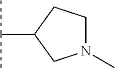 | O | H | H | H | H |
| 166 | 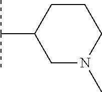 | O | H | H | H | H |
| 167 | 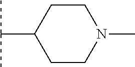 | O | H | H | H | H |
| 168 | 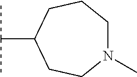 | O | H | H | H | H |
| 169 | 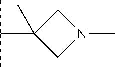 | O | H | H | H | H |
| 170 | 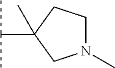 | O | H | H | H | H |
| 171 | 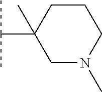 | O | H | H | H | H |
| 172 | 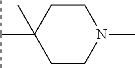 | O | H | H | H | H |
| 173 | 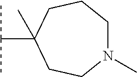 | O | H | H | H | H |
| 174 | 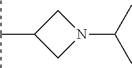 | O | H | H | H | H |
| 175 | 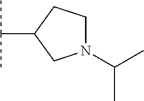 | O | H | H | H | H |
| 176 | 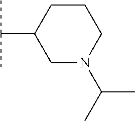 | O | H | H | H | H |
| 177 | 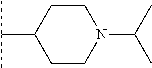 | O | H | H | H | H |
| 178 | 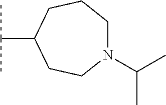 | O | H | H | H | H |
| 179 | 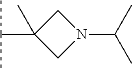 | O | H | H | H | H |
| 180 | 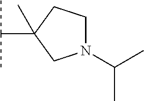 | O | H | H | H | H |
| 181 | 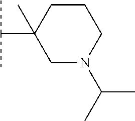 | O | H | H | H | H |
| 182 | 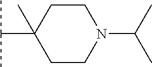 | O | H | H | H | H |
| 183 | 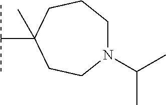 | O | H | H | H | H |
| 184 | 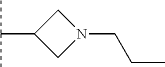 | O | H | H | H | H |
| 185 | 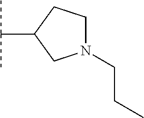 | O | H | H | H | H |
| 186 | 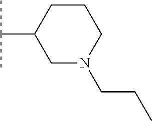 | O | H | H | H | H |
| 187 | 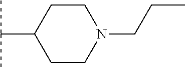 | O | H | H | H | H |
| 188 | 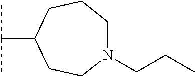 | O | H | H | H | H |
| 189 | 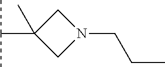 | O | H | H | H | H |
| 190 | 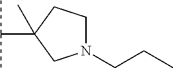 | O | H | H | H | H |
| 191 | 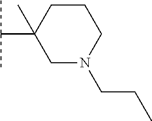 | O | H | H | H | H |
| 192 | 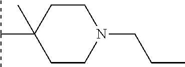 | O | H | H | H | H |
| 193 | 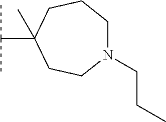 | O | H | H | H | H |
| 194 | 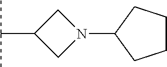 | O | H | H | H | H |
| 195 | 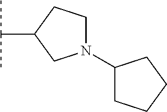 | O | H | H | H | H |
| 196 | 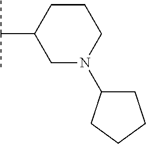 | O | H | H | H | H |
| 197 | 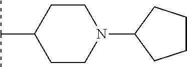 | O | H | H | H | H |
| 198 | 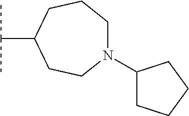 | O | H | H | H | H |
| 199 | 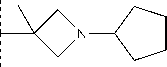 | O | H | H | H | H |
| 200 | 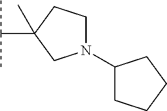 | O | H | H | H | H |
| 201 | 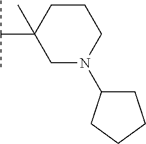 | O | H | H | H | H |
| 202 | 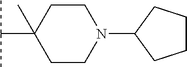 | O | H | H | H | H |
| 203 | 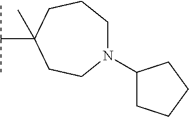 | O | H | H | H | H |
| 204 | 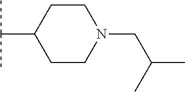 | O | H | H | H | H |
| 205 | 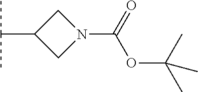 | O | H | H | H | H |
| 206 | 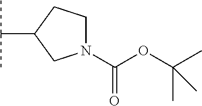 | O | H | H | H | H |
| 207 | 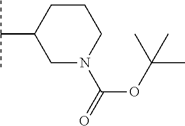 | O | H | H | H | H |
| 208 | 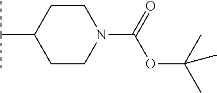 | O | H | H | H | H |
| 209 | 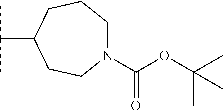 | O | H | H | H | H |
| 210 | 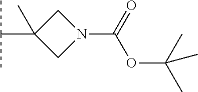 | O | H | H | H | H |
| 211 | 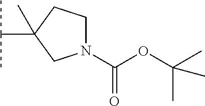 | O | H | H | H | H |
| 212 | 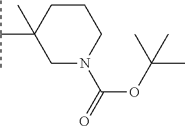 | O | H | H | H | H |
| 213 | 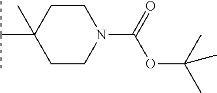 | O | H | H | H | H |
| 214 | 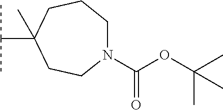 | O | H | H | H | H |
| 215 | 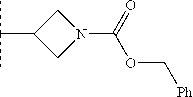 | O | H | H | H | H |
| 216 | 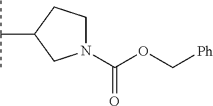 | O | H | H | H | H |
| 217 | 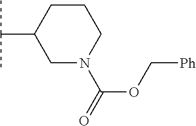 | O | H | H | H | H |
| 218 | 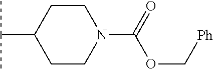 | O | H | H | H | H |
| 219 | 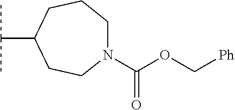 | O | H | H | H | H |
| 220 | 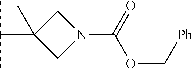 | O | H | H | H | H |
| 221 | 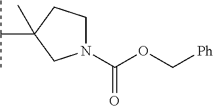 | O | H | H | H | H |
| 222 | 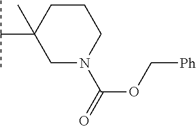 | O | H | H | H | H |
| 223 | 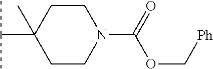 | O | H | H | H | H |
| 224 | 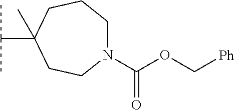 | O | H | H | H | H |
| 225 | 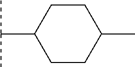 | O | H | H | H | H |
| 226 | 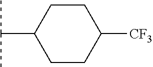 | O | H | H | H | H |
| 227 | 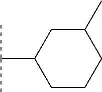 | O | H | H | H | H |
| 228 | 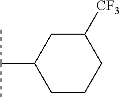 | O | H | H | H | H |
| 229 | 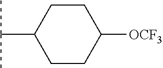 | O | H | H | H | H |
| 230 | 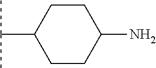 | O | H | H | H | H |
| 231 | 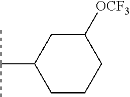 | O | H | H | H | H |
| 232 | 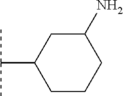 | O | H | H | H | H |
| 233 | 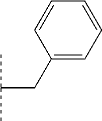 | O | H | H | H | H |
| 234 | 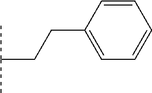 | O | H | H | H | H |
| 235 | 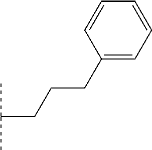 | O | H | H | H | H |
| 236 | 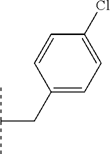 | O | H | H | H | H |
| 237 | 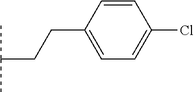 | O | H | H | H | H |
| 238 | 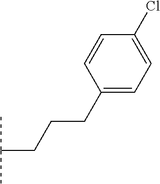 | O | H | H | H | H |
| 239 | 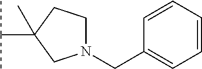 | O | H | H | H | H |
| 240 | 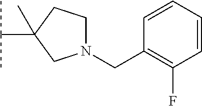 | O | H | H | H | H |
| 241 | 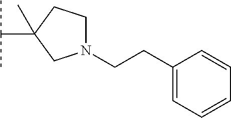 | O | H | H | H | H |
| 242 | 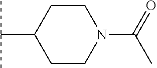 | O | H | H | H | H |
| 243 | 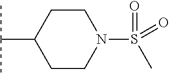 | O | H | H | H | H |
| 244 | 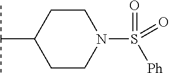 | O | H | H | H | H |
| 245 | 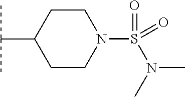 | O | H | H | H | H |
| 246 | 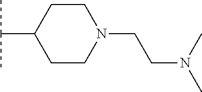 | O | H | H | H | H |
| 247 | 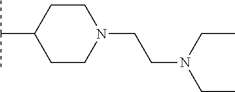 | O | H | H | H | H |
| 248 | 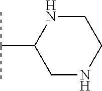 | O | H | H | H | H |
| 249 | 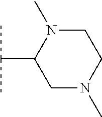 | O | H | H | H | H |
| 250 | 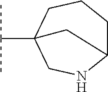 | O | H | H | H | H |
| 251 | 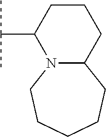 | O | H | H | H | H |
| 252 | 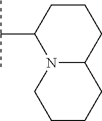 | O | H | H | H | H |
| 253 | 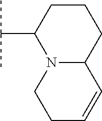 | O | H | H | H | H |
| 254 | 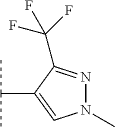 | O | H | H | H | H |
| 255 | 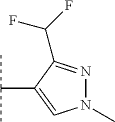 | O | H | H | H | H |
| 256 | 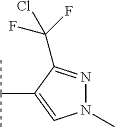 | O | H | H | H | H |
| 257 | 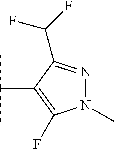 | O | H | H | H | H |
| 258 | 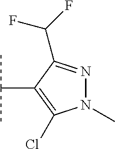 | O | H | H | H | H |
| 259 | 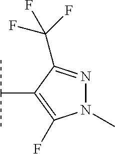 | O | H | H | H | H |
| 260 | 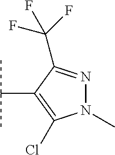 | O | H | H | H | H |
| 261 | 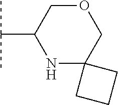 | O | H | H | H | H |
| 262 | 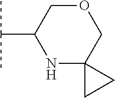 | O | H | H | H | H |
| 263 | 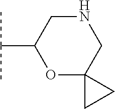 | O | H | H | H | H |
| 264 | 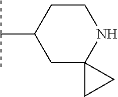 | O | H | H | H | H |
| 265 | 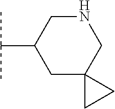 | O | H | H | H | H |
| 266 | 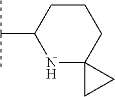 | O | H | H | H | H |
| 267 | 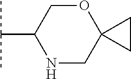 | O | H | H | H | H |
| 268 | 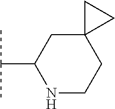 | O | H | H | H | H |
| 269 | 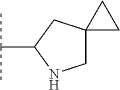 | O | H | H | H | H |
| 270 | 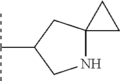 | O | H | H | H | H |
| 271 | 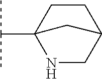 | O | H | H | H | H |
| 272 | 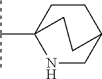 | O | H | H | H | H |
| 273 | 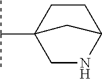 | O | H | H | H | H |
| 274 | 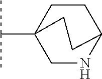 | O | H | H | H | H |
| 275 | 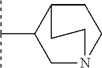 | O | H | H | H | H |
| 276 | 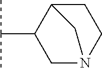 | O | H | H | H | H |
| 277 | 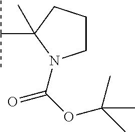 | O | H | H | H | H |
| 278 | 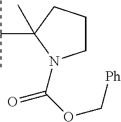 | O | H | H | H | H |
| 279 | 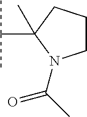 | O | H | H | H | H |
| 280 | 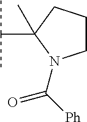 | O | H | H | H | H |
| 281 | 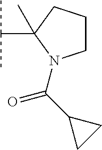 | O | H | H | H | H |
| 282 | 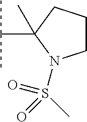 | O | H | H | H | H |
| 283 | 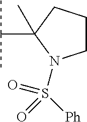 | O | H | H | H | H |
| 284 | 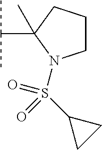 | O | H | H | H | H |
| 285 | 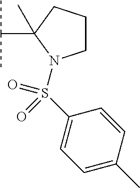 | O | H | H | H | H |
| 286 | 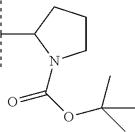 | O | H | H | H | H |
| 287 | 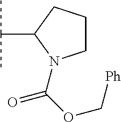 | O | H | H | H | H |
| 288 | 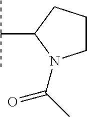 | O | H | H | H | H |
| 289 | 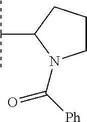 | O | H | H | H | H |
| 290 | 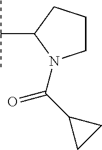 | O | H | H | H | H |
| 291 | 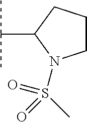 | O | H | H | H | H |
| 292 | 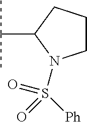 | O | H | H | H | H |
| 293 | 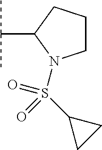 | O | H | H | H | H |
| 294 | 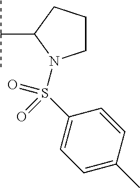 | O | H | H | H | H |
| 295 | 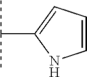 | O | H | H | H | H |
| 296 | 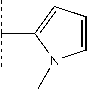 | O | H | H | H | H |
| 297 | 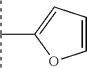 | O | H | H | H | H |
| 298 | 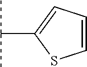 | O | H | H | H | H |
| 299 | 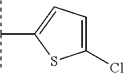 | O | H | H | H | H |
| 300 | 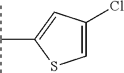 | O | H | H | H | H |
| 301 | 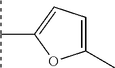 | O | H | H | H | H |
| 302 | 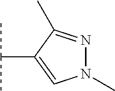 | O | H | H | H | H |
| 303 | 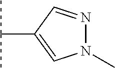 | O | H | H | H | H |
| 304 | 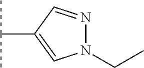 | O | H | H | H | H |
| 305 | 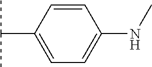 | O | H | H | H | H |
| 306 | 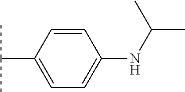 | O | H | H | H | H |
| 307 | 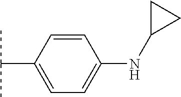 | O | H | H | H | H |
| 308 | 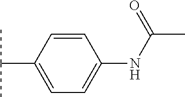 | O | H | H | H | H |
| 309 | 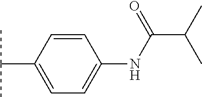 | O | H | H | H | H |
| 310 | 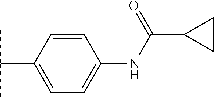 | O | H | H | H | H |
| 311 | 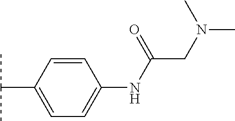 | O | H | H | H | H |
| 312 | 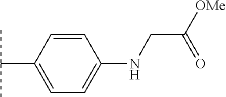 | O | H | H | H | H |
| 313 | 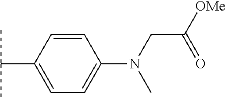 | O | H | H | H | H |
| 314 | 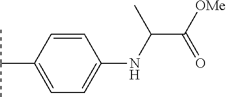 | O | H | H | H | H |
| 315 | 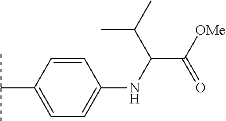 | O | H | H | H | H |
| 316 | 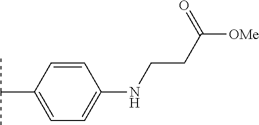 | O | H | H | H | H |
| 317 | 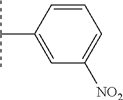 | O | H | H | H | H |
| 318 | 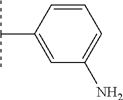 | O | H | H | H | H |
| 319 | 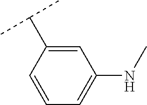 | O | H | H | H | H |
| 320 | 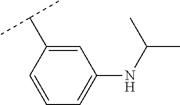 | O | H | H | H | H |
| 321 | 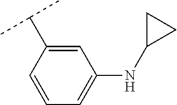 | O | H | H | H | H |
| 322 | 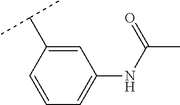 | O | H | H | H | H |
| 323 | 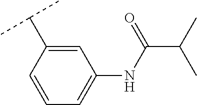 | O | H | H | H | H |
| 324 | 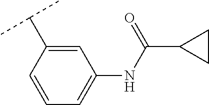 | O | H | H | H | H |
| 325 | 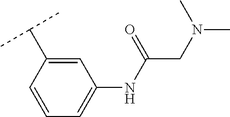 | O | H | H | H | H |
| 326 | 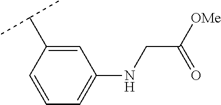 | O | H | H | H | H |
| 327 | 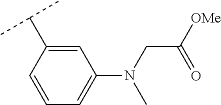 | O | H | H | H | H |
| 328 | 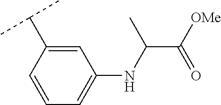 | O | H | H | H | H |
| 329 | 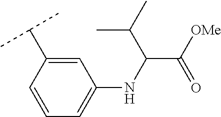 | O | H | H | H | H |
| 330 | 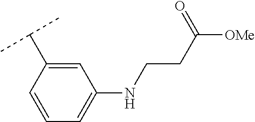 | O | H | H | H | H |
| 331 | 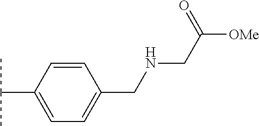 | O | H | H | H | H |
| 332 | 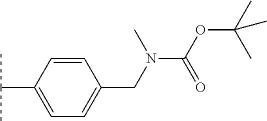 | O | H | H | H | H |
| 333 | 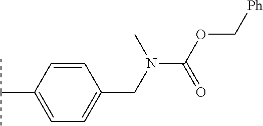 | O | H | H | H | H |
| 334 | 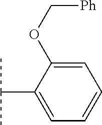 | O | H | H | H | H |
| 335 | 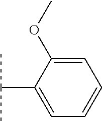 | O | H | H | H | H |
| 336 | 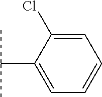 | O | H | H | H | H |
| 337 | 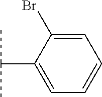 | O | H | H | H | H |
| 338 | 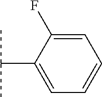 | O | H | H | H | H |
| 339 | 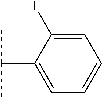 | O | H | H | H | H |
| 340 | 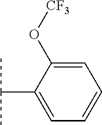 | O | H | H | H | H |
| 341 | 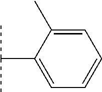 | O | H | H | H | H |
| 342 | 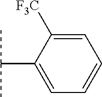 | O | H | H | H | H |
| 343 | 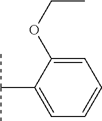 | O | H | H | H | H |
| 344 | 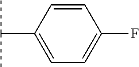 | O | H | H | H | H |
| 345 | 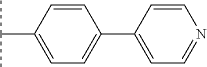 | O | H | H | H | H |
| 346 | 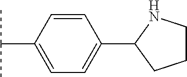 | O | H | H | H | H |
| 347 | 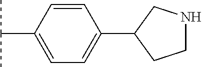 | O | H | H | H | H |
| 348 | 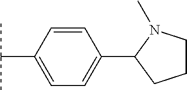 | O | H | H | H | H |
| 349 | 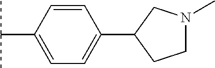 | O | H | H | H | H |
| 350 | 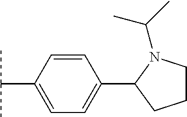 | O | H | H | H | H |
| 351 | 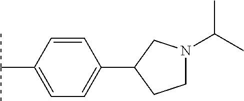 | O | H | H | H | H |
| 352 | 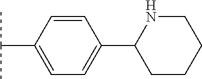 | O | H | H | H | H |
| 353 | 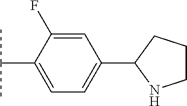 | O | H | H | H | H |
| 354 | 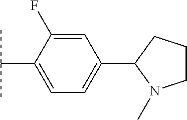 | O | H | H | H | H |
| 355 | 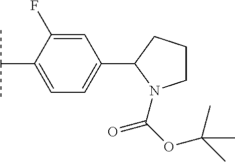 | O | H | H | H | H |
| 356 | 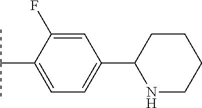 | O | H | H | H | H |
| 357 | 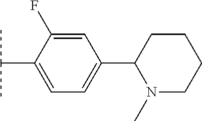 | O | H | H | H | H |
| 358 | 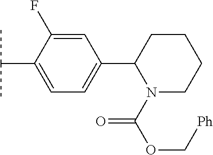 | O | H | H | H | H |
| 359 | 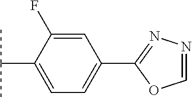 | O | H | H | H | H |
| 360 | 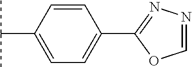 | O | H | H | H | H |
| 361 | 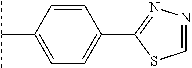 | O | H | H | H | H |
| 362 | 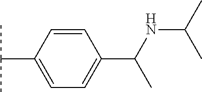 | O | H | H | H | H |
| 363 | 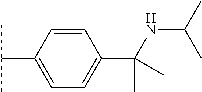 | O | H | H | H | H |
| 364 | 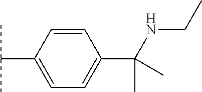 | O | H | H | H | H |
| 365 | 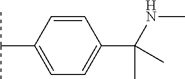 | O | H | H | H | H |
| 366 | 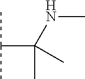 | O | H | H | H | H |
| 367 | 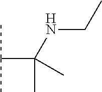 | O | H | H | H | H |
| 368 | 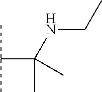 | O | H | H | H | H |
| 369 | 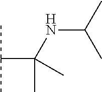 | O | H | H | H | H |
| 370 | 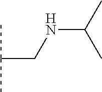 | O | H | H | H | H |
| 371 | 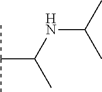 | O | H | H | H | H |
| 372 | 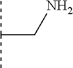 | O | H | H | H | H |
| 373 | 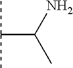 | O | H | H | H | H |
| 374 | 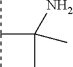 | O | H | H | H | H |
| 375 | 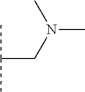 | O | H | H | H | H |
| 376 | 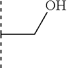 | O | H | H | H | H |
| 377 | 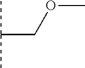 | O | H | H | H | H |
| 378 | 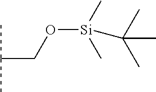 | O | H | H | H | H |
| 379 | 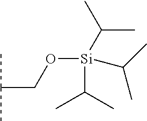 | O | H | H | H | H |
| 380 | 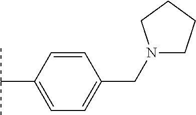 | O | H | H | H | H |
| 381 | 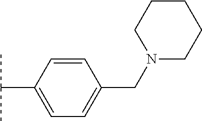 | O | H | H | H | H |
| 382 | 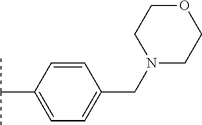 | O | H | H | H | H |
| 383 | 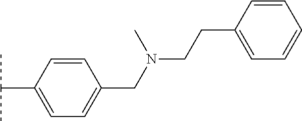 | O | H | H | H | H |
| 384 | 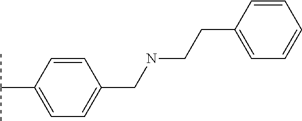 | O | H | H | H | H |
| 385 | 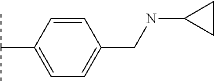 | O | H | H | H | H |
| 386 | 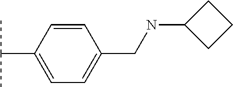 | O | H | H | H | H |
| 387 | 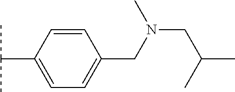 | O | H | H | H | H |
| 388 | 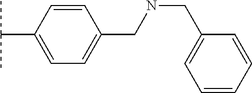 | O | H | H | H | H |
| 389 | 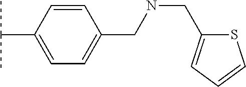 | O | H | H | H | H |
| 390 | 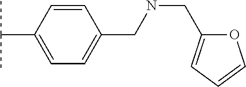 | O | H | H | H | H |
| 391 | 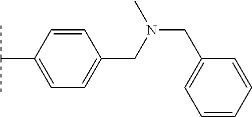 | O | H | H | H | H |
| 392 | 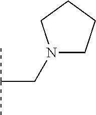 | O | H | H | H | H |
| 393 | 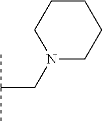 | O | H | H | H | H |
| 394 | 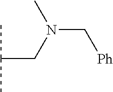 | O | H | H | H | H |
| 395 | 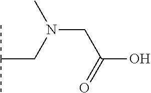 | O | H | H | H | H |
| 396 | 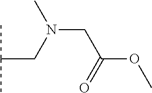 | O | H | H | H | H |
| 397 | 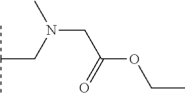 | O | H | H | H | H |
| 398 | 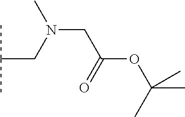 | O | H | H | H | H |
| 399 | 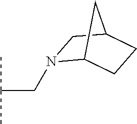 | O | H | H | H | H |
| 400 | 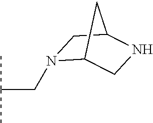 | O | H | H | H | H |
| 401 | 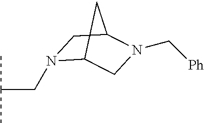 | O | H | H | H | H |
| 402 | 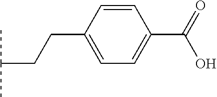 | O | H | H | H | H |
| 403 | 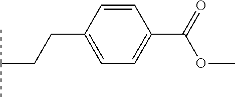 | O | H | H | H | H |
| 404 | 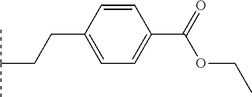 | O | H | H | H | H |
| 405 | 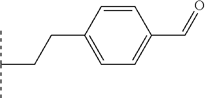 | O | H | H | H | H |
| 406 | 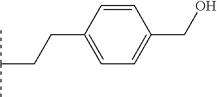 | O | H | H | H | H |
| 407 | 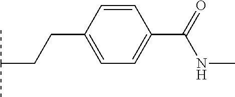 | O | H | H | H | H |
| 408 | 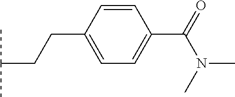 | O | H | H | H | H |
| 409 | 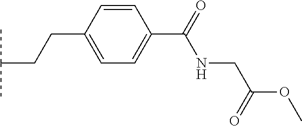 | O | H | H | H | H |
| 410 | 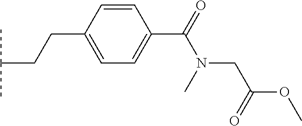 | O | H | H | H | H |
| 411 | 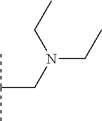 | O | H | H | H | H |
| 412 | 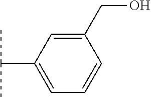 | O | H | H | H | H |
| 413 | 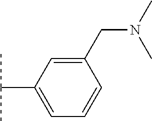 | O | H | H | H | H |
| 414 | 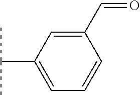 | O | H | H | H | H |
| 415 | 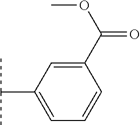 | O | H | H | H | H |
| 416 | 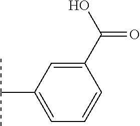 | O | H | H | H | H |
| 417 | 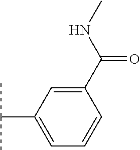 | O | H | H | H | H |
| 418 | 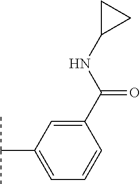 | O | H | H | H | H |
| 419 | 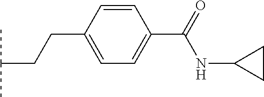 | O | H | H | H | H |
| 420 | 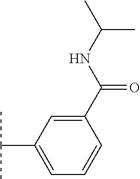 | O | H | H | H | H |
| 421 | 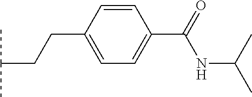 | O | H | H | H | H |
| 422 | 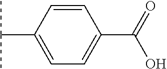 | O | H | H | H | H |
| 423 | 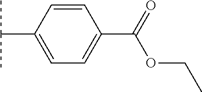 | O | H | H | H | H |
| 424 | 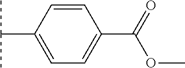 | O | H | H | H | H |
| 425 | 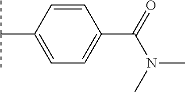 | O | H | H | H | H |
| 426 | 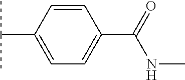 | O | H | H | H | H |
| 427 | 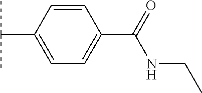 | O | H | H | H | H |
| 428 | 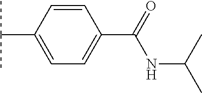 | O | H | H | H | H |
| 429 | 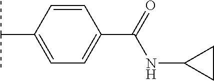 | O | H | H | H | H |
| 430 | 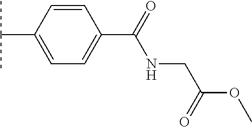 | O | H | H | H | H |
| 431 | 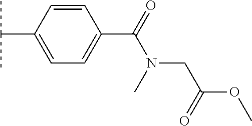 | O | H | H | H | H |
| 432 | 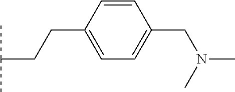 | O | H | H | H | H |
| 433 | 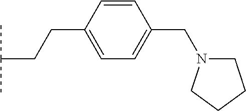 | O | H | H | H | H |
| 434 | 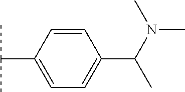 | O | H | H | H | H |
| 435 | 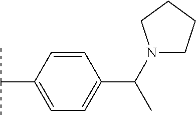 | O | H | H | H | H |
| 436 | 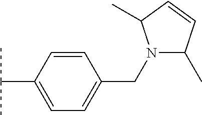 | O | H | H | H | H |
| 437 | 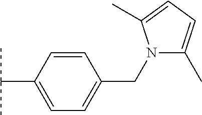 | O | H | H | H | H |
| 438 | 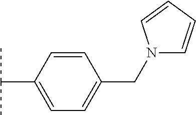 | O | H | H | H | H |
| 439 | 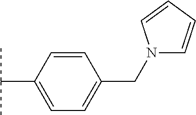 | O | H | H | H | H |
| 440 | 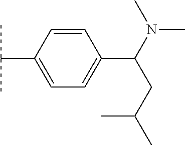 | O | H | H | H | H |
| 441 | 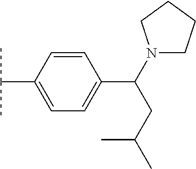 | O | H | H | H | H |
| 442 | 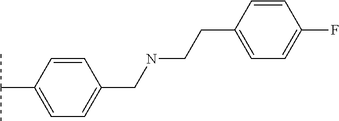 | O | H | H | H | H |
| 443 | 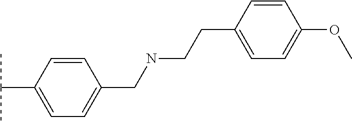 | O | H | H | H | H |
| 444 | 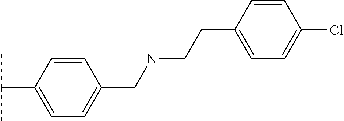 | O | H | H | H | H |
| 445 | 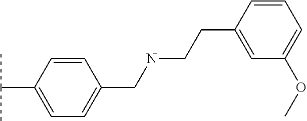 | O | H | H | H | H |
| 446 | 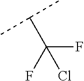 | O | H | H | H | H |
| 447 | 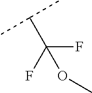 | O | H | H | H | H |
| 448 | 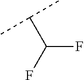 | O | H | H | H | H |
| 449 | 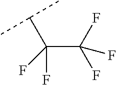 | O | H | H | H | H |
| 450 | 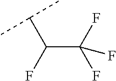 | O | H | H | H | H |
| 451 | 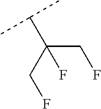 | O | H | H | H | H |
| 452 | 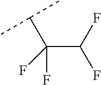 | O | H | H | H | H |
| 453 | 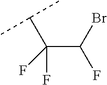 | O | H | H | H | H |
| 454 | 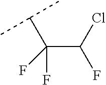 | O | H | H | H | H |
| 455 | 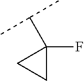 | O | H | H | H | H |
| 456 | 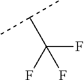 | O | H | H | H | H |
| 457 | 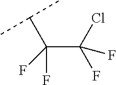 | O | H | H | H | H |
| 458 | 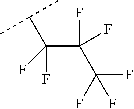 | O | H | H | H | H |
| 459 | 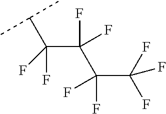 | O | H | H | H | H |
| 460 | 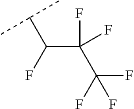 | O | H | H | H | H |
| 461 | 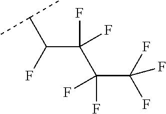 | O | H | H | H | H |
| 462 | 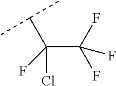 | O | H | H | H | H |
| 463 | 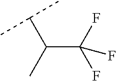 | O | H | H | H | H |
| 464 | 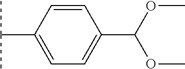 | O | H | H | H | H |
| 465 | 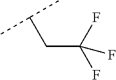 | O | H | H | H | H |
| 466 | 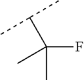 | O | H | H | H | H |
| 467 | 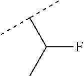 | O | H | H | H | H |
| 468 | 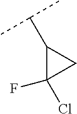 | O | H | H | H | H |
| 469 | 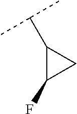 | O | H | H | H | H |
| 470 | 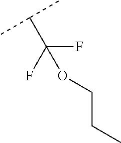 | O | H | H | H | H |
| 471 | 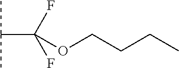 | O | H | H | H | H |
| 472 | 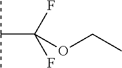 | O | H | H | H | H |
| 473 | 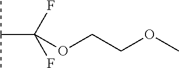 | O | H | H | H | H |
| 474 | 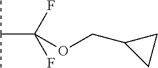 | O | H | H | H | H |
| 475 | 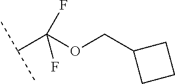 | O | H | H | H | H |
| 476 | 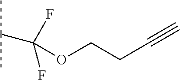 | O | H | H | H | H |
| 477 | 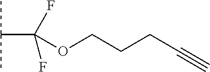 | O | H | H | H | H |
| 478 | 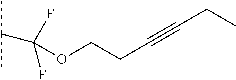 | O | H | H | H | H |
| 479 | 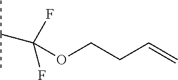 | O | H | H | H | H |
| 480 | 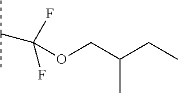 | O | H | H | H | H |
| 481 | 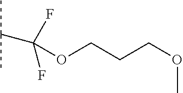 | O | H | H | H | H |
| 482 | 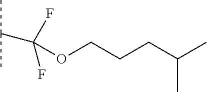 | O | H | H | H | H |
| 483 | 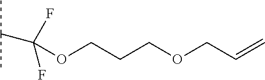 | O | H | H | H | H |
| 484 | 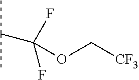 | O | H | H | H | H |
| 485 | 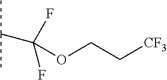 | O | H | H | H | H |
| 486 | 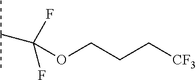 | O | H | H | H | H |
| 487 | 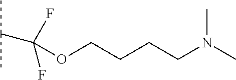 | O | H | H | H | H |
| 488 | 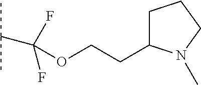 | O | H | H | H | H |
| 489 | 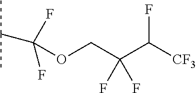 | O | H | H | H | H |
| 490 | 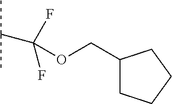 | O | H | H | H | H |
| 491 | 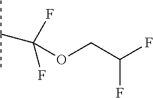 | O | H | H | H | H |
| 492 | 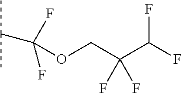 | O | H | H | H | H |
| 493 | 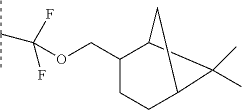 | O | H | H | H | H |
| 494 | 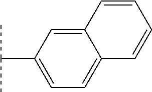 | O | H | H | H | H |
| 495 | 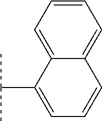 | O | H | H | H | H |
| 496 | 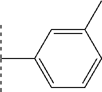 | O | H | H | H | H |
| 497 | 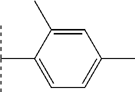 | O | H | H | H | H |
| 498 | 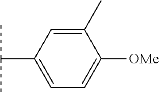 | O | H | H | H | H |
| 499 | 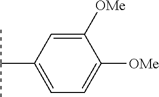 | O | H | H | H | H |
| 500 | 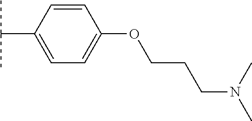 | O | H | H | H | H |
| 501 | 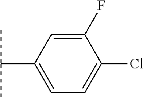 | O | H | H | H | H |
| 502 | 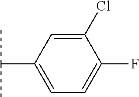 | O | H | H | H | H |
| 503 | 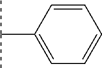 | S | H | H | H | H |
| 504 | 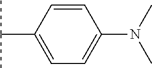 | S | H | H | H | H |
| 505 | 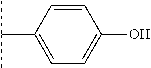 | S | H | H | H | H |
| 506 | 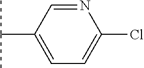 | S | H | H | H | H |
| 507 | 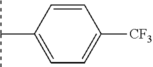 | S | H | H | H | H |
| 508 | 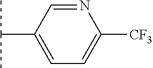 | S | H | H | H | H |
| 509 | 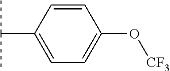 | S | H | H | H | H |
| 510 | 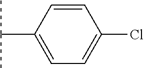 | S | H | H | H | H |
| 511 | 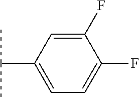 | S | H | H | H | H |
| 512 | 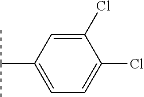 | S | H | H | H | H |
| 513 | 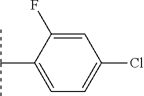 | S | H | H | H | H |
| 514 | 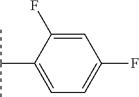 | S | H | H | H | H |
| 515 | 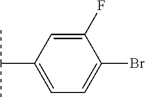 | S | H | H | H | H |
| 516 | 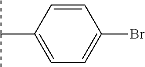 | S | H | H | H | H |
| 517 | 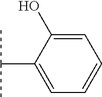 | S | H | H | H | H |
| 518 | 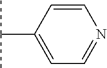 | S | H | H | H | H |
| 519 | 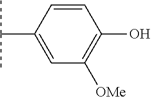 | S | H | H | H | H |
| 520 | 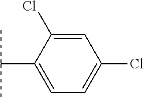 | S | H | H | H | H |
| 521 | 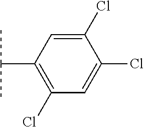 | S | H | H | H | H |
| 522 | 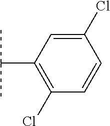 | S | H | H | H | H |
| 523 | 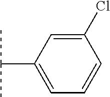 | S | H | H | H | H |
| 524 | 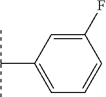 | S | H | H | H | H |
| 525 | 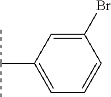 | S | H | H | H | H |
| 526 | 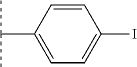 | S | H | H | H | H |
| 527 | 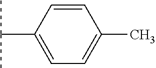 | S | H | H | H | H |
| 528 | 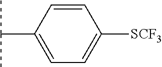 | S | H | H | H | H |
| 529 | 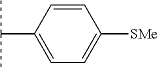 | S | H | H | H | H |
| 530 | 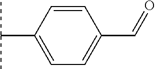 | S | H | H | H | H |
| 531 | 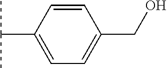 | S | H | H | H | H |
| 532 | 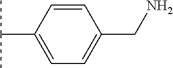 | S | H | H | H | H |
| 533 | 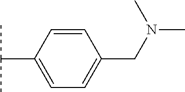 | S | H | H | H | H |
| 534 | 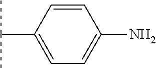 | S | H | H | H | H |
| 535 | 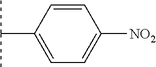 | S | H | H | H | H |
| 536 | 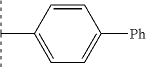 | S | H | H | H | H |
| 537 | 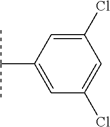 | S | H | H | H | H |
| 538 | 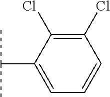 | S | H | H | H | H |
| 539 | 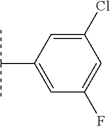 | S | H | H | H | H |
| 540 | 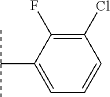 | S | H | H | H | H |
| 541 | 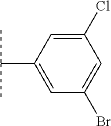 | S | H | H | H | H |
| 542 | 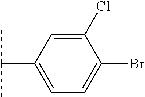 | S | H | H | H | H |
| 543 | 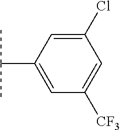 | S | H | H | H | H |
| 544 | 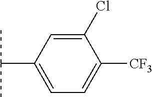 | S | H | H | H | H |
| 545 | 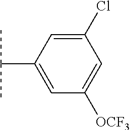 . | S | H | H | H | H |
| 546 | 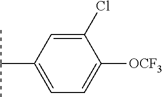 | S | H | H | H | H |
| 547 | 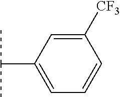 | S | H | H | H | H |
| 548 | 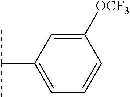 | S | H | H | H | H |
| 549 | 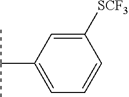 | S | H | H | H | H |
| 550 | 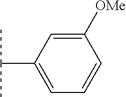 | S | H | H | H | H |
| 551 | 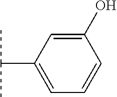 | S | H | H | H | H |
| 552 | 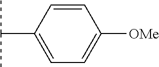 | S | H | H | H | H |
| 553 | 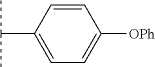 | S | H | H | H | H |
| 554 | 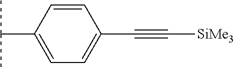 | S | H | H | H | H |
| 555 | 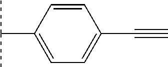 | S | H | H | H | H |
| 556 | 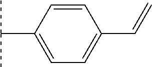 | S | H | H | H | H |
| 557 | 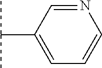 | S | H | H | H | H |
| 558 | 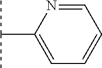 | S | H | H | H | H |
| 559 | 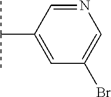 | S | H | H | H | H |
| 560 | 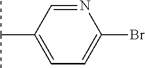 | S | H | H | H | H |
| 561 | 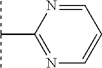 | S | H | H | H | H |
| 562 | 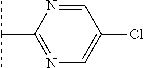 | S | H | H | H | H |
| 563 | 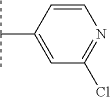 | S | H | H | H | H |
| 564 | 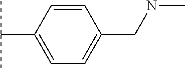 | S | H | H | H | H |
| 565 | 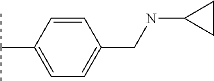 | S | H | H | H | H |
| 566 | 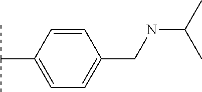 | S | H | H | H | H |
| 567 | 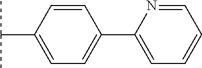 | S | H | H | H | H |
| 568 | 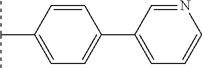 | S | H | H | H | H |
| 569 | 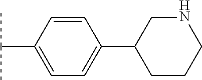 | S | H | H | H | H |
| 570 | 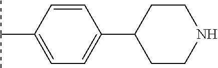 | S | H | H | H | H |
| 571 | 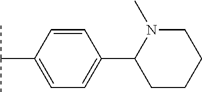 | S | H | H | H | H |
| 572 | 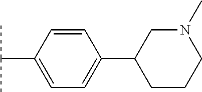 | S | H | H | H | H |
| 573 | 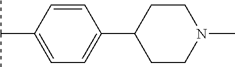 | S | H | H | H | H |
| 574 | 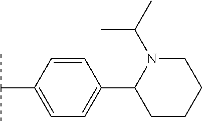 | S | H | H | H | H |
| 575 | 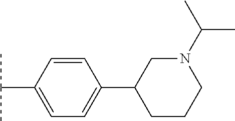 | S | H | H | H | H |
| 576 | 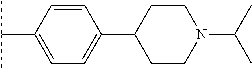 | S | H | H | H | H |
| 577 | 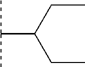 | S | H | H | H | H |
| 578 | 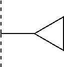 | S | H | H | H | H |
| 579 | 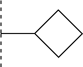 | S | H | H | H | H |
| 580 | 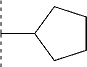 | S | H | H | H | H |
| 581 | 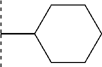 | S | H | H | H | H |
| 582 | 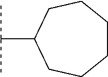 | S | H | H | H | H |
| 583 | 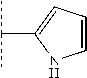 | S | H | H | H | H |
| 584 | 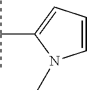 | S | H | H | H | H |
| 585 | 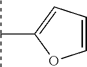 | S | H | H | H | H |
| 586 | 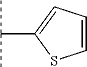 | S | H | H | H | H |
| 587 | 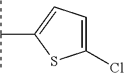 | S | H | H | H | H |
| 588 | 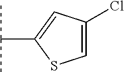 | S | H | H | H | H |
| 589 | 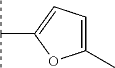 | S | H | H | H | H |
| 590 | 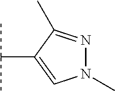 | S | H | H | H | H |
| 591 | 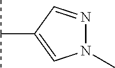 | S | H | H | H | H |
| 592 | 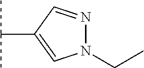 | S | H | H | H | H |
| 593 | 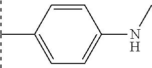 | S | H | H | H | H |
| 594 | 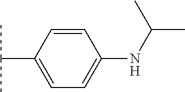 | S | H | H | H | H |
| 595 | 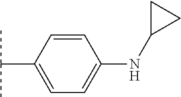 | S | H | H | H | H |
| 596 | 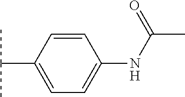 | S | H | H | H | H |
| 597 | 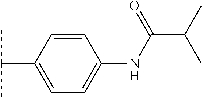 | S | H | H | H | H |
| 598 | 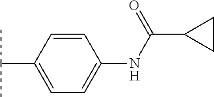 | S | H | H | H | H |
| 599 | 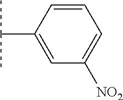 | S | H | H | H | H |
| 600 | 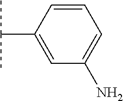 | S | H | H | H | H |
| 601 | 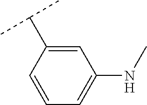 | S | H | H | H | H |
| 602 | 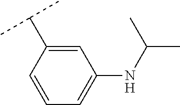 | S | H | H | H | H |
| 603 | 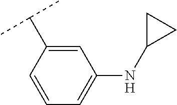 | S | H | H | H | H |
| 604 | 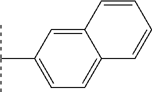 | S | H | H | H | H |
| 605 | 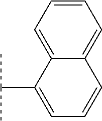 | S | H | H | H | H |
| 606 | 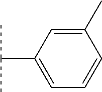 | S | H | H | H | H |
| 607 | 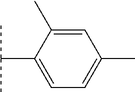 | S | H | H | H | H |
| 608 | 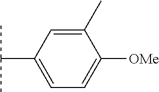 | S | H | H | H | H |
| 609 | 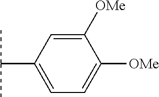 | S | H | H | H | H |
| 610 | 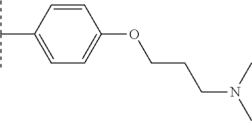 | S | H | H | H | H |
| 611 | 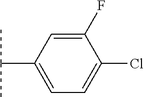 | S | H | H | H | H |
| 612 | 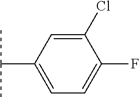 | S | H | H | H | H |
| 613 | 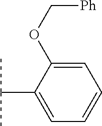 | S | H | H | H | H |
| 614 | 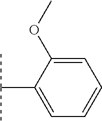 | S | H | H | H | H |
| 615 | 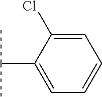 | S | H | H | H | H |
| 616 | 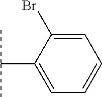 | S | H | H | H | H |
| 617 | 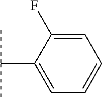 | S | H | H | H | H |
| 618 | 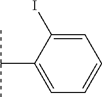 | S | H | H | H | H |
| 619 | 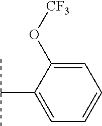 | S | H | H | H | H |
| 620 | 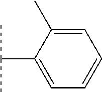 | S | H | H | H | H |
| 621 | 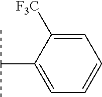 | S | H | H | H | H |
| 622 | 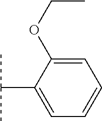 | S | H | H | H | H |
| 623 | 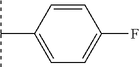 | S | H | H | H | H |
| 624 | 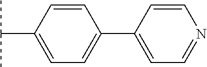 | S | H | H | H | H |
| 625 | 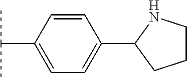 | S | H | H | H | H |
| 626 | 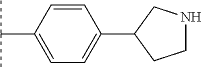 | S | H | H | H | H |
| 627 | 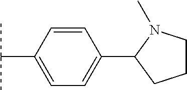 | S | H | H | H | H |
| 628 | 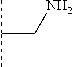 | S | H | H | H | H |
| 629 | 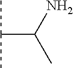 | S | H | H | H | H |
| 630 | 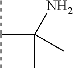 | S | H | H | H | H |
| 631 | 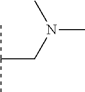 | S | H | H | H | H |
| 632 | 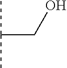 | S | H | H | H | H |
| 633 | 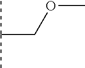 | S | H | H | H | H |
| 634 | 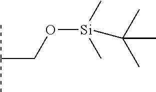 | S | H | H | H | H |
| 635 | 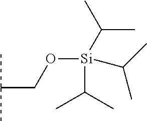 | S | H | H | H | H |
| 636 | 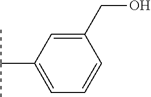 | S | H | H | H | H |
| 637 | 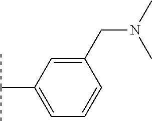 | S | H | H | H | H |
| 638 | 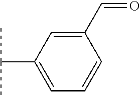 | S | H | H | H | H |
| 639 | 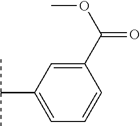 | S | H | H | H | H |
| 640 | 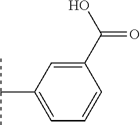 | S | H | H | H | H |
| 641 | 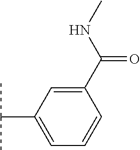 | S | H | H | H | H |
| 642 | 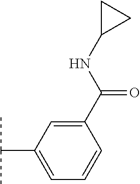 | S | H | H | H | H |
| 643 | 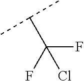 | S | H | H | H | H |
| 644 | 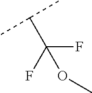 | S | H | H | H | H |
| 645 | 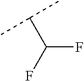 | S | H | H | H | H |
| 646 | 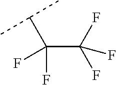 | S | H | H | H | H |
| 647 | 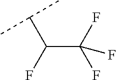 | S | H | H | H | H |
| 648 | 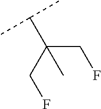 | S | H | H | H | H |
| 649 | 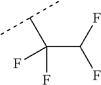 | S | H | H | H | H |
| 650 | 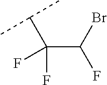 | S | H | H | H | H |
| 651 | 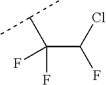 | S | H | H | H | H |
| 652 | 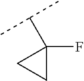 | S | H | H | H | H |
| 653 | 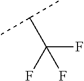 | S | H | H | H | H |
| 654 | 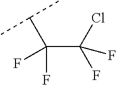 | S | H | H | H | H |
| 655 | 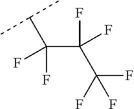 | S | H | H | H | H |
| 656 | 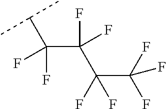 | S | H | H | H | H |
| 657 | 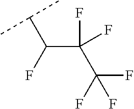 | S | H | H | H | H |
| 658 | 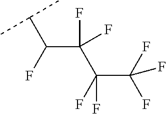 | S | H | H | H | H |
| 659 | 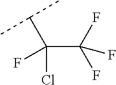 | S | H | H | H | H |
| 660 | 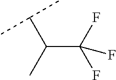 | S | H | H | H | H |
| 661 | 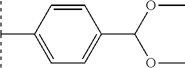 | S | H | H | H | H |
| 662 | 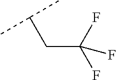 | S | H | H | H | H |
| 663 | 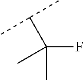 | S | H | H | H | H |
| 664 | 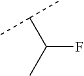 | S | H | H | H | H |
| 665 | 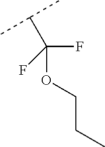 | S | H | H | H | H |
| 666 | 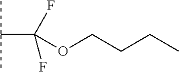 | S | H | H | H | H |
| 667 | 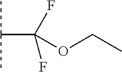 | S | H | H | H | H |
| 668 | 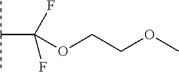 | S | H | H | H | H |
| 669 | 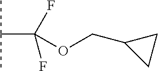 | S | H | H | H | H |
| 670 | 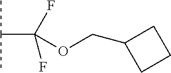 | S | H | H | H | H |
| 671 | 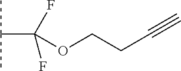 | S | H | H | H | H |
| 672 | 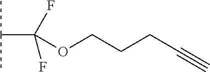 | S | H | H | H | H |
| 673 | 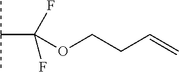 | S | H | H | H | H |
| 674 | 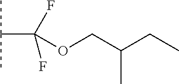 | S | H | H | H | H |
| 675 | 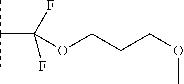 | S | H | H | H | H |
| 676 | 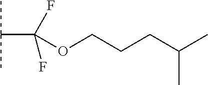 | S | H | H | H | H |
| 677 | 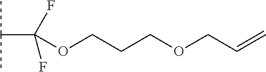 | S | H | H | H | H |
| 678 | 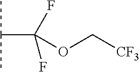 | S | H | H | H | H |
| 679 | 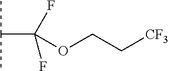 | S | H | H | H | H |
| 680 | 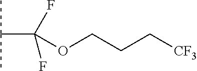 | S | H | H | H | H |
| 681 | 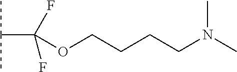 | S | H | H | H | H |
| 682 | 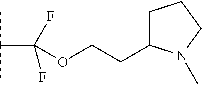 | S | H | H | H | H |
| 683 | 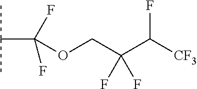 | S | H | H | H | H |
| 684 | 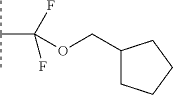 | S | H | H | H | H |
| 685 | 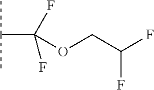 | S | H | H | H | H |
| 686 | 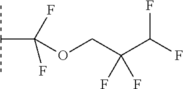 | S | H | H | H | H |
| 687 | 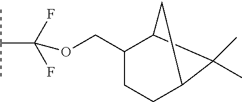 | S | H | H | H | H |
| 688 | 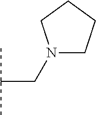 | S | H | H | H | H |
| 689 | 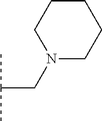 | S | H | H | H | H |
| 690 | 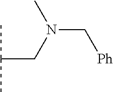 | S | H | H | H | H |
| 691 | 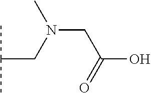 | S | H | H | H | H |
| 692 | 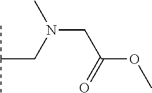 | S | H | H | H | H |
| 693 | 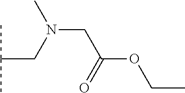 | S | H | H | H | H |
| 694 | 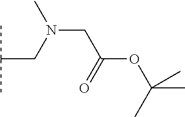 | S | H | H | H | H |
| 695 | 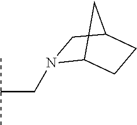 | S | H | H | H | H |
| 696 | 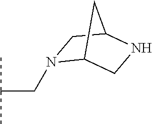 | S | H | H | H | H |
| 697 | 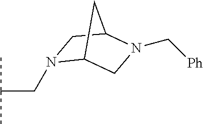 | S | H | H | H | H |
| 698 | 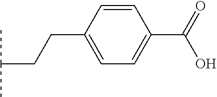 | S | H | H | H | H |
| 699 | 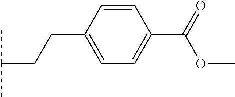 | S | H | H | H | H |
| 700 | 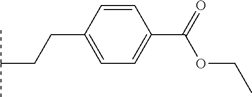 | S | H | H | H | H |
| 701 | 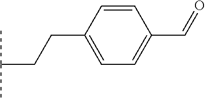 | S | H | H | H | H |
| 702 | 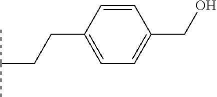 | S | H | H | H | H |
| 703 | 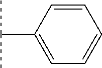 | O | 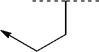 | H | 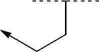 | H |
| 704 | 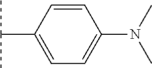 | O | 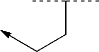 | H | 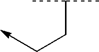 | H |
| 705 | 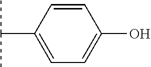 | O | 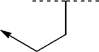 | H | 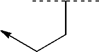 | H |
| 706 | 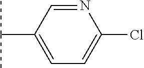 | O | 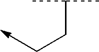 | H | 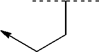 | H |
| 707 | 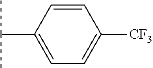 | O | 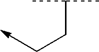 | H | 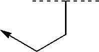 | H |
| 708 | 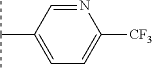 | O | 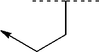 | H | 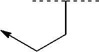 | H |
| 709 | 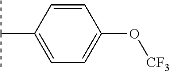 | O | 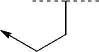 | H | 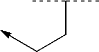 | H |
| 710 | 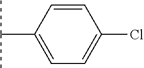 | O | 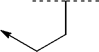 | H | 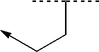 | H |
| 711 | 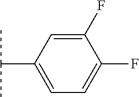 | O | 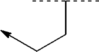 | H | 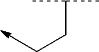 | H |
| 712 | 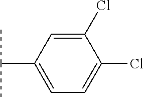 | O | 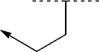 | H | 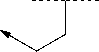 | H |
| 713 | 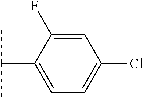 | O | 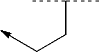 | H | 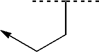 | H |
| 714 | 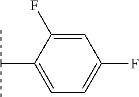 | O | 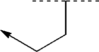 | H | 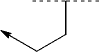 | H |
| 715 | 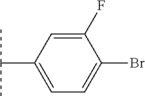 | O | 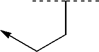 | H | 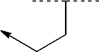 | H |
| 716 | 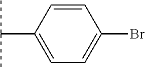 | O | 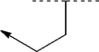 | H | 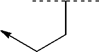 | H |
| 717 | 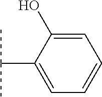 | O | 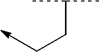 | H | 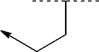 | H |
| 718 | 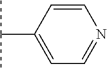 | O | 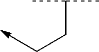 | H | 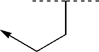 | H |
| 719 | 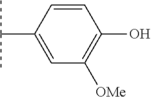 | O | 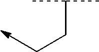 | H | 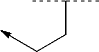 | H |
| 720 | 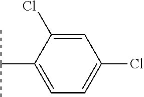 | O | 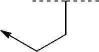 | H | 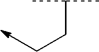 | H |
| 721 | 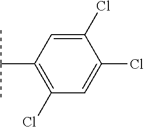 | O | 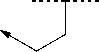 | H | 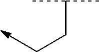 | H |
| 722 | 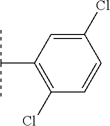 | O | 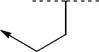 | H | 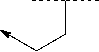 | H |
| 723 | 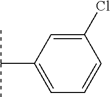 | O | 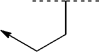 | H | 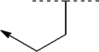 | H |
| 724 | 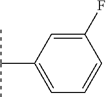 | O | 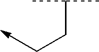 | H | 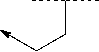 | H |
| 725 | 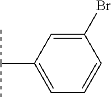 | O | 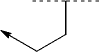 | H | 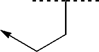 | H |
| 726 | 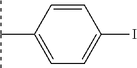 | O | 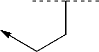 | H | 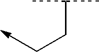 | H |
| 727 | 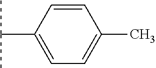 | O | 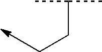 | H | 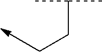 | H |
| 728 | 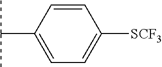 | O | 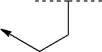 | H | 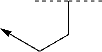 | H |
| 729 | 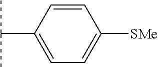 | O | 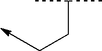 | H | 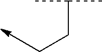 | H |
| 730 | 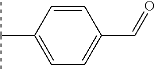 | O | 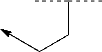 | H | 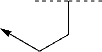 | H |
| 731 | 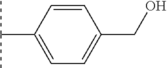 | O | 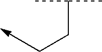 | H | 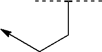 | H |
| 732 | 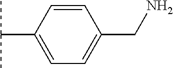 | O | 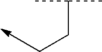 | H | 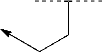 | H |
| 733 | 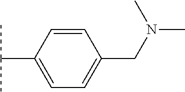 | O | 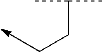 | H | 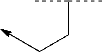 | H |
| 734 | 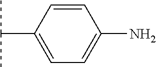 | O | 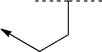 | H | 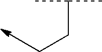 | H |
| 735 | 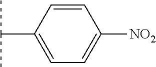 | O | 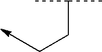 | H | 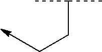 | H |
| 736 | 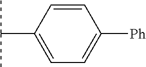 | O | 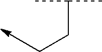 | H | 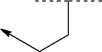 | H |
| 737 | 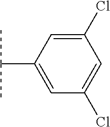 | O | 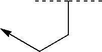 | H | 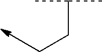 | H |
| 738 | 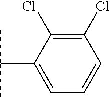 | O | 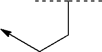 | H | 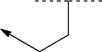 | H |
| 739 | 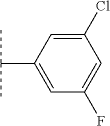 | O | 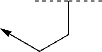 | H | 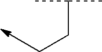 | H |
| 740 | 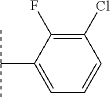 | O | 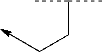 | H | 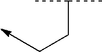 | H |
| 741 | 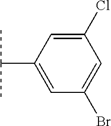 | O | 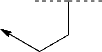 | H | 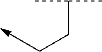 | H |
| 742 | 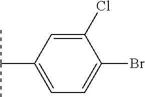 | O | 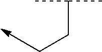 | H | 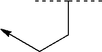 | H |
| 743 | 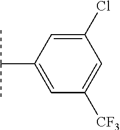 | O | 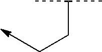 | H | 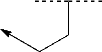 | H |
| 744 | 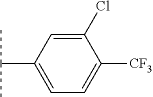 | O | 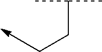 | H | 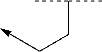 | H |
| 745 | 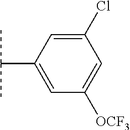 | O | 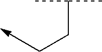 | H | 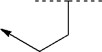 | H |
| 746 | 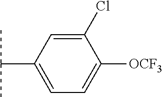 | O | 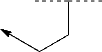 | H | 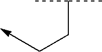 | H |
| 747 | 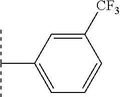 | O | 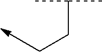 | H | 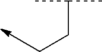 | H |
| 748 | 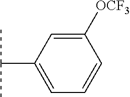 | O | 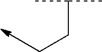 | H | 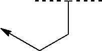 | H |
| 749 | 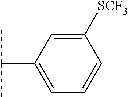 | O | 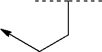 | H | 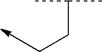 | H |
| 750 | 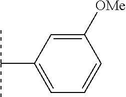 | O | 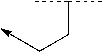 | H | 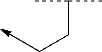 | H |
| 751 | 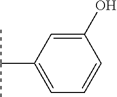 | O | 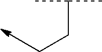 | H | 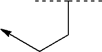 | H |
| 752 | 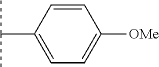 | O | 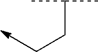 | H | 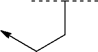 | H |
| 753 | 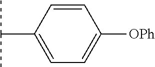 | O | 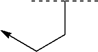 | H | 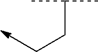 | H |
| 754 | 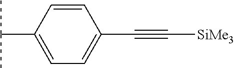 | O | 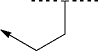 | H | 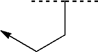 | H |
| 755 | 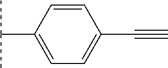 | O | 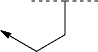 | H | 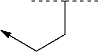 | H |
| 756 | 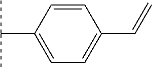 | O | 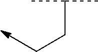 | H | 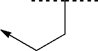 | H |
| 757 | 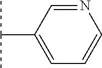 | O | 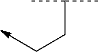 | H | 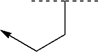 | H |
| 758 | 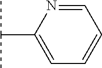 | O | 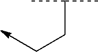 | H | 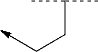 | H |
| 759 | 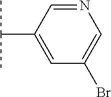 | O | 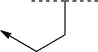 | H | 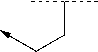 | H |
| 760 | 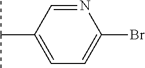 | O | 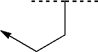 | H | 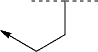 | H |
| 761 | 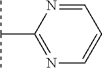 | O | 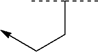 | H | 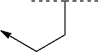 | H |
| 762 | 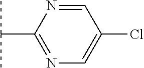 | O | 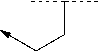 | H | 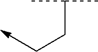 | H |
| 763 | 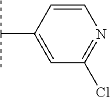 | O | 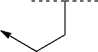 | H | 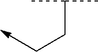 | H |
| 764 | 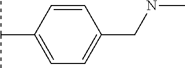 | O | 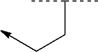 | H | 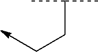 | H |
| 765 | 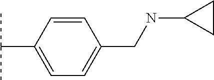 | O | 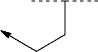 | H | 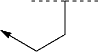 | H |
| 766 | 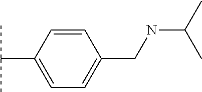 | O | 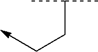 | H | 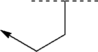 | H |
| 767 | 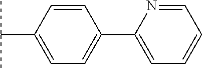 | O | 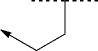 | H | 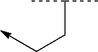 | H |
| 768 | 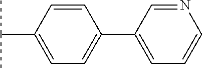 | O | 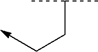 | H | 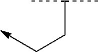 | H |
| 769 | 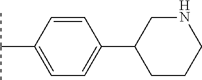 | H | 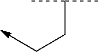 | H | 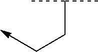 | H |
| 770 | 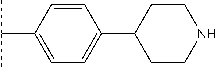 | O | 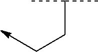 | H | 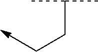 | H |
| 771 | 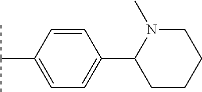 | O | 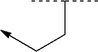 | H | 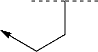 | H |
| 772 | 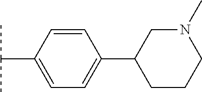 | O | 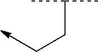 | H | 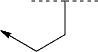 | H |
| 773 | 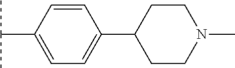 | O | 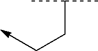 | H | 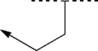 | H |
| 774 | 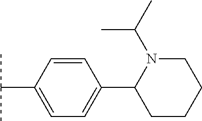 | O | 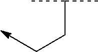 | H | 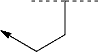 | H |
| 775 | 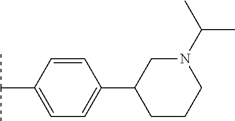 | O | 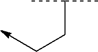 | H | 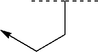 | H |
| 776 | 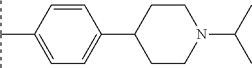 | O | 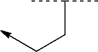 | H | 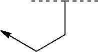 | H |
| 777 | 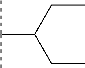 | O | 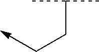 | H | 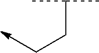 | H |
| 778 | 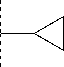 | O | 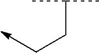 | H | 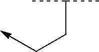 | H |
| 779 | 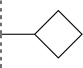 | O | 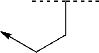 | H | 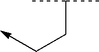 | H |
| 780 | 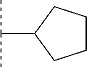 | O | 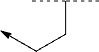 | H | 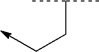 | H |
| 781 | 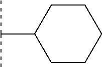 | O | 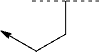 | H | 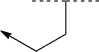 | H |
| 782 | 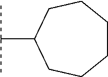 | O | 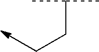 | H | 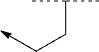 | H |
| 783 | 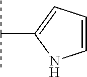 | O | 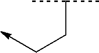 | H | 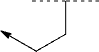 | H |
| 784 | 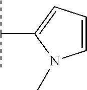 | O | 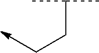 | H | 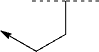 | H |
| 785 | 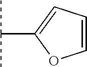 | O | 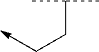 | H | 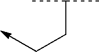 | H |
| 786 | 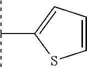 | O | 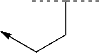 | H | 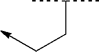 | H |
| 787 | 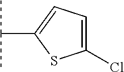 | O | 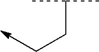 | H | 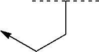 | H |
| 788 | 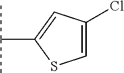 | O | 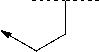 | H | 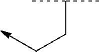 | H |
| 789 | 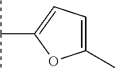 | O | 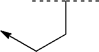 | H | 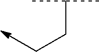 | H |
| 790 | 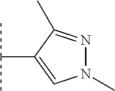 | O | 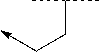 | H | 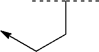 | H |
| 791 | 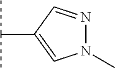 | O | 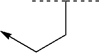 | H | 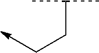 | H |
| 792 | 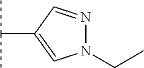 | O | 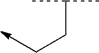 | H | 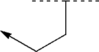 | H |
| 793 | 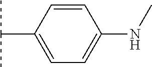 | O | 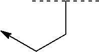 | H | 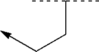 | H |
| 794 | 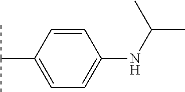 | O | 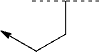 | H | 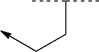 | H |
| 795 | 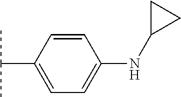 | O | 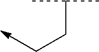 | H | 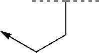 | H |
| 796 | 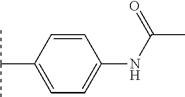 | O | 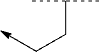 | H | 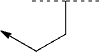 | H |
| 797 | 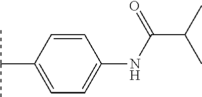 | O | 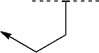 | H | 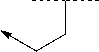 | H |
| 798 | 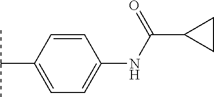 | O | 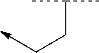 | H | 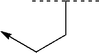 | H |
| 799 | 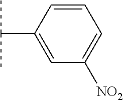 | O | 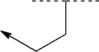 | H | 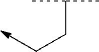 | H |
| 800 | 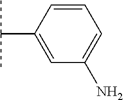 | O | 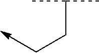 | H | 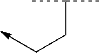 | H |
| 801 | 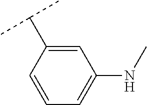 | O | 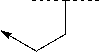 | H | 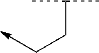 | H |
| 802 | 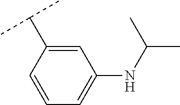 | O | 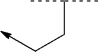 | H | 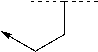 | H |
| 803 | 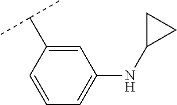 | O | 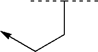 | H | 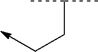 | H |
| 804 | 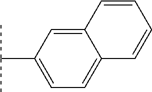 | O | 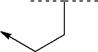 | H | 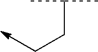 | H |
| 805 | 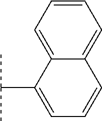 | O | 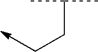 | H | 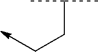 | H |
| 806 | 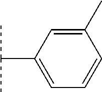 | O | 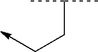 | H | 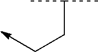 | H |
| 807 | 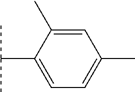 | O | 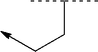 | H | 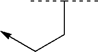 | H |
| 808 | 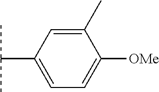 | O | 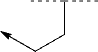 | H | 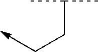 | H |
| 809 | 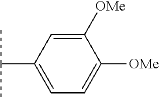 | O | 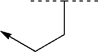 | H | 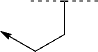 | H |
| 810 | 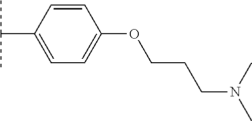 | O | 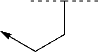 | H | 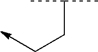 | H |
| 811 | 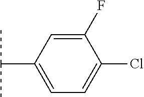 | O | 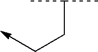 | H | 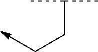 | H |
| 812 | 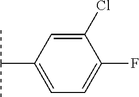 | O | 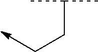 | H | 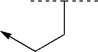 | H |
| 813 | 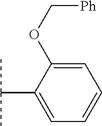 | O | 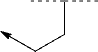 | H | 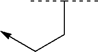 | H |
| 814 | 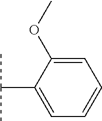 | O | 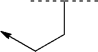 | H | 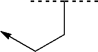 | H |
| 815 | 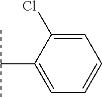 | O | 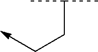 | H | 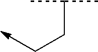 | H |
| 816 | 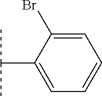 | O | 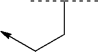 | H | 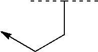 | H |
| 817 | 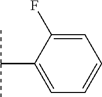 | O | 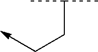 | H | 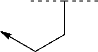 | H |
| 818 | 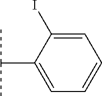 | O | 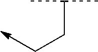 | H | 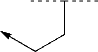 | H |
| 819 | 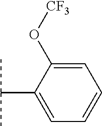 | O | 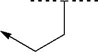 | H | 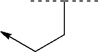 | H |
| 820 | 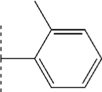 | O | 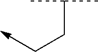 | H | 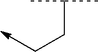 | H |
| 821 | 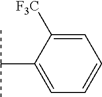 | O | 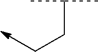 | H | 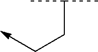 | H |
| 822 | 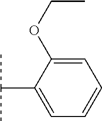 | O | 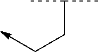 | H | 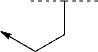 | H |
| 823 | 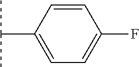 | O | 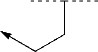 | H | 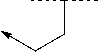 | H |
| 824 | 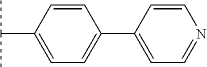 | O | 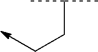 | H | 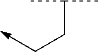 | H |
| 825 | 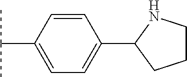 | O | 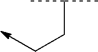 | H | 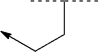 | H |
| 826 | 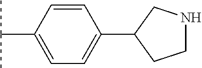 | O | 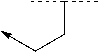 | H | 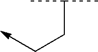 | H |
| 827 | 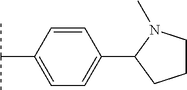 | O | 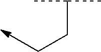 | H | 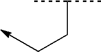 | H |
| 828 | 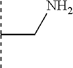 | O | 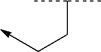 | H | 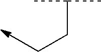 | H |
| 829 | 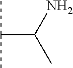 | O | 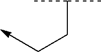 | H | 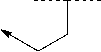 | H |
| 830 | 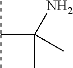 | O | 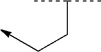 | H | 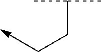 | H |
| 831 | 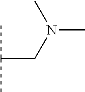 | O | 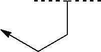 | H | 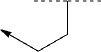 | H |
| 832 | 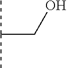 | O | 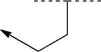 | H | 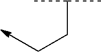 | H |
| 833 | 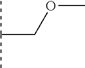 | O | 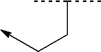 | H | 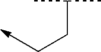 | H |
| 834 | 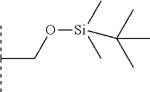 | O | 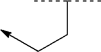 | H | 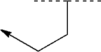 | H |
| 835 | 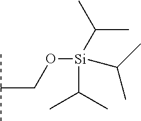 | O | 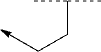 | H | 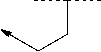 | H |
| 836 | 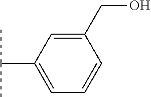 | O | 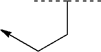 | H | 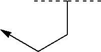 | H |
| 837 | 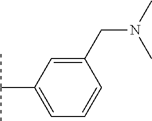 | O | 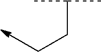 | H | 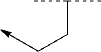 | H |
| 838 | 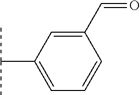 | O | 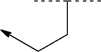 | H | 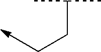 | H |
| 839 | 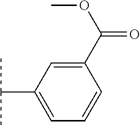 | O | 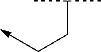 | H | 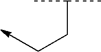 | H |
| 840 | 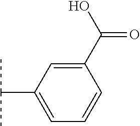 | O | 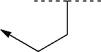 | H | 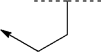 | H |
| 841 | 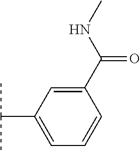 | O | 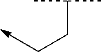 | H | 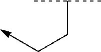 | H |
| 842 | 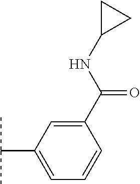 | O | 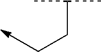 | H | 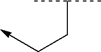 | H |
| 843 | 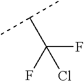 | O | 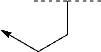 | H | 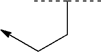 | H |
| 844 | 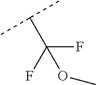 | O | 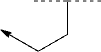 | H | 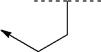 | H |
| 845 | 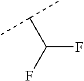 | O | 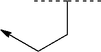 | H | 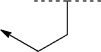 | H |
| 846 | 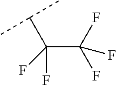 | O | 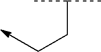 | H | 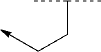 | H |
| 847 | 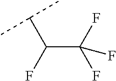 | O | 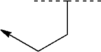 | H | 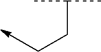 | H |
| 848 | 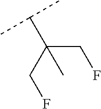 | O | 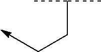 | H | 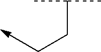 | H |
| 849 | 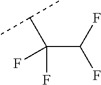 | O | 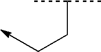 | H | 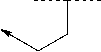 | H |
| 850 | 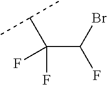 | O | 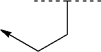 | H | 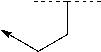 | H |
| 851 | 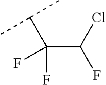 | O | 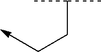 | H | 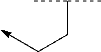 | H |
| 852 | 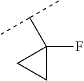 | O | 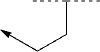 | H | 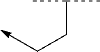 | H |
| 853 | 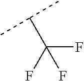 | O | 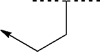 | H | 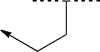 | H |
| 854 | 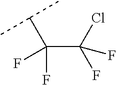 | O | 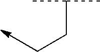 | H | 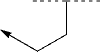 | H |
| 855 | 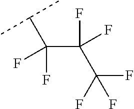 | O | 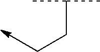 | H | 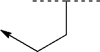 | H |
| 856 | 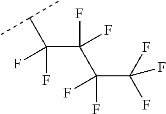 | O | 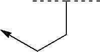 | H | 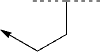 | H |
| 857 | 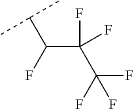 | O | 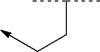 | H | 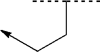 | H |
| 858 | 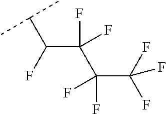 | O | 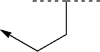 | H | 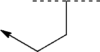 | H |
| 859 | 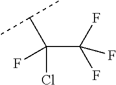 | O | 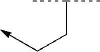 | H | 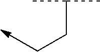 | H |
| 860 | 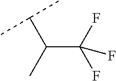 | O | 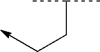 | H | 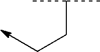 | H |
| 861 | 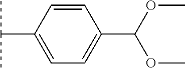 | O | 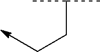 | H | 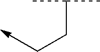 | H |
| 862 | 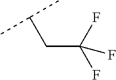 | O | 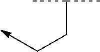 | H | 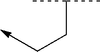 | H |
| 863 | 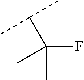 | O | 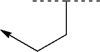 | H | 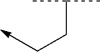 | H |
| 864 | 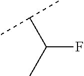 | O | 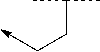 | H | 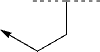 | H |
| 865 | 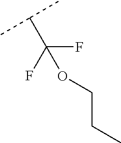 | O | 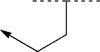 | H | 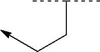 | H |
| 866 | 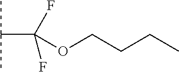 | O | 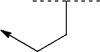 | H | 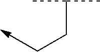 | H |
| 867 | 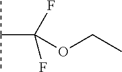 | O | 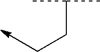 | H | 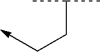 | H |
| 868 | 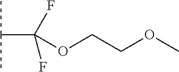 | O | 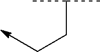 | H | 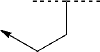 | H |
| 869 | 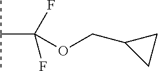 | O | 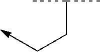 | H | 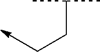 | H |
| 870 | 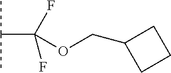 | O | 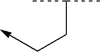 | H | 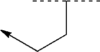 | H |
| 871 | 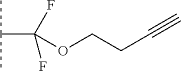 | O | 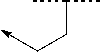 | H | 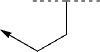 | H |
| 872 | 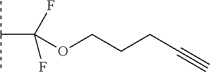 | O | 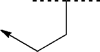 | H | 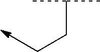 | H |
| 873 | 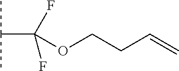 | O | 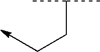 | H | 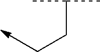 | H |
| 874 | 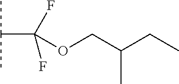 | O | 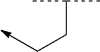 | H | 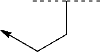 | H |
| 875 | 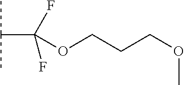 | O | 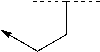 | H | 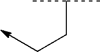 | H |
| 876 | 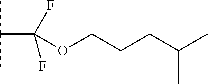 | O | 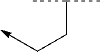 | H | 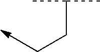 | H |
| 877 | 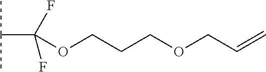 | O | 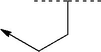 | H | 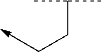 | H |
| 878 | 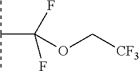 | O | 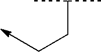 | H | 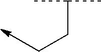 | H |
| 879 | 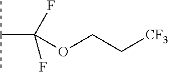 | O | 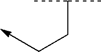 | H | 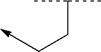 | H |
| 880 | 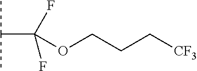 | O | 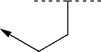 | H | 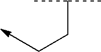 | H |
| 881 | 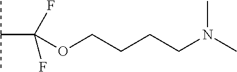 | O | 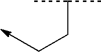 | H | 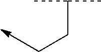 | H |
| 882 | 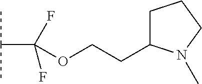 | O | 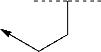 | H | 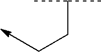 | H |
| 883 | 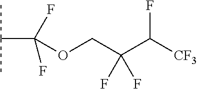 | O | 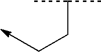 | H | 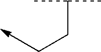 | H |
| 884 | 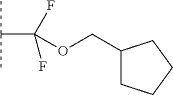 | O | 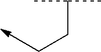 | H | 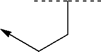 | H |
| 885 | 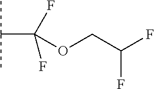 | O | 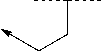 | H | 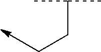 | H |
| 886 | 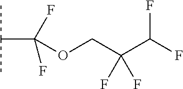 | O | 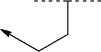 | H | 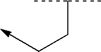 | H |
| 887 | 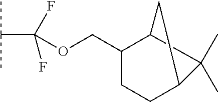 | O | 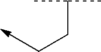 | H | 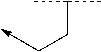 | H |
| 888 | 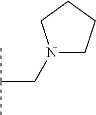 | O | 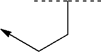 | H | 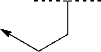 | H |
| 889 | 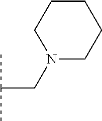 | O | 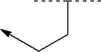 | H | 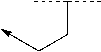 | H |
| 890 | 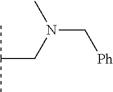 | O | 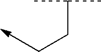 | H | 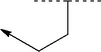 | H |
| 891 | 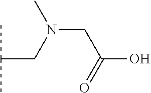 | O | 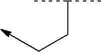 | H | 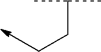 | H |
| 892 | 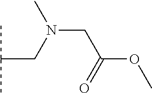 | O | 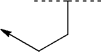 | H | 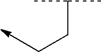 | H |
| 893 | 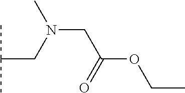 | O | 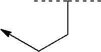 | H | 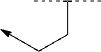 | H |
| 894 | 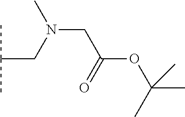 | O | 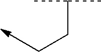 | H | 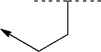 | H |
| 895 | 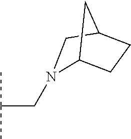 | O | 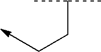 | H | 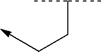 | H |
| 896 | 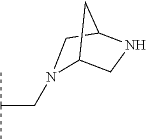 | O | 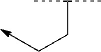 | H | 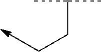 | H |
| 897 | 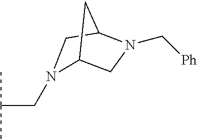 | O | 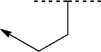 | H | 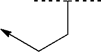 | H |
| 898 | 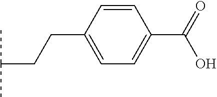 | O | 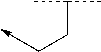 | H | 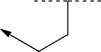 | H |
| 899 | 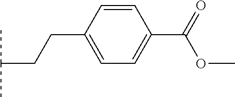 | O | 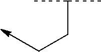 | H | 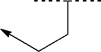 | H |
| 900 | 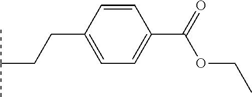 | O | 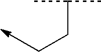 | H | 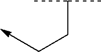 | H |
| 901 | 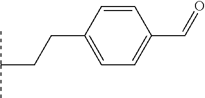 | O | 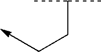 | H | 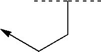 | H |
| 902 | 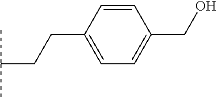 | O | 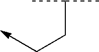 | H | 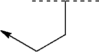 | H |
| 903 | 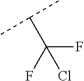 | O | CH₃ | H | H | H |
| 904 | 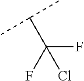 | O | CH₃ | H | CH₃ | H |
| 905 | 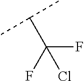 | O | Ph | H | Ph | H |
| 906 | 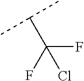 | O | CH₃ | CH₃ | CH₃ | CH₃ |
| 907 | 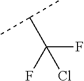 | O | 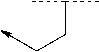 | H | 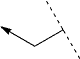 | H |
| 908 | 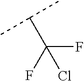 | O | 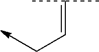 | H | 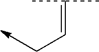 | H |
| 909 | 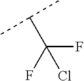 | O | H | H | CH₃ | H |
| 910 | 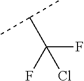 | O | c-Hex | H | c-Hex | H |
| 911 | 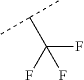 | O | CH₃ | H | H | H |
| 912 | 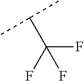 | O | CH₃ | H | CH₃ | H |
| 913 | 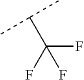 | O | Ph | H | Ph | H |
| 914 | 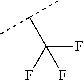 | O | CH₃ | CH₃ | CH₃ | CH₃ |
| 915 | 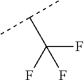 | O | 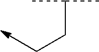 | H | 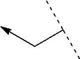 | H |
| 916 | 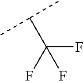 | O | 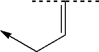 | H | 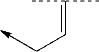 | H |
| 917 | 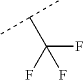 | O | H | H | CH₃ | H |
| 918 | 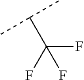 | O | c-Hex | H | c-Hex | H |
| 918 | 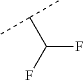 | O | CH₃ | H | H | H |
| 919 | 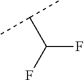 | O | CH₃ | H | CH₃ | H |
| 920 | 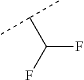 | O | Ph | H | Ph | H |
| 921 | 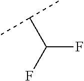 | O | CH₃ | CH₃ | CH₃ | CH₃ |
| 922 | 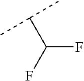 | O | 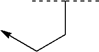 | H | 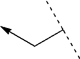 | H |
| 923 | 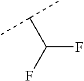 | O | 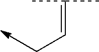 | H | 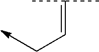 | H |
| 924 | 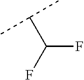 | O | H | H | CH₃ | H |
| 925 | 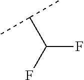 | O | c-Hex | H | c-Hex | H |
| 926 | 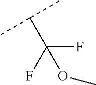 | O | CH₃ | H | H | H |
| 927 | 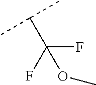 | O | CH₃ | H | CH₃ | H |
| 928 | 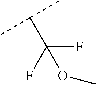 | O | Ph | H | Ph | H |
| 929 | 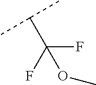 | O | CH₃ | CH₃ | CH₃ | CH₃ |
| 930 | 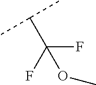 | O | 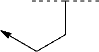 | H | 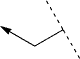 | H |
| 931 | 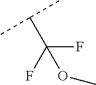 | O | 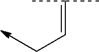 | H | 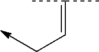 | H |
| 932 | 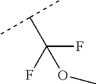 | O | H | H | CH₃ | H |
| 933 | 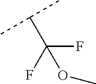 | O | c-Hex | H | c-Hex | H |
| 934 | 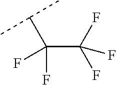 | O | CH₃ | H | H | H |
| 935 | 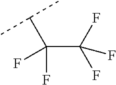 | O | CH₃ | H | CH₃ | H |
| 936 | 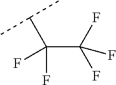 | O | Ph | H | Ph | H |
| 937 | 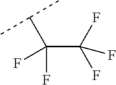 | O | CH₃ | CH₃ | CH₃ | CH₃ |
| 938 | 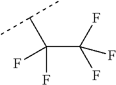 | O | 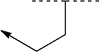 | H | 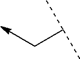 | H |
| 939 | 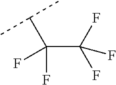 | O | 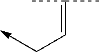 | H | 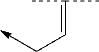 | H |
| 940 | 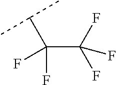 | O | H | H | CH₃ | H |
| 941 | 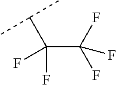 | O | c-Hex | H | c-Hex | H |
| 942 | 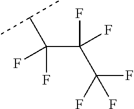 | O | CH₃ | H | H | H |
| 943 | 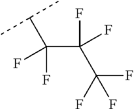 | O | CH₃ | H | CH₃ | H |
| 944 | 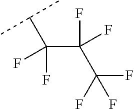 | O | Ph | H | Ph | H |
| 945 | 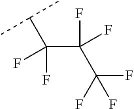 | O | CH₃ | CH₃ | CH₃ | CH₃ |
| 946 | 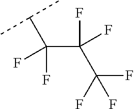 | O | 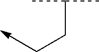 | H | 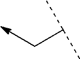 | H |
| 947 | 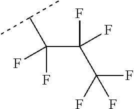 | O | 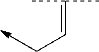 | H | 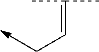 | H |
| 948 | 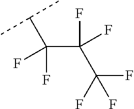 | O | H | H | CH₃ | H |
| 949 | 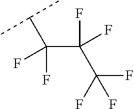 | O | c-Hex | H | c-Hex | H |

B2. Compounds B2-1 to B2-949 of the general formula (Ia) in which R¹ represents fluorine, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B2-1 to B2-949).
B3. Compounds B3-1 to B3-949 of the general formula (Ia) in which R¹ represents chlorine, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B3-1 to B3-949).
B4. Compounds B4-1 to B4-949 of the general formula (Ia) in which R¹ represents methyl, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B4-1 to B4-949).
B5. Compounds B5-1 to B5-949 of the general formula (Ia) in which R¹ represents trifluoromethyl, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B5-1 to B5-949).
B6. Compounds B6-1 to B6-949 of the general formula (Ia) in which R¹ represents trifluoromethylthio, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B6-1 to B6-949).
B7. Compounds B7-1 to B7-949 of the general formula (Ia) in which R¹ represents trifluoromethoxy, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B7-1 to B7-949).
B8. Compounds B8-1 to B8-949 of the general formula (Ia) in which R¹ represents methoxy, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B8-1 to B8-949).
B9. Compounds B9-1 to B9-949 of the general formula (Ia) in which R¹ represents methylthio, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds B9-1 to B9-949).
B10. Compounds B10-1 to B10-949 of the general formula (Ia) in which R² represents fluorine, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B10-1 to B10-949).
B11. Compounds B11-1 to B11-949 of the general formula (Ia) in which R² represents chlorine, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B11-1 to B11-949).
B12. Compounds B12-1 to B12-949 of the general formula (Ia) in which R² represents bromine, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B12-1 to B12-949).
B13. Compounds B13-1 to B13-949 of the general formula (Ia) in which R² represents iodine, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B13-1 to B13-949).
B14. Compounds B14-1 to B14-949 of the general formula (Ia) in which R² represents methyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B14-1 to B14-949).
B15. Compounds B15-1 to B15-949 of the general formula (Ia) in which R² represents trifluoromethyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B15-1 to B15-949).
B16. Compounds B16-1 to B16-949 of the general formula (Ia) in which R² represents trifluoromethylthio, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B16-1 to B16-949).
B17. Compounds B17-1 to B17-949 of the general formula (Ia) in which R² represents trifluoromethoxy, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B17-1 to B17-949).
B18. Compounds B18-1 to B18-949 of the general formula (Ia) in which R² represents methoxy, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B18-1 to B18-949).
B19. Compounds B19-1 to B19-949 of the general formula (Ia) in which R² represents methylthio, R¹, R³ and R⁹ represent hydrogen and Q, W, R⁵, R⁶, R⁷, R⁸ for the individual compound in question correspond to the radical definitions given in Table 1 (Nos 1 to 949; corresponding to Compounds B19-1 to B19-949).
B20. Compounds B20-1 to B20-949 of the general formula (Ia) in which R² represents ethyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B20-1 to B20-949).
B21. Compounds B21-1 to B21-949 of the general formula (Ia) in which R² represents isopropyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B21-1 to B21-949).
B22. Compounds B22-1 to B22-949 of the general formula (Ia) in which R² represents cyclopropyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B22-1 to B22-949).
B23. Compounds B23-1 to B23-949 of the general formula (Ia) in which R² represents phenyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B23-1 to B23-949).
B24. Compounds B24-1 to B24-949 of the general formula (Ia) in which R² represents thiophenyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B24-1 to B24-949).
B25. Compounds B25-1 to B25-949 of the general formula (Ia) in which R² represents p-Cl-phenyl, R¹, R³ and R⁹ represent hydrogen and Q, W, R⁵, R⁶, R⁷, R⁸ for the individual compound in question correspond to the radical definitions given in Table 2 (Nos 1 to 949; corresponding to Compounds B25-1 to B25-949).
B26. Compounds B26-1 to B26-949 of the general formula (Ia) in which R³ represents fluorine, R¹, R² and R⁹ represent hydrogen and Q, W, R⁵, R⁶, R⁷, R⁸ for the individual compound in question correspond to the radical definitions given in Table 2 (Nos 1 to 949; corresponding to Compounds B26-1 to B26-949).
B27. Compounds B27-1 to B27-949 of the general formula (Ia) in which R³ represents methyl, R¹, R² and R⁹ represent hydrogen and Q, W, R⁵, R⁶, R⁷, R⁸ for the individual compound in question correspond to the radical definitions given in Table 2 (Nos 1 to 949; corresponding to Compounds B27-1 to B27-949).
B28. Compounds B28-1 to B28-949 of the general formula (Ia) in which R⁹ represents methyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B28-1 to B28-949).
B29. Compounds B29-1 to B29-949 of the general formula (Ia) in which R⁹ represents ethyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B29-1 to B29-949).
B30. Compounds B30-1 to B30-949 of the general formula (Ia) in which R⁹ represents allyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B30-1 to B30-949).
B31. Compounds B31-1 to B31-949 of the general formula (Ia) in which R⁹ represents CH₂CN, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B31-1 to B31-949).
B32. Compounds B32-1 to B32-949 of the general formula (Ia) in which R⁹ represents n-propyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B32-1 to B32-949).
B33. Compounds B33-1 to B33-949 of the general formula (Ia) in which R⁹ represents C(═O)CH₃, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B33-1 to B33-949).
B34. Compounds B34-1 to B34-949 of the general formula (Ia) in which R⁹ represents C(═O)CH₂CH₃, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B34-1 to B34-949).
B35. Compounds B35-1 to B35-949 of the general formula (Ia) in which R⁹ represents C(═O)t-Bu, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B35-1 to B35-949).
B36. Compounds B36-1 to B36-949 of the general formula (Ia) in which R⁹ represents CH₂CH₂N(CH₃)₂, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B36-1 to B36-949).
B37. Compounds B37-1 to B37-949 of the general formula (Ia) in which R⁹ represents CH₂CH₂N(CH₂CH₃)₂, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B37-1 to B37-949).
B38. Compounds B38-1 to B38-949 of the general formula (Ia) in which R⁹ represents SO₂CH₃, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B38-1 to B38-949).
B39. Compounds B39-1 to B39-949 of the general formula (Ia) in which R⁹ represents SO₂c-Pr, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B39-1 to B39-949).
B40. Compounds B40-1 to B40-949 of the general formula (Ia) in which R⁹ represents CH₂CHF₂, R¹, R² and R³ represent hydrogen and Q, W, R⁵, R⁶, R⁷, R⁸ for the individual compound in question correspond to the radical definitions given in Table 2 (Nos 1 to 949; corresponding to Compounds B40-1 to B40-949).
C1. Compounds C1-1 to C1-949 of the general formula (Ib) in which R¹, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C1-1 to C1-949).

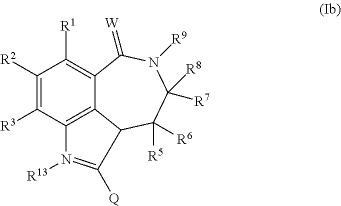

C2. Compounds C2-1 to C2-949 of the general formula (Ib) in which R¹ represents fluorine, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C2-1 to C2-949).
C3. Compounds C3-1 to C3-949 of the general formula (Ib) in which R¹ represents chlorine, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C3-1 to C3-949).
C4. Compounds C4-1 to C4-949 of the general formula (Ib) in which R¹ represents methyl, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C4-1 to C4-949).
C5. Compounds C5-1 to C5-949 of the general formula (Ib) in which R¹ represents trifluoromethyl, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C5-1 to C5-949).
C6. Compounds C6-1 to C6-949 of the general formula (Ib) in which R¹ represents methoxy, R², R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C6-1 to C6-949).
C7. Compounds C7-1 to C7-949 of the general formula (Ib) in which R² represents fluorine, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C7-1 to C7-949).
C8. Compounds C8-1 to C8-949 of the general formula (Ib) in which R² represents chlorine, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C8-1 to C8-949).
C9. Compounds C9-1 to C9-949 of the general formula (Ib) in which R² represents bromine, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C9-1 to C9-949).
C10. Compounds C10-1 to C10-949 of the general formula (Ib) in which R² represents iodine, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C10-1 to C10-949).
C11. Compounds C11-1 to C11-949 of the general formula (Ib) in which R² represents methyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C11-1 to C11-949).
C12. Compounds C12-1 to C12-949 of the general formula (Ib) in which R² represents methoxy, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C12-1 to C12-949).
C13. Compounds C13-1 to C13-949 of the general formula (Ib) in which R² represents trifluoromethyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C13-1 to C13-949).
C14. Compounds C14-1 to C14-949 of the general formula (Ib) in which R² represents trifluoromethoxy, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C14-1 to C14-949).
C15. Compounds C15-1 to C15-949 of the general formula (Ib) in which R² represents methylthio, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C15-1 to C15-949).
C16. Compounds C16-1 to C16-949 of the general formula (Ib) in which R² represents trifluoromethylthio, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C16-1 to C16-949).
C17. Compounds C17-1 to C17-949 of the general formula (Ib) in which R² represents ethyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C17-1 to C17-949).
C18. Compounds C18-1 to C18-949 of the general formula (Ib) in which R² represents isopropyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C18-1 to C18-949).
C19. Compounds C19-1 to C19-949 of the general formula (Ib) in which R² represents cyclopropyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C19-1 to C19-949).
C20. Compounds C20-1 to C20-949 of the general formula (Ib) in which R² represents phenyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C20-1 to C20-949).
C21. Compounds C21-1 to C21-949 of the general formula (Ib) in which R² represents thiophenyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C21-1 to C21-949).
C22. Compounds C22-1 to C22-949 of the general formula (Ib) in which R² represents ethynyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C22-1 to C22-949).
C23. Compounds C23-1 to C23-949 of the general formula (Ib) in which R² represents trimethylsilylethynyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C23-1 to C23-949).
C24. Compounds C24-1 to C24-949 of the general formula (Ib) in which R² represents p-Cl-phenyl, R¹, R³, R⁹ and R¹³ represent hydrogen and Q, W, R⁵, R⁶, R⁷, R⁸ for the individual compound in question correspond to the radical definitions given in Table 2 (Nos 1 to 949; corresponding to Compounds C24-1 to C24-949).
C25. Compounds C25-1 to C25-949 of the general formula (Ib) in which R⁹ represents CH₃, R¹, R², R³ and R¹³ represent hydrogen and Q, W, R⁵, R⁶, R⁷, R⁸ for the individual compound in question correspond to the radical definitions given in Table 2 (Nos 1 to 949; corresponding to Compounds C25-1 to C25-949).
C26. Compounds C26-1 to C26-949 of the general formula (Ib) in which R¹³ represents CH₃, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds C26-1 to C26-949).
C27. Compounds C27-1 to C27-949 of the general formula (Ib) in which R⁹ and R¹³ represent CH₃, R¹, R² and R³ represent hydrogen and Q, W, R⁵, R⁶, R⁷, R⁸ for the individual compound in question correspond to the radical definitions given in Table 2 (Nos 1 to 949; corresponding to Compounds C27-1 to C27-949).
C28. Compounds C28-1 to C28-949 of the general formula (Ib) in which R⁹ represents ethyl, R¹, R², R³ and R¹³ represent hydrogen and Q, W, R⁵, R⁶, R⁷, R⁸ for the individual compound in question correspond to the radical definitions given in Table 2 (Nos 1 to 949; corresponding to Compounds C28-1 to C28-949).
C29. Compounds C29-1 to C29-949 of the general formula (Ib) in which R¹³ represents ethyl, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds C29-1 to C29-949).
C30. Compounds C30-1 to C30-949 of the general formula (Ib) in which R⁹ and R¹³ represent ethyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds C30-1 to C30-949).
C31. Compounds C31-1 to C31-949 of the general formula (Ib) in which R⁹ represents CH₂CHF₂, R¹, R², R³ and R¹³ represent hydrogen and Q, W, R⁵, R⁶, R⁷, R⁸ for the individual compound in question correspond to the radical definitions given in Table 2 (Nos 1 to 949; corresponding to Compounds C31-1 to C31-949).
C32. Compounds C32-1 to C32-949 of the general formula (Ib) in which R¹³ represents CH₂CHF₂, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds C32-1 to C32-949).
C33. Compounds C33-1 to C33-949 of the general formula (Ib) in which R⁹ and R¹³ represent CH₂CHF₂, R¹, R² and R³ represent hydrogen and Q, W, R⁵, R⁶, R⁷, R⁸ for the individual compound in question correspond to the radical definitions given in Table 2 (Nos 1 to 949; corresponding to Compounds C33-1 to C33-949).
C34. Compounds C34-1 to C34-949 of the general formula (Ib) in which R⁹ represents n-propyl, R¹, R², R³ and R¹³ represent hydrogen and Q, W, R⁵, R⁶, R⁷, R⁸ for the individual compound in question correspond to the radical definitions given in Table 2 (Nos 1 to 949; corresponding to Compounds C34-1 to C34-949).
C35. Compounds C35-1 to C35-949 of the general formula (Ib) in which R¹³ represents n-propyl, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds C35-1 to C35-949).
C36. Compounds C36-1 to C36-949 of the general formula (Ib) in which R⁹ and R¹³ represent n-propyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds C36-1 to C36-949).
C37. Compounds C37-1 to C37-949 of the general formula (Ib) in which R⁹ represents allyl, R¹, R², R³ and R¹³ represent hydrogen and Q, W, R⁵, R⁶, R⁷, R⁸ for the individual compound in question correspond to the radical definitions given in Table 2 (Nos 1 to 949; corresponding to Compounds C37-1 to C37-949).
C38. Compounds C38-1 to C38-949 of the general formula (Ib) in which R¹³ represents allyl, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds C38-1 to C38-949).
C39. Compounds C39-1 to C39-949 of the general formula (Ib) in which R⁹ and R¹³ represent allyl, R¹, R² and R³ represent hydrogen and Q, W, R⁵, R⁶, R⁷, R⁸ for the individual compound in question correspond to the radical definitions given in Table 2 (Nos 1 to 949; corresponding to Compounds C39-1 to C39-949).
D1. Compounds D1-1 to D1-949 of the general formula (Id) in which R¹, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D1-1 to D1-949).

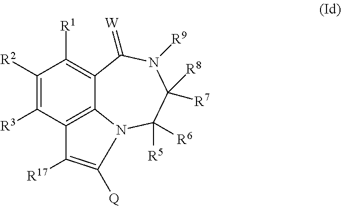

D2. Compounds D2-1 to D2-949 of the general formula (Id) in which R¹ represents fluorine, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D2-1 to D2-949).
D3. Compounds D3-1 to D3-949 of the general formula (Id) in which R¹ represents chlorine, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D3-1 to D3-949).
D4. Compounds D4-1 to D4-949 of the general formula (Id) in which R¹ represents methyl, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D4-1 to D4-949).
D5. Compounds D5-1 to D5-949 of the general formula (Id) in which R¹ represents trifluoromethyl, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D5-1 to D5-949).
D6. Compounds D6-1 to D6-949 of the general formula (Id) in which R¹ represents methoxy, R², R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D6-1 to D6-949).
D7. Compounds D7-1 to D7-949 of the general formula (Id) in which R² represents fluorine, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D7-1 to D7-949).
D8. Compounds D8-1 to D8-949 of the general formula (Id) in which R² represents chlorine, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D8-1 to D8-949).
D9. Compounds D9-1 to D9-949 of the general formula (Id) in which R² represents bromine, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D9-1 to D9-949).
D10. Compounds D10-1 to D10-949 of the general formula (Id) in which R² represents iodine, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D10-1 to D10-949).
D11. Compounds D11-1 to D11-949 of the general formula (Id) in which R² represents methyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D11-1 to D11-949).
D12. Compounds D12-1 to D12-949 of the general formula (Id) in which R² represents methoxy, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D12-1 to D12-949).
D13. Compounds D13-1 to D13-949 of the general formula (Id) in which R² represents trifluoromethyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D13-1 to D13-949).
D14. Compounds D14-1 to D14-949 of the general formula (Id) in which R² represents trifluoromethoxy, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D14-1 to D14-949).
D15. Compounds D15-1 to D15-949 of the general formula (Id) in which R² represents methylthio, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D15-1 to D15-949).
D16. Compounds D16-1 to D16-949 of the general formula (Id) in which R² represents trifluoromethylthio, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D16-1 to D16-949).
D17. Compounds D17-1 to D17-949 of the general formula (Id) in which R² represents ethyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D17-1 to D17-949).
D18. Compounds D18-1 to D18-949 of the general formula (Id) in which R² represents isopropyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D18-1 to D18-949).
D19. Compounds D19-1 to D19-949 of the general formula (Id) in which R² represents cyclopropyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D19-1 to D19-949).
D20. Compounds D20-1 to D20-949 of the general formula (Id) in which R² represents phenyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D20-1 to D20-949).
D21. Compounds D21-1 to D21-949 of the general formula (Id) in which R² represents thiophenyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D21-1 to D21-949).
D22. Compounds D22-1 to D22-949 of the general formula (Id) in which R² represents ethynyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D22-1 to D22-949).
D23. Compounds D23-1 to D23-949 of the general formula (Id) in which R² represents trimethylsilylethynyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D23-1 to D23-949).
D24. Compounds D24-1 to D24-949 of the general formula (Id) in which R² represents p-Cl-phenyl, R¹, R³, R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D24-1 to D24-949).
D25. Compounds D25-1 to D25-949 of the general formula (Id) in which R³ represents fluorine, R¹, R², R⁹ and R¹⁷ represent hydrogen and Q, W, R⁵, R⁶, R⁷, R⁸ for the individual compound in question correspond to the radical definitions given in Table 2 (Nos 1 to 949; corresponding to Compounds D25-1 to D25-949).
D26. Compounds D26-1 to D26-949 of the general formula (Id) in which R⁹ represents CH₃, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D26-1 to D26-949).
D27. Compounds D27-1 to D27-949 of the general formula (Id) in which R⁹ represents ethyl, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D27-1 to D27-949).
D28. Compounds D28-1 to D28-949 of the general formula (Id) in which R⁹ represents n-propyl, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D28-1 to D28-949).
D29. Compounds D29-1 to D29-949 of the general formula (Id) in which R⁹ represents allyl, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D29-1 to D2-949).
D30. Compounds D30-1 to D30-949 of the general formula (Id) in which R⁹ represents CH₂CHF₂, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D30-1 to D30-949).
D31. Compounds D31-1 to D31-949 of the general formula (Id) in which R⁹ represents C(═O)CH₃, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D31-1 to D31-949).
D32. Compounds D32-1 to D31-949 of the general formula (Id) in which R⁹ represents SO₂CH₃, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D32-1 to D32-949).
D33. Compounds D33-1 to D33-949 of the general formula (Id) in which R⁹ represents SO₂i-Pr, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D33-1 to D33-949).
D34. Compounds D34-1 to D34-949 of the general formula (Id) in which R⁹ represents SO₂c-Pr, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D34-1 to D34-949).
D35. Compounds D35-1 to D35-949 of the general formula (Id) in which R⁹ represents SO₂Ph, R¹, R², R³ and R¹⁷ represent hydrogen and Q, W, R⁵, R⁶, R⁷, R⁸ for the individual compound in question correspond to the radical definitions given in Table 2 (Nos 1 to 949; corresponding to Compounds D35-1 to D35-949).
D36. Compounds D36-1 to D36-949 of the general formula (Id) in which R¹⁷ represents Cl, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D36-1 to D36-949).
D37. Compounds D37-1 to D37-949 of the general formula (Id) in which R¹⁷ represents Br, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D37-1 to D37-949).
D38. Compounds D38-1 to D38-949 of the general formula (Id) in which R¹⁷ represents I, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D38-1 to D38-949).
D39. Compounds D39-1 to D39-949 of the general formula (Id) in which R¹⁷ represents CH(═O), R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D39-1 to D39-949).
D40. Compounds D40-1 to D40-949 of the general formula (Id) in which R¹⁷ represents CH(═NOH), R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D40-1 to D40-949).
D41. Compounds D41-1 to D41-949 of the general formula (Id) in which R¹⁷ represents CH(═NOMe), R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D41-1 to D41-949).
D42. Compounds D42-1 to D42-949 of the general formula (Id) in which R¹⁷ represents CH(═NOEt), R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D42-1 to D42-949).
D43. Compounds D43-1 to D43-949 of the general formula (Id) in which R¹⁷ represents CN, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D43-1 to D43-949).
D44. Compounds D44-1 to D44-949 of the general formula (Id) in which R¹⁷ represents C(═O)NH₂, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D44-1 to D44-949).
D45. Compounds D45-1 to D45-949 of the general formula (Id) in which R¹⁷ represents C(═O)CH₃, R¹, R², R³ and R⁹ represent hydrogen and Q, W, R⁵, R⁶, R⁷, R⁸ for the individual compound in question correspond to the radical definitions given in Table 2 (Nos 1 to 949; corresponding to Compounds D45-1 to D45-949).
E1. Compounds E1-1 to E1-250 of the general formula (Ic) in which R¹, R², R³ and R⁴ represent hydrogen and W, R⁷, R⁸, R⁹, R¹⁴, R¹⁵ and R¹⁶ correspond to the definition (Nos 1 to 250; corresponding to Compounds E1-1 to E1-250) in Table 3 below.

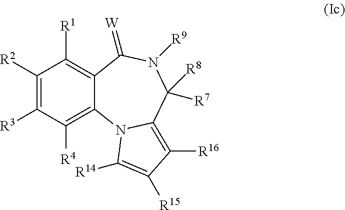

TABLE 3

| No. | W | R⁷ | R⁸ | R⁹ | R¹⁴ | R¹⁵ | R¹⁶ |
|---|---|---|---|---|---|---|---|
| 1 | O | H | H | H | H | H | H |
| 2 | O | CH₃ | H | H | H | H | H |
| 3 | O | CH₃ | CH₃ | H | H | H | H |
| 4 | O | H | H | H | CH₃ | H | H |
| 5 | O | H | H | H | H | CH₃ | H |
| 6 | O | H | H | H | H | H | CH₃ |
| 7 | O | H | H | H | CH₃ | CH₃ | H |
| 8 | O | H | H | H | Et | H | H |
| 9 | O | H | H | H | i-Pr | H | H |
| 10 | O | H | H | H | 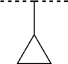 | H | H |
| 11 | O | H | H | H | 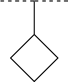 | H | H |
| 12 | O | H | H | H | 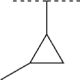 | H | H |
| 13 | O | H | H | H | CH₃ | CO₂Me | H |
| 14 | O | H | H | H | H | CO₂Me | H |
| 15 | O | H | H | H | Ph | H | H |
| 16 | O | H | H | H | CN | H | H |
| 17 | O | H | H | H | H | CN | H |
| 18 | O | H | H | H | H | H | CN |
| 19 | O | Et | H | H | H | H | H |
| 20 | O | n-Pr | H | H | H | H | H |
| 21 | O | i-Pr | H | H | H | H | H |
| 22 | O | 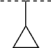 | H | H | H | H | H |
| 23 | O | 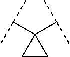 | | H | H | H | H |
| 24 | O | n-Bu | H | H | H | H | H |
| 25 | O | CH₃ | H | H | CH₃ | H | H |
| 26 | O | CH₃ | H | H | H | CH₃ | H |
| 27 | O | CH₃ | H | H | H | H | CH₃ |
| 28 | O | CH₃ | H | H | Cl | H | H |
| 29 | O | CH₃ | H | H | H | Cl | H |
| 30 | O | CH₃ | H | H | H | H | Cl |
| 31 | O | CH₃ | H | H | Et | H | H |
| 32 | O | CH₃ | H | H | H | Et | H |
| 33 | O | CH₃ | H | H | H | H | Et |
| 34 | O | H | H | H | H | Et | H |
| 35 | O | H | H | H | H | H | Et |
| 36 | O | Et | H | H | CH₃ | H | H |
| 37 | O | Et | H | H | H | CH₃ | H |
| 38 | O | Et | H | H | H | H | CH₃ |
| 39 | O | i-Pr | H | H | CH₃ | H | H |
| 40 | O | i-Pr | H | H | H | CH₃ | H |
| 41 | O | i-Pr | H | H | H | H | CH₃ |
| 42 | O | n-Pr | H | H | CH₃ | H | H |
| 43 | O | n-Pr | H | H | H | CH₃ | H |
| 44 | O | n-Pr | H | H | H | H | CH₃ |
| 45 | O | CH₃ | CH₃ | H | CH₃ | H | H |
| 46 | O | CH₃ | CH₃ | H | H | CH₃ | H |
| 47 | O | H | H | H | p-Cl—Ph | H | H |
| 48 | O | H | H | H | p-Cl—Ph | H | H |
| 49 | O | H | H | H | p-Cl—Ph | H | H |
| 50 | O | H | H | H | p-Cl—Ph | H | H |
| 51 | S | H | H | H | H | H | H |
| 52 | S | CH₃ | H | H | H | H | H |
| 53 | S | CH₃ | CH₃ | H | H | H | H |
| 54 | S | H | H | H | CH₃ | H | H |
| 55 | S | H | H | H | H | CH₃ | H |
| 56 | S | H | H | H | H | H | CH₃ |
| 57 | S | H | H | H | CH₃ | CH₃ | H |
| 58 | S | H | H | H | Et | H | H |
| 59 | S | H | H | H | i-Pr | H | H |
| 60 | S | H | H | H | 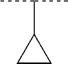 | H | H |
| 61 | S | H | H | H | 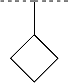 | H | H |
| 62 | S | H | H | H | 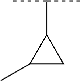 | H | H |
| 63 | S | H | H | H | CH₃ | CO₂Me | H |
| 64 | S | H | H | H | H | CO₂Me | H |
| 65 | S | H | H | H | Ph | H | H |
| 66 | S | H | H | H | CN | H | H |
| 67 | S | H | H | H | H | CN | H |
| 68 | S | H | H | H | H | H | CN |
| 69 | S | Et | H | H | H | H | H |
| 70 | S | n-Pr | H | H | H | H | H |
| 71 | S | i-Pr | H | H | H | H | H |
| 72 | S | 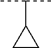 | H | H | H | H | H |
| 73 | S | 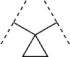 | | H | H | H | H |
| 74 | S | n-Bu | H | H | H | H | H |
| 75 | S | CH₃ | H | H | CH₃ | H | H |
| 76 | S | CH₃ | H | H | H | CH₃ | H |
| 77 | S | CH₃ | H | H | H | H | CH₃ |
| 78 | S | CH₃ | H | H | Cl | H | H |
| 79 | S | CH₃ | H | H | H | Cl | H |
| 80 | S | CH₃ | H | H | H | H | Cl |
| 81 | S | CH₃ | H | H | Et | H | H |
| 82 | S | CH₃ | H | H | H | Et | H |
| 83 | S | CH₃ | H | H | H | H | Et |
| 84 | S | H | H | H | H | Et | H |
| 85 | S | H | H | H | H | H | Et |
| 86 | S | Et | H | H | CH₃ | H | H |
| 87 | S | Et | H | H | H | CH₃ | H |
| 88 | S | Et | H | H | H | H | CH₃ |
| 89 | S | i-Pr | H | H | CH₃ | H | H |
| 90 | S | i-Pr | H | H | H | CH₃ | H |
| 91 | S | i-Pr | H | H | H | H | CH₃ |
| 92 | S | n-Pr | H | H | CH₃ | H | H |
| 93 | S | n-Pr | H | H | H | CH₃ | H |
| 94 | S | n-Pr | H | H | H | H | CH₃ |
| 95 | S | CH₃ | CH₃ | H | CH₃ | H | H |
| 96 | S | CH₃ | CH₃ | H | H | CH₃ | H |
| 97 | S | H | H | H | p-Cl—Ph | H | H |
| 98 | S | H | H | H | p-Cl—Ph | H | H |
| 99 | S | H | H | H | p-Cl—Ph | H | H |
| 100 | S | H | H | H | p-Cl—Ph | H | H |
| 101 | O | H | H | CH₃ | H | H | H |
| 102 | O | CH₃ | H | CH₃ | H | H | H |
| 103 | O | CH₃ | CH₃ | CH₃ | H | H | H |
| 104 | O | H | H | CH₃ | CH₃ | H | H |
| 105 | O | H | H | CH₃ | H | CH₃ | H |
| 106 | O | H | H | CH₃ | H | H | CH₃ |
| 107 | O | H | H | CH₃ | CH₃ | CH₃ | H |
| 108 | O | H | H | CH₃ | Et | H | H |
| 109 | O | H | H | CH₃ | i-Pr | H | H |
| 110 | O | H | H | CH₃ | 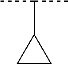 | H | H |
| 111 | O | H | H | CH₃ | 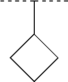 | H | H |
| 112 | O | H | H | CH₃ | 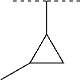 | H | H |
| 113 | O | H | H | CH₃ | CH₃ | CO₂Me | H |
| 114 | O | H | H | CH₃ | H | CO₂Me | H |
| 115 | O | H | H | CH₃ | Ph | H | H |
| 116 | O | H | H | CH₃ | CN | H | H |
| 117 | O | H | H | CH₃ | H | CN | H |
| 118 | O | H | H | CH₃ | H | H | CN |
| 119 | O | Et | H | CH₃ | H | H | H |
| 120 | O | n-Pr | H | CH₃ | H | H | H |
| 121 | O | i-Pr | H | CH₃ | H | H | H |
| 122 | O | 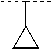 | H | CH₃ | H | H | H |
| 123 | O | 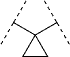 | | | CH₃ | H | H |
| 124 | O | n-Bu | H | CH₃ | H | H | H |
| 125 | O | CH₃ | H | CH₃ | CH₃ | H | H |
| 126 | O | CH₃ | H | CH₃ | H | CH₃ | H |
| 127 | O | CH₃ | H | CH₃ | H | H | CH₃ |
| 128 | O | CH₃ | H | CH₃ | Cl | H | H |
| 129 | O | CH₃ | H | CH₃ | H | Cl | H |
| 130 | O | CH₃ | H | CH₃ | H | H | Cl |
| 131 | O | CH₃ | H | CH₃ | Et | H | H |
| 132 | O | CH₃ | H | CH₃ | H | Et | H |
| 133 | O | CH₃ | H | CH₃ | H | H | Et |
| 134 | O | H | H | CH₃ | H | Et | H |
| 135 | O | H | H | CH₃ | H | H | Et |
| 136 | O | Et | H | CH₃ | CH₃ | H | H |
| 137 | O | Et | H | CH₃ | H | CH₃ | H |
| 138 | O | Et | H | CH₃ | H | H | CH₃ |
| 139 | O | i-Pr | H | CH₃ | CH₃ | H | H |
| 140 | O | i-Pr | H | CH₃ | H | CH₃ | H |
| 141 | O | i-Pr | H | CH₃ | H | H | CH₃ |
| 142 | O | n-Pr | H | CH₃ | CH₃ | H | H |
| 143 | O | n-Pr | H | CH₃ | H | CH₃ | H |
| 144 | O | n-Pr | H | CH₃ | H | H | CH₃ |
| 145 | O | CH₃ | CH₃ | CH₃ | CH₃ | H | H |
| 146 | O | CH₃ | CH₃ | CH₃ | H | CH₃ | H |
| 147 | O | H | H | CH₃ | p-Cl—Ph | H | H |
| 148 | O | H | H | CH₃ | p-Cl—Ph | H | H |
| 149 | O | H | H | CH₃ | p-Cl—Ph | H | H |
| 150 | O | H | H | CH₃ | p-Cl—Ph | H | H |
| 151 | O | H | H | Allyl | H | H | H |
| 152 | O | CH₃ | H | Allyl | H | H | H |
| 153 | O | CH₃ | CH₃ | Allyl | H | H | H |
| 154 | O | H | H | Allyl | CH₃ | H | H |
| 155 | O | H | H | Allyl | H | CH₃ | H |
| 156 | O | H | H | Allyl | H | H | CH₃ |
| 157 | O | H | H | Allyl | CH₃ | CH₃ | H |
| 158 | O | H | H | Allyl | Et | H | H |
| 159 | O | H | H | Allyl | i-Pr | H | H |
| 160 | O | H | H | Allyl | 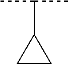 | H | H |
| 161 | O | H | H | Allyl | 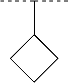 | H | H |
| 162 | O | H | H | Allyl | 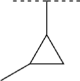 | H | H |
| 163 | O | H | H | Allyl | CH₃ | CO₂Me | H |
| 164 | O | H | H | Allyl | H | CO₂Me | H |
| 165 | O | H | H | Allyl | Ph | H | H |
| 166 | O | H | H | Allyl | CN | H | H |
| 167 | O | H | H | Allyl | H | CN | H |
| 168 | O | H | H | Allyl | H | H | CN |
| 169 | O | Et | H | Allyl | H | H | H |
| 170 | O | n-Pr | H | Allyl | H | H | H |
| 171 | O | i-Pr | H | Allyl | H | H | H |
| 172 | O | 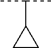 | H | Allyl | H | H | H |
| 173 | O | 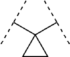 | | | Allyl | H | H |
| 174 | O | n-Bu | H | Allyl | H | H | H |
| 175 | O | CH₃ | H | Allyl | CH₃ | H | H |
| 176 | O | CH₃ | H | Allyl | H | CH₃ | H |
| 177 | O | CH₃ | H | Allyl | H | H | CH₃ |
| 178 | O | CH₃ | H | Allyl | Cl | H | H |
| 179 | O | CH₃ | H | Allyl | H | Cl | H |
| 180 | O | CH₃ | H | Allyl | H | H | Cl |
| 181 | O | CH₃ | H | Allyl | Et | H | H |
| 182 | O | CH₃ | H | Allyl | H | Et | H |
| 183 | O | CH₃ | H | Allyl | H | H | Et |
| 184 | O | H | H | Allyl | H | Et | H |
| 185 | O | H | H | Allyl | H | H | Et |
| 186 | O | Et | H | Allyl | CH₃ | H | H |
| 187 | O | Et | H | Allyl | H | CH₃ | H |
| 188 | O | Et | H | Allyl | H | H | CH₃ |
| 189 | O | i-Pr | H | Allyl | CH₃ | H | H |
| 190 | O | i-Pr | H | Allyl | H | CH₃ | H |
| 191 | O | i-Pr | H | Allyl | H | H | CH₃ |
| 192 | O | n-Pr | H | Allyl | CH₃ | H | H |
| 193 | O | n-Pr | H | Allyl | H | CH₃ | H |
| 194 | O | n-Pr | H | Allyl | H | H | CH₃ |
| 195 | O | CH₃ | CH₃ | Allyl | CH₃ | H | H |
| 196 | O | CH₃ | CH₃ | Allyl | H | CH₃ | H |
| 197 | O | H | H | Allyl | p-Cl—Ph | H | H |
| 198 | O | H | H | Allyl | p-Cl—Ph | H | H |
| 199 | O | H | H | Allyl | p-Cl—Ph | H | H |
| 200 | O | H | H | Allyl | p-Cl—Ph | H | H |
| 201 | O | H | H | 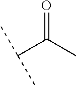 | H | H | H |
| 202 | O | CH₃ | H | 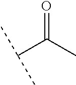 | H | H | H |
| 203 | O | CH₃ | CH₃ | 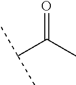 | H | H | H |
| 204 | O | H | H | 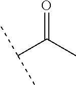 | CH₃ | H | H |
| 205 | O | H | H | 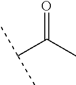 | H | CH₃ | H |
| 206 | O | H | H | 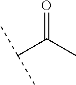 | H | H | CH₃ |
| 207 | O | H | H | 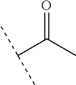 | CH₃ | CH₃ | H |
| 208 | O | H | H | 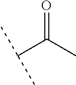 | Et | H | H |
| 209 | O | H | H | 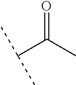 | i-Pr | H | H |
| 210 | O | H | H | 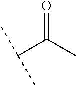 | 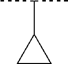 | H | H |
| 211 | O | H | H | 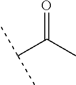 | 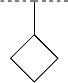 | H | H |
| 212 | O | H | H | 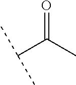 | 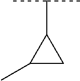 | H | H |
| 213 | O | H | H | 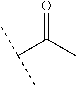 | CH₃ | CO₂Me | H |
| 214 | O | H | H | 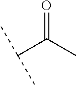 | H | CO₂Me | H |
| 215 | O | H | H | 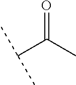 | Ph | H | H |
| 216 | O | H | H | 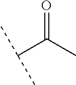 | CN | H | H |
| 217 | O | H | H | 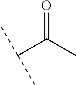 | H | CN | H |
| 218 | O | H | H | 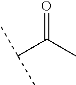 | H | H | CN |
| 219 | O | Et | H | 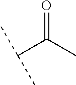 | H | H | H |
| 220 | O | n-Pr | H | 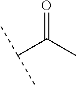 | H | H | H |
| 221 | O | i-Pr | H | 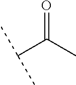 | H | H | H |
| 222 | O | 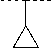 | H | 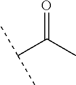 | H | H | H |
| 223 | O | 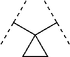 | | 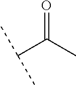 | H | H | H |
| 224 | O | n-Bu | H | 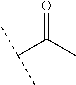 | H | H | H |
| 225 | O | CH₃ | H | 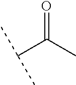 | CH₃ | H | H |
| 226 | O | CH₃ | H | 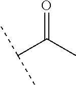 | H | CH₃ | H |
| 227 | O | CH₃ | H | 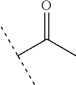 | H | H | CH₃ |
| 228 | O | CH₃ | H | 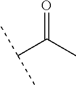 | Cl | H | H |
| 229 | O | CH₃ | H | 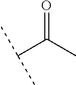 | H | Cl | H |
| 230 | O | CH₃ | H | 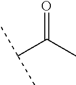 | H | H | Cl |
| 231 | O | CH₃ | H | 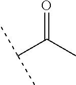 | Et | H | H |
| 232 | O | CH₃ | H | 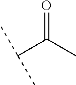 | H | Et | H |
| 233 | O | CH₃ | H | 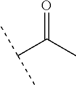 | H | H | Et |
| 234 | O | H | H | 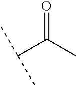 | H | Et | H |
| 235 | O | H | H | 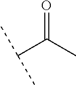 | H | H | Et |
| 236 | O | Et | H | 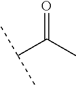 | CH₃ | H | H |
| 237 | O | Et | H | 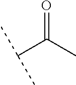 | H | CH₃ | H |
| 238 | O | Et | H | 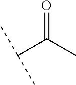 | H | H | CH₃ |
| 239 | O | i-Pr | H | 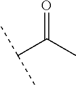 | CH₃ | H | H |
| 240 | O | i-Pr | H | 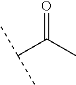 | H | CH₃ | H |
| 241 | O | i-Pr | H | 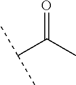 | H | H | CH₃ |
| 242 | O | n-Pr | H | 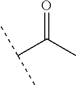 | CH₃ | H | H |
| 243 | O | n-Pr | H | 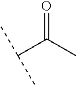 | H | CH₃ | H |
| 244 | O | n-Pr | H | 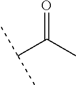 | H | H | CH₃ |
| 245 | O | CH₃ | CH₃ | 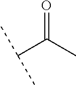 | CH₃ | H | H |
| 246 | O | CH₃ | CH₃ | 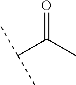 | H | CH₃ | H |
| 247 | O | H | H | 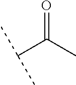 | p-Cl—Ph | H | H |
| 248 | O | H | H | 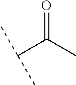 | p-Cl—Ph | H | H |
| 249 | O | H | H | 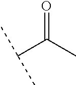 | p-Cl—Ph | H | H |
| 250 | O | H | H | 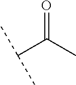 | p-Cl—Ph | H | H |

E2. Compounds E2-1 to E2-250 of the general formula (Ic) in which R¹ represents fluorine, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E2-1 to E2-250).
E3. Compounds E3-1 to E3-250 of the general formula (Ic) in which R¹ represents chlorine, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E3-1 to E3-250).
E4. Compounds E4-1 to E4-250 of the general formula (Ic) in which R¹ represents bromine, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E4-1 to E4-250).
E5. Compounds E5-1 to E5-250 of the general formula (Ic) in which R¹ represents iodine, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E5-1 to E5-250).
E6. Compounds E6-1 to E6-250 of the general formula (Ic) in which R¹ represents methyl, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E6-1 to E6-250).
E7. Compounds E7-1 to E7-250 of the general formula (Ic) in which R¹ represents methoxy, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E7-1 to E7-250).
E8. Compounds E8-1 to E8-250 of the general formula (Ic) in which R¹ represents trifluoromethyl, R², R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E8-1 to E8-250).
E9. Compounds E9-1 to E9-250 of the general formula (Ic) in which R² represents fluorine, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E9-1 to E9-250).
E10. Compounds E10-1 to E10-250 of the general formula (Ic) in which R² represents chlorine, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E10-1 to E10-250).
E11. Compounds E11-1 to E11-250 of the general formula (Ic) in which R² represents bromine, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E11-1 to E11-250).
E12. Compounds E12-1 to E12-250 of the general formula (Ic) in which R² represents iodine, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E12-1 to E12-250).
E13. Compounds E13-1 to E13-250 of the general formula (Ic) in which R² represents methyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E13-1 to E13-250).
E14. Compounds E14-1 to E14-250 of the general formula (Ic) in which R² represents methoxy, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E14-1 to E14-250).
E15. Compounds E15-1 to E15-250 of the general formula (Ic) in which R² represents trifluoromethyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E15-1 to E15-250).
E16. Compounds E16-1 to E16-250 of the general formula (Ic) in which R² represents trifluoromethoxy, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E16-1 to E16-250).
E17. Compounds E17-1 to E17-250 of the general formula (Ic) in which R² represents phenyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E17-1 to E17-250).
E18. Compounds E18-1 to E18-250 of the general formula (Ic) in which R² represents ethyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E18-1 to E18-250).
E19. Compounds E19-1 to E19-250 of the general formula (Ic) in which R² represents hydroxy, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E19-1 to E19-250).
E20. Compounds E20-1 to E20-250 of the general formula (Ic) in which R² represents trimethylsilylethynyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E20-1 to E20-250).
E21. Compounds E21-1 to E21-250 of the general formula (Ic) in which R² represents ethynyl, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E21-1 to E21-250).
E22. Compounds E22-1 to E22-250 of the general formula (Ic) in which R³ represents fluorine, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E22-1 to E22-250).
E23. Compounds E23-1 to E23-250 of the general formula (Ic) in which R³ represents chlorine, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E23-1 to E23-250).
E24. Compounds E24-1 to E24-250 of the general formula (Ic) in which R³ represents bromine, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E24-1 to E24-250).
E25. Compounds E25-1 to E25-250 of the general formula (Ic) in which R³ represents iodine, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E25-1 to E25-250).
E26. Compounds E26-1 to E26-250 of the general formula (Ic) in which R³ represents methyl, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E26-1 to E26-250).
E27. Compounds E27-1 to E27-250 of the general formula (Ic) in which R³ represents methoxy, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E27-1 to E27-250).
E28. Compounds E28-1 to E28-250 of the general formula (Ic) in which R³ represents trifluoromethyl, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E28-1 to E28-250).
E29. Compounds E29-1 to E29-250 of the general formula (Ic) in which R³ represents trifluoromethoxy, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E29-1 to E29-250).
E30. Compounds E30-1 to E30-250 of the general formula (Ic) in which R² represents methylthio, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E30-1 to E30-250).
E31. Compounds E31-1 to E31-250 of the general formula (Ic) in which R² represents trifluoromethylthio, R¹, R³ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E31-1 to E31-250).
E32. Compounds E32-1 to E32-250 of the general formula (Ic) in which R³ represents methylthio, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E32-1 to E32-250).
E33. Compounds E33-1 to E33-250 of the general formula (Ic) in which R³ represents trifluoromethylthio, R¹, R² and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E33-1 to E33-250).
E34. Compounds E34-1 to E34-250 of the general formula (Ic) in which R⁴ represents fluorine, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E34-1 to E34-250).
E35. Compounds E35-1 to E35-250 of the general formula (Ic) in which R⁴ represents chlorine, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E35-1 to E35-250).
E36. Compounds E36-1 to E36-250 of the general formula (Ic) in which R⁴ represents bromine, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E36-1 to E36-250).
E37. Compounds E37-1 to E37-250 of the general formula (Ic) in which R⁴ represents iodine, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E37-1 to E37-250).
E38. Compounds E38-1 to E38-250 of the general formula (Ic) in which R⁴ represents methyl, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E38-1 to E38-250).
E39. Compounds E39-1 to E39-250 of the general formula (Ic) in which R⁴ represents methoxy, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E39-1 to E39-250).
E40. Compounds E40-1 to E40-250 of the general formula (Ic) in which R⁴ represents trifluoromethyl, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E40-1 to E40-250).
E41. Compounds E41-1 to E41-250 of the general formula (Ic) in which R⁴ represents trifluoromethoxy, R¹, R² and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E41-1 to E41-250).
E42. Compounds E42-1 to E42-250 of the general formula (Ic) in which R² and R³ represent methoxy, R¹ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E42-1 to E42-250).
E43. Compounds E43-1 to E43-250 of the general formula (Ic) in which R² and R³ represent methyl, R¹ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E43-1 to E43-250).
E44. Compounds E44-1 to E44-250 of the general formula (Ic) in which R² and R³ represent chlorine, R¹ and R⁴ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E44-1 to E44-250).
E45. Compounds E45-1 to E45-250 of the general formula (Ic) in which R² and R⁴ represent methoxy, R¹ and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E45-1 to E45-250).
E46. Compounds E46-1 to E46-250 of the general formula (Ic) in which R² and R⁴ represent methyl, R¹ and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E46-1 to E46-250).
E47. Compounds E47-1 to E47-250 of the general formula (Ic) in which R² and R⁴ represent chlorine, R¹ and R³ represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E47-1 to E47-250).
E48. Compounds E48-1 to E48-250 of the general formula (Ic) in which R³ and R⁴ represent methoxy, R¹ and R² represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E48-1 to E48-250).
E49. Compounds E49-1 to E49-250 of the general formula (Ic) in which R³ and R⁴ represent methyl, R¹ and R² represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E49-1 to E49-250).
E50. Compounds E50-1 to E50-250 of the general formula (Ic) in which R³ and R⁴ represent chlorine, R¹ and R² represent hydrogen and W, R⁷, R⁸, R⁹, R¹⁴, R¹⁵ and R¹⁶ for the individual compound in question correspond to the radical definitions given in Table 3 (Nos 1 to 250; corresponding to Compounds E50-1 to E50-250).

### Spectroscopic Data of Selected Table Examples

### Example No. A1-121

¹H-NMR (300 MHz, d₆-DMSO δ, ppm) 8.15 (br. t, 1H, NH), 7.78 (d, 1H), 7.73 (d, 1H), 6.43 (dd, 1H), 5.46 (br. t, 1H, NH), 3.49 (m, 2H), 3.26 (m, 2H).

### Example No. A1-281

¹H-NMR (300 MHz, CDCl₃ δ, ppm) 8.04 (d, 1H), 7.52 (m, 3H), 7.09 (m, 2H), 6.78 (m, 1H), 6.61 (br. t, 1H, NH), 5.55 (br. t, 1H, NH), 3.73 (m, 2H), 3.57 (m, 2H).

### Example No. A8-121

¹H-NMR (300 MHz, d₆-DMSO δ, ppm) 8.29 (br. t, 1H, NH), 7.81 (m, 1H), 7.51 (m, 1H), 5.29 (br. t, 1H, NH), 3.45 (m, 2H), 3.26 (m, 2H).

### Example No. A8-281

¹H-NMR (300 MHz, CDCl₃ δ, ppm) 7.76 (d, 1H), 7.51 (m, 2H), 7.21 (m, 1H), 7.09 (m, 2H), 6.68 (br. t, 1H, NH), 5.35 (br. t, 1H, NH), 3.70 (m, 2H), 3.56 (m, 2H).

### Example No. A8-282

¹H-NMR (300 MHz, CDCl₃ δ, ppm) 7.77 (d, 1H), 7.48 (m, 3H), 7.36 (m, 2H), 6.54 (br. t, 1H, NH), 5.34 (br. t, 1H, NH), 3.71 (m, 2H), 3.56 (m, 2H).

### Example No. A8-289

¹H-NMR (300 MHz, CDCl₃ δ, ppm) 7.67 (m, 1H), 7.14 (m, 1H), 6.59 (br. t, 1H, NH), 5.33 (br. t, 1H, NH), 3.77 (m, 2H), 3.53 (m, 2H), 2.68 (m, 1H), 1.90 (m, 2H), 1.76 (m, 2H), 1.59 (m, 2H), 1.40 (m, 4H).

### Example No. B1-11

¹H-NMR (400 MHz, d₆-DMSO δ, ppm) 8.35 (t, 1H, NH), 7.8 (m, 2H), 7.26 (t, 1H), 4.35 (m, 2H), 3.6 (m, 2H), 3.25 (m, 1H), 1.3 (s, 6H).

### Example No. B1-21

¹H-NMR (400 MHz, d₆-DMSO δ, ppm) 8.39 (t, 1H, NH), 7.83 (d, 1H), 7.78 (d, 1H), 7.25 (t, 1H), 4.5 (m, 2H), 3.6 (m, 2H), 1.49 (s, 9H).

### Example No. B1-34

¹H-NMR (300 MHz, CD₃OD δ, ppm) 8.78 (d, 1H), 8.18 (dd, 1H), 7.95 (dd, 1H), 7.85 (dd, 1H), 7.6 (dd, 1H), 7.4 (t, 1H), 4.45 (m, 2H), 3.6 (m, 2H).

### Example No. B1-35

¹H-NMR (300 MHz, CD₃OD δ, ppm) 8.06-7.93 (m, 6H), 7.46 (t, 1H), 4.56 (m, 2H), 3.71 (m, 2H).

### Example No. B1-36

¹H-NMR (300 MHz, CD₃OD δ, ppm) 9.2 (d, 1H), 8.5 (dd, 1H), 8.1-7.97 (m, 3H), 7.5 (t, 1H), 4.6 (m, 2H), 3.7 (m, 2H).

### Example No. B1-39

¹H-NMR (400 MHz, CD₃OD δ, ppm) 8.05 (d, 1H), 7.95 (d, 1H), 7.83 (m, 1H), 7.68 (m, 1H), 7.56-7.43 (m, 2H), 4.55 (m, 2H), 3.7 (m, 2H), 3.32 (s, 1H, NH).

### Example No. B1-40

¹H-NMR (300 MHz, CD₃OD δ, ppm) 8.03 (m, 2H), 7.94 (dd, 1H), 7.78 (s, 2H), 7.43 (t, 1H), 4.59 (s, 1H, NH), 4.5 (m, 2H), 3.67 (m, 2H).

### Example No. B1-42

¹H-NMR (400 MHz, d₆-DMSO δ, ppm) 8.45 (br. t, 1H, NH), 7.95 (m, 2H), 7.85 (m, 1H), 7.58 (m, 1H), 7.41-7.31 (m, 2H), 4.24 (m, 2H), 3.55 (m, 2H).

### Example No. B1-44

¹H-NMR (300 MHz, CD₃OD δ, ppm) 8.03 (d, 1H), 7.94 (d, 1H), 7.78 (m, 4H), 7.43 (t, 1H), 4.58 (s, 1H, NH), 4.5 (m, 2H), 3.65 (m, 2H).

### Example No. B1-48

¹H-NMR (400 MHz, CD₃OD δ, ppm) 8.0 (dd, 1H), 7.86 (dd, 1H), 7.68 (d, 1H), 7.58 (m, 1H), 7.51 (m, 1H), 7.40 (t, 1H), 4.46 (m, 2H), 4.18 (br. s, 1H, NH), 3.61 (m, 2H).

### Example No. B1-51

¹H-NMR (400 MHz, CD₃OD δ, ppm) 8.05 (dd, 1H), 7.95 (dd, 1H), 7.87 (s, 1H), 7.78 (m, 1H), 7.60 (m, 2H), 7.45 (t, 1H), 4.59 (s, 1H, NH), 4.55 (m, 2H), 3.68 (m, 2H).

### Example No. B1-52

¹H-NMR (400 MHz, d₆-DMSO δ, ppm) 8.49 (t, 1H), 7.9 (m, 2H), 7.7 (m, 2H), 7.65 (m, 1H), 7.4 (m, 2H), 4.5 (m, 2H), 3.55 (m, 2H).

### Example No. B1-58

¹H-NMR (400 MHz, d₆-DMSO δ, ppm) 10.15 (s, 1H), 8.48 (br. t, 1H), 8.10 (s, 3H), 7.95 (m, 2H), 7.42 (t, 1H), 4.50 (m, 2H), 3.56 (m, 2H), 3.31 (s, 1H, NH).

### Example No. B1-65

¹H-NMR (400 MHz, d₆-DMSO δ, ppm) 8.51 (t, 1H), 7.90 (m, 4H), 7.87 (d, 1H), 7.42 (t, 1H), 4.50 (m, 2H), 3.55 (m, 2H).

### Example No. B1-67

¹H-NMR (400 MHz, d₆-DMSO δ, ppm) 8.5 (t, 1H), 7.9 (m, 2H), 7.70 (s, 1H), 7.6 (m, 2H), 7.4 (t, 1H), 4.50 (m, 2H), 3.55 (m, 2H).

### Example No. B1-69

¹H-NMR (400 MHz, d₆-DMSO δ, ppm) 8.5 (t, 1H), 8.05 (s, 1H), 7.95 (m, 4H), 7.4 (t, 1H), 4.5 (m, 2H), 3.55 (m, 2H).

### Example No. B1-75

¹H-NMR (300 MHz, CD₃OD δ, ppm) 8.21 (br. s, 1H), 8.10 (m, 2H), 7.90 (m, 2H), 7.80 (m, 1H), 7.45 (t, 1H), 4.58 (m, 2H), 3.70 (m, 2H).

### Example No. B1-76

¹H-NMR (400 MHz, d₆-DMSO δ, ppm) 8.5 (t, 1H), 7.9 (m, 4H), 7.75 (t, 1H), 7.60 (d, 1H), 7.41 (t, 1H), 4.50 (m, 2H), 3.58 (m, 2H).

### Example No. B1-77

¹H-NMR (400 MHz, d₆-DMSO δ, ppm) 8.50 (t, 1H), 8.21 (s, 1H), 8.10 (m, 1H), 7.93 (m, 3H), 7.78 (t, 1H), 7.40 (t, 1H), 4.51 (m, 2H), 3.55 (m, 2H).

### Example No. B1-78

¹H-NMR (400 MHz, d₆-DMSO δ, ppm) 8.45 (br. t, 1H, NH), 7.9 (m, 2H), 7.5 (t, 1H), 7.4 (m, 3H), 7.15 (dd, 1H), 4.48 (m, 2H), 3.85 (s, 3H), 3.55 (m, 2H).

### Example No. B1-80

¹H-NMR (300 MHz, CD₃OD δ, ppm) 8.01 (dd, 1H), 7.90 (dd, 1H), 7.75 (m, 2H), 7.42 (t, 1H), 7.15 (m, 2H), 4.50 (m, 2H), 3.91 (s, 3H), 3.65 (m, 2H).

### Example No. B1-85

¹H-NMR (400 MHz, CDCl₃ δ, ppm) 9.05 (s, 1H), 8.80 (d, 1H), 8.18 (dd, 1H), 8.05 (d, 1H), 7.5 (m, 1H), 7.45 (t, 1H), 6.85 (br. s, 1H, NH), 4.55 (m, 2H), 3.78 (m, 2H).

### Example No. B1-87

¹H-NMR (400 MHz, d₆-DMSO δ, ppm) 9.05 (s, 1H), 8.9 (s, 1H), 8.5 (m, 2H), 7.95 (m, 2H), 7.40 (t, 1H), 4.50 (m, 2H), 3.55 (m, 2H).

### Example No. B1-106

¹H-NMR (400 MHz, d₆-DMSO δ, ppm) 8.39 (t, 1H, NH), 7.83 (d, 1H), 7.78 (d, 1H), 7.25 (t, 1H), 4.5 (m, 2H), 3.6 (m, 2H), 3.4 (m, 1H), 1.2 (d, 4H).

### Example No. B1-108

¹H-NMR (400 MHz, d₆-DMSO δ, ppm) 8.34 (t, 1H, NH), 7.78 (m, 2H), 7.25 (t, 1H), 4.3 (m, 2H), 3.6 (m, 2H), 3.4 (m, 1H), 2.1 (m, 2H), 1.93 (m, 2H), 1.75 (m, 2H), 1.65 (m, 2H).

### Example No. B1-109

¹H-NMR (400 MHz, d₆-DMSO δ, ppm) 8.3 (br. t, 1H, NH), 7.8 (m, 2H), 7.25 (t, 1H), 4.35 (m, 2H), 3.6 (m, 2H), 2.9 (m, 1H), 1.95 (m, 2H), 1.8 (m, 2H), 1.73 (m, 1H), 1.6 (m, 2H), 1.4 (m, 2H), 1.28 (m, 1H).

### Example No. B1-234

¹H-NMR (300 MHz, CD₃OD δ, ppm) 7.85 (dd, 1H), 7.75 (dd, 1H), 7.25 (t, 1H), 7.2-7.0 (m, 5H), 3.95 (m, 2H), 3.4 (m, 2H), 3.15 (m, 2H), 3.05 (m, 2H).

### Example No. B1-295

¹H-NMR (300 MHz, CD₃OD δ, ppm) 7.75 (m, 1H), 7.45 (m, 1H), 7.2 (m, 1H), 6.95 (m, 1H), 6.65 (dd, 1H), 6.23 (m, 1H), 4.4 (m, 2H), 3.6 (m, 2H).

### Example No. B1-296

¹H-NMR (300 MHz, CD₃OD δ, ppm) 7.98 (dd, 1H), 7.9 (dd, 1H), 7.4 (t, 1H), 7.0 (m, 1H), 6.62 (m, 1H), 6.27 (m, 1H), 4.58 (s, 1H, NH), 4.5 (m, 2H), 3.9 (s, 3H), 3.68 (m, 2H).

### Example No. B1-297

¹H-NMR (300 MHz, CD₃OD δ, ppm) 8.00 (dd, 1H), 7.9 (dd, 1H), 7.85 (d, 1H), 7.43 (t, 1H), 7.25 (d, 1H), 6.76 (m, 1H), 4.6 (m, 2H), 3.78 (m, 2H).

### Example No. B1-336

¹H-NMR (400 MHz, CDCl₃ δ, ppm) 8.2 (d, 1H), 8.05 (d, 1H), 7.62 (dd, 1H), 7.55 (m, 2H), 7.45 (t, 2H), 6.7 (br. s, 1H, NH), 4.25 (m, 2H), 3.75 (m, 2H).

### Example No. B1-344

¹H-NMR (400 MHz, CD₃OD δ, ppm) 7.9 (d, 1H), 7.82 (d, 1H), 7.78 (m, 2H), 7.38 (t, 1H), 7.27 (m, 2H), 4.5 (s, 1H, NH), 4.4 (m, 2H), 3.6 (m, 2H).

### Example No. B1-375

¹H-NMR (300 MHz, CD₃OD δ, ppm) 8.06 (d, 1H), 7.95 (d, 1H), 7.43 (t, 1H), 4.59 (s, 2H), 4.45 (m, 2H), 3.77 (m, 2H), 3.0 (s, 6H).

### Example No. B1-376

¹H-NMR (400 MHz, d₆-DMSO δ, ppm) 8.37 (br. s, 1H, NH), 7.85 (d, 2H), 7.8 (d, 1H), 7.30 (t, 1H), 4.71 (s, 2H), 4.4 (s, 1H, OH), 3.6 (m, 2H), 3.15 (m, 2H).

### Example No. B1-378

¹H-NMR (400 MHz, d₆-DMSO δ, ppm) 8.3 (br. t, 1H, NH), 7.8 (m, 2H), 7.25 (t, 1H), 4.88 (s, 2H), 4.3 (m, 2H), 3.53 (m, 2H), 0.8 (s, 9H), 0.0 (s, 6H).

### Example No. B1-446

¹H-NMR (400 MHz, d₆-DMSO δ, ppm) 8.54 (t, 1H, NH), 8.08 (m, 2H), 7.5 (t, 1H), 4.55 (m, 2H), 3.7 (m, 2H).

### Example No. B1-449

¹H-NMR (400 MHz, CDCl₃ δ, ppm) 8.3 (d, 1H), 8.1 (d, 1H), 7.5 (t, 1H), 6.88 (br. s, 1H, NH), 4.65 (m, 2H), 3.85 (m, 2H).

### Example No. B1-456

¹H-NMR (400 MHz, CD₃OD δ, ppm) 8.22 (d, 1H), 8.05 (d, 1H), 7.55 (t, 1H), 3.82 (m, 2H), 3.33 (m, 2H).

### Example No. B1-464

¹H-NMR (400 MHz, CDCl₃ δ, ppm) 8.13 (d, 1H), 8.02 (d, 1H), 7.76 (d, 2H), 7.67 (d, 2H), 7.43 (t, 1H), 7.00 (br. s, 1H, NH), 5.61 (s, 1H), 4.50 (m, 2H), 3.73 (m, 2H), 3.7-3.55 (m, 4H), 1.25 (t, 6H).

### Example No. B1-465

¹H-NMR (400 MHz, d₆-DMSO δ, ppm) 8.39 (br. t, 1H, NH), 7.95 (m, 2H), 7.35 (dd, 1H), 4.40 (m, 2H), 4.21 (m, 2H), 3.62 (m, 2H).

### Example No. B1-494

¹H-NMR (400 MHz, CD₃OD δ, ppm) 8.37 (s, 1H), 8.1-7.9 (m, 6H), 7.6 (m, 2H), 7.45 (t, 1H), 4.6 (m, 2H), 3.65 (m, 2H).

### Example No. B1-496

¹H-NMR (400 MHz, d₆-DMSO δ, ppm) 8.45 (t, 1H), 7.9 (m, 2H), 7.7 (s, 1H), 7.65 (d, 1H), 7.48 (t, 1H), 7.37 (m, 2H), 4.45 (m, 2H), 3.55 (m, 2H).

### Example No. B1-497

¹H-NMR (400 MHz, d₆-DMSO δ, ppm) 8.33 (br. t, 1H, NH), 7.84 (m, 2H), 7.3 (t, 2H), 7.18 (s, 1H), 7.1 (d, 1H), 4.05 (m, 2H), 3.48 (m, 2H), 2.3 (s, 3H), 2.15 (s, 3H).

### Example No. B1-498

¹H-NMR (400 MHz, d₆-DMSO δ, ppm) 8.43 (br. t, 1H, NH), 7.85 (t, 2H), 7.69 (m, 2H), 7.35 (t, 1H), 7.12 (d, 1H), 4.45 (m, 2H), 3.88 (s, 3H), 3.53 (m, 2H), 2.22 (s, 3H).

### Example No. B1-499

¹H-NMR (300 MHz, CD₃OD δ, ppm) 8.0 (dd, 1H), 7.9 (dd, 1H), 7.4 (m, 3H), 7.18 (d, 1H), 4.58 (m, 3H, NH), 4.51 (m, 2H), 3.90 (s, 3H), 3.66 (m, 2H).

### Example No. B1-501

¹H-NMR (400 MHz, d₆-DMSO δ, ppm) 8.49 (t, 1H), 7.9 (m, 3H), 7.83 (t, 1H), 7.75 (d, 1H), 7.40 (t, 1H), 4.51 (m, 2H), 3.55 (m, 2H).

### Example No. B1-502

¹H-NMR (400 MHz, d₆-DMSO δ, ppm) 8.48 (t, 1H), 8.1 (dd, 1H), 7.9 (m, 3H), 7.67 (t, 1H), 7.38 (t, 1H), 4.48 (m, 2H), 3.55 (m, 2H).

### Example No. E1-1

¹H-NMR (300 MHz, CDCl₃ δ, ppm) 8.02 (d, 1H), 7.60 (t, 1H), 7.40 (m, 2H), 7.10 (d, 1H), 6.91 (br. s, 1H, NH), 6.35 (t, 1H), 6.18 (m, 1H), 4.25 (br. s, 2H).

### Example No. E1-4

¹H-NMR (300 MHz, CDCl₃ δ, ppm) 7.88 (d, 1H), 7.45 (m, 2H), 7.35 (t, 1H), 7.20 (d, 1H), 5.93 (s, 2H), 4.05 (d, 2H), 2.25 (s, 3H).

### Example No. E9-4

¹H-NMR (300 MHz, CDCl₃ δ, ppm) 7.60 (m, 1H), 7.20 (m, 2H), 6.50 (br. s, 1H, NH), 5.95 (s, 2H), 4.10 (m, 1H), 2.18 (s, 3H).

### Example No. E10-4

¹H-NMR (300 MHz, CDCl₃ δ, ppm) 8.00 (br. d, 2H), 7.55 (dd, 1H), 7.30 (m, 1H), 6.10 (br. s, 2H), 4.20 (d, 2H), 2.35 (s, 3H).

### Example No. E11-4

¹H-NMR (300 MHz, CDCl₃ δ, ppm) 8.01 (s, 1H), 7.60 (d, 1H), 7.45 (br. s, 1H, NH), 7.15 (d, 1H), 5.95 (s, 2H), 4.10 (m, 2H), 2.25 (s, 3H).

### Example No. E12-4

¹H-NMR (300 MHz, CDCl₃ δ, ppm) 8.20 (s, 1H), 7.70 (d, 1H), 7.01 (br. s, 1H, NH), 6.92 (d, 1H), 5.95 (s, 2H), 4.10 (m, 2H), 2.25 (s, 3H).

### Example No. E13-4

¹H-NMR (300 MHz, CDCl₃ δ, ppm) 7.75 (s, 1H), 7.40 (d, 1H), 7.20 (d, 1H), 6.60 (br. s, 1H, NH), 6.00 (s, 2H), 4.10 (m, 2H), 2.45 (s, 3H), 2.33 (s, 3H).

### Example No. E14-4

¹H-NMR (300 MHz, CDCl₃ δ, ppm) 7.38 (d, 1H), 7.20 (d, 1H), 7.08 (dd, 1H), 6.52 (br. s, 1H, NH), 5.95 (s, 2H), 4.10 (m, 2H), 3.85 (s, 3H), 2.20 (s, 3H).

### Example No. E19-4

¹H-NMR (300 MHz, CD₃OD δ, ppm) 7.18 (m, 2H), 7.00 (dd, 1H), 5.90 (s, 2H), 3.98 (d, 2H), 2.18 (s, 3H).

### Example No. E22-4

¹H-NMR (300 MHz, CDCl₃ δ, ppm) 7.90 (m, 1H), 7.10 (m, 1H), 6.91 (dd, 2H), 6.00 (m, 2H), 4.05 (m, 2H), 2.25 (s, 3H).

### Example No. E23-4

¹H-NMR (300 MHz, CDCl₃ δ, ppm) 7.85 (d, 1H), 7.35 (d, 1H), 7.22 (d, 1H), 6.50 (br. s, 1H, NH), 5.95 (t, 2H), 4.05 (m, 2H), 2.30 (s, 3H).

### Example No. E26-4

¹H-NMR (300 MHz, CDCl₃ δ, ppm) 7.88 (d, 1H), 7.20 (d, 1H), 7.11 (s, 1H), 6.55 (br. s, 1H, NH), 6.03 (m, 2H), 4.10 (m, 2H), 2.46 (s, 3H), 2.33 (s, 3H).

### Example No. E27-4

¹H-NMR (300 MHz, CDCl₃ δ, ppm) 8.10 (d, 1H), 7.10 (d, 1H), 6.91 (d, 1H), 6.72 (br. s, 1H, NH), 6.18 (m, 2H), 4.30 (m, 2H), 4.01 (s, 3H), 2.48 (s, 3H).

### Example No. 34-4

¹H-NMR (300 MHz, CDCl₃ δ, ppm) 7.75 (d, 1H), 7.43 (m, 2H), 6.55 (br. s, 1H, NH), 6.10 (d, 1H), 6.01 (d, 1H), 4.15 (m, 2H), 2.20 (s, 3H).

### Example No. E35-4

¹H-NMR (300 MHz, CDCl₃ δ, ppm) 7.85 (d, 1H), 7.74 (d, 1H), 7.43 (t, 1H), 6.55 (br. s, 1H, NH), 6.15 (d, 1H), 6.05 (d, 1H), 4.10 (m, 2H), 2.25 (s, 3H).

### Example No. E38-4

¹H-NMR (300 MHz, CDCl₃ δ, ppm) 7.70 (d, 1H), 7.45 (d, 1H), 7.37 (t, 1H), 6.80 (br. s, 1H, NH), 6.05 (d, 1H), 5.98 (d, 1H), 4.03 (m, 2H), 2.21 (s, 3H), 2.16 (s, 3H).

### Example No. E39-4

¹H-NMR (300 MHz, CDCl₃ δ, ppm) 7.40 (d, 1H), 7.33 (t, 1H), 7.10 (d, 1H), 6.38 (br. s, 1H, NH), 5.96 (d, 1H), 5.87 (d, 1H), 3.97 (m, 2H), 3.70 (s, 3H), 2.00 (s, 3H).

### Example No. E46-4

¹H-NMR (300 MHz, CDCl₃ δ, ppm) 7.55 (s, 1H), 7.28 (s, 1H), 6.40 (br. s, 1H, NH), 6.02 (m, 2H), 4.10 (m, 2H), 2.42 (s, 3H), 2.20 (s, 3H), 2.11 (s, 3H).

### Example No. E47-4

¹H-NMR (300 MHz, CDCl₃ δ, ppm) 8.10 (s, 1H), 7.90 (s, 1H), 7.12 (br. s, 1H, NH), 6.38 (d, 1H), 6.30 (d, 1H), 4.35 (m, 2H), 2.43 (s, 3H).

### Example No. E49-4

¹H-NMR (300 MHz, CDCl₃ δ, ppm) 7.70 (d, 1H), 7.35 (m, 1H), 6.55 (br. s, 1H, NH), 6.10 (m, 1H), 6.01 (m, 1H), 4.15 (m, 2H), 2.44 (s, 3H), 2.15 (d, 6H).

The present invention thus provides for the use of at least one compound selected from the group consisting of substituted benzodiazepinones and benzazepinones of the general formula (I), and of any mixtures of these substituted benzodiazepinones and benzazepinones of the general formula (I) according to the invention with further agrochemically active compounds, for enhancement of the resistance of plants to abiotic stress factors, preferably drought stress, and for invigoration of plant growth and/or for increasing plant yield.

The present invention further provides a spray solution for treatment of plants, comprising an amount, effective for enhancement of the resistance of plants to abiotic stress factors, of at least one compound selected from the group consisting of substituted benzodiazepinones and benzazepinones of the general formula (I). The abiotic stress conditions which can be relativized may include, for example, heat, drought, cold and aridity stress (stress caused by aridity and/or lack of water), osmotic stress, waterlogging, elevated soil salinity, elevated exposure to minerals, ozone conditions, strong light conditions, limited availability of nitrogen nutrients, limited availability of phosphorus nutrients.

In one embodiment, it is possible to provide, for example, that one or more compounds intended in accordance with the invention, i.e. the appropriate substituted benzodiazepinones and benzazepinones of the general formula (I), are applied by spray application to appropriate plants or plant parts to be treated. The use intended according to the invention of one or more compounds of the general formula (I) or salts thereof is carried out preferably with a dosage between 0.00005 and 3 kg/ha, more preferably between 0.0001 and 2 kg/ha, especially preferably between 0.0005 and 1 kg/ha, specifically preferably between 0.001 and 0.25 kg/ha. If, in the context of the present invention, abscisic acid is used simultaneously with substituted benzodiazepinones and benzazepinones of the general formula (I), for example in the context of a combined preparation or formulation, the addition of abscisic acid is preferably carried out in a dosage between 0.0001 and 3 kg/ha, particularly preferably between 0.001 and 2 kg/ha, especially preferably between 0.005 and 1 kg/ha, specifically preferably between 0.006 and 0.25 kg/ha.

The term “resistance to abiotic stress” is understood in the context of the present invention to mean various kinds of advantages for plants. Such advantageous properties are manifested, for example, in the improved plant characteristics given below: improved root growth with regard to surface area and depth, increased stolon or tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soil and water, enhanced tolerance to ozone stress, improved compatibility with respect to herbicides and other plant treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

More particularly, the use according to the invention of one or more compounds of the general formula (I) exhibits the advantages described in spray application to plants and plant parts. Combinations of the appropriate substituted benzodiazepinones and benzazepinones of the general formula (I) with substances including insecticides, attractants, acaricides, fungicides, nematicides, herbicides, growth regulators, safeners, substances which influence plant maturity, and bactericides can likewise be employed in the control of plant disorders and/or to achieve an increase in yield in the context of the present invention. In addition, the combined use of one or more substituted benzodiazepinones and benzazepinones of the general formula (I) according to the invention with genetically modified cultivars with a view to increased tolerance to abiotic stress is likewise possible.

As is well known, the further various benefits for plants mentioned above can be combined in the form of component parts, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is entirely familiar.

In the context of the present invention, a good effect on resistance to abiotic stress is understood to mean, without limitation,
at least an emergence improved by generally 3%, especially more than 5%, more preferably more than 10%,
at least a yield enhanced by generally 3%, especially more than 5%, more preferably more than 10%,
at least a root development improved by generally 3%, especially more than 5%, more preferably more than 10%,
at least a shoot size rising by generally 3%, especially more than 5%, more preferably more than 10%,
at least a leaf area increased by generally 3%, especially more than 5%, more preferably more than 10%,
at least a photosynthesis performance improved by generally 3%, especially more than 5%, more preferably more than 10%, and/or
at least a flower development improved by generally 3%, especially more than 5%, more preferably more than 10%,
   and the effects may occur individually or else in any combination of two or more effects.

The present invention further provides a spray solution for treatment of plants, comprising an amount, effective for enhancement of the resistance of plants to abiotic stress factors, of at least one compound from the group of the benzodiazepinones and benzazepinones of the general formula (I). The spray solution may comprise other customary constituents, such as solvents, formulation auxiliaries, especially water. Further constituents may include active agrochemical compounds which are described in more detail below.

The present invention further provides for the use of corresponding spray solutions for increasing the resistance of plants to abiotic stress factors. The remarks which follow apply both to the use according to the invention of one or more compounds of the general formula (I) per se and to the corresponding spray solutions.

In accordance with the invention, it has additionally been found that the application, to plants or in their environment, of one or more compounds of the general formula (I) in combination with at least one fertilizer as defined further below is possible.

Fertilizers which can be used in accordance with the invention together with one or more compounds of the general formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention should be made of the NPK fertilizers, i.e. fertilizers which contain nitrogen, phosphorus and potassium, calcium ammonium nitrate, i.e. fertilizers which additionally contain calcium, or ammonia nitrate sulfate (general formula (NH₄)₂SO₄NH₄NO₃), ammonium phosphate and ammonium sulfate. These fertilizers are generally known to the person skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may additionally comprise salts of micronutrients (preferably calcium, sulfur, boron, manganese, magnesium, iron, boron, copper, zinc, molybdenum and cobalt) and of phytohormones (for example vitamin B1 and indole (III)acetic acid) or mixtures of these. Fertilizers used in accordance with the invention may also contain other salts such as monoammonium phosphate (MAP), diammonium phosphate (DAP), potassium sulfate, potassium chloride, magnesium sulfate. Suitable amounts for the secondary nutrients or trace elements are amounts of 0.5% to 5% by weight, based on the overall fertilizer. Further possible compounds are crop protection agents, insecticides or fungicides, growth regulators or mixtures thereof. Further details of these are given further below.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764. The general composition of the fertilizers, which, in the context of the present invention, may take the form of straight and/or compound fertilizers, for example composed of nitrogen, potassium or phosphorus, may vary within a wide range. In general, a content of 1% to 30% by weight of nitrogen (preferably 5% to 20% by weight), 1% to % by weight of potassium (preferably 3% to 15% by weight) and a content of 1% to % by weight of phosphorus (preferably 3% to 10% by weight) is advantageous. The microelement content is usually in the ppm range, preferably in the range from 1 to 1000 ppm.

In the context of the present invention, the fertilizer and one or more compounds of the general formula (I) may be administered simultaneously. However, it is also possible first to apply the fertilizer and then one or more compounds of the general formula (I), or first to apply a compound of the general formula (I) and then the fertilizer. In the case of nonsynchronous application of one or more compounds of the general formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, the compound of the formula (I) according to the invention and the fertilizer are applied within a time frame of less than 1 hour, preferably less than 30 minutes, more preferably less than 15 minutes.

Preference is given to the use of one or more compounds of the general formula (I) on plants from the group of the useful plants, ornamentals, turfgrass types, commonly used trees which are used as ornamentals in the public and domestic sectors, and forestry trees. Forestry trees include trees for the production of timber, cellulose, paper and products made from parts of the trees. The term useful plants as used here refers to crop plants which are used as plants for obtaining foods, animal feeds, fuels or for industrial purposes.

The useful plants include, for example, the following types of plants: triticale, durum (hard wheat), turf, vines, cereals, for example wheat, barley, rye, oats, rice, corn and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cocoa beans and peanuts; cucurbits, for example pumpkin/squash, cucumbers and melons; fiber plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, eggplant, sugar cane, tea, pepper, grapevines, hops, bananas, latex plants and ornamentals, for example flowers, shrubs, deciduous trees and coniferous trees. This enumeration does not constitute a limitation.

The following plants are considered to be particularly suitable target crops for the application of the method according to the invention: oats, rye, triticale, durum, cotton, eggplant, turf, pome fruit, stone fruit, soft fruit, corn, wheat, barley, cucumber, tobacco, vines, rice, cereals, pears, pepper, beans, soybeans, oilseed rape, tomato, bell pepper, melons, cabbage, potatoes and apples.

Examples of trees which can be improved by the method according to the invention include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved by the method according to the invention include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved by the method according to the invention are: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved by the method according to the invention are: horse chestnut, Platanaceae, linden tree and maple tree. The present invention can also be applied to any desired turfgrasses, including cool-season turfgrasses and warm-season turfgrasses. Examples of cool-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);
ryegrasses (Lolium spp.), such as annual ryegrass (Lolium multiflorum Lam.), perennial ryegrass (Lolium perenne L.) and italian ryegrass (Lolium multiflorum Lam.); and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchard grass (Dactylis glomerata L.), weeping alkaligrass (Puccinellia distans (L.) Parl.) and crested dog's-tail (Cynosurus cristatus L.).

Examples of warm-season turfgrasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpet grass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.)). Cool-season turfgrasses are generally preferred for the use according to the invention. Particular preference is given to bluegrass, bentgrass and redtop, fescues and ryegrasses. Bentgrass is especially preferred.

Particular preference is given to using one or more compounds of the general formula (I) according to the invention to treat plants of the respective commercially available or commonly used plant cultivars. Plant cultivars are understood to mean plants which have new properties (“traits”) and which have been bred by conventional breeding, by mutagenesis or with the aid of recombinant DNA techniques. Crop plants may accordingly be plants which can be obtained by conventional breeding and optimization methods or by biotechnological and genetic engineering methods or combinations of these methods, including the transgenic plants and including the plant cultivars which are protectable or non-protectable by plant breeders' rights.

The treatment method according to the invention can thus also be used for the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced into the nuclear, chloroplastic or hypochondrial genome gives the transformed plant new or improved agronomic or other properties by expressing a protein or polypeptide of interest or by downregulating or silencing (an)other gene(s) which is/are present in the plant (using for example antisense technology, cosuppression technology or RNAi technology [RNA interference]). A heterologous gene that is located in the genome is also called a transgene. A transgene that is defined by its specific presence in the plant genome is called a transformation or transgenic event.

Plants and plant varieties which are preferably treated with one or more compounds of the general formula (I) according to the invention include all plants which have genetic material which imparts particularly advantageous, useful traits to these plants (whether obtained by breeding and/or biotechnological means).

Plants and plant varieties which can likewise be treated with the compounds of the general formula (I) according to the invention are those plants which are resistant to one or more abiotic stress factors. Abiotic stress conditions may include, for example, heat, drought, cold and aridity stress, osmotic stress, waterlogging, increased soil salinity, increased exposure to minerals, ozone conditions, strong light conditions, limited availability of nitrogen nutrients, limited availability of phosphorus nutrients or shade avoidance.

Plants and plant cultivars which can likewise be treated with the compounds of the general formula (I) according to the invention are those plants which are characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can also be affected by improved plant architecture (under stress and non-stress conditions), including but not limited to early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and oil composition, nutritional value, reduction in antinutritional compounds, improved processability and better storage stability.

Plants that may also be treated with the compounds of the general formula (I) according to the invention are hybrid plants that already express the characteristics of heterosis, or hybrid effect, which results in generally higher yield, higher vigor, better health and better resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (for example in corn) be produced by detasseling (i.e. mechanical removal of the male reproductive organs or male flowers); however, it is more typical for male sterility to be the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species (WO 92/005251, WO 95/009910, WO 98/27806, WO 05/002324, WO 06/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar (e.g. WO 91/002069).

Plants or plant varieties (obtained by plant biotechnology methods such as genetic engineering) which may also be treated with the compounds of the general formula (I) according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 01/66704). It can also be a mutated EPSPS, as described, for example, in EP-A 0837944, WO 00/066746, WO 00/066747 or WO 02/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described, for example, in WO 02/036782, WO 03/092360, WO 05/012515 and WO 07/024782. Glyphosate-tolerant plants can also be obtained by selecting plants containing naturally occurring mutations of the abovementioned genes, as described, for example, in WO 01/024615 or WO 03/013226.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One such effective detoxifying enzyme is, for example, an enzyme encoding a phosphinothricin acetyltransferase (such as the bar or pat protein from Streptomyces species). Plants expressing an exogenous phosphinothricin acetyltransferase are described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyze the reaction in which para-hydroxyphenylpyruvate (HPP) is converted to homogentizate. Plants tolerant to HPPD inhibitors can be transformed with a gene encoding a naturally-occurring resistant HPPD enzyme, or a gene encoding a mutated HPPD enzyme according to WO 96/038567, WO 99/024585 and WO 99/024586. Tolerance to HPPD inhibitors can also be obtained by transforming plants with genes encoding certain enzymes enabling the formation of homogentizate despite the inhibition of the native HPPD enzyme by the HPPD inhibitor. Such plants and genes are described in WO 99/034008 and WO 2002/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928.

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyoxy(thio)benzoates, and/or sulfonylaminocarbonyltriazolinone herbicides. Different mutations in the ALS enzyme (also known as acetohydroxy acid synthase, AHAS) are known to confer tolerance to different herbicides and groups of herbicides, as described, for example, in Tranel and Wright, Weed Science (2002), 50, 700-712, and also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 96/033270. Further imidazolinone-tolerant plants have also been described, for example, in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example, in WO 2007/024782.

Further plants tolerant to ALS-inhibitors, in particular to imidazolinones, sulfonylureas and/or sulfamoylcarbonyltriazolinones can be obtained by induced mutagenesis, by selection in cell cultures in the presence of the herbicide or by mutation breeding, as described, for example, for soybeans in U.S. Pat. No. 5,084,082, for rice in WO 97/41218, for sugarbeet in U.S. Pat. No. 5,773,702 and WO 99/057965, for lettuce in U.S. Pat. No. 5,198,599 or for sunflower in WO 2001/065922.

Plants or plant cultivars (obtained by plant biotechnology methods such as genetic engineering) which may also be treated with one or more compounds of the general formula (I) according to the invention are insect-resistant transgenic plants, i.e. plants made resistant to attack by certain target insects. Such plants can be obtained by genetic transformation, or by selection of plants containing a mutation imparting such insect resistance.

The term “insect-resistant transgenic plant”, as used herein, includes any plant containing at least one transgene comprising a coding sequence encoding:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein than Bacillus thuringiensis or a portion thereof which is insecticidal in the presence of a second, other crystal protein from Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by corn event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in corn events MON863 or MON88017, or the Cry3A protein in corn event MIR 604; or
5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, an insect-resistant transgenic plant, as used herein, also includes any plant comprising a combination of genes encoding the proteins of any one of the above classes 1 to 8. In one embodiment, an insect-resistant plant contains more than one transgene encoding a protein of any one of the above classes 1 to 8, to expand the range of the target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated with one or more compounds of the general formula (I) according to the invention are tolerant to abiotic stress factors. Such plants can be obtained by genetic transformation, or by selection of plants containing a mutation imparting such stress resistance. Particularly useful stress tolerant plants include:

a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5;
b. plants which contain a stress tolerance-enhancing transgene capable of reducing the expression and/or the activity of the PARG-encoding genes of the plants or plant cells, as described, for example, in WO 2004/090140;
c. plants which contain a stress tolerance-enhancing transgene encoding a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase, as described, for example, in EP 04077624.7 or WO 2006/133827 or PCT/EP07/002433.

Plants or plant varieties (obtained by plant biotechnology methods such as genetic engineering) which may also be treated with the compounds of the general formula (I) according to the invention show altered quantity, quality and/or storage stability of the harvested product and/or altered properties of specific ingredients of the harvested product such as, for example:

1) transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications. These transgenic plants synthesizing a modified starch are described, for example, in EP 0571427, WO 95/004826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 95/26407, WO 96/34968, WO 98/20145, WO 99/12950, WO 99/66050, WO 99/53072, U.S. Pat. No. 6,734,341, WO 2000/11192, WO 98/22604, WO 98/32326, WO 2001/98509, WO 2001/98509, WO 2005/002359, U.S. Pat. No. 5,824,790, U.S. Pat. No. 6,013,861, WO 94/004693, WO 94/009144, WO 94/11520, WO 95/35026 and WO 97/20936.
2) transgenic plants which synthesize non-starch carbohydrate polymers or non-starch carbohydrate polymers with altered properties in comparison to wild type plants without genetic modification. Examples are plants producing polyfructose, especially of the inulin and levan type, as described in EP 0663956, WO 96/001904, WO 96/021023, WO 98/039460 and WO 99/024593, plants producing alpha-1,4-glucans, as described in WO 95/031553, US 2002/031826, U.S. Pat. No. 6,284,479, U.S. Pat. No. 5,712,107, WO 97/047806, WO 97/047807, WO 97/047808 and WO 2000/14249, plants producing alpha-1,6-branched alpha-1,4-glucans, as described in WO 2000/73422, and plants producing alternan, as described in WO 2000/047727, EP 06077301.7, U.S. Pat. No. 5,908,975 and EP 0728213.
3) Transgenic plants which produce hyaluronan, as for example described in WO 06/032538, WO 2007/039314, WO 2007/039315, WO 2007/039316, JP 2006/304779 and WO 2005/012529.

Plants or plant cultivars (obtained by plant biotechnology methods such as genetic engineering) which may also be treated with the compounds of the general formula (I) according to the invention are plants, such as cotton plants, with altered fiber characteristics. Such plants can be obtained by genetic transformation, or by selection of plants containing a mutation imparting such altered fiber characteristics and include:

a) plants, such as cotton plants, which contain an altered form of cellulose synthase genes, as described in WO 98/000549;
b) plants, such as cotton plants, which contain an altered form of rsw2 or rsw3 homologous nucleic acids, as described in WO 2004/053219;
c) plants, such as cotton plants, with an increased expression of sucrose phosphate synthase, as described in WO 2001/017333;
d) plants, such as cotton plants, with increased expression of sucrose synthase as described in WO 02/45485;
e) plants, such as cotton plants, wherein the timing of the plasmodesmatal gating at the basis of the fiber cell is altered, for example through downregulation of fiber-selective β-1,3-glucanase as described in WO 2005/017157;
f) plants, such as cotton plants, which have fibers with altered reactivity, for example through expression of the N-acetylglucosamine transferase gene including nodC and chitin synthase genes, as described in WO 2006/136351.

Plants or plant cultivars (obtained by plant biotechnology methods such as genetic engineering) which may also be treated with the compounds of the general formula (I) according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;
b) plants, such as oilseed rape plants, which produce oil having a low linolenic acid content, as described in U.S. Pat. No. 6,270,828, U.S. Pat. No. 6,169,190 or U.S. Pat. No. 5,965,755;
c) plants, such as oilseed rape plants, which produce oil having a low level of saturated fatty acids, as described, for example, in U.S. Pat. No. 5,434,283.

Particularly useful transgenic plants which may be treated with one or more compounds of the general formula (I) according to the invention are plants containing transformation events, or a combination of transformation events, and that are listed for example in the databases of various national or regional regulatory agencies.

Particularly useful transgenic plants which may be treated with one or more compounds of the general formula (I) according to the invention are, for example, plants which comprise one or more genes which encode one or more toxins and are the transgenic plants available under the following trade names: YIELD GARD® (for example corn, cotton, soybeans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants which may be mentioned are corn varieties, cotton varieties and soya bean varieties which are available under the following trade names: Roundup Ready® (tolerance to glyphosates, for example corn, cotton, soybeans), Liberty Link® (tolerance to phosphinothricin, for example oilseed rape), IMI® (tolerance to imidazolinone) and SCS® (tolerance to sulfonylurea), for example corn. Herbicide-resistant plants (plants bred in a conventional manner for herbicide tolerance) which may be mentioned include the varieties sold under the name Clearfield® (for example corn).

The compounds of the formula (I) to be used in accordance with the invention can be converted to customary formulations, such as solutions, emulsions, wettable powders, water- and oil-based suspensions, powders, dusts, pastes, soluble powders, soluble granules, granules for broadcasting, suspoemulsion concentrates, natural substances impregnated with active compound, synthetic substances impregnated with active compound, fertilizers, and also microencapsulations in polymeric substances. In the context of the present invention, it is especially preferred when one or more compounds of the general formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing one or more compounds of the general formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are produced either in suitable facilities or else before or during application.

The auxiliaries used may be those substances which are suitable for imparting, to the composition itself and/or to preparations derived therefrom (for example spray liquors), particular properties such as particular technical properties and/or else special biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender used is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethyl sulfoxide, and also water.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

Suitable wetting agents which may be present in the formulations which can be used in accordance with the invention are all substances which promote wetting and which are conventionally used for the formulation of agrochemical active substances. Preference is given to using alkyl naphthalenesulfonates, such as diisopropyl or diisobutyl naphthalenesulfonates.

Suitable dispersants and/or emulsifiers which may be present in the formulations which can be used in accordance with the invention are all nonionic, anionic and cationic dispersants conventionally used for the formulation of agrochemically active compounds. Preference is given to using nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristyrylphenol polyglycol ether and their phosphated or sulfated derivatives. Suitable anionic dispersants are especially lignosulfonates, polyacrylic acid salts and arylsulfonate/formaldehyde condensates.

Suitable antifoams which may be present in the formulations which can be used in accordance with the invention are all foam-inhibiting substances conventionally used for the formulation of agrochemical active substances. Silicone antifoams and magnesium stearate can be used with preference.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preferred examples include cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose. Suitable gibberellins which may be present in the formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz-und Schädlingsbekämpfungsmittel”, vol. 2, Springer Verlag, 1970, pp. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01 and 98% by weight, preferably between 0.5 and 90%, of the compound of the general formula (I).

The compounds of the general formula (I) according to the invention may be present in commercially available formulations, and also in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

In addition, the described positive effect of the compounds of the formula (I) on the plants' own defences can be supported by an additional treatment with active insecticidal, fungicidal or bactericidal compounds.

Preferred times for the application of one or more compounds of the general formula (I) according to the invention for enhancing resistance to abiotic stress are treatments of the soil, stems and/or leaves with the approved application rates.

The active compounds of the general formula (I) may generally additionally be present in their commercial formulations and in the use forms prepared from these formulations in mixtures with other active compounds, such as insecticides, attractants, sterilants, acaricides, nematicides, fungicides, bactericides, growth regulators, substances which influence plant maturity, safeners or herbicides. Particularly favorable mixing partners are, for example, the active compounds of the different classes specified below in groups, without any preference arising from the sequence thereof:

### Fungicides:

F1) nucleic acid synthesis inhibitors, for example benalaxyl, benalaxyl-M, bupirimate, chiralaxyl, clozylacon, dimethirimol, ethirimol, furalaxyl, hymexazole, metalaxyl, metalaxyl-M, ofurace, oxadixyl, oxolinic acid;
F2) mitosis and cell division inhibitors, for example benomyl, carbendazim, diethofencarb, fuberidazole, fluopicolid, pencycuron, thiabendazole, thiophanate-methyl, zoxamide and chloro-7-(4-methylpiperidin-1-yl)-6-(2,4,6-trifluorophenyl)[1,2,4]triazolo[1,5-a]pyrimidine;
F3) respiratory chain complex I/II inhibitors, for example diflumetorim, bixafen, boscalid, carboxin, diflumethorim, fenfuram, fluopyram, flutolanil, furametpyr, mepronil, oxycarboxin, penflufen, penthiopyrad, thifluzamid, N-[2-(1,3-dimethylbutyl)phenyl]-5-fluoro-1,3-dimethyl-1H-pyrazole-4-carboxamide, isopyrazam, sedaxan, 3-(difluoromethyl)-1-methyl-N-(3′,4′,5′-trifluorobiphenyl-2-yl)-1H-pyrazole-4-carboxamide, 3-(difluoromethyl)-1-methyl-N-[2-(1,1,2,2-tetrafluoroethoxyl)phenyl]-1H-pyrazole-4-carboxamide, 3-(difluoromethyl)-N-[4-fluoro-2-(1,1,2,3,3,3-hexafluoropropoxyl)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide and corresponding salts;
F4) respiratory chain complex III inhibitors, for example amisulbrom, azoxystrobin, cyazofamid, dimoxystrobin, enestrobin, famoxadon, fenamidon, fluoxastrobin, kresoxim-methyl, metominostrobin, orysastrobin, pyraclostrobin, pyribencarb, picoxystrobin, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(ethoxyimino)-N-methyl-2-(2-{[({(1E)-1-[3-(trifluoromethyl)phenyl]ethylidene}amino)oxy]methyl}phenyl)ethanamide and corresponding salts, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, 2-methyl {2-[({cyclopropyl[(4-methoxyphenyl)imino]methyl}sulfanyl)methyl]phenyl}-3-methoxyacrylate, N-(3-ethyl-3,5,5-trimethylcyclohexyl)-3-(formylamino)-2-hydroxybenzamide and corresponding salts;
F5) decouplers, for example dinocap, fluazinam;
F6) ATP production inhibitors, for example fentin acetate, fentin chloride, fentin hydroxide, silthiofam
F7) amino acid and protein biosynthesis inhibitors, for example andoprim, blasticidin-S, cyprodinil, kasugamycin, kasugamycin hydrochloride hydrate, mepanipyrim, pyrimethanil
F8) signal transduction inhibitors, for example fenpiclonil, fludioxonil, quinoxyfen
F9) lipid and membrane synthesis inhibitors, for example chlozolinate, iprodione, procymidone, vinclozolin, ampropylfos, potassium-ampropylfos, edifenphos, iprobenfos (IBP), isoprothiolane, pyrazophos, tolclofos-methyl, biphenyl, iodocarb, propamocarb, propamocarb hydrochloride
F10) ergosterol biosynthesis inhibitors, for example fenhexamid, azaconazole, bitertanol, bromuconazole, diclobutrazole, difenoconazole, diniconazole, diniconazole-M, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, spiroxamine, tebuconazole, triadimefon, triadimenol, triticonazole, uniconazole, voriconazole, imazalil, imazalilsulfate, oxpoconazole, fenarimol, flurprimidol, nuarimol, pyrifenox, triforin, pefurazoate, prochloraz, triflumizole, viniconazole, aldimorph, dodemorph, dodemorph acetate, fenpropimorph, tridemorph, fenpropidin, naftifin, pyributicarb, terbinafin, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-{1-[(4-methoxyphenoxy)methyl]2,2-dimethylpropyl}-1H-imidazole-1-carbothioate;
F11) cell wall synthesis inhibitors, for example benthiavalicarb, bialaphos, dimethomorph, flumorph, iprovalicarb, polyoxins, polyoxorim, validamycin A
F12) melanine biosynthesis inhibitors, for example capropamide, diclocymet, fenoxanil, phthalide, pyroquilon, tricyclazole
F13) resistance induction, for example acibenzolar-S-methyl, probenazole, tiadinil
F14) multisite, for example captafol, captan, chlorothalonil, copper salts such as: copper hydroxide, copper naphthenate, copper oxychloride, copper sulfate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulfur and sulfur preparations containing calcium polysulfide, thiram, tolylfluanid, zineb, ziram
F15) unknown mechanism, for example amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulfate, diphenylamine, ethaboxam, ferimzone, flumetover, flusulfamide, fluopicolid, fluoroimid, fosatyl-Al, hexachlorobenzene, 8-hydroxyquinoline sulfate, iprodione, irumamycin, isotianil, methasulfocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrolnitrin, quintozene, tecloftalam, tecnazene, triazoxide, trichlamide, zarilamid and 2,3,5,6-tetrachloro-4-(methylsulfonyl)pyridine, N-(4-chloro-2-nitrophenyl)-N-ethyl-4-methylbenzenesulfonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl]-.alpha.-(methoxymethylene)benzacetate, 4-chloro-alpha-propynyloxy-N-[2-[3-methoxy-4-(2-propynyloxyl)phenyl]ethyl]benzacetamide, (2S)—N-[2-[4-[[3-(4-chlorophenyl)-2-propynyl]oxy]-3-methoxyphenyl]ethyl]-3-methyl-2-[(methylsulfonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3-(1-fluoro-2-phenylethyl)oxy]phenyl]ethylidene]amino]oxy]methyl]-alpha-(methoxyimino)-N-methyl-alphaE-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

I1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos (-methyl, -ethyl), cadusafos, chlorethoxyfos, chlorfenvinphos, chlormephos, chlorpyrifos (-methyl), coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion (-methyl), phenthoate, phorate, phosalone, phosmet, phosphamidon, phoxim, pirimiphos (-methyl), profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
I2) GABA-gated chloride channel antagonists, for example organochlorines, e.g. chlordane and endosulfan (alpha-); or fiproles (phenylpyrazoles), e.g. ethiprole, fipronil, pyrafluprole and pyriprole.
I3) Sodium channel modulators/voltage-gated sodium channel blockers, for example pyrethroids, e.g. acrinathrin, allethrin (d-cis-trans, d-trans), bifenthrin, bioallethrin, bioallethrin-S-cyclopentenyl, bioresmethrin, cycloprothrin, cyfluthrin (beta-), cyhalothrin (gamma-, lambda-), cypermethrin (alpha-, beta-, theta-, zeta-), cyphenothrin [(1R)-trans-isomers], deltamethrin, dimefluthrin, empenthrin [(EZ)-(1R)-isomers], esfenvalerate, etofenprox, fenpropathrin, fenvalerate, flucythrinate, flumethrin, fluvalinate (tau-), halfenprox, imiprothrin, metofluthrin, permethrin, phenothrin [(1R)-trans-isomer], prallethrin, profluthrin, pyrethrins (pyrethrum), resmethrin, RU 15525, silafluofen, tefluthrin, tetramethrin [(1R)-isomers], tralomethrin, transfluthrin and ZXI 8901; or DDT; or methoxychlor.
I4) Nicotinergic acetylcholine receptor agonists, for example neonicotinoids, e.g. acetamiprid, clothianidin, dinotefuran, imidacloprid, nitenpyram, thiacloprid, thiamethoxam; or nicotine.
I5) Allosteric acetylcholine receptor modulators (agonists), for example spinosyns, e.g. spinetoram and spinosad.
I6) Chloride channel activators, for example avermectins/milbemycins, e.g. abamectin, emamectin, emamectin benzoate, lepimectin and milbemectin.
I7) Juvenile hormone analogs, e.g. hydroprene, kinoprene, methoprene; or fenoxycarb; pyriproxyfen.
I8) Active compounds with unknown or non-specific mechanisms of action, for example fumigants, for example methyl bromide and other alkyl halides; or chloropicrin; sulfuryl fluoride; borax; tartar emetic.
I9) Selective antifeedants, e.g. pymetrozine; or flonicamid.
I10) Mite growth inhibitors, e.g. clofentezine, diflovidazin, hexythiazox, etoxazole.
I11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins, for example Cry1Ab, Cry1Ac, Cry1Fa, Cry2Ab, mCry3A, Cry3Ab, Cry3Bb, Cry34/35Ab1.
I12) Oxidative phosphorylation inhibitors, ATP disruptors, for example diafenthiuron; or organotin compounds, e.g. azocyclotin, cyhexatin, fenbutatin oxide; or propargite; tetradifon.
I13) Oxidative phosphorylation decouplers through interruption of the H proton gradient, for example chlorfenapyr and DNOC.
I14) Nicotinergic acetylcholine receptor antagonists, for example bensultap, cartap (-hydrochloride), thiocyclam, and thiosultap (-sodium).
I15) Chitin biosynthesis inhibitors, type 0, for example benzoylureas, e.g. bistrifluron, chlorfluazuron, diflubenzuron, flucycloxuron, flufenoxuron, hexaflumuron, lufenuron, novaluron, noviflumuron, teflubenzuron and triflumuron.
I16) Chitin biosynthesis inhibitors, type 1, for example buprofezin.
I17) Moulting disruptors, for example cyromazine.
I18) Ecdysone agonists/disruptors, for example diacylhydrazines, for example chromafenozide, halofenozide, methoxyfenozide and tebufenozide.
I19) Octopaminergic agonists, for example amitraz.
I20) Complex III electron transport inhibitors, for example hydramethylnone; acequinocyl; fluacrypyrim.
I21) Complex I electron transport inhibitors, for example from the group of the METI acaricides, e.g. fenazaquin, fenpyroximate, pyrimidifen, pyridaben, tebufenpyrad, tolfenpyrad; or rotenone (Derris).
I22) Voltage-gated sodium channel blockers, e.g. indoxacarb; metaflumizone.
I23) Inhibitors of acetyl-CoA carboxylase, for example tetronic acid derivatives, e.g. spirodiclofen and spiromesifen; or tetramic acid derivatives, e.g. spirotetramat.
I24) Complex IV electron transport inhibitors, for example phosphines, e.g. aluminum phosphide, calcium phosphide, phosphine, zinc phosphide; or cyanide.
I25) Complex II electron transport inhibitors, for example cyenopyrafen.
I26) Ryanodine receptor effectors, for example diamides, e.g. flubendiamide, chlorantraniliprole (Rynaxypyr), cyantraniliprole (Cyazypyr) and 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) or methyl 2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (known from WO2007/043677).

Further active compounds having an unknown mechanism of action, such as, for example, azadirachtin, amidoflumet, benzoximate, bifenazate, chinomethionat, cryolite, cyflumetofen, dicofol, 5-chloro-2-[(3,4,4-trifluorobut-3-en-1-yl)sulfonyl]-1,3-thiazole, flufenerim, pyridalyl and pyrifluquinazon; furthermore preparations based on Bacillus firmus (1-1582, BioNeem, Votivo) and the following known active compounds 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-chloropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP0539588), 4-{[(6-chloropyrid-3-yl)methyl](methyl)amino}furan-2(5H)-one (known from EP0539588), [1-(6-chloropyridin-3-yl)ethyl](methyl)oxido-λ⁴-sulfanylidenecyanamide (known from WO 2007/149134) and its diastereomers {[(1R)-1-(6-chloropyridin-3-yl)ethyl](methyl)oxido-λ⁶-sulfanylidene}cyanamide and {[(1S)-1-(6-chloropyridin-3-yl)ethyl](methyl)oxido-λ⁶-sulfanylidene}cyanamide (likewise known from WO 2007/149134) and sulfoxaflor (likewise known from WO 2007/149134), 1-[2-fluoro-4-methyl-5-[(2,2,2-trifluoroethyl)sulfinyl]phenyl]-3-(trifluoromethyl)-1H-1,2,4-triazole-5-amine (known from WO 2006/043635), [(3S,4αR,12R,12αS,12βS)-3-[(cyclopropylcarbonyl)oxy]-6,12-dihydroxy-4,12b-dimethyl-11-oxo-9-(pyridin-3-yl)-1,3,4,4α,5,6,6α,12,12α,12β-decahydro-2H,11H-benzo[f]pyrano[4,3-b]chromen-4-yl]methylcyclopropanecarboxylate (known from WO 2006/129714), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluoromethoxy)-N-ethyl-N-methyl-1,2-benzothiazole-3-amine 1,1-dioxide (known from WO2007/057407), N-[1-(2,3-dimethylphenyl)-2-(3,5-dimethylphenyl)ethyl]-4,5-dihydro-1,3-thiazole-2-amine (known from WO2008/104503), {1′-[(2E)-3-(4-chlorophenyl)prop-2-en-1-yl]-5-fluorospiro[indol-3,4′-piperidin]-1 (2H)-yl}(2-chloropyridin-4-yl)methanone (known from WO2003106457), 3-(2,5-dimethylphenyl)-4-hydroxy-8-methoxy-1,8-diazaspiro[4.5]dec-3-en-2-one (known from WO2009049851), 3-(2,5-dimethylphenyl)-8-methoxy-2-oxo-1,8-diazaspiro[4.5]dec-3-en-4-yl ethyl carbonate (known from WO2009049851), 4-(but-2-yn-1-yloxy)-6-(3,5-dimethylpiperidin-1-yl)-5-fluoropyrimidine (known from WO2004099160), (2,2,3,3,4,4,5,5-octafluoropentyl)(3,3,3-trifluoropropyl)malononitrile (known from WO2005063094), (2,2,3,3,4,4,5,5-octafluoropentyl)(3,3,4,4,4-pentafluorobutyl)malononitrile (known from WO2005063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO2007040280/282), 2-ethyl-7-methoxy-3-methyl-6-[(2,2,3,3-tetrafluoro-2,3-dihydro-1,4-benzodioxin-6-yl)oxy]quinolin-4-yl methyl carbonate (known from JP2008110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008110953), PF 364 (Chemical Abstracts No 1204776-60-2, known from JP2010018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO2007075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005085216). Safeners are preferably selected from the group consisting of:

S1) Compounds of the formula (S1)

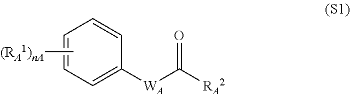

where the symbols and indices are defined as follows:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;

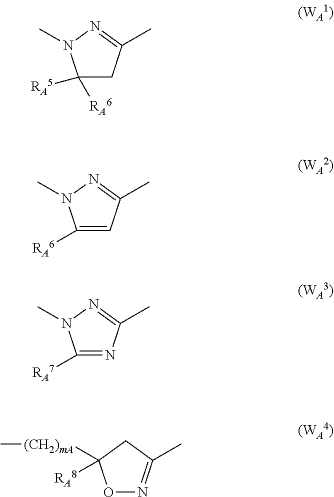

W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴);
m_{A} is 0 or 1;
R_{A}² is OR_{A}³, SR_{A}³ or NR_{A}³RA⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹, where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are the same or different and are each hydrogen, (C₁-C₈)-alkyl, (C₁-C₈)-haloalkyl, (C₃-C₁₂)-cycloalkyl or substituted or unsubstituted phenyl;
preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

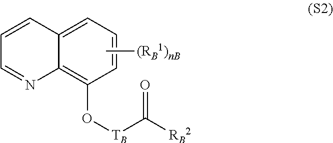

where the symbols and indices are defined as follows:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated
or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbon radical preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2^{a}), preferably 1-methylhexyl(5-chloro-8-quinolinoxy)acetate (“cloquintocet-mexyl”) (S2-1), 1,3-dimethylbut-1-yl(5-chloro-8-quinolinoxy)acetate (S2-2), 4-allyloxybutyl(5-chloro-8-quinolinoxy)acetate (S2-3), 1-allyloxyprop-2-yl(5-chloro-8-quinolinoxy)acetate (S2-4), ethyl (5-chloro-8-quinolinoxy)acetate (S2-5), methyl (5-chloro-8-quinolinoxy)acetate (S2-6), allyl(5-chloro-8-quinolinoxy)acetate (S2-7), 2-(2-propylideneiminoxy)-1-ethyl (5-chloro-8-quinolinoxy)acetate (S2-8), 2-oxoprop-1-yl(5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S₂^{b}), preferably compounds such as diethyl(5-chloro-8-quinolinoxy)malonate, diallyl(5-chloro-8-quinolinoxy)malonate, methyl ethyl (5-chloro-8-quinolinoxy)malonate and related compounds, as described in EP-A-0 582 198.
S3) Compounds of the formula (S3)

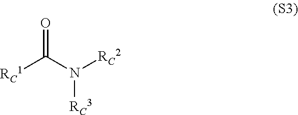

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are the same or different and are each hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring; preferably: active compounds of the dichloroacetamide type which are frequently used as pre-emergence safeners (soil-active safeners), such as, for example, “dichlormid” (N,N-diallyl-2,2-dichloroacetamide) (S3-1), “R-29148” (3-dichloroacetyl-2,2,5-trimethyl-1,3-oxazolidine) from Stauffer (S3-2), “R-28725” (3-dichloroacetyl-2,2-dimethyl-1,3-oxazolidine) from Stauffer (S3-3), “benoxacor” (4-dichloroacetyl-3,4-dihydro-3-methyl-2H-1,4-benzoxazine) (S3-4), “PPG-1292” (N-allyl-N-[(1,3-dioxolan-2-yl)methyl]dichloroacetamide) from PPG Industries (S3-5), “DKA-24” (N-allyl-N-[(allylaminocarbonyl)methyl]dichloroacetamide) from Sagro-Chem (S3-6), “AD-67” or “MON 4660” (3-dichloroacetyl-1-oxa-3-azaspiro[4,5]decane) from Nitrokemia or Monsanto (S3-7), “TI-35” (1-dichloroacetylazepane) from TRI-Chemical RT (S3-8), “diclonon” (dicyclonone) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10); and the (R) isomer thereof (S3-11).
S4) N-Acylsulfonamides of the formula (S4) and salts thereof,

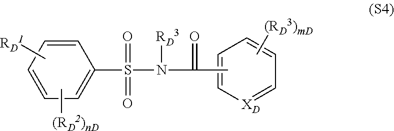

where the symbols and indices are each defined as follows:

### X_{D} is CH or N;

### R_{D}¹ is CO—NR_{D}⁵RD⁶ or NHCO—R_{D}⁷;

R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing V_{D} heteroatoms from the group of nitrogen, oxygen and sulfur, where the seven last-mentioned radicals are substituted by V_{D} substituents from the group of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three last-mentioned radicals are substituted by V_{D} radicals from the group consisting of halogen, hydroxy, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 last-mentioned radicals are substituted by V_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl; n_{D} is 0, 1 or 2;
m_{D} is 1 or 2;

### V_{D} is 0, 1, 2 or 3;

among these, preference is given to compounds of the N-acylsulfonamide type, for example of the formula (S4^{a}) below, which are known, for example, from WO-A-97/45016

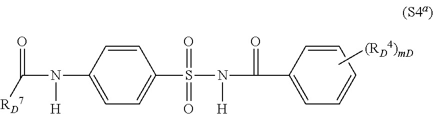

in which
R_{D}⁷ is (C₁-C₆)-alkyl, (C₃-C₆)-cycloalkyl, where the 2 last-mentioned radicals are substituted by vD substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁C₄)-haloalkyl;
R_{D}⁴ is halogen, (C₁-C₄)-alkyl, (C₁-C₄)-alkoxy, CF₃;
m_{D} is 1 or 2;

### V_{D} is 0, 1, 2 or 3;

and also to acylsulfamoylbenzamides, for example of the formula (S4^{b}) below, which are known, for example, from WO-A-99/16744,

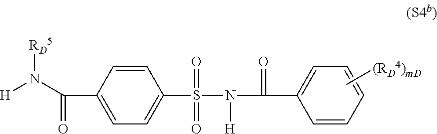

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
RD⁵=isopropyl and (R_{D}⁴)=2-OMe (S4-5)
and to compounds of the N-acylsulfamoylphenylurea type, of the formula (S4^{c}), which are known, for example, from EP-A-365484,

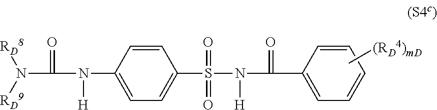

in which
R_{D}⁸ and R_{D}⁹ are each independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
   S5) Active compounds from the class of the hydroxyaromatics and aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
   S6) Active compounds from the class of the 1,2-dihydroquinoxalin-2-ones (S6), for example
   1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
   S7) Compounds of the formula (S7), as described in WO-A-1998/38856,

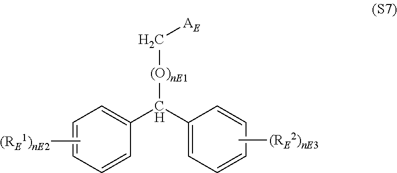

where the symbols and indices are each defined as follows:
R_{E}¹, R_{E}² are each independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro; A_{E} is COOR_{E}³ or COSR_{E}⁴
R_{E}³, R_{E}⁴ are each independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1
n_{E}², n_{E}³ are each independently 0, 1 or 2,
preferably diphenylmethoxyacetic acid, ethyl diphenylmethoxyacetate, methyl diphenylmethoxyacetate (CAS reg. no. 41858-19-9) (S7-1).
S8) Compounds of the formula (S8), as described in WO-A-98/27049,

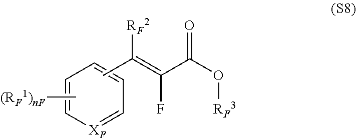

in which

### X_{F} is CH or N,

n_{F} in the case that X_{F}=N is an integer from 0 to 4 and
in the case that X_{F}=CH is an integer from 0 to 5,
   R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
   R_{F}² is hydrogen or (C₁-C₄)-alkyl,
   R_{F}³ is hydrogen, (C₁-C₈)-alkyl, (C₂-C₄)-alkenyl, (C₂-C₄)-alkynyl or aryl, where each of the carbon-containing radicals mentioned above is unsubstituted or substituted by one or more, preferably up to three, identical or different radicals from the group consisting of halogen and alkoxy, or salts thereof,
   preferably compounds in which

### X_{F} is CH,

n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl, or aryl, where each of the aforementioned carbon-containing radicals is unsubstituted or substituted by one or more, preferably up to three identical or different radicals from the group consisting of halogen and alkoxy,
or salts thereof.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS reg. no. 95855-00-8), as described in WO-A-1999/000020.
S10) Compounds of the formula (S10^{a}) or (S10^{b})
as described in WO-A-2007/023719 and WO-A-2007/023764

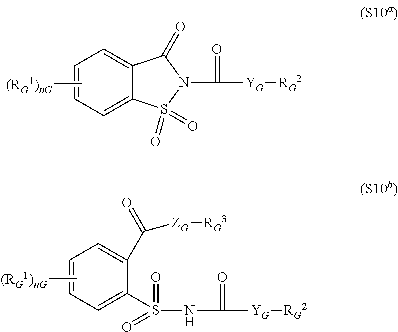

in which
R_{G}¹ is halogen, (C₁-C₄)-alkyl, methoxy, nitro, cyano, CF₃, OCF₃,
Y_{G}, Z_{G} are each independently O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl, R_{G}³ is hydrogen or (C₁-C₆)-alkyl.
S11) Active compounds of the oxyimino compound type (S11), which are known as seed-dressing compositions, for example “oxabetrinil” ((Z)-1,3-dioxolan-2-yl-methoxyimino(phenyl)acetonitrile) (S11-1), which is known as a seed-dressing safener for millet/sorghum against damage by metolachlor, “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against damage by metolachlor, and “cyometrinil” or “CGA-43089” ((Z)-cyanomethoxyimino(phenyl)acetonitrile) (S11-3), which is known as a seed-dressing safener for millet/sorghum against damage by metolachlor.
S12) Active compounds from the class of the isothiochromanones (S12), for example methyl [(3-oxo-1H-2-benzothiopyran-4(3H)-ylidene)methoxy]acetate (CAS reg. no. 205121-04-6) (S12-1) and related compounds from WO-A-1998/13361.
S13) One or more compounds from group (S13): “naphthalic anhydride” (1,8-naphthalenedicarboxylic anhydride) (S13-1), which is known as a seed-dressing safener for corn against damage by thiocarbamate herbicides, “fenclorim” (4,6-dichloro-2-phenylpyrimidine) (S13-2), which is known as a safener for pretilachlor in sown rice, “flurazole” (benzyl 2-chloro-4-trifluoromethyl-1,3-thiazole-5-carboxylate) (S13-3), which is known as a seed-dressing safener for millet/sorghum against damage by alachlor and metolachlor, “CL 304415” (CAS reg. no. 31541-57-8) (4-carboxy-3,4-dihydro-2H-1-benzopyran-4-acetic acid) (S13-4) from American Cyanamid, which is known as a safener for corn against damage by imidazolinones, “MG 191” (CAS reg. no. 96420-72-3) (2-dichloromethyl-2-methyl-1,3-dioxolane) (S13-5) from Nitrokemia, which is known as a safener for corn, “MG-838” (CAS reg. no. 133993-74-5) (2-propenyl 1-oxa-4-azaspiro[4.5]decane-4-carbodithioate) (S13-6) from Nitrokemia, “disulfoton” (O,O-diethyl S-2-ethylthioethyl phosphorodithioate) (S13-7), “dietholate” (O,0-diethyl O-phenylphosphorothioate) (S13-8), “mephenate” (4-chlorophenyl methylcarbamate) (S13-9).
S14) Active compounds which, in addition to herbicidal action against harmful plants, also have safener action on crop plants such as rice, for example “dimepiperate” or “MY-93” (S-1-methyl-1-phenylethylpiperidine-1-carbothioate), which is known as a safener for rice against damage by the herbicide molinate, “daimuron” or “SK 23” (1-(1-methyl-1-phenylethyl)-3-p-tolylurea), which is known as a safener for rice against damage by the herbicide imazosulfuron, “cumyluron”=“JC-940” (3-(2-chlorophenylmethyl)-1-(1-methyl-1-phenylethyl)urea, see JP-A-60087254), which is known as a safener for rice against damage by some herbicides, “methoxyphenone” or “NK 049” (3,3′-dimethyl-4-methoxybenzophenone), which is known as a safener for rice against damage by some herbicides, “CSB” (1-bromo-4-(chloromethylsulfonyl)benzene) from Kumiai, (CAS reg. no. 54091-06-4), which is known as a safener against damage by some herbicides in rice.
S15) Compounds of the formula (S15) or tautomers thereof
as described in WO-A-2008/131861 and WO-A-2008/131860,

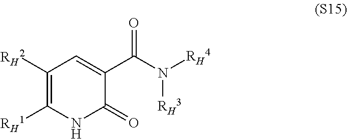

in which
   R_{H}¹ is a (C₁-C₆)-haloalkyl radical and
   R_{H}² is hydrogen or halogen and
   R_{H}³, R_{H}⁴ are each independently of one another hydrogen, (C₁-C₁₆)-alkyl, (C₂-C₁₆)-alkenyl or (C₂-C₁₆)-alkynyl, where each of the last-mentioned 3 radicals is unsubstituted or substituted by one or more radicals from the group of halogen, hydroxy, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted, or (C₃-C₆)-cycloalkyl, (C₄-C₆)-cycloalkenyl, (C₃-C₆)-cycloalkyl which is fused on one side of the ring to a 4- to 6-membered saturated or unsaturated carbocyclic ring, or (C₄-C₆)-cycloalkenyl which is fused on one side of the ring to a 4 to 6-membered saturated or unsaturated carbocyclic ring, where each of the last-mentioned 4 radicals is unsubstituted or substituted by one or more radicals from the group of halogen, hydroxy, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
   R_{H}³ is (C₁-C₄)-alkoxy, (C₂-C₄)-alkenyloxy, (C₂-C₆)-alkynyloxy or (C₂-C₄)-haloalkoxy and
   R_{H}⁴ is hydrogen or (C₁-C₄)-alkyl or
   R_{H}³ and R_{H}⁴ together with the directly bonded nitrogen atom are a four- to eight-membered heterocyclic ring which, in addition to the nitrogen atom, may also contain further ring heteroatoms, preferably up to two further ring heteroatoms from the group consisting of N, O and S and which is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
   S16) Active compounds which are used primarily as herbicides but also have safener action on crop plants, for example (2,4-dichlorophenoxy)acetic acid (2,4-D), (4-chlorophenoxy)acetic acid, (R,S)-2-(4-chloro-o-tolyloxy)propionic acid (mecoprop), 4-(2,4-dichlorophenoxy)butyric acid (2,4-DB), (4-chloro-o-tolyloxy)acetic acid (MCPA), 4-(4-chloro-o-tolyloxy)butyric acid, 4-(4-chlorophenoxy)butyric acid, 3,6-dichloro-2-methoxybenzoic acid (dicamba), 1-(ethoxycarbonyl)ethyl 3,6-dichloro-2-methoxybenzoate (lactidichlor-ethyl).
   Substances which Influence Plant Maturity:

Combination partners usable for the compounds of the general formula (I) in mixture formulations or in a tankmix are, for example, known active compounds based on inhibition of, for example, 1-aminocyclopropane-1-carboxylate synthase, 1-aminocyclopropane-1-carboxylate oxidase and the ethylene receptors, for example ETR1, ETR2, ERS1, ERS2 or EIN4, as described, for example, in Biotechn. Adv. 2006, 24, 357-367; Bot. Bull. Acad. Sin. 199, 40, 1-7 or Plant Growth Reg. 1993, 13, 41-46 and literature cited therein.

Examples of known substances which influence plant maturity and can be combined with the compounds of the general formula (I) include the active compounds which follow (the compounds are designated either by the “common name” according to the International Organization for Standardization (ISO) or by the chemical name or by the code number) and always encompass all use forms, such as acids, salts, esters and isomers, such as stereoisomers and optical isomers. These include, by way of example, one use form and in some cases also a plurality of use forms:

rhizobitoxine, 2-aminoethoxyvinylglycine (AVG), methoxyvinylglycine (MVG), vinylglycine, aminooxyacetic acid, sinefungin, S-adenosylhomocysteine, 2-keto-4-methyl thiobutyrate, 2-(methoxy)-2-oxoethyl(isopropylidene)aminooxyacetate, 2-(hexyloxy)-2-oxoethyl(isopropylidene)aminooxyacetate, 2-(isopropyloxy)-2-oxoethyl(cyclohexylidene)aminooxyacetate, putrescine, spermidine, spermine, 1,8-diamino-4-aminoethyloctane, L-canaline, daminozide, methyl 1-aminocyclopropyl-1-carboxylate, N-methyl-1-aminocyclopropyl-1-carboxylic acid, 1-aminocyclopropyl-1-carboxamide, substituted 1-aminocyclopropyl-1-carboxylic acid derivatives as described in DE3335514, EP30287, DE2906507 or U.S. Pat. No. 5,123,951, 1-aminocyclopropyl-1-hydroxamic acid, 1-methylcyclopropene, 3-methylcyclopropene, 1-ethylcyclopropene, 1-n-propylcyclopropene, 1-cyclopropenylmethanol, carvone, eugenol, sodium cycloprop-1-en-1-ylacetate, sodium cycloprop-2-en-1-ylacetate, sodium 3-(cycloprop-2-en-1-yl)propanoate, sodium 3-(cycloprop-1-en-1-yl)propanoate, jasmonic acid, methyl jasmonate, ethyl jasmonate.
Substances which Influence Plant Health and Germination:

Examples of combination partners usable for the compounds of the general formula (I) in mixture formulations or in a tankmix include known active compounds which influence plant health (the compounds are designated either by the “common name” according to the International Organization for Standardization (ISO) or by the chemical name or by the code number and always encompass all use forms, such as acids, salts, esters and isomers, such as stereoisomers and optical isomers): sarcosine, phenylalanine, tryptophan, N′-methyl-1-phenyl-1-N,N-diethylaminomethanesulfonamide, apio-galacturonans as described in WO2010017956, 4-oxo-4-[(2-phenylethyl)amino]butanoic acid, 4-{[2-(1H-indol-3-yl)ethyl]amino}-4-oxobutanoic acid, 4-[(3-methylpyridin-2-yl)amino]-4-oxobutanoic acid, allantoin, 5-aminolevulic acid, (2S,3R)-2-(3,4-dihydroxyphenyl)-3,4-dihydro-2H-chromene-3,5,7-triol and structurally related catechols as described in WO2010122956, 2-hydroxy-4-(methylsulfanyl)butanoic acid, (3E,3αR,8βS)-3-({[(2R)-4-methyl-5-oxo-2,5-dihydrofuran-2-yl]oxy}methylene)-3,3α,4,8β-tetrahydro-2H-indeno[1,2-b]furan-2-one and analogous lactones as described in EP2248421, abscisic acid, (2Z,4E)-5-[6-ethynyl-1-hydroxy-2,6-dimethyl-4-oxocyclohex-2-en-1-yl]-3-methylpenta-2,4-dienoic acid, methyl (2Z,4E)-5-[6-ethynyl-1-hydroxy-2,6-dimethyl-4-oxocyclohex-2-en-1-yl]-3-methylpenta-2,4-dienoate, 4-phenylbutyric acid, sodium 4-phenylbutanoate, potassium 4-phenylbutanoate.

### Herbicides or Plant Growth Regulators:

Combination partners usable for the compounds of the general formula (I) in mixture formulations or in a tankmix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 14th edition, The British Crop Protection Council and the Royal Soc. of Chemistry, 2006 and literature cited therein.

Examples of known herbicides or plant growth regulators which can be combined with compounds of the general formula (I) include the active compounds which follow (the compounds are designated either by the “common name” according to the International Organization for Standardization (ISO) or by the chemical name or by the code number) and always encompass all use forms, such as acids, salts, esters and isomers, such as stereoisomers and optical isomers. These include, by way of example, one use form and in some cases also a plurality of use forms:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlornitrofen, chlorophthalim, chlorthal-dimethyl, chlortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. O-(2,4-dimethyl-6-nitrophenyl)O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl(2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron esters, monuron, MT-128, i.e. 6-chloro-N-[(2E)-3-chloroprop-2-en-1-yl]-5-methyl-N-phenylpyridazine-3-amine, MT-5950, i.e. N-[3-chloro-4-(1-methylethyl)phenyl]-2-methylpentanamide, NGGC-011, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), sulfentrazone, sulfometuron, sulfometuron-methyl, sulfosate (glyphosate-trimesium), sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryn, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, thiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

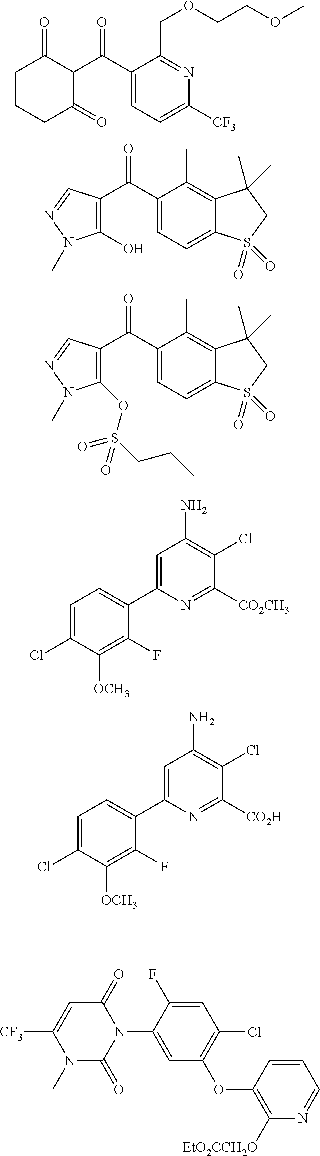

The invention is to be illustrated by the biological examples which follow, but without restricting it thereto.

### Biological Examples

Seeds of monocotyledonous and dicotyledonous crop plants were laid out in sandy loam in wood-fiber pots, covered with soil or sand and cultivated in a greenhouse under good growth conditions. The test plants were treated at the early leaf stage (BBCH10-BBCH13). To assure uniform water supply before commencement of stress, the potted plants were supplied with water by dam irrigation prior to substance application. The compounds according to the invention, formulated in the form of wettable powders (WP), were sprayed onto the green parts of the plants as an aqueous suspension at an equivalent water application rate of 600 l/ha with addition of 0.2% wetting agent (e.g. agrotin). Substance application was followed immediately by stress treatment of the plants. For this purpose, the pots were transferred into plastic inserts in order to prevent them from subsequently drying out too quickly. Drought stress was induced by gradual drying out under the following conditions:

“Day”: 14 hours with illumination at 26° C.
“Night”: 10 hours without illumination at 18° C.

The duration of the respective stress phases was guided mainly by the state of the untreated, stressed control plants and thus varied from crop to crop. It was ended (by re-irrigating and transfer to a greenhouse with good growth conditions) as soon as irreversible damage could be observed on the untreated, stressed control plants. In the case of dicotyledonous crops, for example oilseed rape and soya, the duration of the drought stress phase varied between 3 and 6 days, in the case of monocotyledonous crops, for example wheat, barley or corn, between 6 and 11 days.

The end of the stress phase was followed by an approx. 5-7-day recovery phase, during which the plants were once again kept under good growth conditions in a greenhouse.

In order to rule out any influence of the effects observed by any fungicidal or insecticidal action of the test compounds, it was additionally ensured that the tests proceeded without fungal infection or insect infestation.

After the recovery phase had ended, the intensities of damage were analyzed in visual comparison to untreated, unstressed controls for the same age. The intensity of damage was first recorded as a percentage (100%=plants have died, 0%=like control plants). These values were then used to calculate the efficacy of the test compounds (=percentage reduction in the intensity of damage as a result of substance application) by the following formula:

$$E = \frac{( {DV}_{us} - {DV}_{ts} ) \times 100}{{DV}_{us}}$$

E: efficacy (%)
DVus: damage value of the untreated, stressed control
DVts: damage value of the plants treated with test compound

In each trial, 3 pots per crop and dosage were treated and evaluated; the resulting efficacies are thus averages. The values given in tables A-1 to A-2 below are again averages from one to three independent trials.

### Effects of Selected Compounds of the General Formula (I) Under Drought Stress:

TABLE A-1

| | | | | E |
|---|---|---|---|---|
| No. | Substance | Dosage | Unit | (HORVS) |
| 1 | B1-344 | 250 | g/ha | >5 |
| 2 | B1-456 | 100 | g/ha | >5 |

TABLE A-2

| | | | | E |
|---|---|---|---|---|
| No. | Substance | Dosage | Unit | (BRSNS) |
| 1 | B1-11 | 25 | g/ha | >5 |
| 2 | B1-44 | 25 | g/ha | >5 |
| 3 | B1-51 | 250 | g/ha | >5 |
| 4 | B1-65 | 25 | g/ha | >5 |
| 5 | B1-67 | 25 | g/ha | >5 |
| 6 | B1-296 | 25 | g/ha | >5 |
| 7 | B1-344 | 25 | g/ha | >5 |
| 8 | B1-446 | 25 | g/ha | >5 |
| 9 | B1-456 | 25 | g/ha | >5 |
| 10 | B1-501 | 25 | g/ha | >5 |
| 11 | E10-4 | 25 | g/ha | >5 |
| 12 | E22-4 | 2.5 | g/ha | >5 |
| 13 | E26-4 | 2.5 | g/ha | >5 |
| 14 | E35-4 | 2.5 | g/ha | >5 |

TABLE A-3

| | | | | E |
|---|---|---|---|---|
| No. | Substance | Dosage | Unit | (ZEAMX) |
| 1 | B1-11 | 25 | g/ha | >5 |
| 2 | B1-51 | 25 | g/ha | >5 |
| 3 | B1-65 | 25 | g/ha | >5 |
| 4 | B1-67 | 25 | g/ha | >5 |
| 5 | B1-296 | 25 | g/ha | >5 |
| 6 | B1-344 | 25 | g/ha | >5 |
| 7 | B1-446 | 25 | g/ha | >5 |
| 8 | B1-501 | 25 | g/ha | >5 |

TABLE A-4

| | | | | E |
|---|---|---|---|---|
| No. | Substance | Dosage | Unit | (TRZAS) |
| 1 | B1-296 | 25 | g/ha | >5 |
| 2 | B1-344 | 25 | g/ha | >5 |
| 3 | E10-4 | 25 | g/ha | >5 |

In the above tables:

BRSNS=Brassica napus
HORVS=Hordeum vulgare
TRZAS=Triticum aestivum
ZEAMX=Zea mays

Similar results were also achieved with further compounds of the general formula (I), also in the case of application to different plant species.

1. A substituted benzodiazepinone and/or benzazepinone of formula (I) and/or a salt thereof 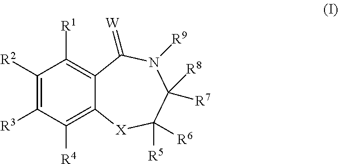
Capable of being used for increasing tolerance to abiotic stress in plants, where
R¹, R², R³ independently of one another represent hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, heteroarylalkoxy, arylalkoxy, heterocyclylalkoxy, cycloalkylalkyl, haloalkenyl, haloalkynyl, heterocyclylalkynyl, halocycloalkoxy, haloalkynyloxy, arylthio, heteroarylthio, alkylsulfinyl, haloalkylsulfinyl, arylsulfinyl, heteroarylsulfinyl, alkylsulfonyl, haloalkylsulfonyl, arylsulfonyl, heteroarylsulfonyl, thiocyanato, isothiocyanato, cycloalkylamino, cycloalkyl(alkyl)amino, alkenylamino, halocycloalkylalkynyl,
R⁴ represents hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, cycloalkylamino, arylamino, alkylcarbonylamino, cycloalkylcarbonylamino, arylcarbonylamino, heteroarylcarbonylamino, alkoxycarbonylamino, alkylcarbonyl(alkyl)amino, arylcarbonyl(alkyl)amino, cycloalkylcarbonyl(alkyl)amino, alkyl(alkyl)amino, haloalkyl(alkyl)amino, alkoxycarbonyl(alkyl)amino, alkoxycarbonylalkyl(alkyl)amino, alkylsulfonylamino, arylsulfonylamino, heteroarylsulfonylamino, cycloalkylsulfonylamino, alkylsulfonyl(alkyl)amino, cycloalkylsulfonyl(alkyl)amino, arylsulfonyl(alkyl)amino, heteroarylsulfonyl(alkyl)amino, alkylcarbonyl(alkyl)amino, arylcarbonyl(alkyl)amino, cycloalkylcarbonyl(alkyl)amino, heteroarylcarbonyl(alkyl)amino, alkenylamino, imino, arylimino, arylalkylimino, heteroarylimino, heterocyclylimino, haloalkylimino, alkylimino, cycloalkylimino, alkoxycarbonylimino, cycloalkoxycarbonylimino, cycloalkylalkoxycarbonylimino, arylalkoxycarbonylimino, alkylaminocarbonylimino, bisalkylaminocarbonylimino, alkylsulfinylimino, arylsulfinylimino, cycloalkylsulfinylimino, alkylthioimino, arylthioimino, cycloalkylthioimino, alkylsulfonylimino, arylsulfonylimino, cycloalkylsulfonylimino, optionally further substituted iminoalkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, alkyl, haloalkyl, cycloalkyl, aryl, alkenyl, alkynyl, heterocyclyl, heteroaryl, alkoxyalkyl or together form an exo methylene group which is optionally substituted further and part of a further ring,
R⁷, R⁸ independently of one another represent hydrogen, alkyl, haloalkyl, cycloalkyl, aryl, alkenyl, alkynyl, heterocyclyl, heteroaryl, alkoxyalkyl,
R⁹ represents hydrogen, hydroxy, alkyl, cycloalkyl, halogen, alkenylalkyl, alkynylalkyl, haloalkyl, alkoxyalkyl, alkynyl, alkenyl, cycloalkylalkyl, cyanoalkyl, nitroalkyl, arylalkyl, heteroarylalkyl, heterocyclylalkyl, aryl, alkylamino, alkylaminoalkyl, bisalkylaminoalkyl, aminocarbonylalkyl, alkylaminocarbonylalkyl, bisalkylaminocarbonylalkyl, alkoxycarbonylalkyl, hydroxycarbonylalkyl, alkylcarbonyl, cycloalkylcarbonyl, haloalkylcarbonyl, alkoxycarbonyl, alkenyloxycarbonyl, alkylaminocarbonyl, cyclopropylaminocarbonyl, alkylsulfonyl, arylsulfonyl, cycloalkylsulfonyl, arylalkylsulfonyl, alkenylsulfonyl, heteroarylsulfonyl, alkynylsulfonyl, alkylsulfinyl, arylsulfinyl, cycloalkylsulfinyl, alkenylsulfinyl, alkynylsulfinyl, arylsulfinyl, heteroarylsulfinyl, arylcarbonyl, heteroarylcarbonyl, alkoxycarbonylalkyl, alkenyloxycarbonylalkyl, hydroxycarbonylalkyl, cyanoalkylaminocarbonyl, alkynylaminocarbonyl, heterocyclylcarbonyl, heteroarylalkylaminocarbonyl, alkenyloxycarbonyl, cycloalkylalkoxycarbonyl, cycloalkylalkoxycarbonylcarbonyl, alkoxycarbonylcarbonyl, cycloalkylalkylaminocarbonyl, arylalkylaminocarbonyl or a negative charge,
R¹⁰ represents alkyl, optionally substituted alkenyl, optionally substituted iminoalkyl, optionally further substituted imino, optionally substituted alkylalkenyl, heteroarylalkenyl, arylalkyl, heteroarylalkyl, heterocyclylalkyl, alkoxycarbonylalkyl, cycloalkoxycarbonylalkyl, arylalkoxycarbonylalkyl, haloalkyl, aminoalkyl, bis-alkylaminoalkyl, alkoxyalkyl, alkylcarbonyl, arylcarbonyl, heteroarylcarbonyl, cycloalkylcarbonyl, alkoxycarbonyl, aryloxycarbonyl, cycloalkoxycarbonyl, alkylsulfonyl, cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford formula (Ia), 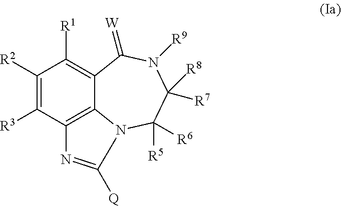
R⁴ and CR¹¹R¹² together with the atoms to which they are attached form a pyrrole ring which is substituted further by R¹³ and Q and thus together with the other substituents according to the definition afford formula (Ib), 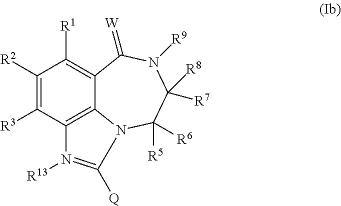
R⁵, R⁶ and N—R¹⁰ together with the atoms to which they are attached form a pyrrole ring which is optionally substituted further and thus together with the other substituents according to the definition afford formula (Ic), 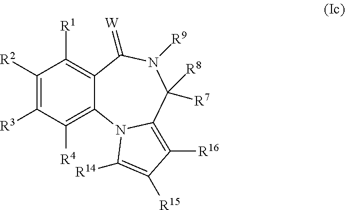
R⁴ and N—R¹⁰ together with the atoms to which they are attached form a pyrrole ring which is substituted further by R¹⁷ and Q and thus together with the other substituents according to the definition afford formula (Id), 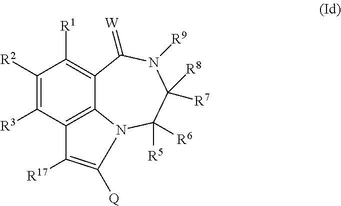
Q represents alkyl, haloalkyl, halocycloalkyl, alkoxyhaloalkyl, haloalkoxyhaloalkyl, alkylthiohaloalkyl, haloalkylthiohaloalkyl, alkoxyalkoxyhaloalkyl, bisalkylaminoalkoxyhaloalkyl, alkenyl, cycloalkyl, cycloalkylalkyl, cycloalkenyl, aryl, arylalkyl, heteroarylalkyl, heterocyclylalkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, arylalkenyl, heteroarylalkenyl, heterocyclylalkenyl, arylalkynyl, heteroarylalkynyl, heterocyclylalkynyl, cycloalkylalkynyl, alkylaminoalkyl, bis-alkylaminoalkyl, hydroxyalkyl, alkoxyalkyl, haloalkoxyalkyl, tris-[alkyl]silyloxyalkyl, bis-[alkyl]arylsilyloxyalkyl, bis-[alkyl]alkylsilyloxyalkyl, bis-alkylaminoalkoxyalkyl, alkoxyalkoxyalkyl, aryloxyalkyl, heteroaryloxyalkyl, alkylthioalkyl, haloalkylthioalkyl, arylthioalkyl, heteroarylthioalkyl, alkoxycarbonyl-N-heterocyclyl, arylalkoxycarbonyl-N-heterocyclyl, alkyl-N-heterocyclyl, alkylsulfonyl-N-heterocyclyl, arylsulfonyl-N-heterocyclyl, heteroarylsulfonyl-N-heterocyclyl, cycloalkylsulfonyl-N-heterocyclyl, haloalkylsulfonyl-N-heterocyclyl, alkylcarbonyl-N-heterocyclyl, arylcarbonyl-N-heterocyclyl, heteroarylcarbonyl-N-heterocyclyl, cycloalkylcarbonyl-N-heterocyclyl, cycloalkyl-N-heterocyclyl, aryl-N-heterocyclyl, arylalkyl-N-heterocyclyl, bis-alkylaminoalkyl-N-heterocyclyl, bis-alkylaminosulfonyl-N-heterocyclyl, heteroaryloxyaryl, heteroaryloxyheteroaryl, aryloxyheteroaryl, alkylsulfinyl, alkylthio, alkylsulfonyl, cycloalkylsulfinyl, cycloalkylthio, cycloalkylsulfonyl, arylsulfinyl, arylthio, arylsulfonyl, amino, alkylamino, bis-alkylamino, arylamino, arylalkylamino, cycloalkylamino, formyl, alkylcarbonyl, arylcarbonyl, iminoalkyl, alkyliminoalkyl, aryliminoalkyl, alkoxycarbonyl, cycloalkoxycarbonyl, cycloalkylalkoxycarbonyl, arylalkoxycarbonyl, arylalkylaminocarbonyl, aminocarbonyl, alkylaminocarbonyl, cycloalkylaminocarbonyl, bis-alkylaminocarbonyl, heterocyclyl-N-carbonyl, imino, alkylimino, arylimino, cycloalkylimino, cycloalkylalkylimino, hydroxyimino, alkoxyimino, cycloalkoxyimino, cycloalkylalkoxyimino, aryloxyimino, arylalkoxyimino, heteroarylalkoxyimino, heteroarylimino, heterocyclylimino, heterocyclylalkylimino, aminoimino, alkylaminoimino, arylaminoimino, heteroarylaminoimino, cycloalkylaminoimino, bis-alkylaminoimino, arylalkylaminoimino, aryl(alkyl)aminoimino, cycloalkyl(alkyl)aminoimino, cycloalkylalkylaminoimino, heterocyclylaminoimino, heteroarylalkoxyalkyl, arylalkoxyalkyl, heterocyclyl-N-alkyl, aryl(alkyl)aminoalkyl, arylalkyl(alkyl)aminoalkyl, alkoxycarbonylalkylaminoalkyl, alkoxycarbonylalkyl(alkyl)aminoalkyl, heteroaryl(alkyl)aminoalkyl, heteroarylalkyl(alkyl)aminoalkyl, cycloalkyl(alkyl)aminoalkyl, cycloalkylaminoalkyl, alkoxy(alkoxy)alkyl heteroarylalkoxyalkyl, arylalkoxyalkylaryl, heterocyclyl-N-alkylaryl, aryl(alkyl)aminoalkylaryl, arylalkyl(alkyl)aminoalkylaryl, alkoxycarbonylalkylaminoalkylaryl, alkoxycarbonylalkyl(alkyl)aminoalkylaryl, heteroaryl(alkyl)aminoalkylaryl, heteroarylalkyl(alkyl)aminoalkylaryl, cycloalkyl(alkyl)aminoalkylaryl, cycloalkylaminoalkylaryl, alkoxy(alkoxy)alkylaryl, alkynyl, alkylalkynyl, haloalkylalkynyl, alkylaminoalkylaryl, alkylaminoalkylarylalkyl, bis-alkylaminoalkylaryl, bis-alkylaminoalkylarylalkyl, heterocyclyl-N-alkylarylalkyl, alkoxycarbonyl-N-heterocyclyl-N-alkyl, arylalkoxycarbonyl-N-heterocyclyl-N-alkyl, alkyl-N-heterocyclyl-N-alkyl, alkylsulfonyl-N-heterocyclyl-N-alkyl, arylsulfonyl-N-heterocyclyl-N-alkyl, heteroarylsulfonyl-N-heterocyclyl-N-alkyl, cycloalkylsulfonyl-N-heterocyclyl-N-alkyl, haloalkylsulfonyl-N-heterocyclyl-N-alkyl, alkylcarbonyl-N-heterocyclyl-N-alkyl, arylcarbonyl-N-heterocyclyl-N-alkyl, heteroarylcarbonyl-N-heterocyclyl-N-alkyl, cycloalkylcarbonyl-N-heterocyclyl-N-alkyl, cycloalkyl-N-heterocyclyl-N-alkyl, alkoxycarbonyl-N-heterocyclylalkyl, arylalkoxycarbonyl-N-heterocyclylalkyl, alkyl-N-heterocyclylalkyl, alkylsulfonyl-N-heterocyclylalkyl, arylsulfonyl-N-heterocyclylalkyl, heteroarylsulfonyl-N-heterocyclylalkyl, cycloalkylsulfonyl-N-heterocyclylalkyl, haloalkylsulfonyl-N-heterocyclylalkyl, alkylcarbonyl-N-heterocyclylalkyl, arylcarbonyl-N-heterocyclylalkyl, heteroarylcarbonyl-N-heterocyclylalkyl, cycloalkylcarbonyl-N-heterocyclylalkyl, cycloalkyl-N-heterocyclylalkyl, arylalkylaminocarbonylaryl, aminocarbonylaryl, alkylaminocarbonylaryl, cycloalkylaminocarbonylaryl, bis-alkylaminocarbonylaryl, arylalkylaminocarbonylarylalkyl, aminocarbonylarylalkyl, alkylaminocarbonylarylalkyl, cycloalkylaminocarbonylarylalkyl, bis-alkylaminocarbonylarylalkyl, arylalkylaminoalkyl, heteroarylalkylaminoalkyl, alkylaminoalkoxyaryl, bis-alkylaminoalkoxyaryl,
R¹³ represents hydrogen, alkyl, cycloalkyl, cycloalkylalkyl, cycloalkenyl, cyanoalkyl, alkenylalkyl, haloalkyl, alkynylalkyl, arylalkyl, heteroarylalkyl, heterocyclylalkyl, aryl, alkylcarbonyl, cycloalkylcarbonyl, cycloalkylalkylcarbonyl, arylcarbonyl, arylalkylcarbonyl, heteroarylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, cycloalkylaminocarbonyl, bis-alkylaminocarbonyl, alkoxyalkyl alkoxycarbonylalkyl,
R¹⁴, R¹⁵, R¹⁶ independently of one another represent hydrogen, alkyl, cycloalkyl, cycloalkylalkyl, haloalkyl, aryl, alkoxycarbonyl, hydroxycarbonyl, aminocarbonyl, alkylaminocarbonyl, cycloalkylaminocarbonyl,
R¹⁷ represents hydrogen, alkyl, cycloalkyl, haloalkyl, arylalkyl, heteroarylalkyl, heterocyclylalkyl, aryl, heteroaryl, heterocyclyl, formyl, imino, hydroxyimino, alkoxyimino, cycloalkoxyimino, halogen, alkoxycarbonyl, hydroxyalkyl, alkoxyalkyl, alkylcarbonyloxyalkyl.

2. The compound as claimed in claim 1, where, in formula (I),
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
R⁴ represents hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis-[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₃-C₈)-cycloalkylamino, arylamino, (C₁-C₈)-alkylcarbonylamino, (C₃-C₈)-cycloalkylcarbonylamino, arylcarbonylamino, heteroarylcarbonylamino, (C₁-C₈)-alkoxycarbonylamino, (C₁-C₈)-alkylcarbonyl[(C₁-C₈)-alkyl]amino, arylcarbonyl[(C₁-C₈)-alkyl]amino, (C₃-C₈)-cycloalkylcarbonyl[(C₁-C₈)-alkyl]amino, (C₁-C₈)-alkyl[(C₁-C₈)-alkyl]amino, (C₁-C₈)-haloalkyl-[(C₁-C₈)-alkyl]amino, (C₁-C₈)-alkoxycarbonyl-[(C₁-C₈)-alkyl]amino, (C₁-C₈)-alkoxycarbonyl-(C₁-C₈)-alkyl-[(C₁-C₈)-alkyl]amino, (C₁-C₈)-alkylsulfonylamino, arylsulfonylamino, heteroarylsulfonylamino, (C₃-C₈)-cycloalkylsulfonylamino, (C₁-C₈)-alkylsulfonyl-[(C₁-C₈)-alkyl]amino, (C₃-C₈)-cycloalkylsulfonyl-[(C₁-C₈)-alkyl]amino, arylsulfonyl-[(C₁-C₈)-alkyl]amino, heteroarylsulfonyl[(C₁-C₈)-alkyl]amino, (C₁-C₈)-alkylcarbonyl[(C₁-C₈)-alkyl]amino, arylcarbonyl-[(C₁-C₈)-alkyl]amino, (C₃-C₈)-cycloalkylcarbonyl-[(C₁-C₈)-alkyl]amino, heteroarylcarbonyl-[(C₁-C₈)-alkyl]amino, (C₂-C₈)-alkenylamino, imino, arylimino, aryl-(C₁-C₈)-alkylimino, heteroarylimino, heterocyclylimino, (C₁-C₈)-haloalkylimino, (C₁-C₈)-alkylimino, (C₃-C₈)-cycloalkylimino, (C₁-C₈)-alkoxycarbonylimino, (C₃-C₈)-cycloalkoxycarbonylimino, (C₃-C₈)-cycloalkyl-(C₁-C₈)-alkoxycarbonylimino, aryl-(C₁-C₈)-alkoxycarbonylimino, (C₁-C₈)-alkylaminocarbonylimino, bis-[(C₁-C₈)-alkyl]aminocarbonylimino, (C₁-C₈)-alkylsulfinylimino, arylsulfinylimino, (C₃-C₈)-cycloalkylsulfinylimino, (C₁-C₈)-alkylthioimino, arylthioimino, (C₃-C₈)-cycloalkylthioimino, (C₁-C₈)-alkylsulfonylimino, arylsulfonylimino, (C₃-C₈)-cycloalkylsulfonylimino, optionally further substituted imino-(C₁-C₈)-alkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
R¹⁰ represents (C₁-C₈)-alkyl, optionally substituted (C₂-C₈)-alkenyl, optionally substituted imino-(C₁-C₈)-alkyl, optionally further substituted imino, optionally substituted (C₁-C₈)-alkyl-(C₂-C₈)-alkenyl, heteroaryl-(C₂-C₈)-alkenyl, aryl-(C₁-C₈)-alkyl, heteroaryl-(C₁-C₈)-alkyl, heterocyclyl-(C₁-C₈)-alkyl, (C₁-C₈)-alkoxycarbonyl-(C₁-C₈)-alkyl, (C₃-C₈)-cycloalkoxycarbonyl-(C₁-C₈)-alkyl, aryl-(C₁-C₈)-alkoxycarbonyl-(C₁-C₈)-alkyl, (C₁-C₈)-haloalkyl, amino-(C₁-C₈)-alkyl, bis-[(C₁-C₈)-alkyl]amino-(C₁-C₈)-alkyl, (C₁-C₈)-alkoxy-(C₁-C₈)-alkyl, (C₁-C₈)-alkylcarbonyl, arylcarbonyl, heteroarylcarbonyl, (C₃-C₈)-cycloalkylcarbonyl, (C₁-C₈)-alkoxycarbonyl, aryloxycarbonyl, (C₃-C₈)-cycloalkoxycarbonyl, (C₁-C₈)-alkylsulfonyl, (C₃-C₈)-cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford formula (Ia), 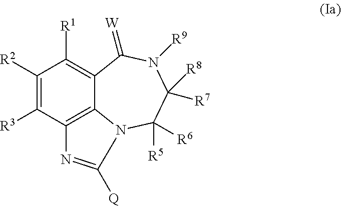
R⁴ and CR¹¹R¹² together with the atoms to which they are attached form a pyrrole ring which is substituted further by R¹³ and Q and thus together with the other substituents according to the definition afford formula (Ib), 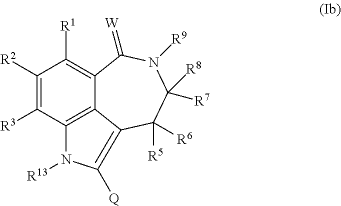
R⁵, R⁶ and N—R¹⁰ together with the atoms to which they are attached form a pyrrole ring which is optionally substituted further and thus together with the other substituents according to the definition afford formula (Ic), 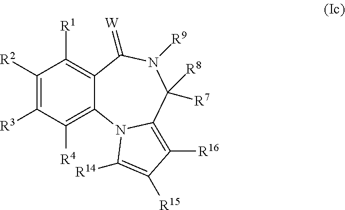
R⁴ and N—R¹⁰ together with the atoms to which they are attached form a pyrrole ring which is substituted further by R¹⁷ and Q and thus together with the other substituents according to the definition afford formula (Id), 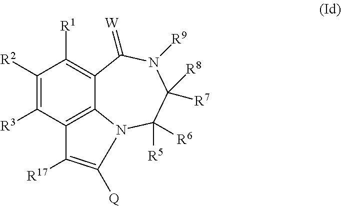
Q represents (C₁-C₈)-alkyl, (C₁-C₈)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl, (C₂-C₈)-alkenyl, (C₃-C₈)-cycloalkyl, (C₃-C₈)-cycloalkyl-(C₁-C₈)-alkyl, (C₄-C₈)-cycloalkenyl, aryl, aryl-(C₁-C₈)-alkyl, heteroaryl-(C₁-C₈)-alkyl, heterocyclyl-(C₁-C₈)-alkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₂-C₈)-alkynyl, (C₃-C₈)-cycloalkyl-(C₂-C₈)-alkynyl, (C₁-C₈)-alkylamino-(C₁-C₈)-alkyl, bis-[(C₁-C₈)-alkyl]amino-(C₁-C₈)-alkyl, hydroxy-(C₁-C₈)-alkyl, (C₁-C₈)-alkoxy-(C₁-C₈)-alkyl, (C₁-C₈)-haloalkoxy-(C₁-C₈)-alkyl, tris-[(C₁-C₈)-alkyl]silyloxy-(C₁-C₈)-alkyl, bis-[(C₁-C₈)-alkyl]arylsilyloxy-(C₁-C₈)-alkyl, bis-[(C₁-C₈)-alkyl]-(C₁-C₈)-alkylsilyloxy-(C₁-C₈)-alkyl, bis-[(C₁-C₈)-alkyl]amino-(C₁-C₈)-alkoxy-(C₁-C₈)-alkyl, (C₁-C₈)-alkoxy-(C₁-C₈)-alkoxy-(C₁-C₈)-alkyl, aryloxy-(C₁-C₈)-alkyl, heteroaryloxy-(C₁-C₈)-alkyl, (C₁-C₈)-alkylthio-(C₁-C₈)-alkyl, (C₁-C₈)-haloalkylthio-(C₁-C₈)-alkyl, arylthio-(C₁-C₈)-alkyl, heteroarylthio-(C₁-C₈)-alkyl, (C₁-C₈)-alkoxycarbonyl-N-heterocyclyl, aryl-(C₁-C₈)-alkoxycarbonyl-N-heterocyclyl, (C₁-C₈)-alkyl-N-heterocyclyl, (C₁-C₈)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₈)-haloalkylsulfonyl-N-heterocyclyl, (C₁-C₈)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₃-C₈)-cycloalkyl-N-heterocyclyl, aryl-N-heterocyclyl, aryl-(C₁-C₈)-alkyl-N-heterocyclyl, bis-[(C₁-C₈)-alkyl]amino-(C₁-C₈)-alkyl-N-heterocyclyl, bis-[(C₁-C₈)-alkyl]aminosulfonyl-N-heterocyclyl, heteroaryloxyaryl, heteroaryloxyheteroaryl, aryloxyheteroaryl, (C₁-C₈)-alkylsulfinyl, (C₁-C₈)-alkylthio, (C₁-C₈)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₈)-alkylamino, bis-[(C₁-C₈)-alkyl]amino, arylamino, aryl-(C₁-C₈)-alkylamino, (C₃-C₈)-cycloalkylamino, formyl, (C₁-C₈)-alkylcarbonyl, arylcarbonyl, Imino-(C₁-C₈)-alkyl, (C₁-C₈)-alkylimino-(C₁-C₈)-alkyl, arylimino-(C₁-C₈)-alkyl, (C₁-C₈)-alkoxycarbonyl, (C₃-C₈)-cycloalkoxycarbonyl, (C₃-C₈)-cycloalkyl-(C₁-C₈)-alkoxycarbonyl, aryl-(C₁-C₈)-alkoxycarbonyl, aryl-(C₁-C₈)-alkylaminocarbonyl, aminocarbonyl, (C₁-C₈)-alkylaminocarbonyl, (C₃-C₈)-cycloalkylaminocarbonyl, bis-[(C₁-C₈)-alkyl]aminocarbonyl heterocyclyl-N-carbonyl, Imino, (C₁-C₈)-alkylimino, arylimino, (C₃-C₈)-cycloalkylimino, (C₃-C₈)-cycloalkyl-(C₁-C₈)-alkylimino, hydroxyimino, (C₁-C₈)-alkoxyimino, (C₃-C₈)-cycloalkoxyimino, (C₃-C₈)-cyloalkyl-(C₁-C₈)-alkoxyimino, aryloxyimino, aryl-(C₁-C₈)-alkoxyimino, heteroaryl-(C₁-C₈)-alkoxyimino, heteroarylimino, heterocyclylimino, heterocyclyl-(C₁-C₈)-alkylimino, aminoimino, (C₁-C₈)-alkylaminoimino, arylaminoimino, heteroarylaminoimino, (C₃-C₈)-cycloalkylaminoimino, bis-[(C₁-C₈)-alkyl]aminoimino, aryl-(C₁-C₈)-alkylaminoimino, aryl[(C₁-C₈)-alkyl]aminoimino, (C₃-C₈)-cycloalkyl[(C₁-C₈)-alkyl]aminoimino, (C₃-C₈)-cycloalkyl-(C₁-C₈)-alkylaminoimino, heterocyclylaminoimino, heteroaryl-(C₁-C₈)-alkoxy-(C₁-C₈)-alkyl, aryl-(C₁-C₈)-alkoxy-(C₁-C₈)-alkyl, heterocyclyl-N—(C₁-C₈)-alkyl, aryl[(C₁-C₈)-alkyl]amino-(C₁-C₈)-alkyl, aryl-(C₁-C₈)-alkyl[(C₁-C₈)-alkyl]amino-(C₁-C₈)-alkyl, (C₁-C₈)-alkoxycarbonyl-(C₁-C₈)-alkylamino-(C₁-C₈)-alkyl, (C₁-C₈)-alkoxycarbonyl-(C₁-C₈)-alkyl[(C₁-C₈)-alkyl]amino-(C₁-C₈)-alkyl, heteroaryl[(C₁-C₈)-alkyl]amino-(C₁-C₈)-alkyl, heteroaryl-(C₁-C₈)-alkyl[(C₁-C₈)-alkyl]amino-(C₁-C₈)-alkyl, (C₃-C₈)-cycloalkyl[(C₁-C₈)-alkyl]amino-(C₁-C₈)-alkyl, (C₃-C₈)-cycloalkylamino-(C₁-C₈)-alkyl, (C₁-C₈)-alkoxy[(C₁-C₈)-alkoxy]-(C₁-C₈)-alkyl, heteroaryl-(C₁-C₈)-alkoxy-(C₁-C₈)-alkyl, aryl-(C₁-C₈)-alkoxy-(C₁-C₈)-alkylaryl, heterocyclyl-N—(C₁-C₈)-alkylaryl, aryl[(C₁-C₈)-alkyl]amino-(C₁-C₈)-alkylaryl, aryl-(C₁-C₈)-alkyl[(C₁-C₈)-alkyl]amino-(C₁-C₈)-alkylaryl, (C₁-C₈)-alkoxycarbonyl-(C₁-C₈)-alkylamino-(C₁-C₈)-alkylaryl, (C₁-C₈)-alkoxycarbonyl-(C₁-C₈)-alkyl[(C₁-C₈)-alkyl]amino-(C₁-C₈)-alkylaryl, heteroaryl[(C₁-C₈)-alkyl]amino-(C₁-C₈)-alkylaryl, heteroaryl-(C₁-C₈)-alkyl[(C₁-C₈)-alkyl]amino-(C₁-C₈)-alkylaryl, (C₃-C₈)-cycloalkyl[(C₁-C₈)-alkyl]amino-(C₁-C₈)-alkylaryl, (C₃-C₈)-cycloalkylamino-(C₁-C₈)-alkylaryl, (C₁-C₈)-alkoxy[(C₁-C₈)-alkoxy]-(C₁-C₈)-alkylaryl, (C₂-C₈)-alkynyl, (C₁-C₈)-alkyl-(C₂-C₈)-alkynyl, (C₁-C₈)-haloalkyl-(C₂-C₈)-alkynyl, (C₁-C₈)-alkylamino-(C₁-C₈)-alkylaryl, (C₁-C₈)-alkylamino-(C₁-C₈)-alkylaryl-(C₁-C₈)-alkyl, bis-[(C₁-C₈)-alkyl]amino-(C₁-C₈)-alkylaryl, bis-[(C₁-C₈)-alkyl]amino-(C₁-C₈)-alkylaryl-(C₁-C₈)-alkyl, heterocyclyl-N—(C₁-C₈)-alkylaryl-(C₁-C₈)-alkyl, (C₁-C₈)-alkoxycarbonyl-N-heterocyclyl-N—(C₁-C₈)-alkyl, aryl-(C₁-C₈)-alkoxycarbonyl-N-heterocyclyl-N—(C₁-C₈)-alkyl, (C₁-C₈)-alkyl-N-heterocyclyl-N—(C₁-C₈)-alkyl, (C₁-C₈)-alkylsulfonyl-N-heterocyclyl-N—(C₁-C₈)-alkyl, arylsulfonyl-N-heterocyclyl-N—(C₁-C₈)-alkyl, heteroarylsulfonyl-N-heterocyclyl-N—(C₁-C₈)-alkyl, (C₃-C₈)-cycloalkylsulfonyl-N-heterocyclyl-N—(C₁-C₈)-alkyl, (C₁-C₈)-haloalkylsulfonyl-N-heterocyclyl-N—(C₁-C₈)-alkyl, alkylcarbonyl-N-heterocyclyl-N-alkyl, arylcarbonyl-N-heterocyclyl-N—(C₁-C₈)-alkyl, heteroarylcarbonyl-N-heterocyclyl-N—(C₁-C₈)-alkyl, (C₃-C₈)-cycloalkylcarbonyl-N-heterocyclyl-N—(C₁-C₈)-alkyl, (C₃-C₈)-cycloalkyl-N-heterocyclyl-N—(C₁-C₈)-alkyl, (C₁-C₈)-alkoxycarbonyl-N-heterocyclyl-(C₁-C₈)-alkyl, aryl-(C₁-C₈)-alkoxycarbonyl-N-heterocyclyl-(C₁-C₈)-alkyl, (C₁-C₈)-alkyl-N-heterocyclyl-(C₁-C₈)-alkyl, (C₁-C₈)-alkylsulfonyl-N-heterocyclyl-(C₁-C₈)-alkyl, arylsulfonyl-N-heterocyclyl-(C₁-C₈)-alkyl, heteroarylsulfonyl-N-heterocyclyl-(C₁-C₈)-alkyl, (C₃-C₈)-cycloalkylsulfonyl-N-heterocyclyl-(C₁-C₈)-alkyl, (C₁-C₈)-haloalkylsulfonyl-N-heterocyclyl-(C₁-C₈)-alkyl, (C₁-C₈)-alkylcarbonyl-N-heterocyclyl-(C₁-C₈)-alkyl, arylcarbonyl-N-heterocyclyl-(C₁-C₈)-alkyl, heteroarylcarbonyl-N-heterocyclyl-(C₁-C₈)-alkyl, (C₃-C₈)-cycloalkylcarbonyl-N-heterocyclyl-(C₁-C₈)-alkyl, (C₃-C₈)-cycloalkyl-N-heterocyclyl-(C₁-C₈)-alkyl, aryl-(C₁-C₈)-alkylaminocarbonylaryl, aminocarbonylaryl, (C₁-C₈)-alkylaminocarbonylaryl, (C₃-C₈)-cycloalkylaminocarbonylaryl, bis-[(C₁-C₈)-alkyl]aminocarbonylaryl, aryl-(C₁-C₈)-alkylaminocarbonylaryl-(C₁-C₈)-alkyl, aminocarbonylaryl-(C₁-18 C₈)-alkyl, (C₁-C₈)-alkylaminocarbonylaryl-(C₁-C₈)-alkyl, (C₃-C₈)-cycloalkylaminocarbonylaryl-(C₁-C₈)-alkyl, bis-[(C₁-C₈)-alkyl]aminocarbonylaryl-(C₁-C₈)-alkyl, aryl-(C₁-C₈)-alkylamino-(C₁-C₈)-alkyl, heteroaryl-(C₁-C₈)-alkylamino-(C₁-C₈)-alkyl, (C₁-C₈)-alkylamino-(C₁-C₈)-alkoxyaryl, bis-[(C₁-C₈)-alkyl]amino-(C₁-C₈)-alkoxyaryl,
R¹³ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylcarbonyl, (C₃-C₈)-cycloalkylcarbonyl, (C₃-C₈)-cycloalkyl-(C₁-C₈)-alkylcarbonyl, arylcarbonyl, aryl-(C₁-C₈)-alkylcarbonyl, heteroarylcarbonyl, (C₁-C₈)-alkoxycarbonyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, bis-[(C₁-C₈)-alkyl]aminocarbonyl, (C₁-C₈)-alkoxy-(C₁-C₈)-alkyl, (C₁-C₈)-alkoxycarbonyl-(C₁-C₈)-alkyl,
R¹⁴, R¹⁵, R¹⁶ independently of one another represent hydrogen, (C₁-C₈)-alkyl, (C₃-C₈)-cycloalkyl, (C₃-C₈)-cycloalkyl-(C₁-C₈)-alkyl, (C₁-C₈)-haloalkyl, aryl, (C₁-C₈)-alkoxycarbonyl, (C₁-C₈)-hydroxycarbonyl, aminocarbonyl, (C₁-C₈)-alkylaminocarbonyl, (C₃-C₈)-cycloalkylaminocarbonyl,
R¹⁷ represents hydrogen, (C₁-C₈)-alkyl, (C₃-C₈)-cycloalkyl, (C₁-C₈)-haloalkyl, aryl-(C₁-C₈)-alkyl, heteroaryl-(C₁-C₈)-alkyl, heterocyclyl-(C₁-C₈)-alkyl, aryl, heteroaryl, heterocyclyl, formyl, imino, hydroxyimino, (C₁-C₈)-alkoxyimino, (C₃-C₈)-cycloalkoxyimino, halogen, (C₁-C₈)-alkoxycarbonyl, hydroxy-(C₁-C₈)-alkyl, (C₁-C₈)-alkoxy-(C₁-C₈)-alkyl, (C₁-C₈)-alkylcarbonyloxy-(C₁-C₈)-alkyl.

3. The compound as claimed in claim 1, where, in formula (I),
R¹, R², R³ independently of one another represent hydrogen, halogen, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, aryl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroraryloxy, (C₁-C₇)-alkoxy-(C₁-C₇)-alkoxy, (C₂-C₇)-alkynyl-(C₁-C₇)-alkoxy, (C₂-C₇)-alkenyloxy, bis[(C₁-C₇)-alkyl]amino-(C₁-C₇)-alkoxy, tris-[(C₁-C₇)-alkyl]silyl, bis-[(C₁-C₇)-alkyl]arylsilyl, bis-[(C₁-C₇)-alkyl]-(C₁-C₇)-alkylsilyl, tris-[(C₁-C₇)-alkyl]silylalkynyl, aryl-(C₂-C₇)-alkynyl, heteroaryl-(C₂-C₇)-alkynyl, (C₁-C₇)-alkyl-(C₁-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis-[(C₁-C₇)-alkyl]amino, (C₁-C₇)-alkylcarbonylamino, (C₃-C₇)-cycloalkylcarbonylamino, arylcarbonylamino, (C₁-C₇)-alkoxycarbonylamino, heteroaryl-(C₁-C₇)-alkoxy, aryl-(C₁-C₇)-alkoxy, heterocyclyl-(C₁-C₇)-alkoxy, (C₃-C₇)-cycloalkyl-(C₁-C₇)-alkyl, (C₂-C₇)-haloalkenyl, (C₂-C₇)-haloalkynyl, heterocyclyl-(C₂-C₇)-alkynyl, (C₃-C₇)-halocycloalkoxy, (C₂-C₇)-haloalkynyloxy, arylthio, heteroarylthio, (C₁-C₇)-alkylsulfinyl, (C₁-C₇)-haloalkylsulfinyl, arylsulfinyl, heteroarylsulfinyl, (C₁-C₇)-alkylsulfonyl, (C₁-C₇)-haloalkylsulfonyl, arylsulfonyl, heteroarylsulfonyl, thiocyanato, isothiocyanato, (C₃-C₇)-cycloalkylamino, (C₃-C₇)-cycloalkyl[(C₁-C₇)-alkyl]amino, (C₂-C₇)-alkenylamino, (C₁-C₇)-halocycloalkyl-(C₂-C₇)-alkynyl,
R⁴ represents hydrogen, halogen, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, aryl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroraryloxy, (C₁-C₇)-alkoxy-(C₁-C₇)-alkoxy, (C₂-C₇)-alkynyl-(C₁-C₇)-alkoxy, (C₂-C₇)-alkenyloxy, bis-[(C₁-C₇)-alkyl]amino-(C₁-C₇)-alkoxy, tris-[(C₁-C₇)-alkyl]silyl, bis-[(C₁-C₇)-alkyl]arylsilyl, bis-[(C₁-C₇)-alkyl]-(C₁-C₇)-alkylsilyl, tris-[(C₁-C₇)-alkyl]silyl-(C₂-C₇)-alkynyl, aryl-(C₂-C₇)-alkynyl, heteroaryl-(C₂-C₇)-alkynyl, (C₁-C₇)-alkyl-(C₂-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis-[(C₁-C₇)-alkyl]amino, (C₃-C₇)-cycloalkylamino, arylamino, (C₁-C₇)-alkylcarbonylamino, (C₃-C₇)-cycloalkylcarbonylamino, arylcarbonylamino, heteroarylcarbonylamino, (C₁-C₇)-alkoxycarbonylamino, (C₁-C₇)-alkylcarbonyl[(C₁-C₇)-alkyl]amino, arylcarbonyl[(C₁-C₇)-alkyl]amino, (C₃-C₇)-cycloalkylcarbonyl[(C₁-C₇)-alkyl]amino, (C₁-C₇)-alkyl[(C₁-C₇)-alkyl]amino, (C₁-C₇)-haloalkyl-[(C₁-C₇)-alkyl]amino, (C₁-C₇)-alkoxycarbonyl-[(C₁-C₇)-alkyl]amino, (C₁-C₇)-alkoxycarbonyl-(C₁-C₇)-alkyl-[(C₁-C₇)-alkyl]amino, (C₁-C₇)-alkylsulfonylamino, arylsulfonylamino, heteroarylsulfonylamino, (C₃-C₇)-cycloalkylsulfonylamino, (C₁-C₇)-alkylsulfonyl-[(C₁-C₇)-alkyl]amino, (C₃-C₇)-cycloalkylsulfonyl-[(C₁-C₇)-alkyl]amino, arylsulfonyl-[(C₁-C₇)-alkyl]amino, heteroarylsulfonyl[(C₁-C₇)-alkyl]amino, (C₁-C₇)-alkylcarbonyl[(C₁-C₇)-alkyl]amino, arylcarbonyl-[(C₁-C₇)-alkyl]amino, (C₃-C₇)-cycloalkylcarbonyl-[(C₁-C₇)-alkyl]amino, heteroarylcarbonyl-[(C₁-C₇)-alkyl]amino, (C₂-C₇)-alkenylamino, imino, arylimino, aryl-(C₁-C₇)-alkylimino, heteroarylimino, heterocyclylimino, (C₁-C₇)-haloalkylimino, (C₁-C₇)-alkylimino, (C₃-C₇)-cycloalkylimino, (C₁-C₇)-alkoxycarbonylimino, (C₃-C₇)-cycloalkoxycarbonylimino, (C₃-C₇)-cycloalkyl-(C₁-C₇)-alkoxycarbonylimino, aryl-(C₁-C₇)-alkoxycarbonylimino, (C₁-C₇)-alkylaminocarbonylimino, bis-[(C₁-C₇)-alkyl]aminocarbonylimino, (C₁-C₇)-alkylsulfinylimino, arylsulfinylimino, (C₃-C₇)-cycloalkylsulfinylimino, (C₁-C₇)-alkylthioimino, arylthioimino, (C₃-C₇)-cycloalkylthioimino, (C₁-C₇)-alkylsulfonylimino, arylsulfonylimino, (C₃-C₇)-cycloalkylsulfonylimino, optionally further substituted imino-(C₁-C₇)-alkyl, iminoaryl, iminoheteroaryl, iminoheterocyclyl,
W represents oxygen, sulfur,
X represents a grouping N—R¹⁰, CR¹¹R¹², where the groups R¹⁰, R¹¹ and R¹² each have the meanings according to the definitions below,
R⁵, R⁶ independently of one another represent hydrogen, (C₁-C₇)-alkyl, (C₁-C₇)-haloalkyl, (C₃-C₇)-cycloalkyl, aryl, (C₂-C₇)-alkenyl, (C₂-C₇)-alkynyl, heterocyclyl, heteroaryl, (C₁-C₇)-alkoxy-(C₁-C₇)-alkyl or together form an exo methylene group which is optionally substituted further and part of a further ring,
R⁷, R⁸ independently of one another represent hydrogen, (C₁-C₇)-alkyl, (C₁-C₇)-haloalkyl, (C₃-C₇)-cycloalkyl, aryl, (C₂-C₇)-alkenyl, (C₁-C₇)-alkynyl, heterocyclyl, heteroaryl, (C₁-C₇)-alkoxy-(C₁-C₇)-alkyl,
R⁹ represents hydrogen, hydroxy, (C₁-C₇)-alkyl, (C₃-C₇)-cycloalkyl, halogen, (C₂-C₇)-alkenyl-(C₁-C₇)-alkyl, (C₂-C₇)-alkynyl-(C₁-C₇)-alkyl, (C₁-C₇)-haloalkyl, (C₁-C₇)-alkoxy-(C₁-C₇)-alkyl, (C₂-C₇)-alkynyl, (C₂-C₇)-alkenyl, (C₃-C₇)-cycloalkyl-(C₁-C₇)-alkyl, cyano-(C₁-C₇)-alkyl, nitro-(C₁-C₇)-alkyl, aryl-(C₁-C₇)-alkyl, heteroaryl-(C₁-C₇)-alkyl, heterocyclyl-(C₁-C₇)-alkyl, aryl, (C₁-C₇)-alkylamino, (C₁-C₇)-alkylamino-(C₁-C₇)-alkyl, bis-[(C₁-C₇)-alkyl]amino-(C₁-C₇)-alkyl, aminocarbonyl-(C₁-C₇)-alkyl, (C₁-C₇)-alkylaminocarbonyl-(C₁-C₇)-alkyl, bis-[(C₁-C₇)-alkyl]aminocarbonyl-(C₁-C₇)-alkyl, (C₁-C₇)-alkoxycarbonyl-(C₁-C₇)-alkyl, hydroxycarbonyl-(C₁-C₇)-alkyl, (C₁-C₇)-alkylcarbonyl, (C₃-C₇)-cycloalkylcarbonyl, (C₁-C₇)-haloalkylcarbonyl, (C₁-C₇)-alkoxycarbonyl, (C₂-C₇)-alkenyloxycarbonyl, (C₁-C₇)-alkylaminocarbonyl, (C₃-C₇)-cyclopropylaminocarbonyl, (C₁-C₇)-alkylsulfonyl, arylsulfonyl, (C₁-C₇)-cycloalkylsulfonyl, aryl-(C₁-C₇)-alkylsulfonyl, (C₂-C₇)-alkenylsulfonyl, heteroarylsulfonyl, (C₂-C₇)-alkynylsulfonyl, (C₁-C₇)-alkylsulfinyl, arylsulfinyl, (C₃-C₇)-cycloalkylsulfinyl, (C₂-C₇)-alkenylsulfinyl, (C₂-C₇)-alkynylsulfinyl, arylsulfinyl, heteroarylsulfinyl, arylcarbonyl, heteroarylcarbonyl, (C₁-C₇)-alkoxycarbonyl-(C₁-C₇)-alkyl, (C₂-C₇)-alkenyloxycarbonyl-(C₁-C₇)-alkyl, hydroxycarbonyl-(C₁-C₇)-alkyl, cyano-(C₁-C₇)-alkylaminocarbonyl, (C₂-C₇)-alkynylaminocarbonyl, heterocyclylcarbonyl, heteroaryl-(C₁-C₇)-alkylaminocarbonyl, (C₂-C₇)-alkenyloxycarbonyl, (C₃-C₇)-cycloalkyl-(C₁-C₇)-alkoxycarbonyl, (C₃-C₇)-cycloalkyl-(C₁-C₇)-alkoxycarbonylcarbonyl, (C₁-C₇)-alkoxycarbonylcarbonyl, (C₃-C₇)-cycloalkyl-(C₁-C₇)-alkylaminocarbonyl, aryl-(C₁-C₇)-alkylaminocarbonyl or a negative charge,
R¹⁰ represents (C₁-C₇)-alkyl, optionally substituted (C₂-C₇)-alkenyl, optionally substituted imino-(C₁-C₇)-alkyl, optionally further substituted imino, optionally substituted (C₁-C₇)-alkyl-(C₂-C₇)-alkenyl, heteroaryl-(C₂-C₇)-alkenyl, aryl-(C₁-C₇)-alkyl, heteroaryl-(C₁-C₇)-alkyl, heterocyclyl-(C₁-C₇)-alkyl, (C₁-C₇)-alkoxycarbonyl-(C₁-C₇)-alkyl, (C₃-C₇)-cycloalkoxycarbonyl-(C₁-C₇)-alkyl, aryl-(C₁-C₇)-alkoxycarbonyl-(C₁-C₇)-alkyl, (C₁-C₇)-haloalkyl, amino-(C₁-C₇)-alkyl, bis-[(C₁-C₇)-alkyl]amino-(C₁-C₇)-alkyl, (C₁-C₇)-alkoxy-(C₁-C₇)-alkyl, (C₁-C₇)-alkylcarbonyl, arylcarbonyl, heteroarylcarbonyl, (C₃-C₇)-cycloalkylcarbonyl, (C₁-C₇)-alkoxycarbonyl, aryloxycarbonyl, (C₃-C₇)-cycloalkoxycarbonyl, (C₁-C₇)-alkylsulfonyl, (C₃-C₇)-cycloalkylsulfonyl, arylsulfonyl, heteroarylsulfonyl,
R¹¹, R¹² together form an exo methylene group which is optionally substituted further and part of a further ring,
R⁴ and N—R¹⁰ together with the atoms to which they are attached form an imidazole ring which is substituted further by Q and thus together with the other substituents according to the definition afford formula (Ia), 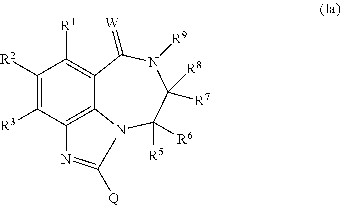
R⁴ and CR¹¹R¹² together with the atoms to which they are attached form a pyrrole ring which is substituted further by R¹³ and Q and thus together with the other substituents according to the definition afford formula (Ib), 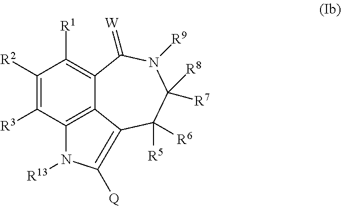
R⁵, R⁶ and N—R¹⁰ together with the atoms to which they are attached form a pyrrole ring which is optionally substituted further and thus together with the other substituents according to the definition afford formula (Ic), 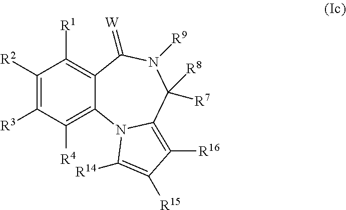
R⁴ and N—R¹⁰ together with the atoms to which they are attached form a pyrrole ring which is substituted further by R¹⁷ and Q and thus together with the other substituents according to the definition afford formula (Id), 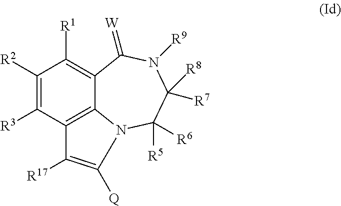
Q represents (C₁-C₇)-alkyl, (C₁-C₇)-haloalkyl, (C₃-C₇)-halocycloalkyl, (C₁-C₇)-alkoxy-(C₁-C₇)-haloalkyl, (C₁-C₇)-haloalkoxy-(C₁-C₇)-haloalkyl, (C₁-C₇)-alkylthio-(C₁-C₇)-haloalkyl, (C₁-C₇)-haloalkylthio-(C₁-C₇)-haloalkyl, (C₁-C₇)-alkoxy-(C₁-C₇)-alkoxy-(C₁-C₇)-haloalkyl, bis-[(C₁-C₇)-alkyl]amino-(C₁-C₇)-alkoxy-(C₁-C₇)-haloalkyl, (C₂-C₇)-alkenyl, (C₃-C₇)-cycloalkyl, (C₃-C₇)-cycloalkyl-(C₁-C₇)-alkyl, (C₄-C₇)-cycloalkenyl, optionally further substituted phenyl, aryl-(C₁-C₇)-alkyl, heteroaryl-(C₁-C₇)-alkyl, heterocyclyl-(C₁-C₇)-alkyl, heteroaryl, heterocyclyl, heterocyclylaryl, heterocyclylheteroaryl, heteroarylheteroaryl, heteroarylaryl, arylaryl, aryloxyaryl, aryloxyheteroaryl, heteroaryloxyaryl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₂-C₇)-alkynyl, (C₃-C₇)-cycloalkyl-(C₂-C₇)-alkynyl, (C₁-Cₐ)-alkylamino-(C₁-C₇)-alkyl, bis-[(C₁-C₇)-alkyl]amino-(C₁-C₇)-alkyl, hydroxy-(C₁-C₇)-alkyl, (C₁-C₇)-alkoxy-(C₁-C₇)-alkyl, (C₁-C₇)-haloalkoxy-(C₁-C₇)-alkyl, tris-[(C₁-C₇)-alkyl]silyloxy-(C₁-C₇)-alkyl, bis-[(C₁-C₇)-alkyl]arylsilyloxy-(C₁-C₇)-alkyl, bis-[(C₁-C₇)-alkyl]-(C₁-C₇)-alkylsilyloxy-(C₁-C₇)-alkyl, bis-[(C₁-C₇)-alkyl]amino-(C₁-C₇)-alkoxy-(C₁-C₇)-alkyl, (C₁-C₇)-alkoxy-(C₁-C₇)-alkoxy-(C₁-C₇)-alkyl, aryloxy-(C₁-C₇)-alkyl, heteroaryloxy-(C₁-C₇)-alkyl, (C₁-C₇)-alkylthio-(C₁-C₇)-alkyl, (C₁-C₇)-haloalkylthio-(C₁-C₇)-alkyl, arylthio-(C₁-C₇)-alkyl, heteroarylthio-(C₁-C₇)-alkyl, (C₁-C₇)-alkoxycarbonyl-N-heterocyclyl, aryl-(C₁-C₇)-alkoxycarbonyl-N-heterocyclyl, (C₁-C₇)-alkyl-N-heterocyclyl, (C₁-C₇)-alkylsulfonyl-N-heterocyclyl, arylsulfonyl-N-heterocyclyl, heteroarylsulfonyl-N-heterocyclyl, (C₃-C₇)-cycloalkylsulfonyl-N-heterocyclyl, (C₁-C₇)-haloalkylsulfonyl-N-heterocyclyl, (C₁-C₇)-alkylcarbonyl-N-heterocyclyl, arylcarbonyl-N-heterocyclyl, heteroarylcarbonyl-N-heterocyclyl, (C₃-C₇)-cycloalkylcarbonyl-N-heterocyclyl, (C₃-C₇)-cycloalkyl-N-heterocyclyl, aryl-N-heterocyclyl, aryl-(C₁-C₇)-alkyl-N-heterocyclyl, bis-[(C₁-C₇)-alkyl]amino-(C₁-C₇)-alkyl-N-heterocyclyl, bis-[(C₁-C₇)-alkyl]aminosulfonyl-N-heterocyclyl, heteroaryloxyaryl, heteroaryloxyheteroaryl, aryloxyheteroaryl, (C₁-C₇)-alkylsulfinyl, (C₁-C₇)-alkylthio, (C₁-C₇)-alkylsulfonyl, (C₃-C₇)-cycloalkylsulfinyl, (C₃-C₇)-cycloalkylthio, (C₃-C₇)-cycloalkylsulfonyl, arylsulfinyl, arylthio, arylsulfonyl, amino, (C₁-C₇)-alkylamino, bis-[(C₁-C₇)-alkyl]amino, arylamino, aryl-(C₁-C₇)-alkylamino, (C₃-C₇)-cycloalkylamino, formyl, (C₁-C₇)-alkylcarbonyl, arylcarbonyl, imino-(C₁-C₇)-alkyl, (C₁-C₇)-alkylimino-(C₁-C₇)-alkyl, arylimino-(C₁-C₇)-alkyl, (C₁-C₇)-alkoxycarbonyl, (C₃-C₇)-cycloalkoxycarbonyl, (C₃-C₇)-cycloalkyl-(C₁-C₇)-alkoxycarbonyl, aryl-(C₁-C₇)-alkoxycarbonyl, aryl-(C₁-C₇)-alkylaminocarbonyl, aminocarbonyl, (C₁-C₇)-alkylaminocarbonyl, (C₃-C₇)-cycloalkylaminocarbonyl, bis-[(C₁-C₇)-alkyl]aminocarbonyl heterocyclyl-N-carbonyl, imino, (C₁-C₇)-alkylimino, arylimino, (C₃-C₇)-cycloalkylimino, (C₃-C₇)-cycloalkyl-(C₁-C₇)-alkylimino, hydroxyimino, (C₁-C₇)-alkoxyimino, (C₃-C₇)-cycloalkoxyimino, (C₃-C₇)-cyloalkyl-(C₁-C₇)-alkoxyimino, aryloxyimino, aryl-(C₁-C₇)-alkoxyimino, heteroaryl-(C₁-C₇)-alkoxyimino, heteroarylimino, heterocyclylimino, heterocyclyl-(C₁-C₇)-alkylimino, aminoimino, (C₁-C₇)-alkylaminoimino, arylaminoimino, heteroarylaminoimino, (C₃-C₇)-cycloalkylaminoimino, bis-[(C₁-C₇)-alkyl]aminoimino, aryl-(C₁-C₇)-alkylaminoimino, aryl[(C₁-C₇)-alkyl]aminoimino, (C₃-C₇)-cycloalkyl[(C₁-C₇)-alkyl]aminoimino, (C₃-C₇)-cycloalkyl-(C₁-C₇)-alkylaminoimino, heterocyclylaminoimino, heteroaryl-(C₁-C₇)-alkoxy-(C₁-C₇)-alkyl, aryl-(C₁-C₇)-alkoxy-(C₁-C₇)-alkyl, heterocyclyl-N—(C₁-C₇)-alkyl, aryl[(C₁-C₇)-alkyl]amino-(C₁-C₇)-alkyl, aryl-(C₁-C₇)-alkyl[(C₁-C₇)-alkyl]amino-(C₁-C₇)-alkyl, (C₁-C₇)-alkoxycarbonyl-(C₁-C₇)-alkylamino-(C₁-C₇)-alkyl, (C₁-C₇)-alkoxycarbonyl-(C₁-C₇)-alkyl[(C₁-C₇)-alkyl]amino-(C₁-C₇)-alkyl, heteroaryl[(C₁-C₇)-alkyl]amino-(C₁-C₇)-alkyl, heteroaryl-(C₁-C₇)-alkyl[(C₁-C₇)-alkyl]amino-(C₁-C₇)-alkyl, (C₃-C₇)-cycloalkyl[(C₁-C₇)-alkyl]amino-(C₁-C₇)-alkyl, (C₃-C₇)-cycloalkylamino-(C₁-C₇)-alkyl, (C₁-C₇)-alkoxy[(C₁-C₇)-alkoxy]-(C₁-C₇)-alkyl, heteroaryl-(C₁-C₇)-alkoxy-(C₁-C₇)-alkyl, aryl-(C₁-C₇)-alkoxy-(C₁-C₇)-alkylaryl, heterocyclyl-N—(C₁-C₇)-alkylaryl, aryl[(C₁-C₇)-alkyl]amino-(C₁-C₇)-alkylaryl, aryl-(C₁-C₇)-alkyl[(C₁-C₇)-alkyl]amino-(C₁-C₇)-alkylaryl, (C₁-C₇)-alkoxycarbonyl-(C₁-C₇)-alkylamino-(C₁-C₇)-alkylaryl, (C₁-C₇)-alkoxycarbonyl-(C₁-C₇)-alkyl[(C₁-C₇)-alkyl]amino-(C₁-C₇)-alkylaryl, heteroaryl[(C₁-C₇)-alkyl]amino-(C₁-C₇)-alkylaryl, heteroaryl-(C₁-C₇)-alkyl[(C₁-C₇)-alkyl]amino-(C₁-C₇)-alkylaryl, (C₃-C₇)-cycloalkyl[(C₁-C₇)-alkyl]amino-(C₁-C₇)-alkylaryl, (C₃-C₇)-cycloalkylamino-(C₁-C₇)-alkylaryl, (C₁-C₇)-alkoxy[(C₁-C₇)-alkoxy]-(C₁-C₇)-alkylaryl, (C₂-C₇)-alkynyl, (C₁-C₇)-alkyl-(C₂-C₇)-alkynyl, (C₁-C₇)-haloalkyl-(C₂-C₇)-alkynyl, (C₁-C₇)-alkylamino-(C₁-C₇)-alkylaryl, (C₁-C₇)-alkylamino-(C₁-C₇)-alkylaryl-(C₁-C₇)-alkyl, bis-[(C₁-C₇)-alkyl]amino-(C₁-C₇)-alkylaryl, bis-[(C₁-C₇)-alkyl]amino-(C₁-C₇)-alkylaryl-(C₁-C₇)-alkyl, heterocyclyl-N—(C₁-C₇)-alkylaryl-(C₁-C₇)-alkyl, (C₁-C₇)-alkoxycarbonyl-N-heterocyclyl-N—(C₁-C₇)-alkyl, aryl-(C₁-C₇)-alkoxycarbonyl-N-heterocyclyl-N—(C₁-C₇)-alkyl, (C₁-C₇)-alkyl-N-heterocyclyl-N—(C₁-C₇)-alkyl, (C₁-C₇)-alkylsulfonyl-N-heterocyclyl-N—(C₁-C₇)-alkyl, arylsulfonyl-N-heterocyclyl-N—(C₁-C₇)-alkyl, heteroarylsulfonyl-N-heterocyclyl-N—(C₁-C₇)-alkyl, (C₃-C₇)-cycloalkylsulfonyl-N-heterocyclyl-N—(C₁-C₇)-alkyl, (C₁-C₇)-haloalkylsulfonyl-N-heterocyclyl-N—(C₁-C₇)-alkyl, alkylcarbonyl-N-heterocyclyl-N-alkyl, arylcarbonyl-N-heterocyclyl-N—(C₁-C₇)-alkyl, heteroarylcarbonyl-N-heterocyclyl-N—(C₁-C₇)-alkyl, (C₃-C₇)-cycloalkylcarbonyl-N-heterocyclyl-N—(C₁-C₇)-alkyl, (C₃-C₇)-cycloalkyl-N-heterocyclyl-N—(C₁-C₇)-alkyl, (C₁-C₇)-alkoxycarbonyl-N-heterocyclyl-(C₁-C₇)-alkyl, aryl-(C₁-C₇)-alkoxycarbonyl-N-heterocyclyl-(C₁-C₇)-alkyl, (C₁-C₇)-alkyl-N-heterocyclyl-(C₁-C₇)-alkyl, (C₁-C₇)-alkylsulfonyl-N-heterocyclyl-(C₁-C₇)-alkyl, arylsulfonyl-N-heterocyclyl-(C₁-C₇)-alkyl, heteroarylsulfonyl-N-heterocyclyl-(C₁-C₇)-alkyl, (C₃-C₇)-cycloalkylsulfonyl-N-heterocyclyl-(C₁-C₇)-alkyl, (C₁-C₇)-haloalkylsulfonyl-N-heterocyclyl-(C₁-C₇)-alkyl, (C₁-C₇)-alkylcarbonyl-N-heterocyclyl-(C₁-C₇)-alkyl, arylcarbonyl-N-heterocyclyl-(C₁-C₇)-alkyl, heteroarylcarbonyl-N-heterocyclyl-(C₁-C₇)-alkyl, (C₃-C₇)-cycloalkylcarbonyl-N-heterocyclyl-(C₁-C₇)-alkyl, (C₃-C₇)-cycloalkyl-N-heterocyclyl-(C₁-C₇)-alkyl, aryl-(C₁-C₇)-alkylaminocarbonylaryl, aminocarbonylaryl, (C₁-C₇)-alkylaminocarbonylaryl, (C₃-C₇)-cycloalkylaminocarbonylaryl, bis-[(C₁-C₇)-alkyl]aminocarbonylaryl, aryl-(C₁-C₇)-alkylaminocarbonylaryl-(C₁-C₇)-alkyl, aminocarbonylaryl-(C₁-C₇)-alkyl, (C₁-C₇)-alkylaminocarbonylaryl-(C₁-C₇)-alkyl, (C₃-C₇)-cycloalkylaminocarbonylaryl-(C₁-C₇)-alkyl, bis-[(C₁-C₇)-alkyl]aminocarbonylaryl-(C₁-C₇)-alkyl, aryl-(C₁-C₇)-alkylamino-(C₁-C₇)-alkyl, heteroaryl-(C₁-C₇)-alkylamino-(C₁-C₇)-alkyl, (C₁-C₇)-alkylamino-(C₁-C₇)-alkoxyaryl, bis-[(C₁-C₇)-alkyl]amino-(C₁-C₇)-alkoxyaryl,
R¹³ represents hydrogen, (C₁-C₇)-alkyl, (C₃-C₇)-cycloalkyl, (C₃-C₇)-cycloalkyl-(C₁-C₇)-alkyl, (C₄-C₇)-cycloalkenyl, cyano-(C₁-C₇)-alkyl, (C₂-C₇)-alkenyl-(C₁-C₇)-alkyl, (C₁-C₇)-haloalkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, aryl, (C₁-C₇)-alkylcarbonyl, (C₃-C₇)-cycloalkylcarbonyl, (C₃-C₇)-cycloalkyl-(C₁-C₇)-alkylcarbonyl, arylcarbonyl, aryl-(C₁-C₇)-alkylcarbonyl, heteroarylcarbonyl, (C₁-C₇)-alkoxycarbonyl, (C₁-C₇)-alkylsulfonyl, (C₃-C₇)-cycloalkylsulfonyl, arylsulfonyl, heteroarylsulfonyl, (C₁-C₇)-alkoxycarbonylcarbonyl, aryl-(C₁-C₇)-alkoxycarbonylcarbonyl, (C₁-C₇)-alkylaminothiocarbonyl, (C₁-C₇)-alkylaminocarbonyl, (C₃-C₇)-cycloalkylaminocarbonyl, bis-[(C₁-C₇)-alkyl]aminocarbonyl, (C₁-C₇)-alkoxy-(C₁-C₇)-alkyl, (C₁-C₇)-alkoxycarbonyl-(C₁-C₇)-alkyl,
R¹⁴, R¹⁵, R¹⁶ independently of one another represent hydrogen, (C₁-C₇)-alkyl, (C₃-C₇)-cycloalkyl, (C₃-C₇)-cycloalkyl-(C₁-C₇)-alkyl, (C₁-C₇)-haloalkyl, aryl, (C₁-C₇)-alkoxycarbonyl, (C₁-C₇)-hydroxycarbonyl, aminocarbonyl, (C₁-C₇)-alkylaminocarbonyl, (C₃-C₇)-cycloalkylaminocarbonyl,
R¹⁷ represents hydrogen, (C₁-C₇)-alkyl, (C₃-C₇)-cycloalkyl, (C₁-C₇)-haloalkyl, aryl-(C₁-C₇)-alkyl, heteroaryl-(C₁-C₇)-alkyl, heterocyclyl-(C₁-C₇)-alkyl, aryl, heteroaryl, heterocyclyl, formyl, imino, hydroxyimino, (C₁-C₇)-alkoxyimino, (C₃-C₇)-cycloalkoxyimino, halogen, (C₁-C₇)-alkoxycarbonyl, hydroxy-(C₁-C₇)-alkyl, (C₁-C₇)-alkoxy-(C₁-C₇)-alkyl, (C₁-C₇)-alkylcarbonyloxy-(C₁-C₇)-alkyl.

4. A treatment for plants, comprising applying a nontoxic amount, effective for increasing resistance of plants to abiotic stress factors, of one or more compound as claimed in claim 1.

5. The treatment as claimed in claim 4, wherein the abiotic stress conditions are one or more conditions selected from the group consisting of aridity, cold stress, heat stress, drought stress, osmotic stress, waterlogging, elevated soil salinity, elevated exposure to minerals, ozone conditions, strong light conditions, limited availability of nitrogen nutrients and limited availability of phosphorus nutrients.

6. The compound as claimed in claim 1 capable of being used in spray application to one or more plants and/or one or more plant parts in combination with one or more active compounds selected from the group consisting of insecticides, attractants, acaricides, fungicides, nematicides, herbicides, growth regulators, safeners, substances which influence plant maturity and bactericides.

7. The compound as claimed in claim 1 capable of being used in spray application to one or more plants and/or one or more parts of plants in combination with one or more fertilizers.

8. The compound as claimed in claim 1 capable of being used for application to one or more genetically modified cultivars, seed thereof, and/or to a cultivated area on which these cultivar grows.

9. A spray solution comprising one or more compounds as claimed in claim 1 capable of being used for enhancing resistance of one or more plants to an abiotic stress factor.

10. A method for increasing stress tolerance in one or more plants selected from the group consisting of useful plants, ornamental plants, turfgrasses and trees, comprising applying a sufficient nontoxic amount of one or more compounds as claimed in claim 1 to the to an area where a corresponding effect is desired, further optionally comprising applying to one or more plants, seed thereof and/or to an area on which a plant grows.

11. The method as claimed in claim 10, wherein resistance of a plant thus treated to abiotic stress is increased by at least 3% compared to an untreated plant under otherwise identical physiological condition.

12. A haloalkyl-substituted benzodiazepinone of formula (Ia) and/or a salt thereof 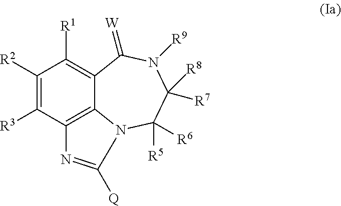
in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, aryl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroraryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris-[(C₁-C₈)-alkyl]silyl, bis-[(C₁-C₈)-alkyl]arylsilyl, bis-[(C₁-C₈)-alkyl]-(C₁-C₈)-alkylsilyl, tris-[(C₁-C₈)-alkyl]silylalkynyl, aryl-(C₂-C₈)-alkynyl, heteroaryl-(C₂-C₈)-alkynyl, (C₁-C₈)-alkyl-(C₁-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis-[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heteroaryl-(C₁-C₈)-alkoxy, aryl-(C₁-C₈)-alkoxy, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino, (C₁-C₈)-halocycloalkyl-(C₂-C₈)-alkynyl,
W represents oxygen, sulfur,
R⁵, R⁶ independently of one another represent hydrogen, (C₁-C₈)-alkyl, (C₁-C₈)-haloalkyl, (C₃-C₈)-cycloalkyl, aryl, (C₂-C₈)-alkenyl, (C₂-C₈)-alkynyl, heterocyclyl, heteroaryl, (C₁-C₈)-alkoxy-(C₁-C₈)-alkyl or together form an exo methylene group which is optionally substituted further and part of a further ring,
R⁷, R⁸ independently of one another represent hydrogen, (C₁-C₈)-alkyl, (C₁-C₈)-haloalkyl, (C₃-C₈)-cycloalkyl, aryl, (C₂-C₈)-alkenyl, (C₁-C₈)-alkynyl, heterocyclyl, heteroaryl, (C₁-C₈)-alkoxy-(C₁-C₈)-alkyl,
R⁹ represents hydrogen, hydroxy, (C₁-C₈)-alkyl, (C₃-C₈)-cycloalkyl, halogen, (C₂-C₈)-alkenyl-(C₁-C₈)-alkyl, (C₂-C₈)-alkynyl-(C₁-C₈)-alkyl, (C₁-C₈)-haloalkyl, (C₁-C₈)-alkoxy-(C₁-C₈)-alkyl, (C₂-C₈)-alkynyl, (C₂-C₈)-alkenyl, (C₃-C₈)-cycloalkyl-(C₁-C₈)-alkyl, cyano-(C₁-C₈)-alkyl, nitro-(C₁-C₈)-alkyl, aryl-(C₁-C₈)-alkyl, heteroaryl-(C₁-C₈)-alkyl, heterocyclyl-(C₁-C₈)-alkyl, aryl, (C₁-C₈)-alkylamino, (C₁-C₈)-alkylamino-(C₁-C₈)-alkyl, bis-[(C₁-C₈)-alkyl]amino-(C₁-C₈)-alkyl, aminocarbonyl-(C₁-C₈)-alkyl, (C₁-C₈)-alkylaminocarbonyl-(C₁-C₈)-alkyl, bis-[(C₁-C₈)-alkyl]aminocarbonyl-(C₁-C₈)-alkyl, (C₁-C₈)-alkoxycarbonyl-(C₁-C₈)-alkyl, hydroxycarbonyl-(C₁-C₈)-alkyl, (C₁-C₈)-alkylcarbonyl, (C₃-C₈)-cycloalkylcarbonyl, (C₁-C₈)-haloalkylcarbonyl, (C₁-C₈)-alkoxycarbonyl, (C₂-C₈)-alkenyloxycarbonyl, (C₁-C₈)-alkylaminocarbonyl, (C₃-C₈)-cyclopropylaminocarbonyl, (C₁-C₈)-alkylsulfonyl, arylsulfonyl, (C₁-C₈)-cycloalkylsulfonyl, aryl-(C₁-C₈)-alkylsulfonyl, (C₂-C₈)-alkenylsulfonyl, heteroarylsulfonyl, (C₂-C₈)-alkynylsulfonyl, (C₁-C₈)-alkylsulfinyl, arylsulfinyl, (C₃-C₈)-cycloalkylsulfinyl, (C₂-C₈)-alkenylsulfinyl, (C₂-C₈)-alkynylsulfinyl, arylsulfinyl, heteroarylsulfinyl, arylcarbonyl, heteroarylcarbonyl, (C₁-C₈)-alkoxycarbonyl-(C₁-C₈)-alkyl, (C₂-C₈)-alkenyloxycarbonyl-(C₁-C₈)-alkyl, hydroxycarbonyl-(C₁-C₈)-alkyl, cyano-(C₁-C₈)-alkylaminocarbonyl, (C₂-C₈)-alkynylaminocarbonyl, heterocyclylcarbonyl, heteroaryl-(C₁-C₈)-alkylaminocarbonyl, (C₂-C₈)-alkenyloxycarbonyl, (C₃-C₈)-cycloalkyl-(C₁-C₈)-alkoxycarbonyl, (C₃-C₈)-cycloalkyl-(C₁-C₈)-alkoxycarbonylcarbonyl, (C₁-C₈)-alkoxycarbonylcarbonyl, (C₃-C₈)-cycloalkyl-(C₁-C₈)-alkylaminocarbonyl, aryl-(C₁-C₈)-alkylaminocarbonyl or a negative charge,
Q represents (C₁-C₁₂)-haloalkyl, (C₃-C₈)-halocycloalkyl, (C₁-C₈)-alkoxy-(C₁-C₈)-haloalkyl, (C₁-C₈)-haloalkoxy-(C₁-C₈)-haloalkyl, (C₁-C₈)-alkylthio-(C₁-C₈)-haloalkyl, (C₁-C₈)-haloalkylthio-(C₁-C₈)-haloalkyl, (C₁-C₈)-alkoxy-(C₁-C₈)-alkoxy-(C₁-C₈)-haloalkyl, bis-[(C₁-C₈)-alkyl]amino-(C₁-C₈)-alkoxy-(C₁-C₈)-haloalkyl,
except for 2-(trifluoromethyl)-5,6-dihydroimidazo[4,5,1-jk][1,4]benzodiazepin-7(4H)-one and 2-(chloromethyl)-5,6-dihydroimidazo[4,5,1-jk][1,4]benzodiazepin-7(4H)-one.

13. The haloalkyl-substituted benzodiazepinone and/or salt as claimed in claim 12 where
R¹, R², R³ independently of one another represent hydrogen, fluorine, chlorine, bromine, iodine, (C₁-C₆)-alkyl, (C₃-C₆)-cycloalkyl, (C₄-C₆)-cycloalkenyl, (C₃-C₆)-halocycloalkyl, (C₂-C₆)-alkenyl, (C₂-C₆)-alkynyl, aryl, aryl-(C₁-C₆)-alkyl, aryl-(C₂-C₆)-alkenyl, heteroaryl, heteroaryl-(C₁-C₆)-alkyl, heterocyclyl, heterocyclyl-(C₁-C₆)-alkyl, (C₁-C₆)-alkoxy-(C₁-C₆)-alkyl, (C₁-C₆)-alkylthio, (C₁-C₆)-haloalkylthio, (C₁-C₆)-haloalkyl, (C₁-C₆)-alkoxy, (C₁-C₆)-haloalkoxy, (C₃-C₆)-cycloalkoxy, (C₃-C₆)-cycloalkyl-(C₁-C₆)-alkoxy, aryloxy, heteroraryloxy, (C₁-C₆)-alkoxy-(C₁-C₆)-alkoxy, (C₂-C₆)-alkynyl-(C₁-C₆)-alkoxy, (C₂-C₆)-alkenyloxy, bis[(C₁-C₆)-alkyl]amino-(C₁-C₆)-alkoxy, tris-[(C₁-C₆)-alkyl]silyl, bis-[(C₁-C₆)-alkyl]arylsilyl, bis-[(C₁-C₆)-alkyl]-(C₁-C₆)-alkylsilyl, tris-[(C₁-C₆)-alkyl]silylalkynyl, aryl-(C₂-C₆)-alkynyl, heteroaryl-(C₂-C₆)-alkynyl, (C₁-C₆)-alkyl-(C₁-C₆)-alkynyl, (C₃-C₆)-cycloalkyl-(C₂-C₆)-alkynyl, (C₁-C₆)-haloalkyl-(C₂-C₆)-alkynyl, heterocyclyl-N—(C₁-C₆)-alkoxy, nitro, cyano, amino, (C₁-C₆)-alkylamino, bis-[(C₁-C₆)-alkyl]amino, (C₁-C₆)-alkylcarbonylamino, (C₃-C₆)-cycloalkylcarbonylamino, arylcarbonylamino, (C₁-C₆)-alkoxycarbonylamino, heteroaryl-(C₁-C₆)-alkoxy, aryl-(C₁-C₆)-alkoxy, heterocyclyl-(C₁-C₆)-alkoxy, (C₃-C₆)-cycloalkyl-(C₁-C₆)-alkyl, (C₂-C₆)-haloalkenyl, (C₂-C₆)-haloalkynyl, heterocyclyl-(C₂-C₆)-alkynyl, (C₃-C₆)-halocycloalkoxy, (C₂-C₆)-haloalkynyloxy, arylthio, heteroarylthio, (C₁-C₆)-alkylsulfinyl, (C₁-C₆)-haloalkylsulfinyl, arylsulfinyl, heteroarylsulfinyl, (C₁-C₆)-alkylsulfonyl, (C₁-C₆)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₆)-alkyl]amino, (C₂-C₆)-alkenylamino, (C₁-C₆)-halocycloalkyl-(C₂-C₆)-alkynyl,
W represents oxygen, sulfur,
R⁵, R⁶ independently of one another represent hydrogen, (C₁-C₆)-alkyl, (C₁-C₆)-haloalkyl, (C₃-C₆)-cycloalkyl, aryl, (C₂-C₆)-alkenyl, (C₂-C₆)-alkynyl, heterocyclyl, heteroaryl, (C₁-C₆)-alkoxy-(C₁-C₆)-alkyl or together form an exo methylene group which is optionally substituted further and part of a further ring,
R⁷, R⁸ independently of one another represent hydrogen, (C₁-C₆)-alkyl, (C₁-C₆)-haloalkyl, (C₃-C₆)-cycloalkyl, aryl, (C₂-C₆)-alkenyl, (C₁-C₆)-alkynyl, heterocyclyl, heteroaryl, (C₁-C₆)-alkoxy-(C₁-C₆)-alkyl,
R⁹ represents hydrogen, hydroxy, (C₁-C₆)-alkyl, (C₃-C₆)-cycloalkyl, halogen, (C₂-C₆)-alkenyl-(C₁-C₆)-alkyl, (C₂-C₆)-alkynyl-(C₁-C₆)-alkyl, (C₁-C₆)-haloalkyl, (C₁-C₆)-alkoxy-(C₁-C₆)-alkyl, (C₂-C₆)-alkynyl, (C₂-C₆)-alkenyl, (C₃-C₆)-cycloalkyl-(C₁-C₆)-alkyl, cyano-(C₁-C₆)-alkyl, nitro-(C₁-C₆)-alkyl, aryl-(C₁-C₆)-alkyl, heteroaryl-(C₁-C₆)-alkyl, heterocyclyl-(C₁-C₆)-alkyl, aryl, (C₁-C₆)-alkylamino, (C₁-C₆)-alkylamino-(C₁-C₆)-alkyl, bis-[(C₁-C₆)-alkyl]amino-(C₁-C₆)-alkyl, aminocarbonyl-(C₁-C₆)-alkyl, (C₁-C₆)-alkylaminocarbonyl-(C₁-C₆)-alkyl, bis-[(C₁-C₆)-alkyl]aminocarbonyl-(C₁-C₆)-alkyl, (C₁-C₆)-alkoxycarbonyl-(C₁-C₆)-alkyl, hydroxycarbonyl-(C₁-C₆)-alkyl, (C₁-C₆)-alkylcarbonyl, (C₃-C₆)-cycloalkylcarbonyl, (C₁-C₆)-haloalkylcarbonyl, (C₁-C₆)-alkoxycarbonyl, (C₂-C₆)-alkenyloxycarbonyl, (C₁-C₆)-alkylaminocarbonyl, (C₃-C₆)-cyclopropylaminocarbonyl, (C₁-C₆)-alkylsulfonyl, arylsulfonyl, (C₁-C₆)-cycloalkylsulfonyl, aryl-(C₁-C₆)-alkylsulfonyl, (C₂-C₆)-alkenylsulfonyl, heteroarylsulfonyl, (C₂-C₆)-alkynylsulfonyl, (C₁-C₆)-alkylsulfinyl, arylsulfinyl, (C₃-C₆)-cycloalkylsulfinyl, (C₂-C₆)-alkenylsulfinyl, (C₂-C₆)-alkynylsulfinyl, arylsulfinyl, heteroarylsulfinyl, arylcarbonyl, heteroarylcarbonyl, (C₁-C₆)-alkoxycarbonyl-(C₁-C₆)-alkyl, (C₂-C₆)-alkenyloxycarbonyl-(C₁-C₆)-alkyl, hydroxycarbonyl-(C₁-C₆)-alkyl, cyano-(C₁-C₆)-alkylaminocarbonyl, (C₂-C₆)-alkynylaminocarbonyl, heterocyclylcarbonyl, heteroaryl-(C₁-C₆)-alkylaminocarbonyl, (C₂-C₆)-alkenyloxycarbonyl, (C₃-C₆)-cycloalkyl-(C₁-C₆)-alkoxycarbonyl, (C₃-C₆)-cycloalkyl-(C₁-C₆)-alkoxycarbonylcarbonyl, (C₁-C₆)-alkoxycarbonylcarbonyl, (C₃-C₆)-cycloalkyl-(C₁-C₆)-alkylaminocarbonyl, aryl-(C₁-C₆)-alkylaminocarbonyl or a negative charge,
Q represents (C₁-C₁₂)-haloalkyl, (C₃-C₆)-halocycloalkyl, (C₁-C₆)-alkoxy-(C₁-C₆)-haloalkyl, (C₁-C₆)-haloalkoxy-(C₁-C₆)-haloalkyl, (C₁-C₆)-alkylthio-(C₁-C₆)-haloalkyl, (C₁-C₆)-haloalkylthio-(C₁-C₆)-haloalkyl, (C₁-C₆)-alkoxy-(C₁-C₆)-alkoxy-(C₁-C₆)-haloalkyl, bis-[(C₁-C₆)-alkyl]amino-(C₁-C₆)-alkoxy-(C₁-C₆)-haloalkyl,
except for 2-(trifluoromethyl)-5,6-dihydroimidazo[4,5,1-jk][1,4]benzodiazepin-7(4H)-one and 2-(chloromethyl)-5,6-dihydroimidazo[4,5,1-jk][1,4]benzodiazepin-7(4H)-one.

14. A spray solution for treatment of plants, comprising an amount, effective for enhancing resistance of one or more plants to an abiotic stress factor, of at least one haloalkyl-substituted benzodiazepinone and/or salt as claimed in claim 12.
